(19) United States
(12) Patent Application Publication
(10) Pub. No.: US 2009/0142377 A1
(43) Pub. Date: Jun. 4, 2009

(54) Immunogenic compositions

(75) Inventors: Martijn Frans Ben Gerard Gebbink, Eemnes (NL); Barend Bouma, Houten (NL); Paulus Johannes Gerardus Maria Steverink, Huizen (NL); Johan Renes, Amersfoort (NL)

Correspondence Address: TRASKBRITT, P.C., P.O. BOX 2550, SALT LAKE CITY, UT, 84110, US

(73) Assignee: Crossbeta Biosciences B.V., Utrecht (NL)

(21) Appl. No.: 12/291,369

(22) Filed: Nov. 7, 2008

(30) Foreign Application Priority Data
Nov. 8, 2007 (EP) 07120303.8

(51) Int. Cl. A61K 9/00; A61K 39/385; G01N 33/53; A61P 37/02; A61K 39/395

(52) U.S. Cl. 424/400; 424/193.1; 435/7.1; 424/130.1

(57) ABSTRACT

Described are means and methods for producing and/or selecting immunogenic compositions, comprising providing the composition with at least one cross-beta structure and testing at least one immunogenic property.

### CROSS-REFERENCE TO RELATED APPLICATION

This application claims priority to European Patent Application Serial No. EP 07120303.8, filed Nov. 8, 2007, the entire contents of which is hereby incorporated herein by this reference.

### TECHNICAL FIELD

The invention relates to the fields of cell biology, immunology, vaccinology, adjuvant technology, and medicine.

### BACKGROUND

Vaccines can be divided in two basic groups, i.e., prophylactic vaccines and therapeutic vaccines. Prophylactic vaccines have been made and/or suggested against essentially every known infectious agent (virus, bacterium, yeast, fungi, parasite, mycoplasm, etc.), which has some pathology in man, pets and/or livestock, which is therefore collectively referred to as pathogen. Therapeutic vaccines have been made and/or suggested for infectious agents as well, but also for treatments of cancer and other aberrancies, as well as for inducing immune responses against other self antigens, as widely ranging as, e.g., LHRH for immunocastration of boars, or for use in preventing graft versus host (GvH) and/or transplant rejections.

In vaccines in general there are two vital issues. Vaccines have to be efficacious and vaccines have to be safe. It often seems that the two requirements are mutually exclusive when trying to develop a vaccine. The most efficacious vaccines so far have been modified live infectious agents. These are modified in a manner that their virulence has been reduced (attenuation) to an acceptable level. The vaccine strain of the infectious agent typically does replicate in the host, but at a reduced level, so that the host can mount an adequate immune response, also providing the host with long term immunity against the infectious agent. The downside of attenuated vaccines is that the infectious agents may revert to a more virulent (and thus pathogenic) form.

This may happen in any infectious agent, but is a very serious problem in fast mutating viruses (such as in particular RNA viruses). Another problem with modified live vaccines is that infectious agents often have many different serotypes. It has proven to be difficult in many cases to provide vaccines which elicit an immune response in a host that protects against different serotypes of infectious agents.

Vaccines in which the infectious agent has been killed are often safe, but often their efficacy is mediocre at best, even when the vaccine contains an adjuvant. In general an immune response is enhanced by adding adjuvants (from the Latin adjuvare, meaning “to help”) to the vaccines. The chemical nature of adjuvants, their proposed mode of action and their reactions (side effect) are highly variable. Some of the side effects can be ascribed to an unintentional stimulation of different mechanisms of the immune system whereas others reflect general adverse pharmacological reactions which are more or less expected. There are several types of adjuvants. Today the most common adjuvants for human use are aluminium hydroxide, aluminium phosphate and calcium phosphate. However, there is a number of other adjuvants based on oil emulsions, products from bacteria (their synthetic derivatives as well as liposomes) or gram-negative bacteria, endotoxins, cholesterol, fatty acids, aliphatic amines, paraffinic and vegetable oils. Recently, monophosphoryl lipid A, ISCOMs with Quil-A, and Syntex adjuvant formulations (SAFs) containing the threonyl derivative or muramyl dipeptide have been under consideration for use in human vaccines. Chemically, the adjuvants are a highly heterogenous group of compounds with only one thing in common: their ability to enhance the immune response—their adjuvanticity. They are highly variable in terms of how they affect the immune system and how serious their adverse effects are due to the resultant hyperactivation of the immune system. The choice of any of these adjuvants reflects a compromise between a requirement for adjuvanticity and an acceptable low level of adverse reactions. The term adjuvant has been used for any material that can increase the humoral and/or cellular immune response to an antigen. In the conventional vaccines, adjuvants are used to elicit an early, high and long-lasting immune response. The newly developed purified subunit or synthetic vaccines (see below) using biosynthetic, recombinant and other modern technology are poor immunogens and require adjuvants to evoke the immune response. The use of adjuvants enables the use of less antigen to achieve the desired immune response, and this reduces vaccine production costs. With a few exceptions, adjuvants are foreign to the body and cause adverse reactions.

A type of vaccine that has received a lot of attention since the advent of modern biology is the subunit vaccine. In these vaccines only one or a few elements of the infectious agent are used to elicit an immune response. Typically a subunit vaccine comprises one, two or three proteins (glycoproteins) and/or peptides present in proteins or fragments thereof, of an infectious agent (from one or more serotypes) which have been purified from a pathogen or produced by recombinant means and/or synthetic means. Although these vaccines in theory are the most promising safe and efficacious vaccines, in practice efficacy has proved to be a major hurdle.

Molecular biology has provided more alternative methods to arrive at safe and efficacious vaccines that theoretically should also provide cross-protection against different serotypes of infectious agents. Carbohydrate structures derived from infectious agents have been suggested as specific immune response eliciting components of vaccines, as well as lipopolysaccharide structures, and even nucleic acid complexes have been proposed. Although these component vaccines are generally safe, their efficacy and cross-protection over different serotypes has been generally lacking. Combinations of different kinds of components have been suggested (carbohydrates with peptides/proteins and lipopolysaccharide (LPS) with peptides/proteins, optionally with carriers), but so far the safety vs. efficacy issue remains.

Another approach to provide cross-protection is to make hybrid infectious agents which comprise antigenic components from two or more serotypes of an infectious agent. These can be and have been produced by modern molecular biology techniques. They can be produced as modified live vaccines, or as vaccines with inactivated or killed pathogens, but also as subunit vaccines. Cocktail or combination vaccines comprising antigens from completely different infectious agents are also well known. In many countries children are routinely vaccinated with cocktail vaccines against, e.g., diphtheria, whooping cough, tetanus and polio. Recombinant vaccines comprising antigenic elements from different infectious agents have also been suggested. For instance for poultry a vaccine based on a chicken anemia virus has been suggested to be complemented with antigenic elements of Marek disease virus (MDV), but many more combinations have been suggested and produced.

Another important advantage of modern recombinant vaccines is that they have provided the opportunity to produce marker vaccines. Marker vaccines have been provided with an extra element that is not present in wild type infectious agent, or marker vaccines lack an element that is present in wild type infectious agent. The response of a host to both types of marker vaccines can be distinguished (typically by serological diagnosis) from the response against an infection with wild type.

An efficient way of producing immunogenic compositions, or improving the immunogenicity of immunogenic compositions, has been provided in WO 2007/008070. This patent application discloses that the immunogenicity of a composition which comprises amino acid sequences is enhanced by providing the composition with at least one cross-beta structure. A cross-beta structure is a structural element of peptides and proteins, comprising stacked beta sheets, as will be discussed in more detail below. According to WO 2007/008070, the presence of cross-beta structure enhances the immunogenicity of a composition comprising an amino acid sequence. An immunogenic composition is thus prepared by producing a composition which comprises an amino acid sequence, such as a protein containing composition, and administrating (protein comprising) cross-beta structures to the composition. Additionally, or alternatively, cross-beta structure formation in the composition is induced, for instance by changing the pH, salt concentration, reducing agent concentration, temperature, buffer and/or chaotropic agent concentration, and/or combinations of these parameters.

### SUMMARY OF THE INVENTION

Provided are improved means and methods for producing and/or improving immunogenic compositions. Further provided are compositions with enhanced immunogenicity for use as vaccines. Also provided are compositions with enhanced immunogenicity for use to obtain vaccines. Further provided are means and methods for producing compositions with enhanced capability of, at least in part, preventing and/or counteracting a pathology and/or a disorder for use as passive vaccines. Further provided are compositions for use as passive vaccines.

Provided herein are improved methods for providing an immunogenic composition comprising providing an amino acid sequence containing composition with at least one cross-beta structure and subsequently testing at least one, preferably at least two, immunogenic properties of the resulting composition. Thus provided is a way for controlling a process for the production of an immunogenic composition, so that immunogenic compositions with preferred immunogenic properties are produced and/or selected.

Provided is a method wherein an immunogenic composition comprising at least one amino acid sequence such as, but not limited to, a peptide, polypeptide, protein, glycoprotein, protein-DNA complex, protein-membrane complex and/or lipoprotein, and here collectively referred to as “protein,” is provided with at least one cross-beta structure, where after at least one of the following properties is tested:
whether a binding compound capable of specifically binding an epitope of the peptide, polypeptide, protein, glycoprotein, protein-DNA complex, protein-membrane complex and/or lipoprotein is capable of specifically binding the immunogenic composition;
whether the degree of multimerization of the peptide, polypeptide, protein, glycoprotein, protein-DNA complex, protein-membrane complex and/or lipoprotein in the composition allows recognition of an epitope of the peptide, polypeptide, protein, glycoprotein, protein-DNA complex, protein-membrane complex and/or lipoprotein by an animal's immune system;
whether between 4-75% of the peptide, polypeptide, protein, glycoprotein, protein-DNA complex, protein-membrane complex and/or lipoprotein content of the composition is in a conformation comprising cross-beta structures; and/or
whether the at least one cross-beta structure comprises a property allowing recognition of an epitope of the peptide, polypeptide, protein, glycoprotein, protein-DNA complex, protein-membrane complex and/or lipoprotein by an animal's immune system. This is outlined below in more detail.

Cross-beta structures are present in a subset of misfolded proteins such as for instance amyloid. A misfolded protein is defined herein as a protein with a structure other than a native, non-amyloid, non-cross-beta structure. Hence, a misfolded protein is a protein having a non-native three dimensional structure, and/or a cross-beta structure, and/or an amyloid structure.

Misfolded proteins tend to multimerize and can initiate fibrillization. This can result in the formation of amorphous aggregates that can vary greatly in size. In certain cases misfolded proteins are more regular and fibrillar in nature. The term amyloid has initially been introduced to define the fibrils, which are formed from misfolded proteins, and which are found in organs and tissues of patients with the various known misfolding diseases, collectively termed amyloidoses. Commonly, amyloid appears as fibrils with undefined length and with a mean diameter of 10 nm, is deposited extracellularly, stains with the dyes Congo red and Thioflavin T (ThT), shows characteristic green birefringence under polarized light when Congo red is bound, comprises β-sheet secondary structure, and contains the characteristic cross-beta conformation (see below) as determined by X-ray fiber diffraction analysis. However, since it has been determined that protein misfolding is a more general phenomenon and since many characteristics of misfolded proteins are shared with amyloid, the term amyloid has been used in a broader scope. Now, the term amyloid is also used to define intracellular fibrils and fibrils formed in vitro. Also the terms amyloid-like and amylog are used to indicate misfolded proteins with properties shared with amyloids, but that do not fulfill all criteria for amyloid, as listed above.

In conclusion, misfolded proteins are highly heterogeneous in nature, ranging from monomeric misfolded proteins, to small oligomeric species, sometimes referred to as protofibrils, larger aggregates with amorphous appearance, up to large highly ordered fibrils, all of which appearances can share structural features reminiscent to amyloid. As used herein, the term “misfoldome” encompasses any collection of misfolded proteins.

Amyloid and misfolded proteins that do not fulfill all criteria for being identified as amyloid can share structural and functional features with amyloid and/or with other misfolded proteins. These common features are shared among various misfolded proteins, independent of their varying amino acid sequences. Shared structural features include for example the binding to certain dyes, such as Congo red, ThT, Thioflavin S, accompanied by enhanced fluorescence of the dyes, multimerization, and the binding to certain proteins, such as tissue-type plasminogen activator (tPA), the receptor for advanced glycation end-products (RAGE) and chaperones, such as heat shock proteins, like BiP (grp78 or immunoglobulin heavy chain binding protein). Shared functional activities include the activation of tPA and the induction of cellular responses, such as inflammatory responses and an immune response, and induction of cell toxicity.

A unique hallmark of a subset of misfolded proteins such as for instance amyloid is the presence of the cross-beta conformation or a precursor form of the cross-beta conformation.

A cross-beta structure is a secondary structural element in peptides and proteins. A cross-beta structure (also referred to as a “cross-β,” a “cross-beta” or a “cross-β structure”) is defined as a part of a protein or peptide, or a part of an assembly of peptides and/or proteins, which comprises single beta-strands (stage 1) and a(n ordered) group of beta-strands (stage 2), and typically a group of beta-strands, preferably composed of 5-10 beta-strands, arranged in a beta-sheet (stage 3). A cross-beta structure often comprises in particular a group of stacked beta-sheets (stage 4), also referred to as “amyloid.” Typically, in cross-beta structures the stacked beta sheets comprise flat beta sheets in a sense that the screw axis present in beta sheets of native proteins, is partly or completely absent in the beta sheets of stacked beta sheets. A cross-beta structure is formed following formation of a cross-beta structure precursor form upon protein misfolding like for example denaturation, proteolysis or unfolding of proteins. A cross-beta structure precursor is defined as any protein conformation that precedes the formation of any of the aforementioned structural stages of a cross-beta structure. These structural elements present in cross-beta structure (precursor) are typically absent in globular regions of (native parts of) proteins. The presence of cross-beta structure is for example demonstrated with X-ray fiber diffraction or binding of ThT or binding of Congo red, accompanied by enhanced fluorescence of the dyes.

A typical form of a cross-beta structure precursor is a partially or completely misfolded protein. A typical form of a misfolded protein is a partially or completely unfolded protein, a partially refolded protein, a partially or completely aggregated protein, an oligomerized or multimerized protein, or a partially or completely denatured protein. A cross-beta structure or a cross-beta structure precursor can appear as monomeric molecules, dimeric, trimeric, up to oligomeric assemblies of molecules and can appear as multimeric structures and/or assemblies of molecules.

Cross-beta structure (precursor) in any of the aforementioned states can appear in soluble form in aqueous solutions and/or organic solvents and/or any other solutions. Cross-beta structure (precursor) can also be present as solid state material in solutions, like for example as insoluble aggregates, fibrils, particles, like for example as a suspension or separated in a solid cross-beta structure phase and a solvent phase.

Protein misfolding, formation of cross-beta structure precursor, formation of aggregates or multimers and/or cross-beta structure can occur in any composition comprising peptides with a length of at least 2 amino acids, and/or protein(s). The term “peptide” is intended to include oligopeptides as well as polypeptides, and the term “protein” includes proteinaceous molecules including peptides, with and without post-translational modifications such as for instance glycosylation, citrullination, oxidation, acetylation and glycation. It also includes lipoproteins and complexes comprising a proteinaceous part, such as for instance protein-nucleic acid complexes (RNA and/or DNA), membrane-protein complexes, etc. As used herein, the term “protein” also encompasses proteinaceous molecules, peptides, oligopeptides and polypeptides. Hence, the use of “protein” or “protein and/or peptide” in this application have the same meaning.

A typical form of stacked beta-sheets is in a fibril-like structure in which the beta-strands are oriented in either the direction of the fiber axis or perpendicular to the direction of the fiber axis. The direction of the stacking of the beta-sheets in cross-beta structures is perpendicular to the long fiber axis.

A cross-beta structure conformation is a signal that triggers a cascade of events that induces clearance and breakdown of the obsolete protein or peptide. When clearance is inadequate, unwanted proteins and/or peptides aggregate and form toxic structures ranging from soluble oligomers up to precipitating fibrils and amorphous plaques. Such cross-beta structure conformation comprising aggregates underlie various diseases and disorders, such as for instance, Huntington's disease, amyloidosis type disease, atherosclerosis, cardiovascular disease, diabetes, bleeding, thrombosis, cancer, sepsis and other inflammatory diseases, rheumatoid arthritis, transmissible spongiform encephalopathies such as Creutzfeldt-Jakob disease, multiple sclerosis, auto-immune diseases, uveitis, ankylosing spondylitis, diseases associated with loss of memory such as Alzheimer's disease, Parkinson's disease and other neuronal diseases (epilepsy), encephalopathy and systemic amyloidoses.

A cross-beta structure is, for instance, formed during unfolding and refolding of proteins and peptides. Unfolding of peptides and proteins occur regularly within an organism. For instance, peptides and proteins often unfold and refold spontaneously at the end of their life cycle. Moreover, unfolding and/or refolding is induced by environmental factors such as for instance pH, glycation, oxidative stress, citrullination, ischeamia, heat, irradiation, mechanical stress, proteolysis and so on. As used herein, the terms “cross-beta” and “cross-beta structure” also encompasses any cross-beta structure precursor and any misfolded protein, even though a misfolded protein does not necessarily comprise a cross-beta structure. The term “cross-beta binding molecule” or “molecule capable of specifically binding a cross-beta structure” also encompasses a molecule capable of specifically binding any misfolded protein.

The terms unfolding, refolding and misfolding relate to the three-dimensional structure of a protein or peptide. Unfolding means that a protein or peptide loses at least part of its three-dimensional structure. The term refolding relates to the coiling back into some kind of three-dimensional structure. By refolding, a protein or peptide can regain its native configuration, or an incorrect refolding can occur. The term “incorrect refolding” refers to a situation when a three-dimensional structure other than a native configuration is formed. Incorrect refolding is also called misfolding. Unfolding and refolding of proteins and peptides involves the risk of cross-beta structure formation. Formation of cross-beta structures sometimes also occurs directly after protein synthesis, without a correctly folded protein intermediate.

In certain embodiments, a method is provided wherein an immunogenic composition comprising at least one peptide, polypeptide, protein, glycoprotein, protein-DNA complex, protein-membrane complex and/or lipoprotein is provided with at least one cross-beta structure. This is performed in various ways. For instance, a peptide, polypeptide, protein, glycoprotein, protein-DNA complex, protein-membrane complex and/or lipoprotein is subjected to a cross-beta inducing procedure, preferably a change of pH, salt concentration, temperature, buffer, reducing agent concentration and/or chaotropic agent concentration. These procedures are known to induce and/or enhance cross-beta formation. In certain embodiments, the peptide, polypeptide, protein, glycoprotein, protein-DNA complex, protein-membrane complex and/or lipoprotein is subjected to a cross-beta inducing procedure before it is used for the preparation of an immunogenic composition. It is, however, also possible to subject the peptide, polypeptide, protein, glycoprotein, protein-DNA complex, protein-membrane complex and/or lipoprotein to a cross-beta inducing procedure while it is already present in an immunogenic composition.

Additionally, or alternatively, a peptide, polypeptide, protein, glycoprotein, protein-DNA complex, protein-membrane complex and/or lipoprotein is provided with a (peptide or protein comprising a) cross-beta structure, either before it is used for the preparation of an immunogenic composition or after it has been used for the preparation of an immunogenic composition.

After an immunogenic composition according to the invention has been provided with cross-beta structures, one or more immunogenic properties of the resulting composition are tested.

In certain embodiments, it is tested whether a binding compound capable of specifically binding an epitope of the peptide, polypeptide, protein, glycoprotein, protein-DNA complex, protein-membrane complex and/or lipoprotein is capable of specifically binding the resulting immunogenic composition. In principle, induction and/or administration of a cross-beta structure into a composition could result in a diminished availability of an epitope of interest. For instance, if a cross-beta structure is induced in a region of a peptide or protein wherein an epitope is present, the epitope is at risk of being shielded. The conformation of the epitope is also at risk of being disturbed. Alternatively, if a peptide sequence of a composition is coupled to a cross-beta containing peptide or protein, the coupling could take place at the site of an epitope of interest, thereby reducing its availability for an animal's immune system. In short, the availability of an epitope of interest for an animal's immune system could be diminished after an immunogenic composition has been provided with cross-beta structures. This is in certain embodiments, tested by determining whether a binding compound which is capable of specifically binding an epitope of interest, such as for instance an antibody or a functional fragment or a functional equivalent thereof, is still capable of binding the immunogenic composition after the composition has been provided with cross-beta structure. If the binding compound is capable of specifically binding the resulting immunogenic composition, it shows that the epitope is still available for an animal's immune system.

In another embodiment it is tested whether the degree of multimerization of the peptide, polypeptide, protein, glycoprotein, protein-DNA complex, protein-membrane complex and/or lipoprotein in the immunogenic composition allows recognition of an epitope of the peptide, polypeptide, protein, glycoprotein, protein-DNA complex, protein-membrane complex and/or lipoprotein by an animal's immune system. Proteins comprising cross-beta structures tend to multimerize. Hence, after an immunogenic composition has been provided with cross-beta structures, multimerization of the peptide, polypeptide, protein, glycoprotein, protein-DNA complex, protein-membrane complex and/or lipoprotein in the immunogenic composition will occur. According to the invention, it is tested whether the degree of multimerization, if occurred at all, is such that an animal's immune system is still capable of recognizing an epitope (of interest). For instance, too much multimerization will result in the formation of a fibril wherein epitopes of interest are shielded from the immune system.

Preferably monomers and/or multimers of the peptide, polypeptide, protein, glycoprotein, protein-DNA complex, protein-membrane complex and/or lipoprotein in the immunogenic composition have dimensions in the range of 0.5 nm to 1000 μm, and more preferably, in the range of 0.5 nm to 100 μm, and even more preferably in the range of 1 nm to 5 μm, and even more preferably in the range of 3-2000 nm. This range of dimensions is determined by the number of proteinaceous molecules per multimer, with a given number of amino acid residues per proteinaceous molecule. Therefore, the dimensions are alternatively or additively expressed in terms of number of proteinaceous molecule monomers per multimer.

In another embodiment, it is tested whether between 4-75% of the peptide, polypeptide, protein, glycoprotein, protein-DNA complex, protein-membrane complex and/or lipoprotein content of the composition is in a conformation comprising cross-beta structures. According to the invention, even though cross-beta structure enhances immunogenicity, the presence of too many cross-beta structures negatively influences immunogenicity. A cross-beta content between (and including) 4 and 75% is preferred. It is possible to determine the ratio between total cross-beta structure and total protein content. In certain embodiments, however, the cross-beta content within single proteins is determined. Preferably, individual proteins have a cross-beta content of between (and including) 4 and 75%, so that at least one epitope remains available for an animal's immune system. Most preferably, at least 70% of the individual proteins each have a cross-beta content of between (and including) 4 and 75%.

In another embodiment, it is tested whether the at least one cross-beta structure comprises a property allowing recognition of an epitope of the peptide, polypeptide, protein, glycoprotein, protein-DNA complex, protein-membrane complex and/or lipoprotein by an animal's immune system. Recognition of a cross-beta structure by a component of an animal's immune system, for instance by a multiligand receptor, results in (the initiation of) an immunogenic reaction against a peptide, polypeptide, protein, glycoprotein, protein-DNA complex, protein-membrane complex and/or lipoprotein of an immunogenic composition according to the invention (see for instance FIG. 20). It is therefore preferably tested whether a cross-beta structure of an immunogenic composition according to the invention has a desired (binding) property.

In certain embodiments, at least two of the above mentioned tests are carried out. Of course, any combination of tests is possible. In certain embodiments, at least three of the above mentioned tests are carried out.

The invention thus provides a method for producing an immunogenic composition, the composition comprising at least one peptide, polypeptide, protein, glycoprotein, protein-DNA complex, protein-membrane complex and/or lipoprotein, the method comprising providing the composition with at least one cross-beta structure and determining: whether a binding compound capable of specifically binding an epitope of the peptide, polypeptide, protein, glycoprotein, protein-DNA complex, protein-membrane complex and/or lipoprotein is capable of specifically binding the immunogenic composition; whether the degree of multimerization of the peptide, polypeptide, protein, glycoprotein, protein-DNA complex, protein-membrane complex and/or lipoprotein in the composition allows recognition of an epitope of the peptide, polypeptide, protein, glycoprotein, protein-DNA complex, protein-membrane complex and/or lipoprotein by an animal's immune system; whether between 4-75% of the peptide, polypeptide, protein, glycoprotein, protein-DNA complex, protein-membrane complex and/or lipoprotein content of the composition is in a conformation comprising cross-beta structures; and/or whether the at least one cross-beta structure comprises a property allowing recognition of an epitope of the peptide, polypeptide, protein, glycoprotein, protein-DNA complex, protein-membrane complex and/or lipoprotein by an animal's immune system.

In certain embodiments, it is determined whether monomers and/or multimers of the peptide, polypeptide, protein, glycoprotein, protein-DNA complex, protein-membrane complex and/or lipoprotein in the immunogenic composition have dimensions in the range of 0.5 nm to 1000 μm, and more preferably, in the range of 0.5 nm to 100 μm, and even more preferably in the range of 1 nm to 5 μm, and even more preferably in the range of 3-2000 nm. This range of dimensions is determined by the number of proteinaceous molecules per multimer, with a given number of amino acid residues per proteinaceous molecule. Therefore, the dimensions are alternatively or additively expressed in terms of number of proteinaceous molecule monomers per multimer.

An animal comprises any animal having an immune system, preferably a mammal. In certain embodiments, the animal comprises a human individual.

A protein-membrane complex is defined as a compound or composition comprising an amino acid sequence as well as a lipid molecule, and/or a fragment thereof, and/or a derivative thereof, for example assembled in a membrane and/or vesicle and/or liposome type of arrangement.

An immunogenic composition comprising at least one peptide, polypeptide, protein, glycoprotein, protein-DNA complex, protein-membrane complex and/or lipoprotein is defined herein as a composition comprising at least one amino acid sequence, which composition is capable of eliciting and/or enhancing an immune response in an animal, preferably a mammal, against at least part of the peptide, polypeptide, protein, glycoprotein, protein-DNA complex, protein-membrane complex and/or lipoprotein after administration of the immunogenic composition to the animal. The immune response preferably comprises a humoral immune response and/or a cellular immune response. The immune response needs not be protective, and/or therapeutic and/or capable of diminishing a consequence of disease. An immunogenic composition according to the invention is preferably capable of inducing and/or enhancing the formation of antibodies, and/or activating B-cells and/or T-cells which are capable of specifically binding an epitope of the peptide, polypeptide, protein, glycoprotein, protein-DNA complex, protein-membrane complex and/or lipoprotein.

In certain embodiments, an antibody, or a functional fragment or a functional equivalent thereof, is used in order to determine whether an epitope of interest is still available for an animal's immune system after an immunogenic composition has been provided with cross-beta structures. Further provided is therefore a method according to the invention, comprising determining whether an antibody or a functional fragment or a functional equivalent thereof, capable of specifically binding an epitope of the peptide, polypeptide, protein, glycoprotein, protein-DNA complex, protein-membrane complex and/or lipoprotein, is capable of specifically binding the immunogenic composition. A functional fragment of an antibody is defined as a fragment which has at least one same property as the antibody in kind, not necessarily in amount. The functional fragment is preferably capable of binding the same antigen as the antibody, albeit not necessarily to the same extent. A functional fragment of an antibody preferably comprises a single domain antibody, a single chain antibody, a Fab fragment or a F(ab′)₂ fragment. A functional equivalent of an antibody is defined as a compound which is capable of specifically binding the same antigen as the antibody. A functional equivalent for instance comprises an antibody which has been altered such that the antigen-binding property of the resulting compound is essentially the same in kind, not necessarily in amount. A functional equivalent is provided in many ways, for instance through conservative amino acid substitution, whereby an amino acid residue is substituted by another residue with generally similar properties (size, hydrophobicity, etc), such that the overall functioning is likely not to be seriously affected.

In another embodiment it is determined whether the immunogenic composition and/or cross-beta structure is capable of specifically binding a cross-beta structure binding compound, preferably at least one compound selected from the group consisting of tPA, BiP, factor XII, hepatocyte growth factor activator, fibronectin, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, preferably cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR-BI), SR-A, chrysamine G, a chaperone, a heat shock protein, HSP70, HSP60, HSP90, gp95, calreticulin, a chaperonin, a chaperokine and a stress protein.

If the immunogenic composition appears to be capable of specifically binding such cross-beta binding compound, it shows that the immunogenic composition comprises a cross-beta structure which is capable of inducing and/or activating an animal's immune system.

Molecular chaperones are a diverse class of proteins comprising heat shock proteins, chaperonins, chaperokines and stress proteins, that are contributing to one of the most important cell defense mechanisms that facilitates protein folding, refolding of partially denatured proteins, protein transport across membranes, cytoskeletal organization, degradation of disabled proteins, and apoptosis, but also act as cytoprotective factors against deleterious environmental stresses. Individual members of the family of these specialized proteins bind non-native states of one or several or whole series or classes of proteins and assist them in reaching a correctly folded and functional conformation. Alternatively, when the native fold cannot be achieved, molecular chaperones contribute to the effective removal of misfolded proteins by directing them to the suitable proteolytic degradation pathways. Chaperones selectively bind to non-natively folded proteins in a stable non-covalent manner. To direct correct folding of a protein from a misfolded form to the required native conformation, mostly several chaperones work together in consecutive steps.

Chaperonins are molecular machines that facilitate protein folding by undergoing energy (ATP)-dependent movements that are coordinated in time and space by complex allosteric regulation. Examples of chaperones that facilitate refolding of proteins from a misfolded conformation to a native form are heat shock protein (hsp) 90, hsp60 and hsp70. Chaperones also participate in the stabilization of unstable protein conformers and in the recovery of proteins from aggregates. Molecular chaperones are mostly heat- or stress-induced proteins (hsps), that perform critical functions in maintaining cell homeostasis, or are transiently present and active in regular protein synthesis. Hsps are among the most abundant intracellular proteins. Chaperones that act in an ATP-independent manner are for example the intracellular small hsps, calreticulin, calnexin and extracellular clusterin. Under stress conditions such as elevated temperature, glucose deprivation and oxidation, small hsps and clusterin efficiently prevent the aggregation of target proteins. Interestingly, both types of hsps can hardly chaperone a misfolded protein to refold back to its native state. In patients with Creutzfeldt-Jakob, Alzheimer's disease and other diseases related to protein misfolding and accumulation of amyloid, increased expression of clusterin and small hsps has been seen. Molecular chaperones are essential components of the quality control machineries present in cells. Due to the fact that they aid in the folding and maintenance of newly translated proteins, as well as in facilitating the degradation of misfolded and destabilized proteins, chaperones are essentially the cellular sensors of protein misfolding and function. Chaperones are therefore the gatekeepers in a first line of defense against deleterious effects of misfolded proteins, by assisting a protein in obtaining its native fold or by directing incorrectly folded proteins to a proteolytic breakdown pathway. Notably, hsps are over-expressed in many human cancers. It has been established that hsps play a role in tumor cell metastasis, proliferation, differentiation, invasion, death, and in triggering the immune system during cancer.

One of the key members of the quality control machinery of the cell is the ubiquitous molecular chaperone hsp90. Hsp90 typically functions as part of large complexes, which include other chaperones and essential cofactors that regulate its function. Different cofactors seem to target hsp90 to different sets of substrates. However, the mechanism of hsp90 function in protein misfolding biology remains poorly understood.

Intracellular pathways that are involved in sensing protein misfolding comprise the unfolded protein response machinery (UPR) in the endoplasmic reticulum (ER). Accumulation of unfolded and/or misfolded proteins in the ER induces ER stress resulting in triggering of the UPR. Environmental factors can transduce the stress response, like for example changes in pH, starvation, reactive oxygen species. During these episodes of cellular stress, intracellular heat shock proteins levels increase to provide cellular protection. Activation of the UPR includes the attenuation of general protein synthesis and the transcriptional activation of the genes encoding ER-resident chaperones and molecules involved in the ER-associated degradation (ERAD) pathway. The UPR reduces ER stress by restoration of the protein-folding capacity of the ER. A key protein acting as a sensor of protein misfolding is the chaperone BiP (also referred to as grp78; Immunoglobulin heavy chain-binding protein/Endoplasmic reticulum luminal Ca²⁺-binding protein).

After testing of at least one immunogenic property of an immunogenic composition according to the invention, an immunogenic composition with a desired property is preferably selected. If a desired property, such as the availability of an epitope of interest, appears not to be present (anymore) after a composition comprising at least one peptide, polypeptide, protein, glycoprotein, protein-DNA complex, protein-membrane complex and/or lipoprotein has been provided with cross-beta structures, another batch of the same kind of composition is preferably provided with cross-beta structures and tested again. If needed, this procedure is repeated until an immunogenic composition with at least one desired property/properties is obtained.

In certain embodiments, an immunogenic composition is selected with a degree of multimerization of the peptide, polypeptide, protein, glycoprotein, protein-DNA complex, protein-membrane complex and/or lipoprotein which allows recognition of an epitope by an animal's immune system. Further provided is therefore a method according to the invention, further comprising selecting an immunogenic composition wherein the degree of multimerization of the peptide, polypeptide, protein, glycoprotein, protein-DNA complex, protein-membrane complex and/or lipoprotein in the composition allows recognition of an epitope of the peptide, polypeptide, protein, glycoprotein, protein-DNA complex, protein-membrane complex and/or lipoprotein by an animal's immune system.

In another embodiment, an immunogenic composition is selected with a cross-beta content of between 4-75% so that the immunogenicity is enhanced, while at least one epitope remains available for an animal's immune system. The term immunogenicity is defined herein as the capability of a compound or a composition to activate an animal's immune system. Of course, if it is intended that an animal's immune system is, at least in part, directed against an epitope of interest, the epitope of interest should be available for the animal's immune system. Further provided is therefore a method according to the invention, further comprising selecting an immunogenic composition wherein between 4-75% of the peptide, polypeptide, protein, glycoprotein, protein-DNA complex, protein-membrane complex and/or lipoprotein content of the composition is in a conformation comprising cross-beta structures.

In yet another embodiment an immunogenic composition is selected which comprises a cross-beta structure having a binding property which allows (the initiation of) an immunogenic reaction against a peptide, polypeptide, protein, glycoprotein, protein-DNA complex, protein-membrane complex and/or lipoprotein of an immunogenic composition according to the invention. Further provided is therefore a method according to the invention, further comprising selecting an immunogenic composition which comprises a cross-beta structure which is capable of specifically binding a cross-beta structure binding compound, preferably tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, preferably cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR-BI), SR-A, chrysamine G, a chaperone, a heat shock protein, HSP70, HSP60, HSP90, gp95, calreticulin, a chaperonin, a chaperokine and/or a stress protein.

In a particularly preferred embodiment, an immunogenic composition is selected which is capable of specifically binding an antibody, or a functional fragment or a functional equivalent thereof, which is capable of specifically binding an epitope of interest. Preferably, an immunogenic composition is selected which is capable of specifically binding an antibody, or a functional fragment or a functional equivalent thereof, which is capable of specifically binding a functional, native epitope which is exposed on an natively folded antigen molecule. Such immunogenic composition comprises an epitope of interest which is available for an animal's immune system after the immunogenic composition has been provided with cross-beta structures.

Further provided is therefore a method according to the invention, further comprising selecting an immunogenic composition which is capable of specifically binding an antibody or a functional fragment or a functional equivalent thereof which is capable of specifically binding an epitope of the peptide, polypeptide, protein, glycoprotein, protein-DNA complex, protein-membrane complex and/or lipoprotein. The epitope of the peptide, polypeptide, protein, glycoprotein, protein-DNA complex, protein-membrane complex and/or lipoprotein is preferably surface-exposed when the peptide, polypeptide, protein, glycoprotein, protein-DNA complex, protein-membrane complex and/or lipoprotein is in its native conformation so that, after administration to a suitable host, an immune response against the native form of the peptide, polypeptide, protein, glycoprotein, protein-DNA complex, protein-membrane complex and/or lipoprotein is elicited.

In certain embodiments, for selection of an immunogenic composition having a greater chance of being capable of eliciting a protective prophylactic immune response and/or a therapeutic immune response in vivo, as compared to other immunogenic compositions of a given plurality of immunogenic compositions, antibodies, or functional fragments or equivalents thereof, are used which are capable of at least in part preventing and/or counteracting a pathology and/or a disorder against which an immunogenic composition is produced. The pathology and/or disorder for example being caused by a pathogen, tumor, cardiovascular disease, atherosclerosis, amyloidosis, autoimmune disease, graft-versus-host rejection and/or transplant rejection in the target species for which an immunogenic composition according to the invention is designed. The target species for example comprises a mammal, preferably a human individual. The antibodies, or the B-cells producing these antibodies, are preferably isolated from individuals who successfully combated and/or counteracted the pathology and/or disorder, for example a viral infection. These antibodies, or the B-cells producing these antibodies, are preferably originating from the target species for which the immunogenic composition is designed. Alternatively, these antibodies are originating from a different animal species, and are for example modified to obtain antibodies more closely resembling antibodies of a target species. A non-limiting example of such modified antibody is a humanized antibody from murine origin, which is particularly suitable when the immunogenic composition is designed for human use. Collectively, these aforementioned antibodies are referred to as functional antibodies (in vivo). Non-limiting examples of these functional antibodies are those described in the art against H5N1 influenza virus, tetanospasmin of Clostridium tetani, rabies, Hepatitis B, Hepatitis A, antisera against snake venoms, for example against the poisonous snake venom proteins, and against toxic poisonous insect proteins. For example, these functional antibodies have proven efficacy when applied in passive immunization strategies.

In an alternative embodiment, antibodies are used for selection of an immunogenic composition that is capable of preventing and/or counteracting, at least in part, a disorder against which an immunogenic composition is sought. The antibodies are preferably selected using a passive immunization approach including actively inflicting the pathology and/or disorder against which protection is sought, termed challenge, after treatment of individuals with the antibodies. This approach is termed Reverse Vaccine Development. For the Reverse Vaccine Development approach, firstly antibodies are selected that have the ability to protect and/or cure an individual from an infection and/or disorder upon application of the antibodies to the individual, and/or antibodies are selected that have the ability to modulate the response of an individual to an infection and/or disorder upon application of the antibodies to the individual. Next, these antibodies are used for selection of an immunogenic composition that comprises at least one epitope for these functional antibodies, combined with immunogenic cross-beta adjuvant, preferably in the context of an optimal multimeric size. The term cross-beta adjuvant refers to an amino-acid sequence with an appearance of a cross-beta conformation which is capable, upon introduction of the cross-beta conformation to an animal, of activating the immune system of the receiving animal. As stated before, a capability of activating the immune system of the receiving animal is referred to as immunogenicity. Preferably, antibodies originating from the target species are used for passive immunization of individuals of the same species. Alternatively, antibodies from a different, preferably closely related species, are used for cross-species passive immunizations. For example, murine antibodies for passive immunization of ferrets in an influenza virus challenge model, or murine antibodies in a CSFV challenge model with pigs. Preferably, individuals of the target species for whom the immunogenic composition is meant, are treated with the antibodies or, alternatively, individuals of other species are treated, for example individuals of closely related species such as for example macaques when the target species are humans. Passive immunizations are preferably performed according to methods known to a person skilled in the art for their efficacy. Passive immunization is for example performed by intravenous administration, and/or by intradermal administration, and/or by intramuscular administration. Antibodies used for passive immunizations are preferably selected based on their known ability to modify the response of in vitro (testing) systems. Non-limiting examples of such in vitro experiments are virus neutralization tests, for example for influenza virus or CSFV, hemagglutination inhibition tests, for example for influenza virus, bactericidal activity test, for example for Neisseria meningitides, antibody dependent cell-mediated cytotoxicty (ADCC) and blood coagulation tests, for example for determination of FVIII inhibitors in Haemophilia patient samples. Collectively, these aforementioned antibodies are referred to as functional antibodies (in vitro). Alternatively, other antibodies without (known) functional activity in in vitro (testing) systems that are capable of binding the antigen of choice for incorporation in an immunogenic composition, are used for the described passive immunization approaches. These antibodies are either originating from the animal species for which the immunogenic composition is meant, or are originating from a different species. Following this (cross-species) passive immunization approach, and a subsequent challenge, functional antibodies (in vivo) are selected from the pool of applied antibodies and used for passive vaccination. These functional antibodies (in vivo) are then preferably subsequently used for selection of immunogenic compositions having a greater chance of being capable of eliciting a protective prophylactic immune response and/or a therapeutic immune response in vivo, as compared to other immunogenic compositions of a given plurality of immunogenic compositions.

In certain embodiments, immunogenic compositions selected based on binding of (one or more) antibodies that are proven to be functional antibodies (in vivo), that is to say, the antibodies have the ability to protect, diminish and/or cure an individual from an infection and/or disorder upon passive vaccination (Step 1), are used as active vaccine (product I). In another embodiment these immunogenic compositions, referred to as product I, are used in an immunization approach followed by actively inflicting the pathology and/or disorder against which protection is sought, i.e., a challenge, after immunization of individuals, or followed by a naturally occurring infection or disorder against which protection is sought after the immunization (Step 2-a). When conducting a challenge approach, the pathogen isolate used for the challenge and the antigen in the immunogenic composition are either homologous, or heterologous. Alternatively, these immunogenic compositions, referred to as product I, are used in an immunization approach with individuals who suffer from an infection or disorder, against which an immunogenic composition is sought that diminishes symptoms related to the infection or disorder, and/or that cures an individual from the infection or disorder (Step 2-b). When the individuals that received the immunogenic composition are protected against the infection or disorder, or have diminished symptoms related to the infection or disorder, and/or are cured from an infection or disorder, reconvalescent serum is collected from the individuals (Step 3). Reconvalescent serum is defined as the serum obtained from an individual recovering and/or recovered from a disorder, disease or infection. This serum is analyzed for the presence of antibodies that bind the native antigen and the antigen used in the immunogenic composition, for example using an ELISA with antigen and antigen in the used immunogenic composition immobilized onto an 96-wells plate, which is then incubated with a dilution series of sera, followed by detecting whether antibodies from the sera bound to the antigen, and to which extent (Step 4). In addition, the binding of antibodies in the sera to antigen is compared to the binding of functional antibodies (in vivo), that were initially used for the passive immunization (Step 5). Comparison is for example done in a competition ELISA. In the competition ELISA, for example a fixed amount of functional antibody (in vivo) that gives sub-maximal binding to the immobilized antigen, is mixed with a concentration series of reconvalescent serum, and the extent of binding of the functional antibodies (in vivo) is assessed. When antibodies are present in the reconvalescent serum that bind to the same or similar epitopes as the functional antibodies (in vivo), decreased binding of functional antibodies (in vivo) will be measured with increasing serum concentration. Steps 1-5 is termed Reverse Vaccine Development. In certain embodiments, the above mentioned reconvalescent serum comprising antibodies that bind to the same and/or similar epitopes that are capable of being bound by functional antibodies (in vivo) is subsequently used for passive immunization, followed by a challenge (Step A). Reconvalescent serum is then preferably selected that is capable of at least in part preventing and/or counteracting a pathology and/or a disorder against which an immunogenic composition is sought (Step B). Preferably, this latter reconvalescent serum has an increased capability of at least in part preventing and/or counteracting a pathology and/or a disorder against which an immunogenic composition is sought when compared to the functional antibodies (in vivo) used initially for passive immunizations. Then, this reconvalescent serum with improved capability of at least in part preventing and/or counteracting a pathology and/or a disorder, termed product II, is preferably used in passive immunization strategies (Step C). The improved reconvalescent serum is in another embodiment further refined towards an even more improved reconvalescent serum in an iterative process, by subjecting this improved reconvalescent serum to the aforementioned Steps 1-5, A, B, and, when an acceptable further improved reconvalescent serum is achieved, as product II in Step C. Thus, in Step 1, the functional antibodies (in vivo) are replaced by the improved reconvalescent serum, followed by Step 2 and further. Functional antibodies (in vivo) are isolated from reconvalescent serum using standard affinity based purification procedures, for example by subjecting the serum to an affinity matrix comprising immobilized native antigen, separating the antibodies that bind to the native antigen from the serum, and collecting the antibodies that bound to the native antigen. These purified functional antibodies (in vivo) can be used as product II in Step C, and/or can be subjected to Steps 1-5, A, C for further improvement of the functional antibodies (in vivo). With the functional antibodies (in vivo), which is polyclonal of nature, in the reconvalescent serum, collectively termed functional antibody passive immunization (FAPI) for, for example use in passive immunization strategies, having similar or improved capacities when compared to the functional antibody and/or antibodies (in vivo) originally used in Step 1, FAPI is preferably used for passive immunization purposes. In this way the Reversed Vaccine Development technology provides for two products; product I, an (optimized) immunogenic composition capable of eliciting a protective prophylactic immune response and/or a therapeutic immune response in vivo, and product II, FAPI vaccine with improved capability of at least in part preventing and/or counteracting a pathology and/or a disorder. FAPI is also preferably used coupled to a preferred antigen in immune complex vaccination. The immune complex vaccine products are therefore also herewith provided.

One embodiment therefore provides a reconvalescent serum and/or an antibody capable of at least in part preventing and/or counteracting a pathology and/or a disorder, obtainable by immunizing an animal with an immunogenic composition according to the invention and, subsequently, harvesting reconvalescent serum and/or an antibody from the animal. The reconvalescent serum and/or antibody is preferably used as a passive vaccine. A use of a reconvalescent serum and/or an (improved) antibody according to the invention as a vaccine, or for the preparation of a vaccine, is therefore also herewith provided.

One embodiment provides an (optimized) immune complex vaccine capable of eliciting a protective prophylactic immune response and/or a therapeutic immune response in vivo, obtainable with a method according to the invention. Another embodiment provides a FAPI vaccine with improved capability of at least in part preventing and/or counteracting a pathology and/or a disorder.

Further provided is a method for obtaining a reconvalescent serum and/or an antibody capable of at least in part preventing and/or counteracting a pathology and/or a disorder, the method comprising: producing and/or selecting an immunogenic composition with a method according to the invention, preferably using an antibody, or a functional part thereof, which is capable of specifically binding an epitope of interest of the peptide, polypeptide, protein, glycoprotein, protein-DNA complex, protein-membrane complex and/or lipoprotein present in the immunogenic composition; immunizing an animal with the immunogenic composition; and harvesting reconvalescent serum and/or an antibody from the animal.

In certain embodiments, the animal comprises a non-human animal. It is, however, also possible to use an immunogenic composition according to the invention for vaccination of human individual; and to obtain serum and/or antibodies from the human individual.

Further provided is a use of an immunogenic composition according to the invention for obtaining functional antibodies and/or reconvalescent serum. As described above, the functional antibodies and/or reconvalescent serum, preferably with a higher affinity for an antigen of interest as compared to antibodies which were originally used for the preparation of the immunogenic composition, are particularly suitable for the preparation of an improved composition meant for passive immunization and/or for preparation of immune complexes. A use of the reconvalescent serum and/or functional antibodies, or a functional fragment or functional equivalent thereof, for the preparation of a composition for passive immunization and/or for preparation of immune complexes is also herewith provided. The composition meant for passive immunization and/or for preparation of immune complexes is preferably a vaccine. The reconvalescent serum and/or functional antibodies, or a functional fragment or functional equivalent thereof, is preferably used for the preparation of a prophylactic and/or therapeutic vaccine for the prophylaxis and/or treatment of a disorder caused by a pathogen, tumor, cardiovascular disease, atherosclerosis, amyloidosis, autoimmune disease, graft-versus-host rejection and/or transplant rejection.

In certain embodiments, functional antibodies are used directly for selection of immunogenic compositions that have a greater chance, as compared to other immunogenic compositions of a given plurality of immunogenic compositions, for at least in part preventing, diminishing and/or counteracting the pathology and/or disorder against which an immunogenic composition is sought, thereby not pre-selecting the functional antibodies (in vitro) in order to obtain functional antibodies (in vivo).

Alternatively, antibodies that are not functional antibodies and for which functional activity in in vitro disorder models is not known, and that are capable of binding an antigen of choice for incorporation in an immunogenic composition, are used for selection of immunogenic compositions having a greater chance of being capable of eliciting a protective prophylactic immune response and/or a therapeutic immune response in vivo, as compared to other immunogenic compositions of a given plurality of immunogenic compositions.

In all of the above approaches, preferably monoclonal antibodies or combinations of antibodies are used. Combinations of antibodies for example comprise combined monoclonal antibodies, and/or sera or plasma, and/or polyclonal antibodies isolated from serum or plasma, preferably sera or plasma from mammals known to have developed an immune response, preferably an effective immune response, i.e., reconvalescent serum/plasma.

In all of the above approaches either antibodies of a single class or compositions of antibodies of plural classes are used. For example, for selection of immunogenic compositions, in certain embodiments, only antibodies of the IgG, or IgA, or IgM, or IgD class are used, or combinations of these classes of antibodies, either separately for each class, or in mixtures of antibodies of combined classes. For example, in certain embodiments, combined IgG and IgM antibodies are used. When IgGs are considered, in certain embodiments, IgGs of a single isotype are used, or IgGs of plural isotypes are used, either separately, or in combined compositions of IgGs. For example, murine IgG₁, or IgG₂ₐ is used separately, or murine immune serum comprising all IgG isotypes is used.

In all of the above descriptions, the term antibody refers to any molecule comprising an affinity region, originating from any species. An affinity region influences the affinity with which a protein or peptide binds to an epitope and is herein defined as at least part of an antibody that is capable of specifically binding to an epitope. The affinity region for instance comprises at least part of an immunoglobulin, at least part of a monoclonal antibody and/or at least part of a humanized antibody. The affinity region preferably comprises at least part of a heavy chain and/or at least part of a light chain of an antibody. In certain embodiments, the affinity region comprises a double F(ab′)₂ or single form Fab fragment. Non-limiting examples of molecules with affinity regions are mouse monoclonal antibodies, human immune serum comprising a collection of immunoglobulins, and llama, camel, alpaca or camelid antibodies, also referred to as nanobodies.

In preferred embodiments, either one kind of monoclonal antibody, or a combination of antibodies, or a series of individual monoclonal antibodies, or a series of combinations of antibodies, or a combined series of individual monoclonal antibodies and combinations of antibodies, is used for selection of immunogenic compositions having a greater chance of being capable of eliciting a protective prophylactic immune response and/or a therapeutic immune response in vivo, as compared to other immunogenic compositions of a given plurality of immunogenic compositions. Preferably, 1 to 15 monoclonal antibodies and/or combinations of antibodies are used for these screenings, and even more preferably, 3 to 10 monoclonal antibodies and/or combinations of antibodies are used for the selections. These multiple antibodies preferably have varying affinity for, for example identical and/or similar and/or overlapping epitopes on the antigen. These multiple antibodies preferably bind to distinct epitopes on the antigen.

A method according to the invention is particularly suitable for selecting, from a plurality of immunogenic compositions, one or more immunogenic compositions having a greater chance of being capable of eliciting a protective prophylactic immune response and/or a therapeutic immune response in vivo, as compared to the other immunogenic compositions of the plurality of immunogenic compositions. One or more immunogenic compositions are selected which appear to have a desired property in any of the aforementioned tests. Further provided is therefore an in vitro method for selecting, from a plurality of immunogenic compositions comprising at least one peptide and/or polypeptide and/or protein and/or glycoprotein and/or lipoprotein and/or protein-DNA complex and/or protein-membrane complex with a cross-beta structure, one or more immunogenic compositions having a greater chance of being capable of eliciting a protective prophylactic immune response and/or a therapeutic immune response in vivo, as compared to the other immunogenic compositions of the plurality of immunogenic compositions, the method comprising: selecting, from the plurality of immunogenic compositions, an immunogenic composition: which is capable of specifically binding an antibody or a functional fragment or a functional equivalent thereof which is capable of specifically binding an epitope of the peptide, polypeptide, protein, glycoprotein, protein-DNA complex, protein-membrane complex and/or lipoprotein; wherein the degree of multimerization of the peptide, polypeptide, protein, glycoprotein, protein-DNA complex, protein-membrane complex and/or lipoprotein in the composition allows recognition of an epitope of the peptide, polypeptide, protein, glycoprotein, protein-DNA complex, protein-membrane complex and/or lipoprotein by an animal's immune system; wherein between 4-75% of the peptide, polypeptide, protein, glycoprotein, protein-DNA complex, protein-membrane complex and/or lipoprotein content of the composition is in a conformation comprising cross-beta structures; and/or which comprises a cross-beta structure which is capable of specifically binding a cross-beta structure binding compound, preferably tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, preferably cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR-BI), SR-A, chrysamine G, a chaperone, a heat shock protein, HSP70, HSP60, HSP90, gp95, calreticulin, a chaperonin, a chaperokine and/or a stress protein.

In a particularly preferred embodiment, a method according to the invention is performed wherein an immunogenic composition is selected which is capable of specifically binding at least two antibodies, or functional fragments or functional equivalents thereof, which are capable of specifically binding at least two different epitopes, and/or which are capable of specifically binding the same epitope although with varying affinities, of the peptide, polypeptide, protein, glycoprotein, protein-DNA complex, protein-membrane complex and/or lipoprotein. Such immunogenic composition comprises at least two different epitopes which are available for an animal's immune system and, therefore, is particularly immunogenic. In a more preferred embodiment an immunogenic composition is selected which is capable of specifically binding at least three antibodies, or functional fragments or functional equivalents thereof, which are capable of specifically binding at least three different epitopes, and/or which are capable of specifically binding the same epitope although with varying affinities, of the peptide, polypeptide, protein, glycoprotein, protein-DNA complex, protein-membrane complex and/or lipoprotein. The at least two or three different epitopes may be partially overlapping.

As already described above, a method according to the invention preferably comprises selecting an immunogenic composition which is capable of specifically binding at least one antibody, or a functional fragment or functional equivalent thereof, which is capable of providing a protective prophylactic and/or a therapeutic immune response in vivo.

A composition comprising at least one peptide, polypeptide, protein, glycoprotein, protein-DNA complex, protein-membrane complex and/or lipoprotein is provided with at least one cross-beta structure in various ways. In certain embodiments, the cross-beta structure is induced in at least part of the peptide, polypeptide, protein, glycoprotein, protein-DNA complex, protein-membrane complex and/or lipoprotein. Various methods for inducing a cross-beta structure are known in the art. For instance, the peptide, polypeptide, protein, glycoprotein, protein-DNA complex, protein-membrane complex and/or lipoprotein is at least in part misfolded. In certain embodiments, an immunogenic composition comprising at least one peptide, polypeptide, protein, glycoprotein, protein-DNA complex, protein-membrane complex and/or lipoprotein is subjected to a cross-beta inducing procedure. The cross-beta inducing procedure preferably comprises a change of pH, salt concentration, temperature, buffer, reducing agent concentration and/or chaotropic agent concentration. A method according to the invention, wherein at least one peptide, polypeptide, protein, glycoprotein, protein-DNA complex, protein-membrane complex and/or lipoprotein is subjected to a cross-beta inducing procedure, preferably a change of pH, salt concentration, reducing agent concentration, temperature, buffer and/or chaotropic agent concentration, is therefore also provided. Non-limiting examples of cross-beta inducing procedures are heating, chemical treatments with, e.g., high salts, acid or alkaline materials, pressure and other physical treatments. A preferred manner of introducing cross-beta structures in an antigen is by one or more treatments, either in combined fashion or sequentially, of heating, freezing, reduction, oxidation, glycation pegylation, sulphatation, exposure to a chaotropic agent (the chaotropic agent preferably being urea or guanidinium-HCl), phosphorylation, partial proteolysis, chemical lysis, preferably with HCl or cyanogenbromide, sonication, dissolving in organic solutions, preferably 1,1,1,3,3,3-hexafluoro-2-propanol and trifluoroacetic acid, or a combination thereof.

In a particularly preferred embodiment, the immunogenic composition comprising at least one peptide, polypeptide, protein, glycoprotein, protein-DNA complex, protein-membrane complex and/or lipoprotein is coupled to a cross-beta comprising compound. For instance, the peptide, polypeptide, protein, glycoprotein, protein-DNA complex, protein-membrane complex and/or lipoprotein is linked to a peptide or protein comprising a cross-beta structure. It is, however, also possible to administer a cross-beta comprising compound to a composition according to the invention, without linking the cross-beta comprising compound to the peptide, polypeptide, protein, glycoprotein, protein-DNA complex, protein-membrane complex and/or lipoprotein. Preferably the cross-beta comprising compound is an otherwise inert compound. Inert is defined as not eliciting an unwanted immune response or another unwanted biochemical reaction in a host, at least not to an unacceptable degree, preferably only to a negligible degree.

A cross-beta structure comprising compound may be added to a composition by itself, but it is also useful to use the cross-beta structure comprising compound as a carrier to which elements of the infectious agent(s) and/or antigen(s) of an immunogenic composition according to the invention are linked. This linkage can be provided through chemical linking (direct or indirect) or, for instance, by expression of the relevant antigen(s) and the cross-beta comprising compound as a fusion protein. In both cases linkers between the two may be present. In both cases dimers, trimers and/or multimers of the antigen (or one or more epitopes of a relevant antigen) may be coupled to a cross-beta comprising compound. However, normal carriers comprising relevant epitopes or antigens coupled to them may also be used. The simple addition of a cross-beta comprising compound will enhance the immunogenicity of such a complex. This is more or less generally true. An immunogenic composition according to the invention may typically comprise a number or all of the normal constituents of an immunogenic composition (in particular a vaccine), supplemented with a cross-beta structure (conformation) comprising compound.

In certain embodiments, the cross-beta structure comprising compound is itself a vaccine component, also referred to in this text as cross-beta antigen (i.e., derived from an infectious agent and/or antigen against which an immune response is desired).

An immunogenic composition according to the invention is preferably used for the preparation of a vaccine. A method according to the invention, further comprising producing a vaccine comprising the selected immunogenic composition, is therefore also herewith provided. Preferably a prophylactic and/or therapeutic vaccine is produced. In certain embodiments, a subunit vaccine is produced.

In certain embodiments, an immunogenic composition which is produced and/or selected with a method according to the invention is used as a vaccine. No other carriers, adjuvants and/or diluents are necessary because of the presence of cross-beta structures. However, if desired, such carriers, adjuvants and/or diluents may be administered to the vaccine composition at will. Further provided is therefore a use of an immunogenic composition produced and/or selected with a method according to the invention as a vaccine, preferably as a prophylactic and/or therapeutic vaccine. In certain embodiments, the vaccine comprises a subunit vaccine.

The invention further provides an immunogenic composition selected and/or produced with a method according to the invention. The immunogenic composition preferably comprises a vaccine, more preferably a prophylactic and/or therapeutic vaccine. An immunogenic composition according to the invention is particularly suitable for the preparation of a vaccine for the prophylaxis and/or treatment of a disorder caused by a pathogen, tumor, cardiovascular disease, atherosclerosis, amyloidosis, autoimmune disease, graft-versus-host rejection and/or transplant rejection. A use of an immunogenic composition according to the invention for the preparation of a vaccine for the prophylaxis and/or treatment of a disorder caused by a pathogen, tumor, cardiovascular disease, atherosclerosis, amyloidosis, autoimmune disease, graft-versus-host rejection and/or transplant rejection is therefore also herewith provided.

Further provided are uses of such immunogenic compositions for at least in part preventing and/or counteracting such disorders. One embodiment provides a method for at least in part preventing and/or counteracting a disorder caused by a pathogen, tumor, cardiovascular disease, atherosclerosis, amyloidosis, autoimmune disease, graft-versus-host rejection and/or transplant rejection, comprising administering to a subject in need thereof a therapeutically effective amount of an immunogenic composition according to the invention. The animal may be a human individual.

A method according to the invention is particularly suitable for producing and/or selecting an immunogenic composition with desired, preferably improved, immunogenic properties. It is, however, also possible to perform a method according to the invention for improving existing immunogenic compositions. Further provided is therefore a method for improving an immunogenic composition, the composition comprising at least one peptide, polypeptide, protein, glycoprotein, protein-DNA complex, protein-membrane complex and/or lipoprotein, the method comprising providing the composition with at least one cross-beta structure and selecting an immunogenic composition: which is capable of specifically binding an antibody or a functional fragment or a functional equivalent thereof which is capable of specifically binding an epitope of the peptide, polypeptide, protein, glycoprotein, protein-DNA complex, protein-membrane complex and/or lipoprotein; wherein the degree of multimerization of the peptide, polypeptide, protein, glycoprotein, protein-DNA complex, protein-membrane complex and/or lipoprotein in the composition allows recognition of an epitope of the peptide, polypeptide, protein, glycoprotein, protein-DNA complex, protein-membrane complex and/or lipoprotein by an animal's immune system; wherein between 4-75% of the peptide, polypeptide, protein, glycoprotein, protein-DNA complex, protein-membrane complex and/or lipoprotein content of the composition is in a conformation comprising cross-beta structures; and/or which is capable of specifically binding a cross-beta structure binding compound, preferably tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, preferably cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR-BI), SR-A, chrysamine G, a chaperone, a heat shock protein, HSP70, HSP60, HSP90, gp95, calreticulin, a chaperonin, a chaperokine and/or a stress protein.

A method according to the invention is particularly suitable for producing and/or selecting an immunogenic composition which is capable of eliciting an immune response in an animal. It is, however, also possible to use the teaching of the invention in order to avoid the use of immunogenic compounds. For instance, if a composition comprising at least one peptide, polypeptide, protein, glycoprotein, protein-DNA complex, protein-membrane complex and/or lipoprotein is used for a non-immunogenic purpose, for instance as a medicament, immunological reactions after administration of the composition to an animal are undesired. In such cases, it is not intended to induce cross-beta structures in the composition comprising at least one peptide, polypeptide, protein, glycoprotein, protein-DNA complex, protein-membrane complex and/or lipoprotein. However, cross-beta structures may form anyway. Therefore, in order to test such compositions for non-immunogenic use, a composition comprising at least one peptide, polypeptide, protein, glycoprotein, protein-DNA complex, protein-membrane complex and/or lipoprotein is subjected to any of the tests described hereinbefore. If a composition appears to have become too immunogenic, it is not used. Instead, another batch of the same kind of composition is preferably tested with a method according to the invention. If needed, this procedure is repeated until a composition with no, or an acceptable, immunogenic property has been obtained. For applications wherein compositions comprising at least one peptide, polypeptide, protein, glycoprotein, protein-DNA complex, protein-membrane complex and/or lipoprotein are tested, reference is for instance made to WO 2007/008069 (quality control of medicaments) and WO 2007/008071 (quality control of other kinds of compositions), the contents of each of which are incorporated herein by this reference.

One embodiment therefore provides a method according to the invention, comprising selecting an immunogenic composition which is not, or to an acceptable extent, capable of specifically binding an antibody or a functional fragment or a functional equivalent thereof which is capable of specifically binding an epitope of the peptide, polypeptide, protein, glycoprotein, protein-DNA complex, protein-membrane complex and/or lipoprotein.

Another embodiment provides a method according to the invention, comprising selecting an immunogenic composition wherein the degree of multimerization of the peptide, polypeptide, protein, glycoprotein, protein-DNA complex, protein-membrane complex and/or lipoprotein in the composition does not, or to an acceptable extent, allow recognition of an epitope of the peptide, polypeptide, protein, glycoprotein, protein-DNA complex, protein-membrane complex and/or lipoprotein by an animal's immune system.

Another embodiment provides a method according to the invention, comprising selecting an immunogenic composition wherein less than 4% of the peptide, polypeptide, protein, glycoprotein, protein-DNA complex, protein-membrane complex and/or lipoprotein content of the composition is in a conformation comprising cross-beta structures.

Another embodiment provides a method according to the invention, comprising selecting an immunogenic composition which is not, or to an acceptable extent, capable of specifically binding a cross-beta structure binding compound, preferably tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, preferably cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR-BI), SR-A, chrysamine G, a chaperone, a heat shock protein, HSP70, HSP60, HSP90, gp95, calreticulin, a chaperonin, a chaperokine and/or a stress protein.

A method according to the invention is particularly suitable for producing and/or selecting an immunogenic composition which is capable of eliciting a humoral and/or cellular immune response. For a schematic overview of a humoral and cellular immune response, reference is made to FIG. 20. In certain embodiments, a method according to the invention is used for producing and/or selecting an immunogenic composition which is specifically adapted for eliciting a humoral immune response. In another embodiment, a method according to the invention is used for producing and/or selecting an immunogenic composition which is specifically adapted for avoiding a humoral immune response. In another embodiment, a method according to the invention is used for producing and/or selecting an immunogenic composition which is specifically adapted for eliciting both a humoral and a cellular immune response. In another embodiment, a method according to the invention is used for producing and/or selecting an immunogenic composition which is specifically adapted for eliciting a cellular immune response.

In order to produce and/or select an immunogenic composition which is specifically adapted for avoiding a humoral immune response, a method according to the invention preferably comprises the following steps: selecting, from a plurality of immunogenic compositions, an immunogenic composition: which is not capable of specifically binding an antibody or a functional fragment or a functional equivalent thereof which is capable of specifically binding an epitope of the peptide, polypeptide, protein, glycoprotein, protein-DNA complex, protein-membrane complex and/or lipoprotein; wherein the degree of multimerization of the peptide, polypeptide, protein, glycoprotein, protein-DNA complex, protein-membrane complex and/or lipoprotein in the composition does not allow recognition of an epitope of the peptide, polypeptide, protein, glycoprotein, protein-DNA complex, protein-membrane complex and/or lipoprotein by an animal's immune system; wherein less than 4% of the peptide, polypeptide, protein, glycoprotein, protein-DNA complex, protein-membrane complex and/or lipoprotein content of the composition is in a conformation comprising cross-beta structures; and/or which comprises a cross-beta structure which is not capable of specifically binding a cross-beta structure binding compound, preferably tPA, BiP, factor XII, fibronectin, at least one finger domain of tPA, at least one finger domain of factor XII, hepatocyte growth factor activator, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, preferably cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR-BI), SR-A, chrysamine G, a chaperone, a heat shock protein, HSP70, HSP60, HSP90, gp95, calreticulin, a chaperonin, a chaperokine and/or a stress protein, at a detectable level.

In order to produce and/or select an immunogenic composition which is suitable for activating T-cells and/or a T-cell response, a method according to the invention preferably comprises the following steps: determining whether a peptide, polypeptide, protein, glycoprotein, protein-DNA complex, protein-membrane complex and/or lipoprotein comprises a T-cell epitope motif; selecting a peptide, polypeptide, protein, glycoprotein, protein-DNA complex, protein-membrane complex and/or lipoprotein comprising a T-cell epitope motif; providing a composition comprising the selected peptide, polypeptide, protein, glycoprotein, protein-DNA complex, protein-membrane complex and/or lipoprotein; and providing the composition with at least one cross-beta structure.

In certain embodiments, a method according to the invention also comprises the production of an immunogenic composition which is capable of activating T-cells and/or a T-cell response, the composition comprising at least one peptide, polypeptide, protein, glycoprotein, protein-DNA complex, protein-membrane complex and/or lipoprotein comprising a T-cell epitope and/or a T-cell epitope motif, the method comprising providing the composition with at least one cross-beta structure and determining: whether the degree of multimerization of the peptide, polypeptide, protein, glycoprotein, protein-DNA complex, protein-membrane complex and/or lipoprotein in the composition allows recognition, binding, excision, processing and/or presentation of a T-cell epitope of the peptide, polypeptide, protein, glycoprotein, protein-DNA complex, protein-membrane complex and/or lipoprotein by an animal's immune system; whether between 4-75% of the peptide, polypeptide, protein, glycoprotein, protein-DNA complex, protein-membrane complex and/or lipoprotein content of the composition is in a conformation comprising cross-beta structures; whether the at least one cross-beta structure comprises a property allowing recognition, binding, excision, processing and/or presentation of a T-cell epitope of the peptide, polypeptide, protein, glycoprotein and/or lipoprotein by an animal's immune system; and/or whether a compound capable of specifically recognizing, binding, excising, processing and/or presenting a T-cell epitope of the peptide, polypeptide, protein, glycoprotein, protein-DNA complex, protein-membrane complex and/or lipoprotein is capable of specifically recognizing, binding, excising, processing and/or presenting the T-cell epitope. The compound capable of specifically recognizing, binding, excising, processing and/or presenting a T-cell epitope preferably comprises a T-cell receptor (TCR), an MHC complex, and/or a component of the MHC antigen processing pathway.

In certain embodiments, it is determined whether a component of the MHC antigen processing pathway is capable of recognizing, binding, excising, processing and/or presenting a T-cell epitope of the peptide, polypeptide, protein, glycoprotein, protein-DNA complex, protein-membrane complex and/or lipoprotein.

In order to produce and/or select an immunogenic composition which is suitable for activating T-cells and/or a T-cell response, an immunogenic composition whereby a component of the MHC antigen processing pathway is capable of recognizing, binding, excising, processing and/or presenting a T-cell epitope of the peptide, polypeptide, protein, glycoprotein, protein-DNA complex, protein-membrane complex and/or lipoprotein is preferably selected.

The invention is further explained in the following examples. These examples do not limit the scope of the invention, but merely serve to clarify the invention.

### BRIEF DESCRIPTION OF THE DRAWINGS

FIG. 1. Coomassie stained SDS-PA gel and Western blot with nE2 and nE2-FLAG-His. Lane 1: Coomassie nE2-FLAG-His (non-reducing); Lane 2: Western blot nE2-FLAG-His (non-reducing; anti-FLAG antibody); Lane 3: Coomassie nE2 in culture medium (non-reducing); Lane 4: Western blot nE2 in culture medium (non-reducing; mix of 3 monoclonal antibodies); Lane 5: Coomassie nE2 dialyzed to PBS and concentrated (non-reducing); Lane 6: Western blot nE2 dialyzed to PBS and concentrated (non-reducing; mix of 3 monoclonal antibodies); Lane 7: Coomassie nE2-FLAG-His (reducing); Lane 8: Western blot nE2-FLAG-His (reducing; anti-FLAG antibody); Lane 9: Coomassie nE2 in culture medium (reducing); Lane 10: Western blot nE2 in culture medium (reducing; mix of 3 monoclonal antibodies); Lane 11: molecular weight marker.

FIG. 2. Structure analyses of non-treated E2 and misfolded E2. E2 expressed in Sf9 cells and in cell culture medium was dialyzed against PBS and approximately tenfold concentrated, designated as nE2. Misfolded cross-beta E2 (cE2) was obtained by cyclic heating of nE2 (see text for details). A. Thioflavin T fluorescence enhancement assay with nE2 and cE2 at 100 μg/ml. Standard is 100 μg/ml dOVA. The fluorescence measured with dOVA standard is arbitrarily set to 100%. Buffer control was PBS. B. tPA/plasminogen chromogenic activation assay with nE2 and cE2 at 12.5 and 50 μg/ml in the assay. C. Transmission electron microscopy image of nE2. The scale bar is given in the image. D. TEM image of cE2.

FIG. 3. Transmission electron microscopy image of misfolded ovalbumin at 1 mg/ml.

FIG. 4. Coomassie-stained gel and Western blot with the H5 variants nH5-1, nH5-2, CH5-A, CH5-B. A. Non reducing SDS NuPage gel applied with nH5-1, nH5-2, CH5-A, CH5-B originating from H5-FLAG-His of H5N1 strain A/HK/156/97. Marker: 6 μl/lane, Precision Plus Protein Dual Color Standards, BioRad, Cat.#161-0374. Gel:NuPage 4-12% Bis-Tris Gel, 1.0 mm×10 well, Invitrogen, Cat.# NPO321BOX. M=Marker; nH5-1, 2 μg; nH5-2, 0.66 μg; CH5-A, 2 μg; CH5-B, 2 μg. B. Western blot with the H5 variants nH5-1, nH5-2, CH5-A, CH5-B, analyzed with peroxidase-labeled anti-FLAG antibody. In each indicated lane 30 ng H5 is loaded. H5 is of H5N1 strain A/Hong kong/156/97. Marker: 6 μl/lane, Precision Plus Protein Dual Color Standards, BioRad, Cat.# 161-0374. Gel:NuPage 4-12% Bis-Tris Gel, 1.0 mm×10 well, Invitrogen, Cat.# NP0321 BOX.

FIG. 5. Size exclusion chromatography analysis with non-treated H5 and H5 subjected to a misfolding procedure. The non-treated H5-FLAG-His, nH5-1, and this sample incubated at 37° C. with 100 mM DTT (CH5-B), originating from H5 of H5N1 strain A/HK/156/97, were subjected to a SEC column for analysis of the size distribution of H5 multimers, observed on Coomassie-stained SDS-PA gels applied with non-reducing conditions.

FIG. 6. Identification of soluble oligomers in H5 samples, of H5N1 strain A/HK/156/97, using ultracentrifugation. The H5 samples originating from H5N1 strain A/HK/156/97, as indicated in the graphs, were subjected to centrifugation for 10 minutes at 16,000*g (nH5-2, indicated with “16 k*g”), or for 60 minutes at 100,000*g (nH5-2, CH5-A, CH5-B, indicated with “100 k*g”). A. Protein concentration in the three H5 samples before and after centrifugation at the indicated times/g-forces. Relative concentrations are given for comparison. B. ThT fluorescence of four-fold diluted H5 samples before and after centrifugation at the indicated times/g-forces.

FIG. 7. Analysis of cross-beta structure in H5-FLAG-His samples. The H5 originates from H5N1 strain A/HK/156/97 and comprises a C-terminal FLAG-tag, followed by a His-tag. A. ThT fluorescence of the two non-treated H5 forms (nH5-1, nH5-2) and the two forms obtained after applying different misfolding procedures (CH5-A, CH5-B), tested at the indicated concentrations. Standard: 100 μg/ml cross-beta dOVA; fluorescence arbitrarily set to 100%. B. Congo red fluorescence of the non-treated H5 forms nH5-1 and CH5-A, CH5-B, tested at the indicated concentrations. Standard: 100 μg/ml cross-beta dOVA; fluorescence arbitrarily set to 100%. C. tPA/plasminogen activation assay using chromogenic plasmin substrate and depicted H5 solutions at the indicated concentrations. Standard: 40 μg/ml cross-beta dOVA; activity arbitrarily set to 100%. D. Transmission electron microscopy image of non-treated H5 form nH5-1. The bar indicates the scale of the image. E. Transmission electron microscopy image of nH5-2. F. Transmission electron microscopy image of CH5-A obtained after applying a misfolding procedure, as indicated in the text.

FIG. 8. Coomassie stained gel with a concentration series of H5 of H5N1 A/Vietnam/1203/04, under reduced and non-reduced conditions. H5 protein of H5N1 A/VN/1203/04 under reducing (sample 1-4) and non-reducing (sample 5-8) conditions. M=marker, lane 1, 5=4 μg H5, lane 2, 6=2 μg H5, lane 3, 7=1 μg H5, lane 4, 8=0.5 μg H5. Marker: 6 μl/lane, Precision Plus Protein Dual Color Standards, BioRad, Cat.# 161-0374. Gel:NuPage 4-12% Bis-Tris Gel, 1.0 mm×10 well, Invitrogen, Cat.# NP0321 BOX.

FIG. 9. TEM images of non-treated H5 of H5N1 A/VN/1203/04, and accompanying misfolded H5 variants CH5-1-4, comprising cross-beta. TEM analysis of nH5 (A.) shows amorphous aggregates. The incidence of aggregates is reduced to ˜5 aggregates/mesh in CH5-1 (B.), but the aggregates are larger in size, more dense and the morphology is changed compared to nH5. A high incidence of dense aggregates was observed in CH5-2 (C.). In the preparation of CH5-3 (D.), aggregates of similar morphology compared to CH5-2 were observed, but with reduced incidence. Lower aggregate count and dissimilar morphology of aggregates was observed for CH5-4 (E.).

FIG. 10. ThT and Congo red fluorescence enhancement measurements for non-treated and misfolded H5 (recombinantly produced H5 of H5N1 strain A/Vietnam/1203/04). Thioflavin T (A.) and Congo red fluorescence enhancement measurements (B.) of H5 show elevated fluorescence for the preparations CH5-1, CH5-2 and CH5-3 that were subjected to conditions favoring protein misfolding. Reduction in fluorescence intensity was observed in preparation CH5-4. The preparation CH5-1 was slightly turbid with some visible precipitates after heat treatment, which could explain the high standard deviation. C. tPA mediated plasminogen activation assay of non-treated and misfolded H5 variants originating from recombinantly produced H5 of H5N1 strain A/VN/1203/04. cH5-2 (150% of standard) and cH5-3 (200% of standard) are more potent cofactors for the activation of tPA/plasminogen compared to the starting material of nH5 (140% of standard). Lower activations were observed with cH5-1 (50% of standard) and cH5-4 (37% of standard) compared to the starting material. Substantial activation is observed with the starting material nH5, indicating that this H5 preparation already harbors misfolded proteins to some extent.

FIG. 11. Measurement of cross-beta parameters with misfolded FVIII variants. A. ThT fluorescence enhancement assay with the nine FVIII preparations. Standard (stand.) is 100 μg/ml dOVA. Freshly dissolved FVIII (9) was either diluted in the ThT assay solution directly, or after 10 minutes centrifugation at 16,000*g (sample 9(+)). FVIII is measured at 50 IE/ml, 12.5 IE/ml (2× diluted stock). The dotted line depicts the value measured for non-treated FVIII, sample 9. B. Congo red fluorescence enhancement assay, applied similar to the ThT fluorescence assay. C. ANS fluorescence assay applied similar to the ThT and Congo red fluorescence assays, shown in A. and B. D., E. The tPA/Plg chromogenic activation assay with the nine FVIII variants. The signal obtained with the plasmin activity induced by dOVA at 40 μg/ml (standard) is arbitrarily set to 100%. The lower dotted line depicts the maximum tPA/Plg activation activity as obtained with non-treated FVIII. The upper dotted line depicts the 100% tPA/Plg activity achieved with dOVA. F. Codes 1-9 for the 9 FVIII variants. Due to the necessity to adjust the pH twice in FVIII variants 7 and 8, the total volume increased 10%. Therefore, the analyses are performed at 9% lower concentration with respect to FVIII preparations 1-6 and 9, for all four assays depicted in A.-E.

FIG. 12. Identification of soluble oligomers in FVIII samples, before and after subjecting FVIII to misfolding procedures, using ultracentrifugation. The FVIII samples, as indicated in the graphs, were subjected to centrifugation for 60 minutes at 100,000*g, indicated with “100 k*g.” ThT fluorescence of two-fold diluted FVIII samples before and after centrifugation is shown. Fluorescence is normalized to 100 μg/ml dOVA standard. Buffer negative control was PBS.

FIG. 13. Binding of classical swine fever virus neutralizing mouse monoclonal antibodies to various appearances of E2, before and after misfolding. A.-C. Mouse monoclonal antibodies CediCon CSFV 21.2, 39.5 and 44.3 neutralize CSFV in vitro (information from the manufacturer) and are shown to bind to non-treated E2-FLAG-His (nE2-FLAG-His) expressed in 293 cells, non-treated E2 (nE2) expressed in Sf9 cells, and misfolded E2 comprising cross-beta, derived from nE2, to various extent.

FIG. 14. Binding of anti-H5 antibodies, that neutralize H5N1 A/VN/1203/04 virus and inhibit hemagglutination by H5N1, to variants of H5 of H5N1 A/HK/156/97. In an ELISA binding of four mouse monoclonal antibodies to four different appearances of H5 of H5N1 strain A/HK/156/97 is assessed; non-treated nH5-1 and nH5-2, and two H5 variants after subjecting nH5-1 to two different misfolding procedures (CH5-A, CH5-B). The antibodies are elicited against H5 of H5N1 A/VN/1203/04 and neutralize virus of this strain, as well as inhibit hemagglutination by this strain. For comparison, non-treated H5 of H5N1 A/VN/1203/04 (nH5) is incorporated in the analyses. A. Coat control with 1 and 0.1 μg/ml coated H5. Binding is detected using anti-FLAG antibody. H5 of H5N1 A/HK/156/97 comprises a FLAG-tag. B.-E. ELISAs with dilution series of the indicated antibodies. In B., nH5 was overlayed with a 1:2000 dilution of antibody 200-301-975, resulting in a signal of approximately 1.5. For clarity, this is not shown.

FIG. 15. Binding of anti-H5 functional antibodies (in vitro) to non-treated and misfolded forms of H5 of H5N1 strain A/VN/1203/04. A.-H. In an ELISA, binding of indicated dilution series of mouse monoclonal anti-H5 antibodies Rockland 200-301-975 to 979, raised against H5 of H5N1 strain A/VN/1203/04 and with hemagglutination inhibition activity and virus neutralizing activity, and HyTest 8D2, 17C8 and 15A6, with hemagglutination inhibition activity, was assessed using non-treated H5 of H5N1 strain A/VN/1203/04, and four misfolded variants, coded CH51 to 4 (see text for details), as indicated.

FIG. 16. Test ELISA for determination of anti-FVIII titers in hemophilia patient plasma. For determination of anti-FVIII titers in human hemophilia patient plasma, Helixate FVIII was immobilized in ELISA plates and overlayed with plasma dilutions ranging from 1:16 to 1:65536 (fourfold dilution), diluted in PBS/0.1% Tween20. A.-D. Patients A-D are hemophilia patients with FVIII inhibiting anti-FVIII antibody titers, whereas patients E-G are hemophilia patients that do not have circulating inhibiting antibodies (E.-G.). H. As a negative control, plasma of a healthy donor was incorporated in the titer determination.

FIG. 17. Binding of Hemophilia patient antibodies to factor VIII applied to nine different treatments. A-D. Plasma, diluted 1:200, of four different Hemophilia patients A-D with known factor VIII inhibitory antibody titers, is used for detection of anti-FVIII antibody binding to FVIII treated in nine different ways (see box in F.). E. Control plasma of a healthy donor exposed to the different types of FVIII. F. FVIII type sample codes, as depicted in A.-E. Plasma of patients E and F at 1:200 dilution, were also incorporated in the analyses, and for all nine FVIII variants no binding of antibodies for those two patient plasma's lacking FVIII inhibiting antibodies, was detected (data not shown).

FIG. 18. Coat control for immobilization of factor VIII types obtained after various treatments, to wells of an ELISA plate, using polyclonal peroxidase labeled anti-human factor VIII antibody SAF8C. See for the codes of the nine FVIII types the legend in FIG. 17F.

FIG. 19. Assessment of the binding of anti-FVIII antibodies from Hemophilia patients to non-treated FVIII and misfolded FVIII. A. Coat control for the various indicated FVIII preparations, using polyclonal anti-FVIII antibody SAF8C. B.-D. As depicted similarly in FIG. 17, binding of anti-FVIII antibodies from plasma of patients B-D, with known anti-FVIII inhibitory antibody titers, to various forms of non-treated FVIII (1) and misfolded FVIII (4-7) was assessed using an ELISA approach with immobilized FVIII types and 1:100 diluted plasma. E. Plasma of a healthy donor was used as a negative control. See for the codes of the five FVIII types the legend in FIG. 17F.

FIG. 20. Schematic overview of humoral immune response and cellular immune response.

FIG. 21. SEC elution pattern of dH5-0 and melting curve of cdH5-0, as determined by measuring Sypro Orange fluorescence during increasing temperature. A. SEC elution pattern of dH5-0. Approximately 65% of the dH5-0 elutes as a 33 kDa protein. B. Melting curve of cdH5-0. Half of the cdH5-0 molecules are molten at T=52.5° C.

FIG. 22. H5 forms analyzed on SDS-PA gel under reducing and non-reducing conditions. A. Lane M, marker with indicated molecular weights in kDa; lane 1 and 7, dH5-0; lane 2 and 8, cdH5-0; lane 3 and 9, fdH5-0; lane 4 and 10, dH5-I; lane 5 and 11, dH5-II; lane 6 and 12, dH5-III. Samples in lanes 1-6 are pre-incubated in non-reducing buffer (disulphide bonds stay intact), samples 7-12 are pre-heated in buffer comprising reducing agent dithiothreitol (DTT). B. SDS-PAGE analysis with non-reducing conditions, with various H5 samples, before/after ultracentrifugation.

FIG. 23. Enhancement of Thioflavin T fluorescence (A.) and Sypro orange fluorescence (B.) under influence of various H5 forms.

FIG. 24: Binding of Fn F4-5 to various forms of H5, as determined in an ELISA with immobilized H5.

FIG. 25. Binding of tPA to various structural variants of H5 and results of a tPA-mediated plasminogen activation assay with non-treated, misfolded and ultracentrifuged H5 samples, determined at 50 μg/ml H5. A.-D. In an ELISA the binding of tPA to H5 forms was tested. To avoid putative binding of the tPA kringle 2 domain to exposed lysine and arginine residues, the binding experiment is performed in the presence of an excess ε-amino caproic acid. In A, B and D, binding of tPA is shown, whereas in C binding of the negative control K2P tPA, which lacks the cross-beta binding finger domain, is shown. E. tPA/Plg activating potential was tested for the six different H5 forms. The activating potential of misfolded ovalbumin standard at 30 μg/ml is set to 100%; at 10 and 50 μg/ml, tPA/plg activation is 100% and 85%, respectively. H5 samples are all tested at 50 μg/ml.

FIG. 26. Example curves showing the relative binding of functional monoclonal anti-H5 antibody Rockland 977 to various structural variants of H5 of strain H5N1 A/VN/1203/04. Data for all nine functional antibodies is summarized in Table 1 and 2.

FIG. 27. Weight and survival curves of mice challenged with H5N1 virus. In Panels A-I, during 14 days post challenge for each individual mouse in groups 1-9, the weight and its survival are depicted. A weight of 0 gramme depicts that the mouse died that day. For clarity, the antigen for each group is indicated. X-axis, days post challenge with H5N1 virus; Y-axis, weight of mice in grammes.

FIG. 28. Elution pattern of cE2 after size exclusion chromatography, and analysis of SEC fractions on Coomassie stained gel after SDS-PAGE and on Western blot using monoclonal anti-E2 antibody 39.5. A. SEC elution pattern of cE2. The first peak are cE2 aggregates that are not retained by the SEC column. The peak in the middle comprises cE2 dimers and some monomers, as subsequently seen on SDS-PA gel. The third peak comprises E2 monomers. B. top figure; Coomassie stained SDS-PA gel with E2 samples from the SEC run, as indicated in the legend. Bottom figure; Western blot with the same pooled E2 fractions from the SEC run, using monoclonal anti-E2 antibody 39.5. C. SDS-PAGE gel with four cross-beta comprising E2 samples cE2, SEC-E2, cE2-A, cE2-B. All four E2 samples were loaded after heating under non-reducing (sample 1-4) and reducing (sample 5-8) conditions. The molecular weight marker is indicated.

FIG. 29. Fluorescence enhancement signals of ThT and Sypro Orange with the four cross-beta comprising E2 samples. The dOVA standard and E2 samples are measured at 50 μg/ml in the ThT assay and at 25 μg/ml in the Sypro Orange assay. In addition, the standard is measured at 100 and 25 μg/ml in the ThT fluorescence enhancement assay. Negative control is dilution buffer PBS. Fluorescence signals are normalized to the signal obtained with the standard; at 100 μg/ml for ThT, at 25 μg/ml for Sypro Orange.

FIG. 30. tPA mediated conversion of plasminogen in plasmin under influence of the E2 forms at 50 μg/ml, and Binding of Fibronectin finger 4-5 (Fn F4-5) to the four E2 forms, and binding of tPA and K2P tPA. A. The potency of the cross-beta structure in the four E2 forms to stimulate the formation of plasmin from plasminogen by tPA, is tested in a chromogenic assay in which plasmin generation is measured by recording plasmin substrate conversion in time. E2 samples are tested at 50 μg/ml. The standard is 30 μg/ml dOVA standard, and data is normalized to its activation potency. B. In an ELISA the binding of finger domain to cross-beta in E2 forms was assessed. C. Binding of tPA to four structural forms of E2: cE2, SEC-E2, cE2-A and cE2-B. D. Binding of K2P tPA to four structural forms of E2.

FIG. 31. Binding of anti-E2 antibodies to cE2 used for immunizations of pigs. Binding of mouse functional monoclonal antibodies 21.1, 39.5 and 44.4, which neutralize CSFV, to the four E2 forms (A.-C.), and binding of pig anti-E2 IgG antibodies from pooled serum of six pigs, which were obtained upon immunization of pigs with placebo/PBS (D.), cross-beta E2 (cE2, E.), E2 covalently coupled to ovalbumin and subsequently misfolded (E2-OVA, F.) and E2 adjuvated with a double oil in water emulsion according to a commercialized procedure (E2-DOE, G.). Binding of virus neutralizing mouse monoclonal antibodies 39.5 and 44.3 to cE2 under influence of a dilution series of pooled pig serum obtained after immunization with placebo/PBS or with cE2 adjuvated with double oil in water emulsion according to a commercialized protocol (H., I.). The immune sera were obtained during an immunization/CSFV challenge trial as outlined in patent application WO2007008070.

FIG. 32. Body temperature of each individual pig, and averaged per group of pigs. Clinical scoring of pigs. See for codes the outline in the main text. “Braaksel”=vomit, “stal”=shed, “diarree”=diarrhea, “geen mest”=no excrements. Sep. 22, 2008 is the start of the challenge period of 14 days. “dpc”=days post challenge. In FIGS. 32-35, the first column at the left refers to the group of pigs, the second column depicts the unique pig identifier for each pig in each group.

FIG. 33. Anti-E2 titer data and anti-viral Ems titer data. day 0=−42, 7=−35, 14=−28, 21=−21, 28=−14, 35=−7, 42=0, 44=2, 46=4, 49=7, 51-9, 53=11, 56=14. “positef”=positive, “negatief”=negative, “geeuthanaseerd”=euthanized.

FIG. 34. Virus isolation from pig leucocytes and oropharyngal swabs.

FIG. 35. White blood cell count and thrombocyte count at indicated time points (“dpc”=days post challenge).

FIG. 36. Enhancement of ThT fluorescence and activation of tPA and plasminogen in a chromogenic plasmin assay upon exposure to various cross-beta factor VIII preparations. The factor VIII concentration is 50 IE/ml, or 10 μg/ml (40 μg/ml stocks). A. Thioflavin T fluorescence enhancement assay. The fluorescence of the dOVA standard stock at 100 μg/ml is set to 100 a.u., for comparison. B. tPA/plasminogen activation by cross-beta factor VIII forms 1, 3, 5 and 12 is tested at 10 μg/ml in the assay and compared to a concentration series of dOVA standard at 100, 33.3 and 11.1 μg/ml. With the dOVA standard lot used, 100 μg/ml corresponds to 100% tPA/plasminogen activation.

FIG. 37. TEM images of cross-beta factor VIII forms 1, 3 and 5, and buffer control PBS. Cross-beta Factor VIII form 1 is kept at 4° C. after dissolving lyophilized protein, before storage at −80° C. before use. Cross-beta form 3 is incubated at 37° C., cross-beta form 5 is incubated at 95° C. For cross-beta factor VIII form 5, the image before and after ultracentrifugation is given. Negative control: PBS buffer.

FIG. 38. Appearance of cross-beta factor VIII structural variants on SDS-PA gel. Cross-beta Factor VIII form 3 is incubated at 37° C. for 20 hours, and comparable to form 12, which is incubated at 37° C. for seven days, before storage at 4° C.

FIG. 39. Binding of functional factor VIII inhibiting anti-factor VIII antibodies in 200-fold diluted human haemophilia patient plasma to the indicated factor VIII forms. A., B. Binding of functional antibodies from patient plasma B and C. C. Control plasma lacking functional anti-fVIII antibodies. D. Control for coating of the factor VIII forms. A mixture of three mouse monoclonal anti-factor VIII antibodies is used.

FIG. 40. SDS-PAGE analysis with non-reducing conditions, with various cross-beta OVA samples. For preparation of various OVA and description of the analysis see text.

FIG. 41. Enhancement of Thioflavin T fluorescence under influence of various OVA forms. Various forms of dOVA comprise cross-beta structure, with little to no cross-beta structure in nOVA (see also text and Table 14 for further description).

FIG. 42. Enhancement of Sypro Orange fluorescence under influence of various OVA forms. It is seen that dOVA forms have increased cross-beta structure when compared to nOVA (see also text and Table 15).

FIG. 43. tPA-mediated plasminogen activation assay with OVA samples. tPA activation potential was determined at the indicated concentration of 80, 25 and 10 μg/ml OVA. Right and left panel are graphs of two experiments. It is seen that cross-beta structure inducing methods induces cross-beta structure (for further details see text and Table 16).

FIG. 44. Binding of Fn F4-5 to various forms of OVA, as determined in an ELISA with immobilized OVA. It is seen that Fn 4-5 has increased binding to dOVA forms compared to nOVA. See also text and Table 17.

FIG. 45. anti-OVA IgG/IgM titer after immunization with cross-beta structure variants of OVA. 13 C57BL-6 mice were immunized on day 0, 7, 14 and 21 with 5 μg OVA subcutaneously. At day 25 serum was collected and total IgG was determined by ELISA. Results are expressed as Log¹⁰ of the OD50+/−SEM. See also Table 21.

### DETAILED DESCRIPTION

### Examples

abbreviations: ADCC, antibody dependent cell-mediated cytotoxicty; AFM, atomic force microscopy; ANS, 1-anilino-8-naphthalene sulfonate; aPMSF, 4-Amidino-Phenyl)-Methane-Sulfonyl Fluoride; BCA, bicinchoninic acid; bis-ANS, 4,4′-dianilino-1,1′-binaphthyl-5,5′-disulfonic acid; CD, circular dichroism; CR, Congo red; CSFV, Classical Swine Fever Virus; DLS, dynamic light scattering; DNA, Deoxyribonucleic acid; dOVA, misfolded ovalbumin comprising cross-beta; ELISA, enzyme linked immuno sorbent assay; ESI-MS, electron spray ionization mass spectrometry; FPLC, fast protein liquid chromatography; FVIII, coagulation factor VIII; g6p, glucose-6-phosphate; GAHAP, alkaline-phosphatase labeled goat anti-human immunoglobulin antibody; h, hour(s); H#, hemagglutinin protein of influenza virus, number #; HBS, HEPES buffered saline; HCV, hepatitis C virus; HGFA, Hepatocyte growth factor activator; HK, Hong kong; HPLC, high performance, or high-pressure liquid chromatography; HRP, horseradish peroxidase; hrs, hours; Ig, immunoglobulin; IgG, immunoglobulin of the class ′G; IgIM, immunoglobulins intramuscular; IgIV, immunoglobulins intravenous; kDa, kilo Dalton; LAL, Limulus Amoebocyte Lysate; MDa, mega Dalton; NMR, nuclear magnetic resonance; OVA, ovalbumin; PBS, phosphate buffered saline; Plg, plasminogen; RAGE, receptor for advanced glycation end-products; RAMPO, peroxidase labeled rabbit anti-mouse immunoglobulins antibody; RNA, ribonucleic acid; RSV, respiratory syncytial virus; RT, room temperature; SDS-PAGE, sodium-dodecyl sulphate-polyacryl amide gel electrophoresis; SEC, size exclusion chromatography; SWARPO, peroxidase labeled swine anti-rabbit immunoglobulins antibody; TEM, transmission electron microscopy; ThS, Thioflavin S; ThT, Thioflavin T; tPA, tissue type plasminogen activator; VN, Vietnam; W, tryptophan.

### Detection of Proteins Comprising Cross-Beta

Cross-Beta Detection Assays

Congo red fluorescence. Congo red is a relatively small molecule (chemical name: C₃₂H₂₂N₆Na₂O₆S₂) that is commonly used as histological dye for detection of amyloid. The specificity of this staining results from Congo red's affinity for binding to fibrillar proteins enriched in beta-sheet conformation and comprising cross-beta. Congo red is also used to selectively stain protein aggregates with amyloid properties that do not necessarily form fibrils. Congo red is also used in a fluorescence enhancement assay to identify proteins with cross-beta in solution. This assay, also termed Congo red fluorescence measurement, is for example performed as described in patent application WO2007008072, paragraph [101], the contents of which are incorporated herein by this reference. Fluorescence can be read on various readers, for example fluorescence is read on a Gemini XPS microplate reader (Molecular Devices).

Thioflavin T fluorescence. Thioflavin T, like Congo red, is also used by pathologists to visualize plaques composed of amyloid. It also binds to beta sheets, such as those in amyloid oligomers. The dye undergoes a characteristic 115 nm red shift of its excitation spectrum that may be selectively excited at 442 nm, resulting in a fluorescence signal at 482 nm. This red shift is selectively observed if structures of amyloid fibrillar nature are present. It will not undergo this red shift upon binding to precursor monomers or small oligomers, or if there is a high beta sheet content in a non-amyloid context. If no amyloid fibrils are present in solution, excitation and emission occur at 342 and 430 nm respectively. Thioflavin T is often used to detect cross-beta in solutions. For example, the Thioflavin T fluorescence enhancement assay, also termed ThT fluorescence measurement, is performed as described in patent application WO2007008072, paragraph [101]. Fluoresence can de read on various readers, for example fluorescence is read on a Gemini XPS microplate reader (Molecular Devices).

Thioflavin S fluorescence. Thioflavin S, is a dye similar to Thioflavin T and the fluorescence assay is performed essentially similar to ThT and CR fluorescence measurements.

tPA binding ELISA. tPA binding ELISA with immobilized misfolded proteins; is performed as described in patent application WO2007008070, paragraph [35-36]. One of our first discoveries was that tPA binds specifically to misfolded proteins comprising cross-beta. Binding of tPA to misfolded proteins is mediated by its finger domain. Other finger domains and proteins comprising homologous finger domains are also applicable in a similar ELISA setup (see below).

BiP binding ELISA. BiP binding ELISA with immobilized misfolded proteins; is performed as described in patent application WO2007108675, section “Binding of BiP to misfolded proteins with cross-beta structure,” the contents of which are incorporated herein by this reference, with the modification that BiP purified from cell culture medium using Ni²⁺ based affinity chromatography, is used in the ELISAs. It has been demonstrated previously that chaperones like for example BiP bind specifically to misfolded proteins comprising cross-beta. Other heat shock proteins, such as hsp70, hsp90 are also applicable in a similar ELISA setup.

IgIV binding ELISA. Immunoglobulins intravenous (IgIV) binding ELISA with immobilized misfolded proteins; is performed as described in patent application WO2007094668, paragraph [0115-0117], the contents of the entirety of which are incorporated herein by this reference. Alternatively, IgIV that is enriched using an affinity matrix with immobilized protein(s) comprising cross-beta, is used for the binding ELISA with immobilized misfolded proteins (see patent application WO2007094668, paragraph [0143]). It has been demonstrated previously that a subset of immunoglobulins in IgIV bind selectively and specifically to misfolded proteins comprising cross-beta. Other antibodies directed against misfolded proteins are also applicable in a similar ELISA setup.

Finger binding ELISA using fibronectin finger domains. Fibronectin finger 4-5 binding ELISA with immobilized misfolded proteins; is performed as described in patent application WO2007008072. It has been demonstrated previously that finger domains of fibronectin selectively and specifically bind to misfolded proteins comprising cross-beta. In addition to, or alternative to finger domains of fibronectin, finger domains of tPA and/or factor XII and/or hepatocyte growth factor activator are used.

Factor XII activation assay. Factor XII/prekallikrein activation assay is performed as described in patent application WO2007008070, paragraph [31-34]. It has been demonstrated previously that factor XII selectively and specifically bind to misfolded proteins comprising cross-beta, resulting in its activation.

tPA/plasminogen activation assay. Enhancement of tPA/plasminogen activity upon exposure of the two serine proteases to misfolded proteins was determined using a standardized chromogenic assay (see for example patent application WO2006101387, paragraph [0195], patent application WO2007008070, paragraph [31-34], and [Kranenburg et al., 2002, Curr. Biology 12(22), pp. 1833)]. Both tPA and plasminogen act in the Cross-beta Pathway. Enhancement of the activity of the cross-beta binding proteases is a measure for the presence of misfolded proteins comprising cross-beta structure. 4-Amidinophenylmethanesulfonyl fluoride hydrochloride (aPMSF, Sigma, A6664) was added to protein solutions to a final concentration of 1.25 mM from a 5 mM stock. Protein solutions with added aPMSF were kept at 4° C. for 16 h before use in a tPA/plasminogen activation assay. In this way, proteases that are putatively present in protein solutions to be analyzed, and that may act on tPA, plasminogen, plasmin and/or the chromogenic substrate for plasmin, are inactivated, to prevent interference in the assay.

Binding assays. Apart from the above described binding assays using cross-beta binding compounds, additional cross-beta binding compounds are used in binding assays for determination of the presence and extent of cross-beta in a sample of a peptide, polypeptide, protein, glycoprotein, protein-DNA complex, protein-membrane complex and/or lipoprotein. In general, cross-beta binding compounds useful for these determinations are tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor such as RAGE or CD36 or CD40 or LOX-1 or TLR2 or TLR4, a cross-beta-specific antibody, preferably cross-beta-specific IgG and/or cross-beta-specific IgM, IgIV, an enriched fraction of IgIV capable of specifically binding a cross-beta structure, Low density lipoprotein Related Protein (LRP), LRP Cluster II, LRP Cluster IV, Scavenger Receptor B-I (SR-BI), SR-A, chrysamine G, a chaperone, a heat shock protein, HSP70, HSP60, HSP90, gp95, calreticulin, a chaperonin, a chaperokine and/or a stress protein. In addition, as disclosed previously in patent application WO2007008072, cross-beta binding compounds for use for the aforementioned determinations are 2-(4′-(methylamino)phenyl)-6-methylbenzothiaziole, styryl dyes, BTA-1, Poly(thiophene acetic acid), conjugated polyelectrolyte, PTAA-Li, Dehydro-glaucine, Ammophedrine, isoboldine, Thaliporphine, thalicmidine, Haematein, ellagic acid, Ammophedrine HBr, corynanthine, Orcein.

### Measurements of Protein Refolding and Changes in Protein Conformation & Multimer Size and Multimer Size Distribution Analysis

Turbidity of protein solutions. With turbidity measurements the diffraction of light scattered by protein particles in the sample is detected. Light is scattered by the solid particles and absorbed by dissolved protein. In a turbidity measurement the amount of insoluble particles in a solution is determined. This aspect is used to determine the amount of insoluble protein in samples of protein that is subjected to misfolding conditions, compared to the fraction of insoluble protein in the non-treated reference sample.

Recording changes in binding characteristics of binding partners for a protein. Antibodies specific for a protein in a certain conformation are used to measure the amount of this protein present in this specific state. Upon treatment of the protein using misfolding conditions, binding of antibodies is inhibited or diminished, which is used as a measure for the progress and extent of misfolding. In addition or alternatively, antibodies are used that are specific for certain conformations and/or post-translational modifications, for example glycation, oxidation, citrullination (gain of binding to the protein subjected to misfolding conditions). When for example glycation and/or oxidation and/or citrullination procedures is/are part of the misfolding procedure, the effect of the treatment with respect to the occurrence of modified amino-acid residues is recorded by determining the relative binding of the antibodies, compared to the non-treated reference protein. Alternatively or in addition to the use of antibodies, any binding partner and/or ligand of the non-treated protein is used similarly, and/or any binding partner and/or ligand other than antibodies, of the misfolded protein is used. When a protein changes conformation ligands or binding partners express altered binding characteristics, which is used as a measure for the extent of protein modification and/or extent of misfolding. This binding of antibodies, ligands and/or binding partners is measured using various techniques, such as direct and/or indirect ELISA, surface plasmon resonance, affinity chromatography and immuno-precipitation approaches.

Differential scanning calorimetry/micro DSC for detecting changes in protein conformation. Differential scanning calorimetry (DSC) is a thermo-analytical technique in which the difference in the amount of heat required to increase the temperature of a sample and a reference is measured as a function of temperature. The temperature is linearly increased over time. When the protein in the sample changes its conformation, more or less heat (depending on if it is an endo- or exothermic reaction) will be required to increase the temperature at the same rate as the reference sample. In this way the conformational changes as a result of an increase in temperature can be measured.

Particle analyzer. A particle analyzer measures the diffraction of a laser beam when targeted at a sample. The resulting data is transformed by a Fourier transformation and gives information about particle size and shape. When applied to protein solutions, putatively present protein aggregates are detected, when larger than the lower detection limit of the apparatus, for example in the sub-micron range.

Direct light microscope. With a regular direct-light microscope with a preferable magnification range of 10×-100×, one can determine visually if there are any protein aggregates present in a sample.

Photon correlation spectroscopy (dynamic light scattering spectroscopy). Photon correlation spectroscopy can be used to measure particle size distribution in a sample in the nm-μm range.

Nuclear magnetic resonance spectroscopy. Nuclear Magnetic Resonance Spectroscopy (NMR) can be used to assess the electromagnetic properties of certain nuclei in proteins. With this technique the resonance frequency and energy absorption of protons in a molecule are measured. From this data structural information about the protein, like angles of certain chemical bonds, the lengths of these bonds and which parts of the protein are internally buried, can be obtained. This information can then be used to calculate the complete three dimensional structure of a protein. This method however is normally restricted to relatively small molecules. However with special techniques like incorporation of specific isotopes and transverse relaxation optimized spectroscopy, much larger proteins can now be studied with NMR.

X-ray diffraction. In X-ray diffraction with protein crystals, the elastic scattering of X-rays from a crystallized protein is measured. In this way the arrangement of the atoms in the protein can be determined, resulting in a three-dimensional structural model of the protein. First a protein is crystallized and then a diffraction pattern is measured by irradiating the crystallized protein with an X-ray beam. This diffraction pattern is a representation of how the X-ray beam is scattered from the electrons in the crystal. By gradually rotating the crystal in the X-ray beam, the different atomic positions in the crystal can be determined. This results in an electron density map, with which a complete three-dimensional atomic model of the crystallized protein can be calculated, regularly at the 1-3 Å scale. In this model it can be deduced whether protein molecules underwent conformational changes upon treatment with misfolding conditions, when compared to the structural model of the non-treated protein. In addition, modifications of amino-acid residues become apparent in the structural model, as well as whether the protein molecule forms ordered multimers of a defined size, like for example in the range of dimers-octamers.

Determination of the presence of cross-beta in fibers comprising crystallites, and/or in other appearances of protein aggregates comprising at least a fraction of the protein molecules in a crystalline ordering, can be assessed using X-ray fiber diffraction, as for example shown in [Bouma et al., J. Biol. Chem. V278, No. 43:41810-41819, 2003, “Glycation Induces Formation of Amyloid Cross-beta Structure in Albumin”].

Fourier Transform infrared spectroscopy. Detection of protein secondary structure in Fourier Transform Infrared Spectroscopy (FTIR), an infrared beam is split in two separate beams. One beam is reflected on a fixed mirror, the second on a moving mirror. These two beams together generate an interferogram which consists of every infrared frequency in the spectrum. When transmitted through a sample specific functional groups in the protein adsorb infrared of a specific wavelength. The resulting interferogram must be Fourier transformed, before it can be interpreted. This Fourier transformed interferogram gives a plot of al the different frequencies plotted against their adsorption. This interferogram is specific for the structure of a protein, like a “molecular fingerprint,” and provides information on types of atomic bonds present in the molecule, as well as the spatial arrangement of atoms in for example alpha-helices or beta-sheets.

8-Anilino-1-naphthalenesulfonic acid fluorescence enhancement assay. 8-Anilino-1-naphthalenesulfonic acid (ANS) fluorescence enhancement assay, or ANS fluorescence measurement; was performed as described in patent application WO2007094668. Modification: fluorescence is read on a Gemini XPS microplate reader (Molecular Devices).

ANS is a chemical binds to hydrophobic surfaces of a protein and its fluorescence spectrum shifts upon binding. When proteins are in an unfolded state, they generally display more hydrophobic sites, resulting in an increased ANS shift compared to the protein in its native more globular state. ANS can therefore be used to measure protein unfolding.

bis-ANS fluorescence enhancement assay. 4,4′ dianilio-1,1′ binaphthyl-5,5′ disulfonic acid di-potassium salt (Bis-ANS) fluorescence enhancement assay; is performed as described in patent application WO2007094668. Essentially, bis-ANS has characteristics comparable to ANS, and bis-ANS is also used to probe for differences in solvent exposure of hydrophobic patches of proteins, when measuring bis-ANS binding with a reference protein samples, and with a protein sample subjected to a misfolding procedure.

Gel electrophoresis. Gel electrophoresis using sodium dodecyl-sulphate polyacryl amide gels (SDS-PAGE) and Coomassie stain, with various gels with resolutions between for example 100 Da up to several thousands of kDa, provides information on the occurrence of protein modifications and on the occurrence of multimers. Multimers that are not covalently coupled may also appear as monomers upon the assay conditions applied, i.e., heating protein samples in assay buffer comprising SDS. Samples are heated in the presence or absence of a reducing agent like for example dithiothreitol (DTT), when the protein amino-acid sequence comprises cysteines, that can form disulphide bonds upon subjecting the protein to misfolding conditions.

Western blot. When antibodies are available that bind to epitopes on the protein under the denaturing conditions as applied during SDS-PAGE, Western blotting is performed with the same protein samples as applied for SDS-PAGE with Coomassie stain, using the same molecular weight cut-off gels, and using the same protein sample handling approaches.

Centrifugation. Centrifugation and subsequent comparing the protein concentration in the supernatant with respect to the concentration before centrifugation provides insight into the presence of insoluble precipitates in a protein sample. Upon applying increasing g-forces for a constant time, and/or upon applying fixed or increasing g-forces for an increasing time frame, to a protein solution, with analyzing the protein content in between each step, information is gathered about the presence of insoluble multimers. For example, protein solutions are subjected for 10 minutes to 16,000*g, or for 60 minutes to 100,000*g. The first approach is commonly used to prepare protein solutions for, for example use on FPLC columns or in biological assays, with the aim of pelleting insoluble protein aggregates and using the supernatant with soluble protein. It is generally accepted that after applying 100,000*g for 60 minutes to a protein solution, only soluble multimers are left in the supernatant. As multimers ranging from monomers up to huge multimers comprising thousands of protein monomers may all have a density equal to the density of the buffer solution, applying these g-forces to protein solutions does not separate exclusively on size, but on density differences between the solution and the protein multimers.

Electron spray ionization mass spectrometry. Electron spray ionization mass spectrometry (ESI-MS) with protein solutions provides information on the multimer size distribution when sizes range from tens of Da up to the MDa range.

Ultrasonic spectrometry. Ultrasonic spectroscopy analysis, for example using an Ichos-II (Process Analysis and Automation, Ltd), provides insight into protein conformation and changes in tertiary structure are measured. In addition the technique can provide information on particle size of protein assemblies, and allows for monitoring protein concentration.

Dialysis (membranes with increasing molecular weight cut-off). Using one or a series of dialysis membranes with varying molecular weight cut-offs, size distribution/multimer distribution of protein can be assessed at the sub-oligomer scale, depending on the molecular weight of the monomer. Protein concentration analysis between each dialysis step with gradually increasing pore size (suitable for molecular weight ranges between approximately 1000-50000 Da). Protein concentration is for example monitored using BCA or Coomassie+determinations (Pierce), and/or absorbance measurements at 280 nm, using for example the nanodrop technology (Attana).

Filtration (filters with increasing molecular weight cut-off). Filtration using a series of filters with gradually increasing MW cut-offs, ranging from the monomer size of the protein under investigation up to the largest MW cut-off available, reveals information on the distribution and presence of protein molecules in multimers in the range from monomers, lower-order multimers and large multimers comprising several hundreds of monomers. For example, filters with a MW cut-off of I kDa up to filters with a cut-off of 5 μm (MWs for example 1/3/10/30/50/100 kDa, completed with filters with cut-offs of for example 200/400/1000/5000 nm). In between each subsequent filtration step, protein concentration is assessed using for example the BCA or Coomassie+method (Pierce), and/or visualization on SDS-PA gel stained with Coomassie.

Transmission electron microscopy. Transmission electron microscopy (TEM) is a imaging technique that provides structural information of proteins at a nm to μm scale. With this resolution it is possible to identify the occurrence of protein assemblies ranging from monomers up to multimers of several thousands molecules, depending on the molecular weight of the parent protein molecule. Furthermore, TEM imaging provides insight into the structural appearance of protein multimers. For example, protein multimers appear as rods, globular structures, strings of globular structures, amorphous assemblies, unbranched fibers, commonly termed fibrils, branched fibrils, and/or combinations thereof.

In the current studies, TEM images were collected using a Jeol 1200 EX transmission electron microscope (Jeol Ltd., Tokyo, Japan) at an excitation voltage of 80 kV. For each sample, the formvar and carbon-coated side of a 100-mesh copper or nickel grid was positioned on a 5 μl drop of protein solution for 5 minutes. Afterwards, it was positioned on a 100 μl drop of PBS for 2 minutes, followed by three 2-minute incubations with a 100 μl drop of distilled water. The grids were then stained for 2 minutes with a 100 μl drop of 2% (m/v) methylcellulose with 0.4% uranyl acetate pH 4. Excess fluid was removed by streaking the side of the grids over filter paper, and the grids were subsequently dried under a lamp. Samples were analyzed at a magnification of 10 K.

Atomic force microscopy. Similar to TEM imaging, atomic force microscopy provides insights into the structural appearance of protein molecules at the protein monomer level up to the macroscopic level of large multimers of protein molecules.

Size exclusion chromatography, or gel filtration chromatography. With size exclusion chromatography (SEC) using HPLC and/or FPLC, a qualitative and quantitative insight is obtained about the distribution of protein molecules over monomers up to multimers, with a detectable size limit of the multimers restricted by the type of SEC column that is used. SEC columns are available with the ability to separate molecular sizes in the sub kDa range up to in the MDa range. The type of column is selected based on the molecular weight of the analyzed protein, and on any indicative information at forehand about the expected range of multimeric sizes. Preferably, a reference non-treated protein is compared to a protein that is subjected to misfolding procedures.

Tryptophan fluorescence. Assessment of differences in tryptophan (W) fluorescence intensity between two appearances of the same protein provides information on the occurrence of protein folding differences. In general, in globular proteins W residues are mostly buried in the interior of the globular fold. Upon unfolding, refolding, misfolding, W residues tend to become more solvent exposed, which is recorded in the W fluorescence measurement as a change in fluorescent intensity compared to the protein with a more native fold.

Dynamic Light Scattering. With the Dynamic Light Scattering (DLS) technique, particle size and particle size distribution is assessed. When protein solutions are considered distribution of proteins over a range of multimers ranging from monomers up to multimers is measured, with the upper limit of detected multimer size limited by the resolution of the DLS technique.

Circular dichroism spectropolarimetry. With circular dichroism spectropolarimetry (CD) the relative presence of protein secondary structural elements is determined. Therefore, this technique allows for the comparison of the relative occurrence of alpha-helix, beta-sheet and random coil between a reference protein that is non-treated, and the protein that is subjected to misfolding conditions. An example of a CD experiment for assessment of conformational changes in proteins upon treatment with misfolding conditions is given in [Bouma et al., J. Biol. Chem. V278, No. 43:41810-41819, 2003, “Glycation Induces Formation of Amyloid Cross-beta Structure in Albumin”].

Native gel electrophoresis. Distribution over multimers in the range of approximately monomers up to 100-mers is assessed by applying native gel electrophoresis. For this purpose a reference non-treated protein sample is compared to a protein sample which is subjected to a misfolding procedure. When misfolding procedures are applied that introduce modifications on amino-acid residues, like for example but not limited to, glycation or oxidation or citrullination, these changes are becoming apparent on native gels, as well.

Examples of Proteins that are Used for Preparation of Immunogenic Compositions

Envelope protein E2 of Classical Swine Fever Virus. The envelope protein E2 of Classical Swine Fever Virus (CSFV) strain Brescia 456610 is used as a prototype subunit vaccine candidate for examples described below. Currently, a subunit vaccine that provides protection in pigs against CSF comprises recombinantly produced E2 antigen in cell culture medium, adjuvated with a double emulsion of water-in-oil-in-water, comprising PBS, Marcol 52, Montanide 80. The vaccine comprises at least 32 μg E2/dose of 2 ml, and is injected intramuscularly.

E2 was recombinantly produced in insect Sf9 cells (Animal Sciences Group, Lelystad, The Netherlands) or in human embryonic kidney 293 cells (293) (ABC-Protein Expression facility, University of Utrecht, The Netherlands), as described in patent application WO2007008070. E2 produced in Sf9 cells and lacking any tags is in PBS after dialysis of cell culture medium (storage of aliquots at −20° C. or at −80° C.), or in cell culture medium (storage at −20° C.). Cell culture medium is SF900 II medium with 0.2% pluronic (serum free). After culturing of cells, the cell culture medium is micro-filtrated. Virus is inactivated with 8-12 mM 2-bromo-ethyl-ammonium bromide. The E2 produced in 293 cells comprises a C-terminal FLAG-tag followed by a His-tag, and is purified using Ni²⁺-based affinity chromatography. Concentration and purity of E2 from both sources is determined as follows. Quantification of the total protein concentration is performed with the BCA method (Pierce) or with the Coomassie+method (Pierce). E2 specific bands on a Western blot are visualized using anti-FLAG antibody (mouse antibody, M2, peroxidase conjugate; Sigma, A-8592) for the E2-FLAG-His construct, and a 1:1:1 mixture of three horseradish peroxidase (HRP) tagged mouse monoclonal anti-E2 antibodies (CediCon CSFV 21.2, 39.5 and 44.3; Prionics Lelystad) for the E2-FLAG-His construct and the E2 construct from Sf9 cells. The purity of E2 batches was determined by densitometry with a Coomassie stained sodium dodecyl sulphate-polyacryl amide (SDS-PA) gel after electrophoresis.

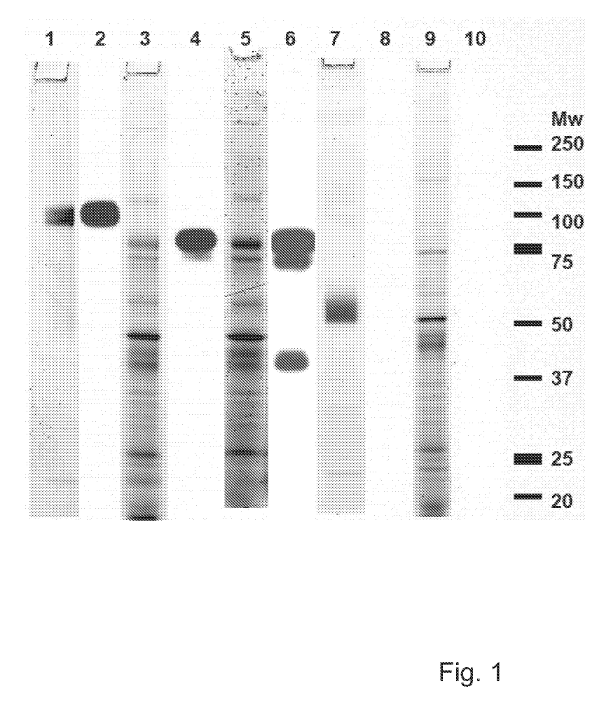

In FIG. 1, SDS-PA gels and Western blots with E2 produced in Sf9 cells and E2-FLAG-His produced in 293 cells are shown, with reducing and non-reducing conditions. It is clearly seen that the main fraction of both E2 batches appears as dimers on the gel and blot, when applied with non-reducing sample buffer. Apparently, those dimers are covalently coupled, since treatment of E2 from 293 cells with DTT reveals monomers at the expected molecular weight of approximately 47 kDa. No E2 bands are visualized on the blot when analyzing E2 from Sf9 cells under reducing conditions. The observation that E2 appears as at least two monomer and dimer bands is most likely related to the presence of glycosylation isoforms.

Before use in misfolding procedures, cross-beta analyses, multimer analyses and/or immunization, non-treated E2 solution was warmed to 37° C. for 10-30 minutes, left on a roller device for 10-30 minutes, at room temperature, warmed again at 37° C. for 0-30 minutes and left again on a roller device for 0-30 minutes. Alternatively, non-treated E2 solutions were quickly thawed at 37° C. and directly kept on wet ice until further use.

ovalbumin. Ovalbumin is incorporated as a candidate ingredient of immunogenic compositions comprising cross-beta structure. The ovalbumin is either serving as the antigen itself, to which an immune response should be directed, or ovalbumin is used as the cross-beta adjuvant part in immunogenic compositions, comprising a target antigen with a different amino-acid sequence. For this latter use, ovalbumin comprising cross-beta is combined with the target antigen, to which an immune response is desired. Cross-beta adjuvated ovalbumin is for example covalently coupled to the antigen of choice, using coupling techniques known to a person skilled in the art. When ovalbumin is the target antigen itself, non-treated ovalbumin and cross-beta-adjuvated ovalbumin are used in a similar way, in immunogenic composition preparations.

Lyophilized ovalbumin, or chicken egg-white albumin (OVA, Sigma, A5503 or A7641) is dissolved as follows. OVA is gently dissolved at indicated concentration in phosphate buffered saline (PBS; 140 mM sodium chloride, 2.7 mM potassium chloride, 10 mM disodium hydrogen phosphate, 1.8 mM potassium dihydrogen phosphate, pH 7.3; local pharmacy), avoiding any foam formation, stirring, vortexing or the like. OVA is dissolved by gently swirling, 10 minutes rolling on a roller device, 10 minutes warming in a 37° C.-water bath, followed by 10 minutes rolling on a roller device. Aliquots in Eppendorf tubes are frozen at −80° C. Before use, OVA solution is either prepared freshly, or thawed from −80° C. to 0° C., or after thawing kept at 37° C. for 30 minutes. Furthermore, an OVA solution is applied to an endotoxin affinity matrix for removal of endotoxins present in the OVA preparation. Before and after applying OVA to the matrix, endotoxin levels are determined using an Endosafe apparatus (Charles River), and/or using a chromogenic assay for determining endotoxin levels (Cambrex), both using Limulus Amoebocyte Lysate (LAL). Misfolded OVA, termed dOVA, is prepared as indicated below (see Section “Protocols for introducing cross-beta in proteins”).

Hemagglutinin 5 protein of H5N1 virus strain A/Hong kong/156/97. Hemagglutinin 5 protein (H5) of H5N1 virus strain A/Hong kong/156/97 (A/HK/156/97) is expressed in 293 cells with a C-terminal FLAG tag and His tag, and purified using Ni²⁺-based affinity chromatography as described in patent application WO/2007/008070, the contents of which are incorporated herein by this reference. In addition, the recombinantly produced H5-FLAG-His construct is purified using affinity chromatography with the anti-FLAG antibody M2 immobilized on a matrix (Sigma, A2220), according to the manufacturer's recommendations and using FLAG peptide (Sigma, F3290) for elution of H5-FLAG-His from the matrix. Protein solutions are stored at −80° C. for a long term and after micro filtration at 4° C., for a short term. In this example, upon purification using anti-FLAG antibody based affinity chromatography, two batches of H5 were obtained. One batch of H5-FLAG-His is termed non-treated H5, batch 2 (“nH5-2,” concentration 30 μg/ml). A second batch of H5-FLAG-His was subsequently subjected to size-exclusion chromatography (SEC) using a HiLoad 26/60 Superdex 200 column on an Äkta Explorer (GE Healthcare; used at the ABC-protein expression facilities of the University of Utrecht, Dr R. Romijn & Dr. W. Hemrika). For this purpose, H5-FLAG-His solution in PBS is concentrated on Macrosep Centrifugal Devices 10K Omega (Pall Life Sciences) or CENTRIPREP Centrifugal Filter Devices YM-300 (Amicon). Running buffer was PBS. The H5 batch after the SEC run, termed non-treated H5, batch 1 (“nH5-1”), was stored at 4° C. after micro filtration (concentration 400 μg/ml, as determined with the BCA method). This batch nH5-1 is used for misfolding procedures described below.

H5 of H5N1 strain A/Vietnam/1203/04. H5 of H5N1 strain A/Vietnam/1203/04 (A/VN/1203/04) is purchased from Protein Sciences, and consists mainly of HA2, with relatively lower amounts of HA1 and HA0. Purity is 90%, as determined with densitometry, according to the manufacturer's information. Buffer and excipients are 10 mM sodium phosphate, 150 mM NaCl, 0.005% Tween80, pH 7.2. The H5 concentration is 922 μg/ml (lot 45-05034-2) or 83 μg/ml (lot 45-05034RA-2). This non-treated H5 is termed “nH5” and stored at 4° C. or at −80° C.

Factor VIII. Factor V111 (FVIII) of human plasma origin or recombinantly produced based on cDNA coding for human FVIII is used. Examples of suitable FVIII preparations are Helixate (Nexgen), Kogenate (Bayer), Advate (Baxter), Recombinate (Baxter), ReFacto (FVIII in which the B-domain is deleted; Wyeth), which are all recombinantly produced, and AAfact (Sanquin) and Haemate P (Aventis Behring), which are purified from blood. FVIII preparations are dissolved according to the manufacturer's recommendations. For the examples disclosed below, Helixate (NexGen 250 IE/vial, lot 80A0777, exp. date: 03.2007) is used, termed non-treated FVIII and designated as “FVIII.”

### Other Antigens

The proteins described above are used for preparation of immunogenic compositions. However, the disclosed technologies are by no means restricted to the generation of immunogenic compositions comprising OVA, FVIII, H5 of A/VN/1203/04 or A/HK/156/97, or E2. Examples that further disclose the described technologies and their applications are also generated using other and/or additional peptides, polypeptides, proteins, glycoproteins, protein-DNA complexes, protein-membrane complexes and/or lipoproteins as a basis for immunogenic compositions. These peptides, polypeptides, proteins, glycoproteins, protein-DNA complexes, protein-membrane complexes and/or lipoproteins are the antigen component, the cross-beta-adjuvated component or both the antigen component and the cross-beta-adjuvated component of immunogenic compositions. The peptides, polypeptides, proteins, glycoproteins, protein-DNA complexes, protein-membrane complexes and/or lipoproteins are for instance originating from amino-acid sequences unrelated to pathogens and/or diseases, when used as the cross-beta-adjuvated ingredient of an immunogenic composition, or are for instance originating from amino-acid sequences that are related to and/or involved in and/or are part of pathogens, tumors, cardiovascular diseases, atherosclerosis, amyloidosis, autoimmune diseases, graft-versus-host rejection and/or transplant rejection, when they are part of the target antigen and/or are the cross-beta-adjuvated ingredient of an immunogenic composition. In fact, the disclosed technologies are applicable to any amino-acid sequence, either of the antigen, or of the cross-beta-adjuvant.

Non-limiting examples of peptides, polypeptides, proteins, glycoproteins, protein-DNA complexes, protein-membrane complexes and/or lipoproteins that are used as antigen and/or as cross-beta-adjuvant are for example virus surface proteins, bacterial surface proteins, pathogen surface exposed proteins, gp120 of HIV, proteins of human papilloma virus, any of the neuramidase proteins or hemagglutinin proteins or any of the other proteins of any influenza strain, surface proteins of blue tongue virus, proteins of foot- and mouth disease virus, bacterial membrane proteins, like for example PorA of Neisseria meningitides, oxidized low density lipoprotein, tumor antigens, tumor specific antigens, amyloid-beta, antigens related to rheumatoid arthritis, B-cell surface proteins CD19, CD20, CD21, CD22, proteins suitable for serving as target for immunocastration, proteins of hepatitis C virus (HCV), proteins of respiratory syncytial virus (RSV), proteins specific for non small cell lung carcinoma, malaria antigens, proteins of hepatitis B virus.

### Protocols and Procedures for Misfolding Proteins and Introducing Cross-Beta in Proteins

Peptides, polypeptides, proteins, glycoproteins, protein-DNA complexes, protein-membrane complexes and/or lipoproteins, in summary referred to as “protein” throughout this section, are misfolded with the occurrence of cross-beta structure after subjecting them to various cross-beta-inducing procedures. Below, a summary is given of a non-limiting series of those procedures, which are preferably applied to the proteins used in immunogenic compositions.

Misfolding of proteins with the occurrence of cross-beta is induced using selected combinations of several parameters. The following parameters settings are applied for proteins:
a. protein concentrations ranging from 10 μg/ml to 30 mg/ml, and preferably between 25 μg/ml and 10 mg/ml,
b. pH between 0 and 14, and preferably at pH 1.5-2.5 and/or pH 6.5-7.5 and/or 11.5-12.5 and or at the iso-electric point (IEP) of a protein, and for example induced with HCl or NaOH, for example using 2-5 M stock solutions.
c. NaCl concentrations between 0 and 5000 mM, and preferably 125-175 mM
d. buffer selected from PBS, HEPES-buffered saline (20 mM HEPES, 137 mM NaCl, 4 mM KCl, pH 7,4), or no buffer (H₂O),
e. a reducing agent like dithiothreitol (DTT) or β-mercaptoethanol is incorporated in the reaction mixture, and
f. temperature gradients and temperature end-points for an indicated time frame, that are applied for selected time frames of 10 seconds up to 24 h, and with selected ranges between 0 and 120° C., and preferably between 4 and 95° C., with preferably steps of 0.1-5° C./minute for gradients.

Furthermore, protein misfolding is induced for example by, but not limited to, post-translational modifications like for example glycation, using for example carbohydrates, oxidation, using for example CuSO₄, citrullination, using for example using peptidylarginine deiminases, acetylation, sulfatation, (partial) de-sulfatation, (partial) de-glycosylation, enzymatic cleavage, polymerization, exposure to chaotropic agents like urea (for example 0.1-8 M) or guanidinium-HCl (for example 0.1-7 M).

Misfolding of proteins with appearance of cross-beta is also achieved upon subjecting proteins to exposure to adjuvants currently in use or under investigation for future use in immunogenic compositions. Proteins are exposed to adjuvants only, or the exposure to adjuvants is part of a multi-parameter misfolding procedure, designed based on the aforementioned parameters and conditions. Non-limiting examples of adjuvants that are implemented in protocols for preparation of immunogenic compositions comprising cross-beta are alum (aluminium-hydroxide and/or aluminium-phosphate), MF59, QS21, ISCOM matrix, ISCOM, saponin, QS27, CpG-ODN, flagellin, virus like particles, IMO, ISS, lipopolysaccharides, lipid A and lipid A derivatives, complete Freund's adjuvant, incomplete Freund's adjuvant, calcium-phosphate, Specol.

A typical method for induction of cross-beta conformation in a protein is designed as follows in a matrix format, from which preferably subsets of parameter settings are selected.
i. protein concentration is 40/200/1000 μg/ml
ii. pH is 2, 7, 12 and at the IEP of the protein
iii. DTT concentration is 0 or 200 mM
iv. NaCl concentration is 0 or 150 mM
v. urea concentration is 0/2/8 M
vi. buffer is PBS or HBS (with adjusted NaCl concentration and/or pH, when indicated)
vii. temperature gradient is
   a. constantly at 4° C./22° C.-37° C./65° C. for an indicated time
   b. from room temperature to 65° C./85° C., for 1 to 5 cycles

Subsets of selected parameter settings are for example as follows.
A. 1 mg/ml protein in PBS, pH 7.3, 200 mM DTT, 150 mM NaCl, kept at 37° C. for 60 minutes
B. 200 μg/ml protein in PBS, 150 mM NaCl, heated in a cyclic manner for three cycli from 25° C. to 85° C., at 0.5° C./minute, with varying pHs.

Misfolding of E2. E2 protein is misfolded accompanied by introduction of cross-beta, by applying various parameter ranges, selected from described parameters a-f (see above). For example, E2 concentration ranges from 50 μg/ml to 2 mg/ml; selected pH is 2, 7.0-7.4 and 12; selected NaCl concentration is 0-500 mM, for example 0/50/150/500 mM; selected buffer is PBS or HBS or no buffer (H₂O); selected temperature gradient is for example as described for OVA, below. For example, E2 at approximately 300 μg/ml in PBS, heated in PCR cups in a PTC-200 thermal cycler (MJ Research, Inc.): 25° C. for 20 seconds and subsequently heated (0.1° C./second) from 25° C. to 85° C. followed by cooling to 4° C. for 2 minutes. This cycle is for example repeated twice (total number of cycles is 3). For example, E2 is subsequently stored at −20° C.

For the examples described below, non-treated E2 (nE2) at approximately 280 μg/ml in PBS was incubated at 25° C. for 20 seconds and was subsequently gradiently heated (0.1° C./second) from 25° C. to 85° C. followed by cooling at 4° C. for 2 minutes. This cycle was repeated twice and then, the E2 solution, referred to as cross-beta E2 (cE2) was stored at −20° C.

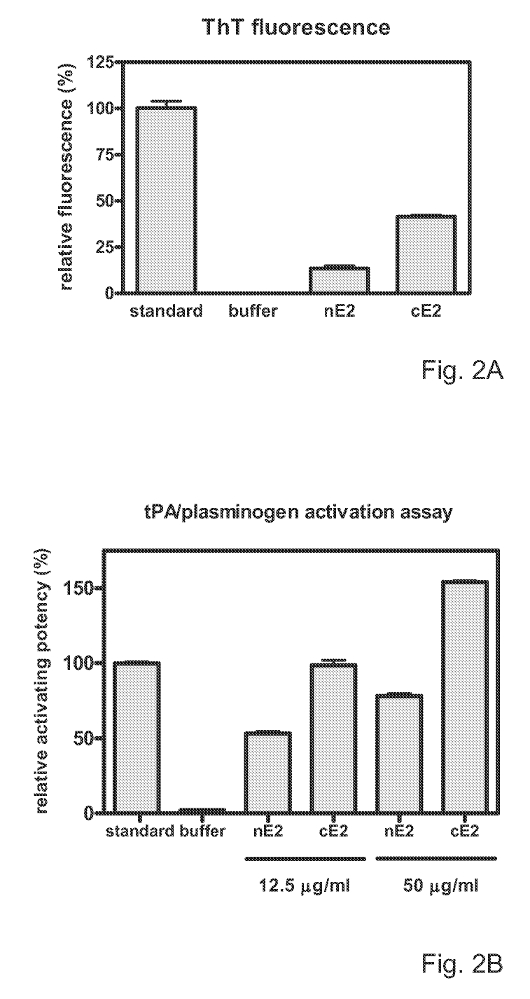

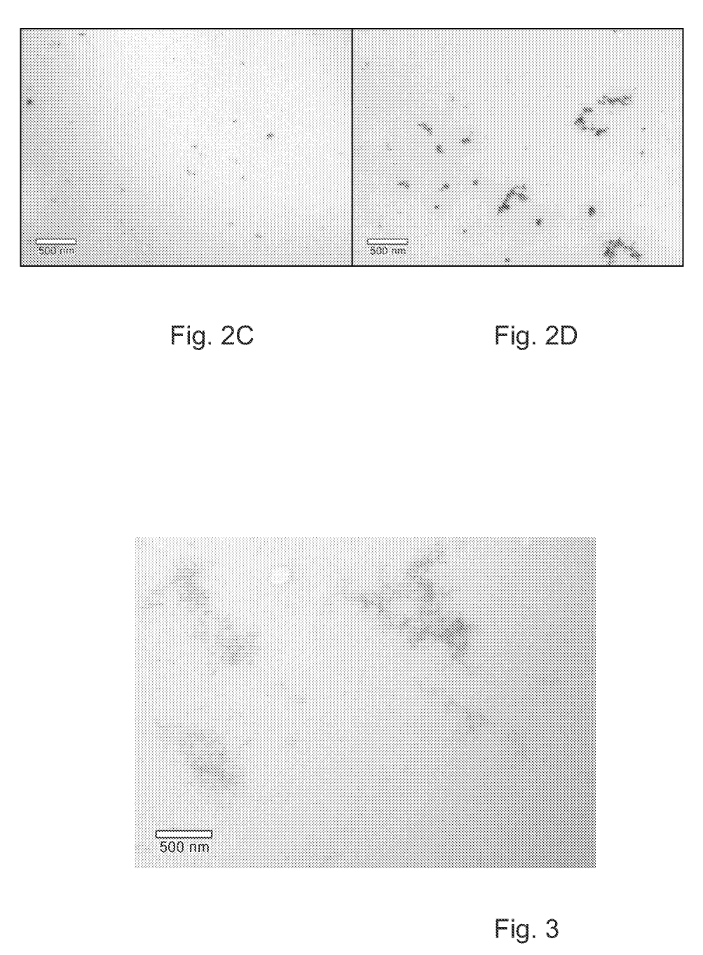

Structural differences and differences in cross-beta content between nE2 and cE2 were assessed using ThT fluorescence measurement, tPA/Plg activation analysis and TEM imaging. See FIG. 2. From these graphs and figures, it is clearly seen that the content of cross-beta in cE2 is increased when compared to nE2; both ThT fluorescence and tPA/Plg activating potential are increased. On the TEM images it is seen that cE2 appears as clustered and relatively large multimers with various sizes, whereas also nE2 displays assemblies of protein, though with smaller size and not clustered. Further analysis of cross-beta content and appearance, and further analysis of multimeric size and multimeric size distribution is assessed by subjecting the E2 samples to various of the aforementioned analyses for cross-beta determination and molecular structure and size determinations. Furthermore, various additional appearances of cE2 variants are generated by subjecting nE2 and/or nE2-FLAG-His to selected misfolding procedures as depicted above. For example, nE2 is used at 0.1 and 1 mg/ml, at pH 2/7/12, with/without DTT, for cyclic heat-gradients running from 4 to 85° C., for 1 to 5 cycles, resulting in 60 variants of cE2. These variants are subjected to analysis of binding of antibodies, for selecting those cE2 variants that combine the ability to bind functional antibodies (see below) with the presence of potent immunogenic cross-beta conformation. In addition, nE2 is for example coupled to dOVA standard and/or a different variant of misfolded OVA with proven potent cross-beta-adjuvating properties (see the section on OVA misfolding and OVA immunizations).

Misfolding of OVA. OVA is for example misfolded with introduction of cross-beta using the following misfolding procedures:
1. 10 mg/ml OVA in PBS, heating from 25 to 85° C., 5° C./minute
2. 1 mg/ml OVA in PBS, heating from 25 to 85° C., 5° C./minute
3. 0.1 mg/ml OVA in PBS, heating from 25 to 85° C., 5° C./minute
4. 10 mg/ml OVA in HBS, heating from 25 to 85° C., 5° C./minute
5. 1 mg/ml OVA in HBS, heating from 25 to 85° C., 5° C./minute
6. 0.1 mg/ml OVA in HBS, heating from 25 to 85° C., 5° C./minute
7. similar to the above six methods 1-6, now with a cooling step from 85 back to 25° C., and again heating to 85° C. (repeated twice)
8. similar to the above six methods 1-6, now with a heating rate of 0.1° C./minute, and a cooling step from 85 back to 25° C. (1-5 cycles)
9. addition of a final concentration of 1% SDS to 1 mg/ml OVA; incubation at room temperature for 30 minutes-16 h
10. addition of urea to 0.1-10 mg/ml OVA, to a final concentration of 2-8 M. Incubation for preferably 1-16 h at preferably 4-65° C. OVA solution is dialyzed against preferably H₂O or PBS or HBS, before further use.
11. constantly heating of preferably 0.1-10 mg/ml OVA in preferably PBS or HBS or H₂O, for preferably 1-72 h at preferably 4-100° C. For example 0.1 and 1 mg/ml in PBS, for 20 h at 65° C.
12. constantly heating of preferably 0.1-10 mg/ml OVA in PBS, for 10 minutes at 100° C. For example 0.1 and 1 and 10 mg/ml.
13. addition of a final concentration of 0.5% SDS to 1 mg/ml OVA; incubation for preferably 1-16 h at preferably 4-37° C., for example 1 h at room temperature.
14. Oxidation: addition of CuSO₄ to a final concentration of 1 mM and incubation for 24 h at 37° C. The oxidized OVA is dialyzed before further use.
15. incubation of 300 μg/ml OVA with 4 mM ascorbic acid, 40 μM CuCl₂, for 3 h, in NaPi buffer pH 7.4. Oxidation is stopped by adding EDTA from a 100 mM stock, to 1 mM final concentration. The oxidized OVA is dialyzed before further use.
16. pH of an OVA solution at 600 μg/ml in HBS is lowered to pH 2 by adding a suitable amount of HCl from a 5 M stock. The solution is subsequently kept at 37° C. for 30 minutes. Then, the pH is adjusted with NaOH to pH 7-7.4.
17. pH of an OVA solution at 600 μg/ml in HBS is raised to pH 12 by adding a suitable amount of NaOH solution from a 5 M stock. The solution is subsequently kept at 37° C. for 30 minutes. Then, the pH is adjusted with HCl back to pH 7-7.4.
18. For comparison with methods 16 and 17, the same final amount of NaCl is added, which is finally added to the solutions described in 16 and 17 by adding HCl/NaOH or NaOH/HCl, to OVA solution, after incubation for 30 minutes at 37° C.

OVA was subjected to the following misfolding procedure for inducing cross-beta conformation. OVA was dissolved in PBS to a concentration of 1.0 mg/ml. The solution was put on a roller device for 10 minutes at room temperature (RT), than 10 minutes at 37° C. in a water bath and subsequently again for 10 minutes on the roller device (RT). Then, 200 μl aliquots of OVA solution was heat-treated in a PTC-200 PCR machine (MJ Research) as follows: five cycles of heating from 30° C. to 85° C. at 5° C./minute; cooling back to 30° C. After five cycles misfolded OVA, termed dOVA, was cooled to 4° C. and subsequently stored at −80° C. This preparation of dOVA is used as a standard reference, termed “standard,” with cross-beta content that results in a maximal signal (arbitrarily set to 100%) in indicated cross-beta detecting assays, at a given concentration.

Cross-beta analyses are performed with dOVA standard at a regular basis in our laboratories. For example in FIGS. 2, 6, 7 and 10, dOVA standard is analyzed for its capacity to enhance ThT fluorescence, Congo red fluorescence, tPA/Plg activation. Furthermore, dOVA standard appears as clusters or strings of aggregated molecules with various sizes on TEM images (FIG. 3). Further cross-beta analyses and multimeric distribution analyses using described methods are applied to the dOVA standard preparation and to additionally produced misfolded OVA variants, as depicted above.

Misfolding of H5 of H5N1 strain A/HK/156/97. The H5-FLAG-His batch nH5-1, obtained after anti-FLAG antibody affinity chromatography and size exclusion chromatography, was subjected to two misfolding procedures.
A. A batch of 2 mg of nH5-1 (400 μg/ml in PBS, filtered through a 0.22 μm filter) was misfolded as follows. Aliquots of 120 μl of nH5-1 in PCR strips were incubated at 25° C. for 20 seconds and subsequently heated (0.1° C./second) from 25° C. to 85° C., followed by cooling at 4° C. for 2 minutes. This cycle was repeated twice. Then, the H5 sample was pooled and stored at 4° C., and referred to as “CH5-A.”
B. A second batch of 2 mg of nH5-1 was subjected to the following misfolding procedure. DTT was added from a sterile 1 M stock in H₂O to a final concentration of 100 mM. The sample was mixed by vortexing, and incubated for 1 h at 37° C. (stove). Subsequently, the H5 samples was dialyzed three times for ˜3 h against 31 PBS under sterile conditions, at 4° C. For dialysis, Slide-a-lyzers with a molecular weight cut-off of <10 kDa (Pierce) were used. The volume of the H5 sample, referred to as “CH5-B,” after recovery was unchanged with respect to the starting volume.

For structure analyses and for formulation of vaccine candidate solution, before use the nH5-1 and nH5-2 were centrifuged for 10 minutes at 16,000*g at room temperature. CH5-A and CH5-B were used without the centrifugation step.

The nH5-1 and CH5-B samples were analyzed on an analytical SEC column (U-Express Proteins, Utrecht, The Netherlands). For this purpose, approximately 80 μl of the 400 μg/ml stocks was applied to a Superdex200 10/30 column, connected to an Äkta Explorer (GE Healthcare). Running buffer was PBS. Samples were centrifuged for 20 minutes at 13,000*g before loading onto the column. The samples were run at a flow rate of 0.2 ml/minute and elution of protein was recorded by measuring absorbance at 280 nm.

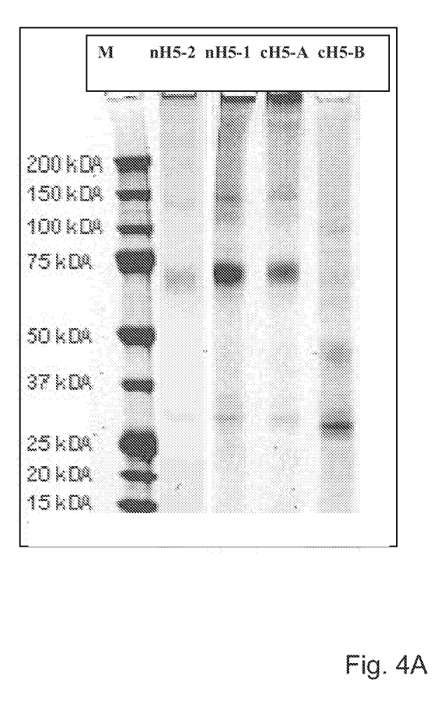

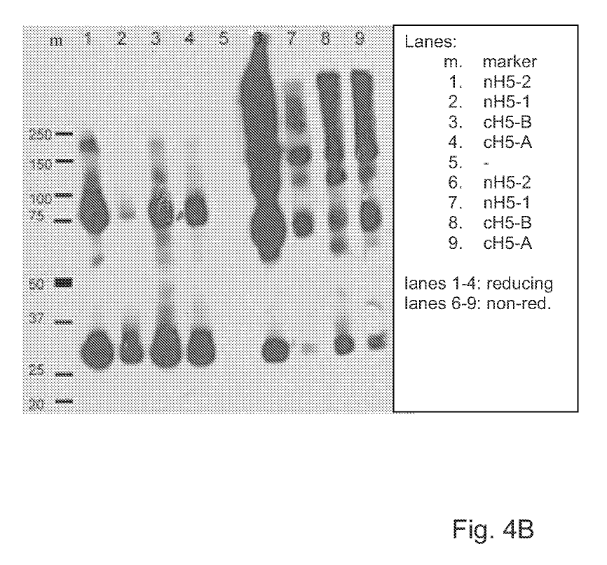

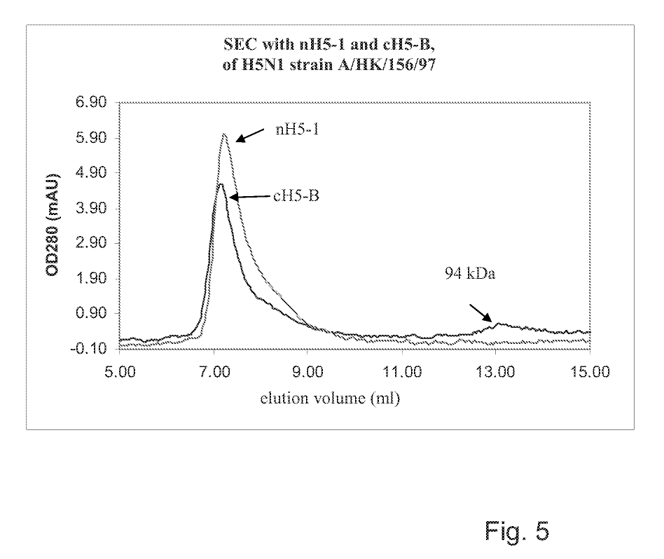

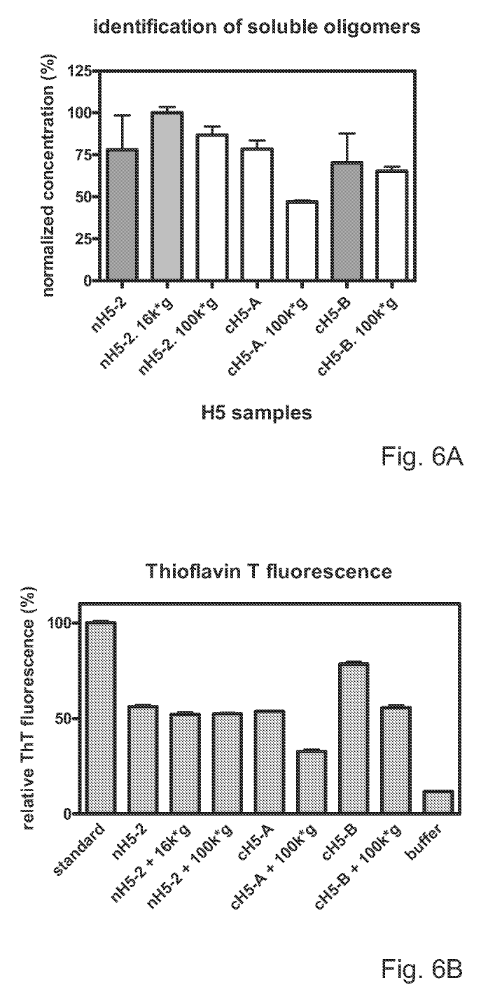

The nH5-1 and nH5-2 preparations appear on SDS-PA gel and Western blot as multimers ranging from monomer up till aggregates that do not enter the gel (FIG. 4). Upon treatment with DTT, these multimers monomerize, indicative for the covalent coupling of nH5 molecules through disulfide bonds (See FIG. 4B). The CH5-A preparation appears with a similar pattern on gel and blot compared to the non-treated variants (FIG. 4). In contrast, the CH5-B variant appears predominantly as monomers on gel and blot, with also dimers and oligomers present, but to a far lesser extent than seen in nH5-1, nH5-2 and CH5-A (FIG. 4). This observation is reflected in the elution patterns of nH5-1 and CH5-B from the SEC column, depicted in FIG. 5. The nH5-1 elutes as one peak in the flow-through of the column, whereas CH5-B elutes predominantly as a peak in the flow-through with a small peak at approximately the H5 monomer size. In conclusion, it appears that CH5-B comprises predominantly multimers that are more readily separated into smaller multimers and monomers, when compared to nH5-1, nH5-2 and CH5-A. Ultracentrifugation for 1 h at 100,000*g, which is used as a method to separate soluble oligomers of proteins from multimers that are precipitated in the pellet fraction, was applied to nH5-1, CH5-A and CH5-B (FIG. 6). It appears that when nH5-1 is subjected to the g-forces, no molecules that contribute to the ThT fluorescence are pelleted, indicative for the presence of soluble oligomers comprising cross-beta, and the absence of insoluble aggregates with cross-beta. In contrast, by applying 100,000*g for 1 h on CH5-A and CH5-B, a fraction of the ThT fluorescence enhancement is lost, indicative for the removal of insoluble multimers with cross-beta from the solution. The remaining fraction of both H5 variants apparently comprises soluble multimers with cross-beta conformation. TEM images of nH5-1, nH5-2 and CH5-A, as depicted in FIG. 6, show that all three H5 variants comprise multimers to a certain extent. The nH5-2 concentration is about 13-fold lower than the nH5-1 and CH5-A concentration, reflected in the lower density of multimers. When comparing nH5-1 and CH5-A, it is observed that CH5-A comprises less multimers but a higher number of larger multimers. These analyses of multimer size and size distribution are extended using more of the aforementioned techniques, and by incorporating more appearances of H5 after subjecting H5 solutions to various alternative misfolding procedures.

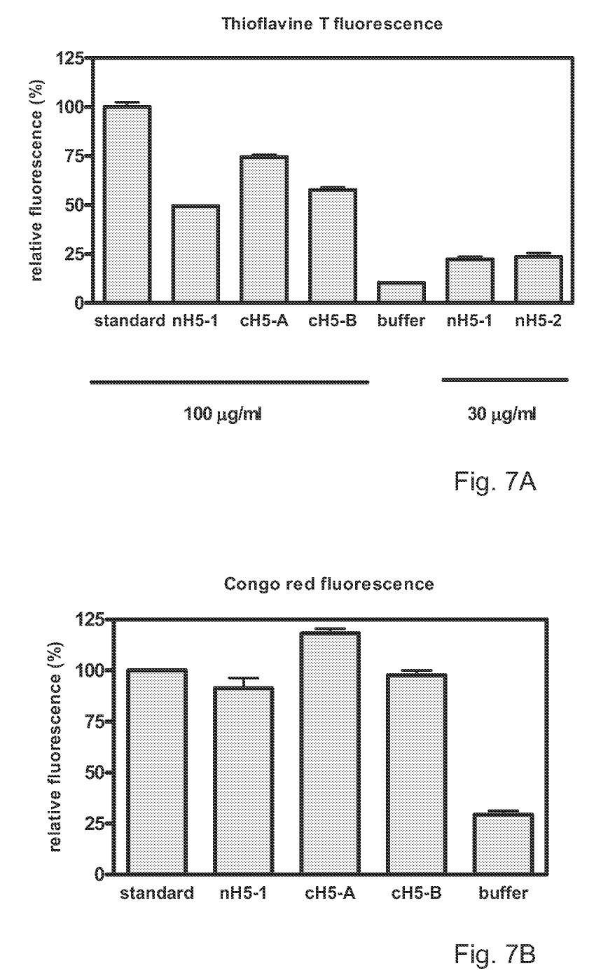

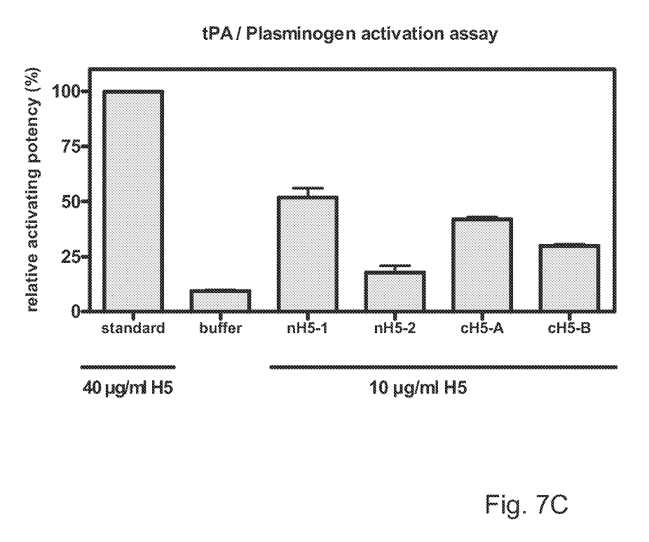

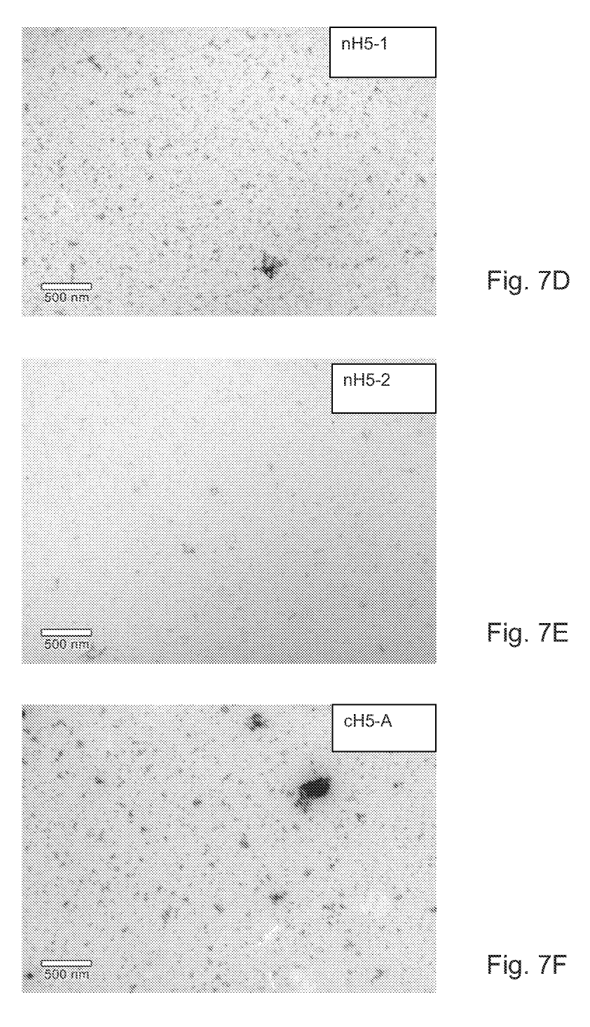

The nH5-1 and nH5-2 preparations comprise a considerable amount of cross-beta conformation, as depicted in FIG. 7, showing ThT fluorescence enhancement, Congo red fluorescence enhancement and the ability to increase tPA/Plg activity for both non-treated H5 variants. When comparing CH5-A with CH5-B it is clear that CH5-A displays higher signals in the three cross-beta detecting assays. When comparing the patterns of the signals obtained in the three assays with the four H5 variants, it is seen that all four variants display a unique combination of signals, indicating that four different appearances and/or contents of cross-beta are present. H5 variants are subjected to further cross-beta analyses in order to obtain more insight in the different appearances of cross-beta upon subjecting H5 to varying misfolding conditions.

Misfolding of H5 of H5N1 strain A/VN/1203/04. H5 of H5N1 strain A/VN/1203/04, as obtained from Protein Sciences, was subjected to four misfolding procedures, as indicated below.

1. nH5

For comparison, NaCl from a 5 M stock was added to non-treated H5 stock (922 μg/ml, 4° C., 150 mM NaCl), to a final concentration of 171 mM NaCl, and subsequently aliquoted in Eppendorf cups and stored at −20° C. Endotoxin level: <0.05 EU/10 μg/ml solution nH5 (determined using an Endosafe pts apparatus (Charles River). The solution was clear and colorless. For structure analyses and for formulation of vaccine candidate solution, before use the nH5 was centrifuged for 10 minutes at 16,000*g at room temperature.

2. CH5-1

Aliquots of nH5 in PCR strips (Roche) were incubated at 25° C. for 20 seconds and subsequently gradiently heated (0.1° C./second) from 25° C. to 85° C. followed by cooling back to 4° C., and kept at 4° C. for 2 minutes. This heat cycle was repeated twice. Then, aliquots in Eppendorf 500 μL cups were stored at −20° C. Code: “CH5-1.” The preparation CH5-1 was slightly turbid with some visible precipitates after heat treatment.

3. CH5-2

The pH of the nH5 stock kept at 4° C., was lowered to pH 2 by adding HCl from a 15% v/v stock. Then, aliquots of 100 μL/cup in PCR strips were heated in a PTC-200 thermal cycler, as follows. The samples were incubated at 25° C. for 20 seconds and subsequently gradiently heated (0.1° C./second) from 25° C. to 85° C. followed by cooling back to 4° C., and kept at 4° C. for 2 minutes. This heat cycle was repeated twice. Subsequently, the pH was adjusted to pH 7 by adding a volume NaOH solution from a 5 M stock. Then, aliquots in Eppendorf 500 μL cups were stored at −20° C. Code: “CH5-2.” The solution was clear and colorless.

4. CH5-3

The pH of nH5 kept at 4° C., was elevated to pH 12 by adding a volume NaOH solution from a 5 M stock. Then, aliquots of 100 μL/cup in PCR strips were treated as follows in a PTC-200 thermal cycler. The samples were incubated at 25° C. for 20 seconds and subsequently gradiently heated (0.1° C./second) from 25° C. to 85° C. followed by cooling back to 4° C., and kept at 4° C. for 2 minutes. This heat cycle was repeated twice. Subsequently the pH was adjusted to pH 7 by adding a volume HCl solution from a 5 M stock. Then, aliquots in Eppendorf 500 μL cups were stored at −20° C. Code: “CH5-3.” The solution was clear and colorless.

5. CH5-4

D-Glucose-6-phosphate disodium salt hydrate (g6p, Sigma; G7250) was added from a 2 M stock in PBS to nH5 to a final concentration of 100 mM g6p (20-fold dilution). Then it was incubated for 67 h at 80° C. The solution was intensively dialyzed against PBS, aliquoted in Eppendorf 500 μL cups, and stored at −20° C. The solution was light brown with white precipitates, visible by eye.

For structure analyses and for formulation of vaccine candidate solution, before use the nH5 was centrifuged for 10 minutes at 16,000*g at room temperature. CH5-1 to 4 were used without the centrifugation step.

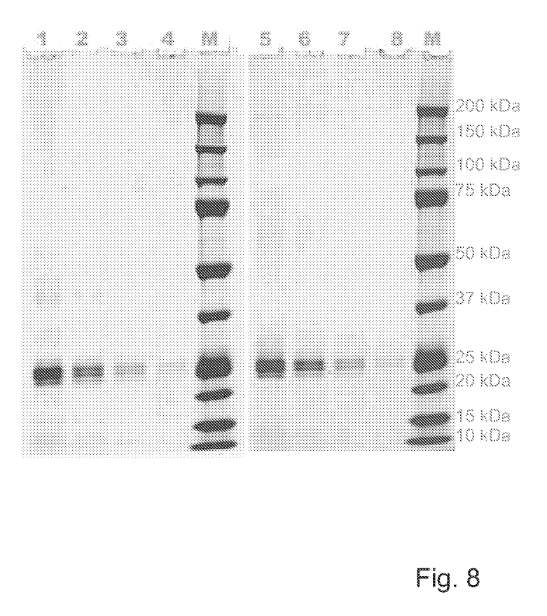

The nH5 protein, as purchased from Protein Sciences, appears predominantly as the approximately 25 kDa HA2 fragment, with a smaller content of HA0 (full-length H5) and HA1 (molecular weight approximately 50 kDa) on reducing and non-reducing SDS-PA gels, stained with Coomassie (FIG. 8).

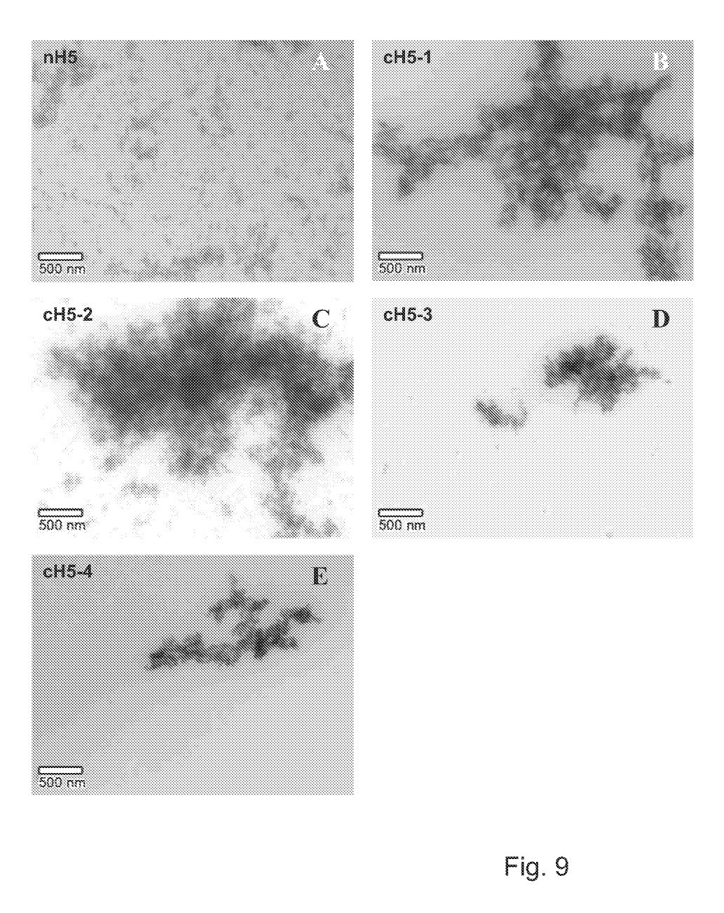

The nH5 appears on a TEM image as amorphous multimers which are relatively small in size and which tend to aggregate into clusters, as seen in the supernatant after 10 minutes centrifugation at 16,000*g (FIG. 9). In contrast, the four misfolded forms of H5, CH5-1 to 4, all appear as larger aggregates. The aggregates observed for CH5-1 and CH5-2 are similar in size and larger than the aggregates seen for CH5-3 and 4. Aggregates in CH5-2 seem to be more amorphous than the aggregates seen in CH5-2.

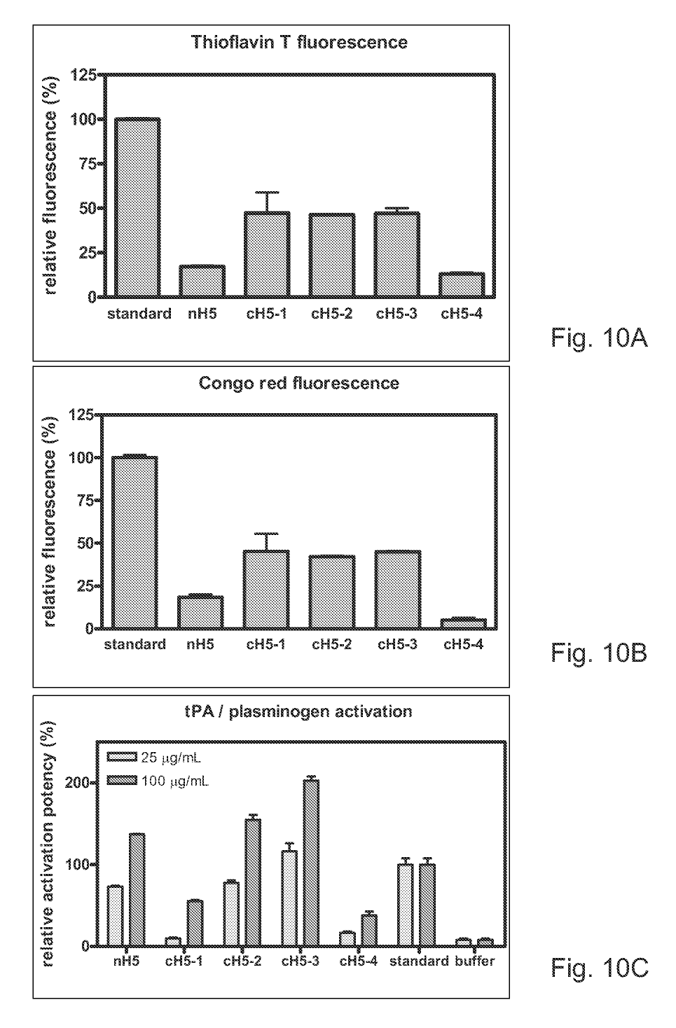

ThT fluorescence is enhanced with CH5-1 to 3, when compared to nH5 (FIG. 10A). The non-treated nH5 still displays a significant ThT fluorescent signal. The signal is decreased for CH5-4, when compared to nH5. A similar pattern is seen for Congo red fluorescence (FIG. 10B). The relative tPA/Plg activation potency of nH5 and CH5-1 to 4 displays a different pattern. CH5-2 and 3 enhance tPA/Plg activation to a somewhat larger extent than nH5, whereas CH5-1 and CH5-4 are less potent activators of tPA/Plg when compared to nH5 (FIG. 10C). The five H5 forms are subjected to extended cross-beta analyses and extended multimer size and distribution analyses, in order to obtain more detailed information about the structural appearances.

Misfolding of FVIII. FVIII is for example misfolded by using from the above listed spectrum of misfolding procedures parameter combinations as follows. Helixate sterile stock solution is preferably prepared according to the manufacturer's recommendations (100 IE/ml) and is subsequently used directly as freshly dissolved ingredient for immunogenic compositions, termed “FVIII” and numbered “9,” and used as non-treated FVIII.

For preparation of immunogenic compositions FVIII was subjected to the following procedures:

1) FVIII kept at 4° C. for 20 hours, in the dark, followed by storage at −80° C.→referred to as cross-beta FVIII-1 (cFVIII-1), or 1

2) FVIII kept at room temperature for 20 hours, in the dark, followed by storage at −80° C.→cFVIII-2, or 2

3) FVIII kept at 37° C. for 20 hours, in the dark, followed by storage at −80° C.→cFVIII-3, or 3

4) FVIII kept at 65° C. for 20 hours, in the dark, followed by storage at −80° C.→cFVIII-4, or 4

5) FVIII kept at 95° C. for 5 minutes, in the dark, followed by storage at −80° C.→cFVIII-5, or 5

6) FVIII with a pH lowered to pH 2, using a 5 M HCl stock, and kept at 65° C. for 20 hours, in the dark; the pH is raised to 7 afterwards by adding NaOH solution from a 5 M stock, followed by storage at −80° C.→cFVIII-6, or 6

7) FVIII with a pH raised to pH 12, using a 5 M NaOH stock, and kept at 65° C. for 20 hours, in the dark; the pH is lowered to 7 afterwards by adding HCl solution from a 5 M stock, followed by storage at −80° C.→cFVIII-7, or 7

8) FVIII dissolved freshly and subsequently stored at 4° C. for indicated times→cFVIII-8, or 8

9) freshly dissolved FVIII, used and analyzed within 8 hours after dissolving lyophilized sample→FVIII, or 9

Based on the aforementioned set of parameters a-f that parameters are preferably chosen for design of additional protein misfolding procedures, FVIII is for example misfolded in a selection of alternative ways. For example, FVIII is misfolded using prolonged incubation of FVIII at 4° C. and/or room temperature and/or 37° C., preferably in the dark. Alternatively, FVIII is for example subjected to exposure to 1-100 mM CuCl₂ for 1-16 hours at room temperature or 37° C., followed by dialysis against PBS.

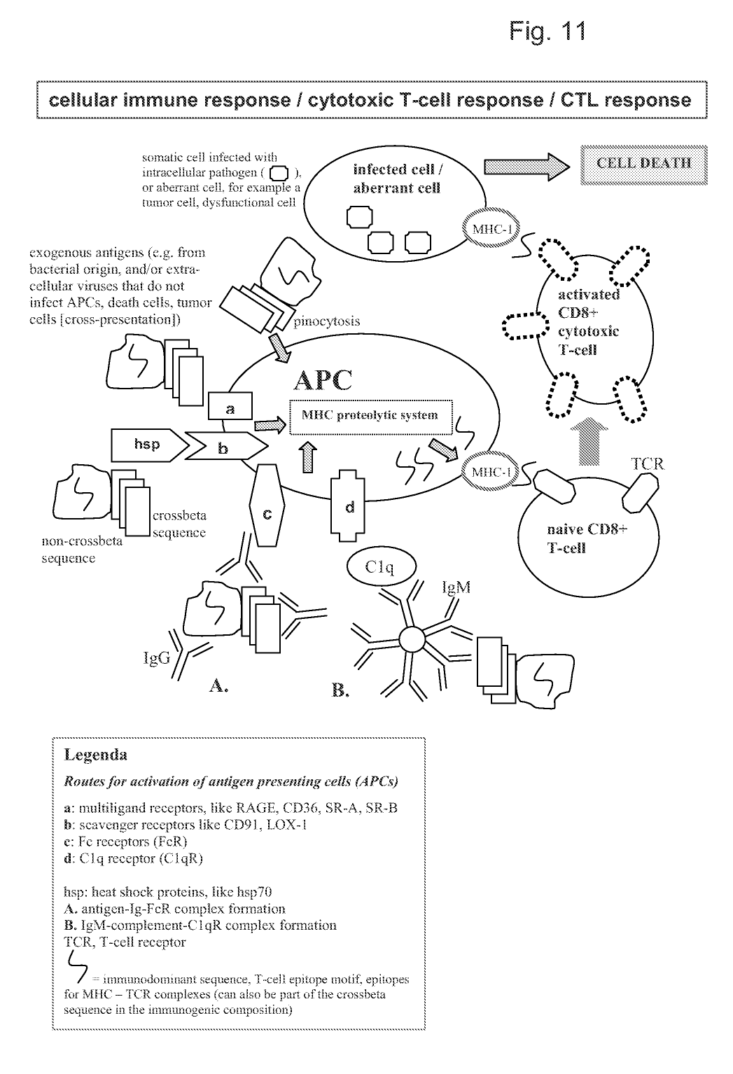

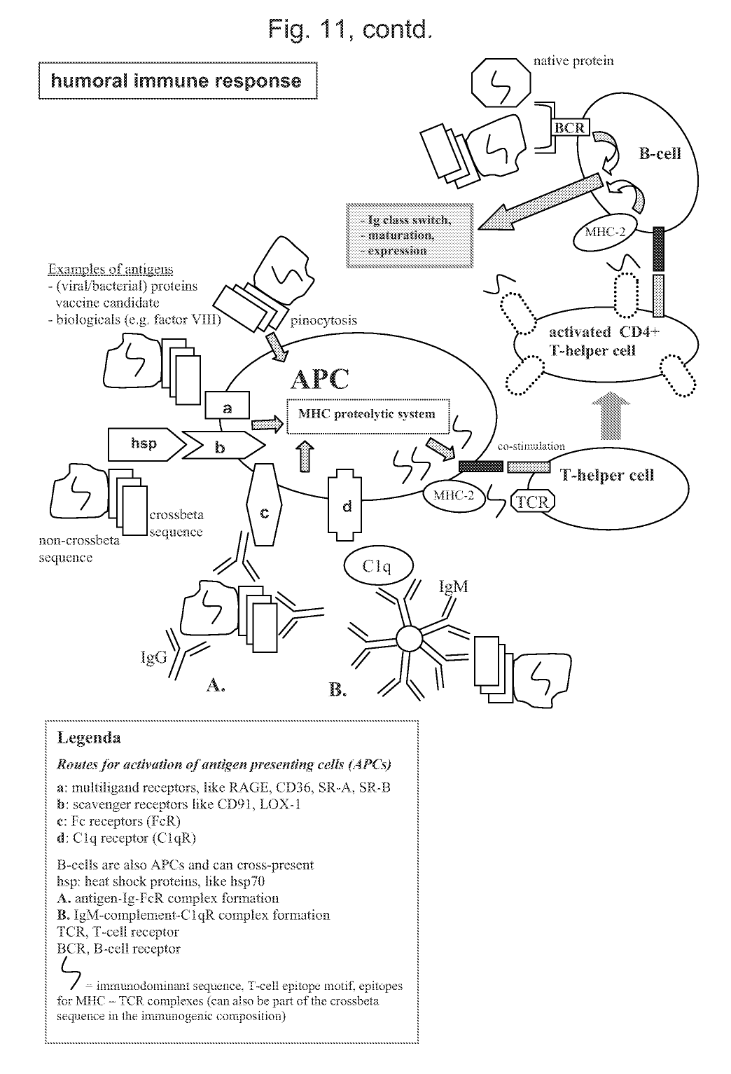

FVIII subjected to the misfolding conditions 1-8, giving FVIII variants cFVIII-1 to 8, were subsequently analyzed for the presence and extent of cross-beta conformation. For this purpose, ThT fluorescence enhancement, Congo red fluorescence enhancement and tPA/plasminogen activation were determined using two-fold diluted samples in the assay. See FIG. 11A-E. It is clearly seen that FVIII samples 4-6 comprise increased amounts of cross-beta, compared to FVIII (9), as shown in all three assays. cFVIII-7 shows values indicative for large conformational changes. Perhaps, cFVIII-7 is heavily aggregated, which is preferably assessed by TEM imaging and SEC, and/or is precipitated, perhaps to the wall of the vial, which is preferably assessed by protein quantification (BCA method, Coomassie+ method) and SDS-PAGE analysis with Coomassie stained gel. The FVIII variants cFVIII-1-3 and 8, and non-treated FVIII (9) all display similar extents of cross-beta content in the three assays. In addition, analysis of the relative presence of exposed hydrophobic patches on the FVIII molecules, as is preferably assessed by measuring ANS fluorescence (FIG. 11C), again shows that cFVIII-4 to 6 have a different conformation than FVIII. When comparing all four analyses, it is observed that cFVIII-3 perhaps also comprises slightly different amounts of cross-beta, when compared to FVIII.

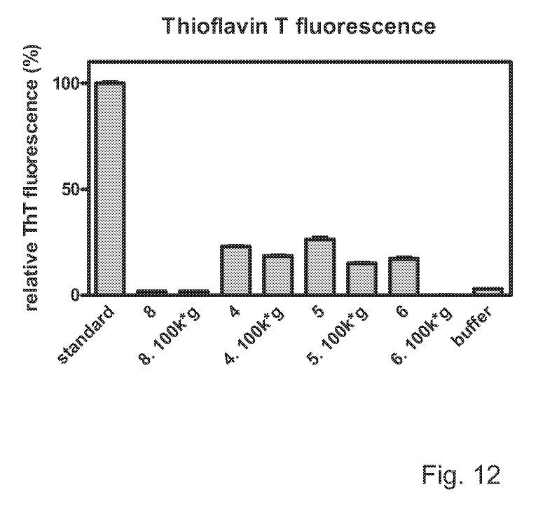

The FVIII samples 1-9 all appeared as clear and colorless solutions. In order to investigate whether soluble oligomers are present in the preparations, the FVIII solutions 4-6 and 8 were subjected to ultracentrifugation for 1 h at 100,000*g. As the, protein that remains in the supernatant after applying these g-forces to the solution is considered as “soluble oligomers,” including soluble monomers. After ultracentrifugation, ThT fluorescence was measured with two-fold dilutions of FVIII samples (FIG. 12). With sample 8, no difference is observed in ThT fluorescence before and after centrifugation. When FVIII samples 4, 5 and 6 are considered, ThT fluorescence intensity is decreased approximately 20, 45 and 100%, respectively. This shows that about these percentages of cross-beta conformation is present in insoluble oligomers, that are pelleted upon ultracentrifugation. The remaining cross-beta conformation, as indicated by the remaining ThT fluorescence intensity, is considered as being present in soluble FVIII oligomers.

To further analyze the structural aspects with respect to cross-beta formation and multimer size distribution, FVIII samples are preferably subjected to TEM imaging, ThS fluorescence analysis, bis-ANS fluorescence analysis, tPA binding ELISA, BiP binding ELISA, fibronectin finger 4-5 binding ELISA, IgIV binding ELISA, SDS-PAGE followed by Western blotting and/or Coomassie stain, circular dichroism analysis, analysis under a direct light microscope with 10-100× magnification, dynamic light scattering analysis, particle analysis in solution, and SEC analysis.

### Antibodies Suitable for Inclusion in Vanishing Epitope Scanning Strategies

For example, for selection of immunogenic compositions having a greater chance of being capable of eliciting a protective prophylactic immune response against infection with CSFV, for example strain Brescia 456610, in animals, for example in mice and/or in pigs, the following mouse monoclonal antibodies are implicated in the screenings.
CediCon CSFV 21.2,
CediCon CSFV 39.5 and
Cedicon CSFV 44.3,

purchased from Prionics-Lelystad, and which neutralize CSFV in vitro (information from the manufacturer). The antibodies are more preferably subjected to passive immunizations of animals, for example mice and/or pigs, followed by a challenge infection with CSFV, for example strain Brescia 456610. Then, antibodies that provide at least in part protection against the challenge viral infection are selected for selection of immunogenic compositions.

For example, for selection of immunogenic compositions having a greater chance of being capable of eliciting an immune response against a protein, for example OVA, in animals, for example in mice and/or in rabbits, the following mouse monoclonal antibodies and polyclonal antibodies are implicated in the screenings.
mouse HYB 099-01 (IgG1), 1 mg/ml, affinity purified; shows high affinity for native OVA and not for denatured OVA, according to the datasheet. The epitope specificity differs from that of HYB 099-02 and HYB 099-09, according to the datasheet.
mouse HYB 099-02 (IgG1), 1 mg/ml, affinity purified; shows high affinity for native OVA and not for denatured OVA, according to the datasheet. The epitope specificity differs from that of HYB 099-01 and HYB 099-09, according to the datasheet.
mouse HYB 099-09 (IgG1), 1 mg/ml, affinity purified; shows high affinity for native OVA and not for denatured OVA, according to the datasheet.
goat IgG fraction 55303, 5 mg/ml (MP Biomedicals)
rabbit IgG fraction 55304, 4 mg/ml (MP Biomedicals)

For example, for selection of immunogenic compositions having a greater chance of being capable of eliciting a protective prophylactic immune response against infection with influenza virus H5N1 strain A/VN/1203/04 or strain A/HK/156/97 in mice and/or in ferrets, the following mouse monoclonal antibodies, that are affinity purified, are implicated in the screenings.
a. Rockland anti-H5 A/VN/1203/04 catalogue number 200-301-975, 1 mg/ml (Tebu-bio 12467)
b. Rockland anti-H5 A/VN/1203/04 catalogue number 200-301-976, 1 mg/ml (Tebu-bio 12468)
c. Rockland anti-H5 A/VN/1203/04 catalogue number 200-301-977, 1 mg/ml (Tebu-bio 12469)
d. Rockland anti-H5 A/VN/1203/04 catalogue number 200-301-978, 1 mg/ml (Tebu-bio 12470)
e. Rockland anti-H5 A/VN/1203/04 catalogue number 200-301-979, 1 mg/ml (Tebu-bio 12471)
f. HyTest IgG2a clone 8D2, 3.2 mg/ml
g. HyTest clone 17C8, 6.7 mg/ml
h. HyTest IgG2a clone 15A6, 4.1 mg/ml

The anti-H5 antibodies purchased from Rockland (a-e) inhibit hemagglutination and neutralize H5N1 A/VN/1203/04 virus, according to the supplied datasheets. Antibodies purchased from HyTest (f-h) inhibit hemagglutination when H5N1 of the strains A/VN/1203/04 or A/HK/156/97 is used, according to information from the manufacturer. The antibodies are more preferably subjected to passive immunizations of animals, for example mice and/or ferrets, followed by a challenge infection with influenza virus, for example an H5N1 strain, most preferably the A/HK/156/97 strain and/or the A/VN/1203/04 strain. Then, antibodies that provide at least in part protection against the challenge viral infection are selected for selection of immunogenic compositions.

For example, for selection of FVIII compositions having a smaller chance of being capable of eliciting an undesired immune response against FVIII upon, for example intravenous, introduction to an animal, for example a human individual, antibodies which inhibit FVIII in coagulation assays are implicated in the screenings of FVIII compositions. These antibodies are for example monoclonal antibodies, for example from human or murine origin, and most preferably these monoclonal antibodies are of human origin when FVIII compositions are sought for human use. Alternatively, polyclonal antibodies are implicated in the selection. For example polyclonal antibodies in immune serum and/or plasma, for example of murine origin, and most preferably from human origin, are used when FVIII compositions are sought for human use. Antibodies which inhibit FVIII in coagulation assays are routinely determined in human plasma samples of Haemophilia patients, using for example the Bethesda assay, known to a person skilled in the art. For selection of FVIII compositions having a smaller chance of being capable of eliciting an undesired immune response against FVIII, those FVIII compositions are selected that show lowest or preferably no binding of the FVIII inhibiting antibodies, when cross-beta adjuvant is detected in the composition. Of course, most preferably, no cross-beta conformation is detected in FVIII compositions meant for therapeutic use, at all, thereby having at forehand a smaller chance of the FVIII composition being capable of eliciting an undesired immune response against FVIII, due to the absence of cross-beta adjuvant.

### Vanishing Epitope Scanning

For the detection of antibody binding, for example an ELISA setup is used. For example, the cross-beta antigen is preferably coated and subsequent the binding of the antibody is detected. Alternatively, the native protein is coated and detected with the antibody, and the ability of cross-beta antigens or immunogenic compositions comprising cross-beta conformation and epitopes for antibodies, to compete with this binding is tested. Preferably, in this setup such amount of antibody is used that results in approximately half-maximal binding. For example such analyses are performed as described in more detail below. In both ways for selecting immunogenic compositions, those cross-beta antigens or immunogenic compositions comprising cross-beta conformation and epitopes for antibodies are selected which have either lost certain amount of epitopes for the antibody or which have remained their epitopes.

E2

### Detection of Antibody Binding to Non-Treated E2 and Cross-Beta E2

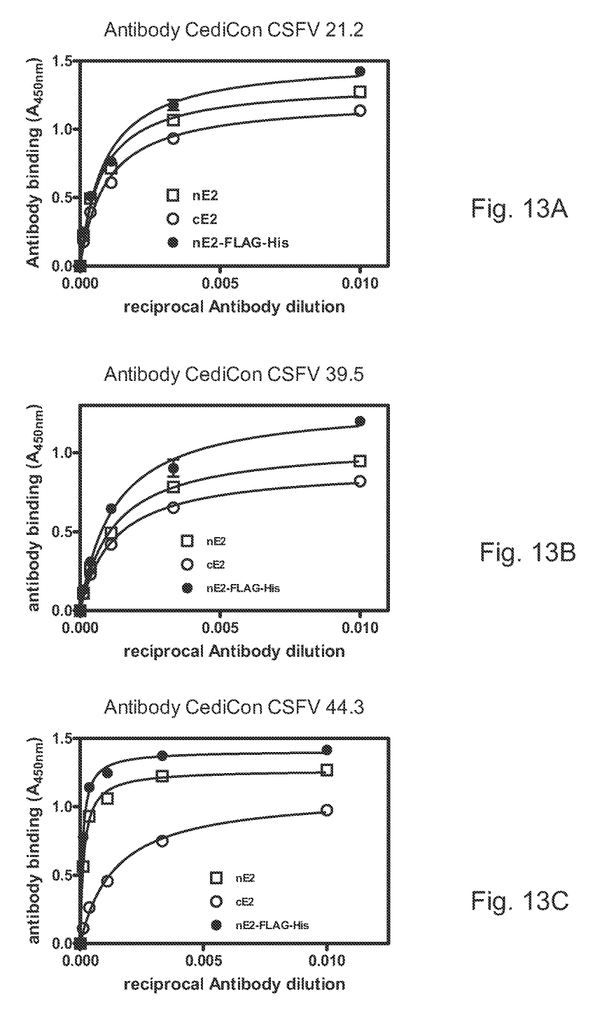

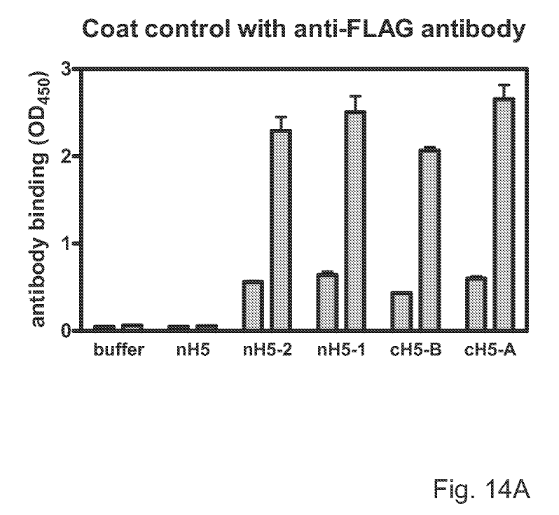

For analysis of relative binding of three mouse monoclonal horseradish peroxidase-labeled anti-E2 antibodies CediCon CSFV 21.2, CediCon CSFV 39.5 and Cedicon CSFV 44.3 (Prionics-Lelystad, The Netherlands) to non-treated E2 (nE2) and misfolded E2 comprising increased content of cross-beta (cE2), ELISAs were conducted. For this purpose, nE2, cE2 and nE2-FLAG-His were coated to Microlon high-binding plates (Greiner) and overlayed with dilution series of the three antibodies. For control purposes, non-coated wells were overlayed with the antibody dilutions as well, and E2-coated wells were overlayed with binding buffer only. See FIG. 13. It is observed that the increased cross-beta content of cE2 when compared to nE2 is accompanied by some decreased binding of antibodies 21.2 and 39.5, whereas binding of 44.3 is decreased to a relatively larger extent. Apparently, by inducing cross-beta conformation upon the subjected misfolding procedure still epitopes are exposed, that are recognized by the three antibodies. Since the three antibodies neutralize Brescia 456610 CSFV, cE2 is incorporated in an immunization trial with mice, as binding of the antibodies to antigen which comprises cross-beta adjuvant predicts that upon using cE2 as an antigen, protection against CSFV infection is inflicted.

### Immunization of Mice for Detection of Virus Neutralizing Antibodies

To analyze whether cE2 is inducing CSFV neutralizing antibodies in mice, the following immunization trial was conducted.

Start of the study: day −1. Four groups of five female BalbC mice were incorporated in the study. Blood was drawn at day −1 and 7, and is drawn at day 14, 21, 28 for preparation of serum. At day 28, the study is terminated and mice are sacrificed (final blood draw by heart puncture under anesthesia). Mice were immunized at day 0 with: group 1, placebo; group 2, 100% nE2, 3 μg/mouse; group 3, 100% cE2, 3 μg/mouse; group 4, 50% nE2+50% cE2, 1.5 μg nE2/mouse+1.5 μg cE2/mouse. Dose: 500 μl, 6 μg E2/ml in PBS, or PBS (placebo). At day 0 mice were immunized subcutaneously (s.c.) in the neck. At day 14, mice are immunized for a second time, using the same doses. Now, mice are immunized s.c. in the left flank. When the immunization is terminated after 29 days, most preferably the following analyses are conducted with the sera or plasma. Total IgG/IgM titers against nE2-FLAG-His are assessed for sera or plasma of each individual mouse and for pooled sera or plasma for each of the four groups. In addition, for each individual serum and for pooled serum for each group, IgG1 and IgG2a titers are determined as a measure for the occurrence of a humoral response and/or a cellular response. Virus neutralization titers using CSFV strain Brescia 456610 are also conducted to analyze the relative virus neutralizing capacity amongst in sera or plasma of the four groups of mice. Finally, the ability of the dilution series of the sera or plasma to compete for binding of the antibodies CediCon CSFV 21.2, CediCon CSFV 39.5 and Cedicon CSFV 44.3 to nE2-FLAG-His immobilized on an ELISA plate is assessed. In this way information is gathered on whether antibodies induced in mice upon immunization with nE2 and cE2 recognize the same or similar epitopes compared to those epitopes recognized by CediCon CSFV 21.2, CediCon CSFV 39.5 and Cedicon CSFV 44.3, and to which relative extent nE2 and cE2 induce antibodies. In this way, data is collected that is compared to the data obtained with the series of cross-beta measurements and to the data obtained with the series of multimer size and distribution measurements.

A typical challenge experiment with CSFV in pigs, after immunization with immunogenic compositions comprising cross-beta adjuvant and exposed epitopes for functional antibodies, is for example conducted as follows. For example, a vaccination-challenge experiment is conducted with five groups of for example 3-9 pigs, and preferably 5-6 pigs, for example approximately 6 weeks of age at the start of the experiment. Blood is drawn at day −1, for collection of pre-immune serum. All pigs are clinically observed each day, throughout the whole study period. For control purposes, E2 vaccine is prepared according to the procedures applied to E2 to obtain the commercially available water-in-oil-in-water CSFV vaccine. At day 0, pigs are immunized intramuscularly. Typical immunogenic compositions consist of: group 1, placebo; group 2, non-treated E2; group 3, cross-beta-adjuvated E2 with exposed epitopes for antibodies; group 4, cross-beta-adjuvated E2 lacking exposed epitopes for antibodies; group 5, non-treated E2+cross-beta-adjuvated E2 with exposed epitopes for antibodies. Blood is drawn for serum preparation at day 7, 14, 21, 28, 25, 42. A second immunization is performed at day 21. Virus neutralization tests are performed with serum collected at day −1/7/14/21/28/35, and CSFV strain Brescia 456610. Rectal temperature is measured from day 40 on, at each day of the remaining period of the study. Furthermore, signs of CSF like anorexia and paresis are noted. At day 42, all pigs are challenged intranasally with CSFV strain Brescia 456610. From day 42 on, for 15 days up till day 56 of the study, the pigs are monitored daily with respect to the following parameters: leucopenia test, thrombocytopenia test. In addition, virus secretion is measured with samples collected for example at day 42/44/47/49/51/54/56. Pigs are euthanized in case of critical illness. Virus content of white blood cells is for example assessed with samples collected at day 42/44/47/49/51/54/56.

OVA

The series of OVA variants obtained by subjecting OVA to the misfolding procedures outlined before are analyzed for their type and relative content of cross-beta appearance, their multimeric size and multimer distribution, and their relative ability to bind the antibodies as described above. Based on combinations of cross-beta appearance and content, and multimer size, cross-beta dOVA variants are subjected to analyses for binding of monoclonal and/or polyclonal antibodies. Based on these analyses, OVA variants are selected that combine the occurrence of cross-beta in the context of a multimer size of preferably the size of a monomer, up to the size of multimers with dimensions of for example in the range of 1-10 μm, and more preferably a multimer size of monomers up to 1000-mers, with the binding of antibodies or with inhibited antibody binding or the lack of antibody binding. This selected series of OVA variants is then used as immunogenic composition in immunization trials in animals, preferably in mice. Subsequently, in sera or plasma the presence of anti-OVA antibodies is analyzed. In addition, the ability of the antibodies in the sera or plasma to compete for binding of the monoclonal antibodies that only bind native OVA and not denatured OVA, to native OVA is assessed. For this purpose, the monoclonal antibodies are preferably tagged or labeled, for example with biotin, peroxidase or alkaline phosphatase. Most preferably, a series of OVA variants is selected for the immunizations, that span the parameter windows to a large extent. For example, OVA variants with no or extreme large cross-beta content are selected. For example, OVA monomers up to large aggregates visible by eye are selected, with OVA variants comprising various multimer sizes in between. For example, OVA variants that display as high-affinity binding partners for the antibodies are incorporated in the immunization studies, as well as OVA variants that expose antibody epitopes to an intermediate extent, and as well as OVA variants that do not expose antibody epitopes at all.

H5 of H5N1 strain A/HK/156/97

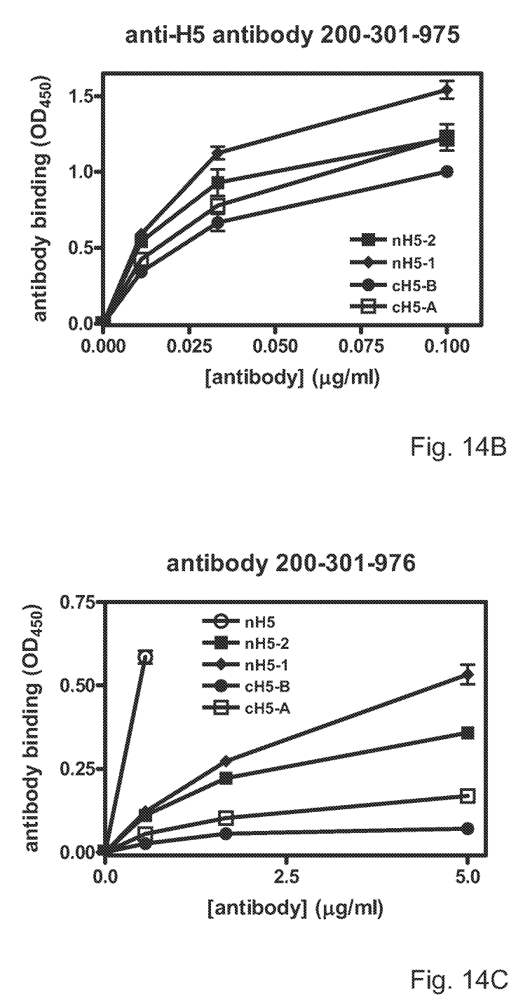

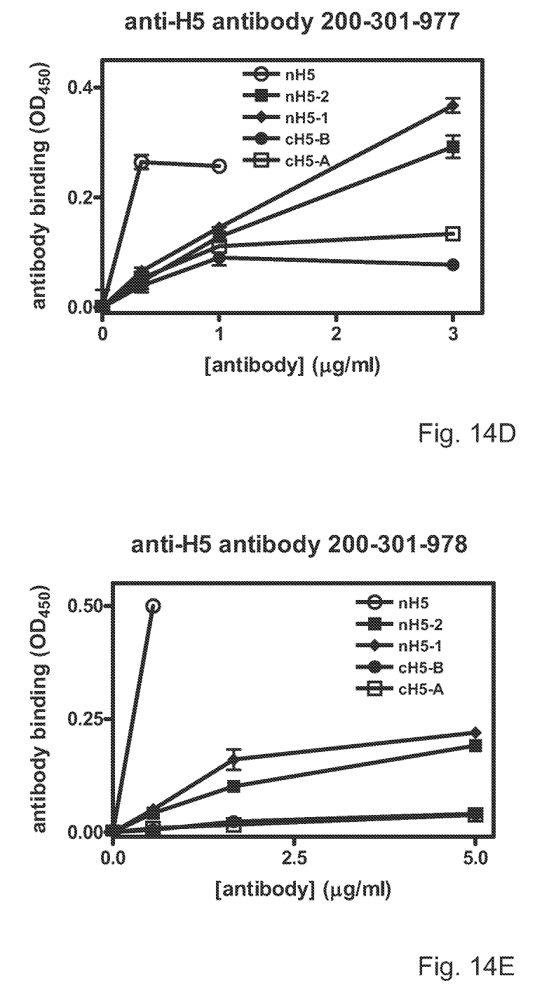

The four variant of H5 of H5N1 virus strain A/HK/156/97 comprise varying cross-beta contents and multimer size distributions. The nH5-1, nH5-2, CH5-A and CH5-B variants are subjected to antibody binding analyses in ELISAs, using four mouse monoclonal antibodies 200-301-975 to -978 (Rockland). These antibodies are raised against H5N1 A/VN/1203/04, neutralize virus of this strain, and inhibit hemagglutination induced by this virus. In FIG. 14 it is shown that the four antibodies bind to different extents to the four H5 variants originating from H5N1 A/HK/156/97. A similar pattern is seen with nH5 (A/VN/1203/04). For all four antibodies tested, binding to CH5-A and CH5-B is decreased when compared to the two non-treated H5 variants nH5-1 and nH5-2. The four H5 variants are subjected to a vaccination experiment with mice, followed by a challenge with H5N1 A/HK/156/97. An example of a vaccination experiment with immunogenic compositions comprising cross-beta-adjuvated H5 and H5 molecules that expose epitopes for antibodies that have the capacity to inhibit virus induced hemagglutination and to neutralize virus, is depicted below.

Nine groups of 8 female Balb/c mice are included in the experiment. Pre-immune serum is collected before the first immunization, and serum is collected four times more between one week after the first immunization and the day of the viral challenge (day 42). Mice are immunized subcutaneously at day 0 and day 21 with doses of 500 μl/mouse, according to the following scheme of test items per group:
1. placebo, PBS
2. 1 μg/mouse nH5-2
3. 5 μg/mouse nH5-1
4. 5 μg/mouse CH5-A
5. 5 μg/mouse CH5-B
6. 1 μg/mouse nH5-2+4 μg/mouse nH5-1
7. 1 μg/mouse nH5-2+4 μg/mouse CH5-A
8. 1 μg/mouse nH5-2+4 μg/mouse CH5-B
9. H5N₂ Nobilis flu (Intervet).

In the period before the challenge, mice are clinically observed daily, and putative occurrence of injection site reactions is monitored twice to thrice a week. At day 42, mice are inoculated with H5N1 virus of strain A/HK/156/97. From day 41 till the end of the study at day 56, mice are clinically observed for clinical signs of influenza, and body weight is measured daily. Serum is analyzed for the presence of virus neutralizing antibodies, using H5N1 A/HK/156/97, and hemagglutination inhibition titers are determined. Total IgG/IgM titers are determined using ELISA with non-treated H5 of H5N1 A/HK/156/97 and/or using H5 of H5N1 A/VN/1203/04. In addition, IgG1 and IgG2a titers are determined. Finally, the capacity of the anti-H5 antibodies in the sera or plasma to compete for binding of the series of monoclonal anti-H5 antibodies listed above is assessed in competition ELISAs. These listed antibodies neutralize H5N1 and inhibit hemagglutination by H5N₁. Most preferably, antibodies that provide protection against H5N1 infection upon passive vaccination, are used for the ELISAs. For the ELISAs, biotinylated mouse monoclonal antibodies are used. Serum dilution series are prepared with biotinylated anti-H5 antibodies incorporated in the dilution series at a concentration that gives sub-optimal binding when assessed in the absence of immune serum. In the ELISA, binding of biotinylated anti-H5 antibody is determined using Streptavidin.

H5 of H5N1 strain A/VN/1203/04

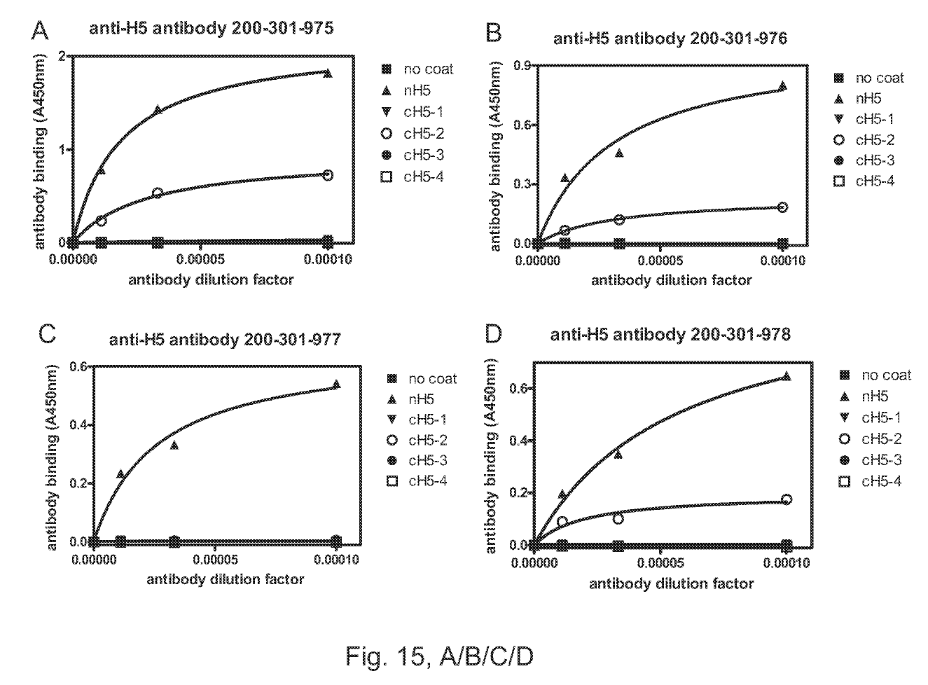

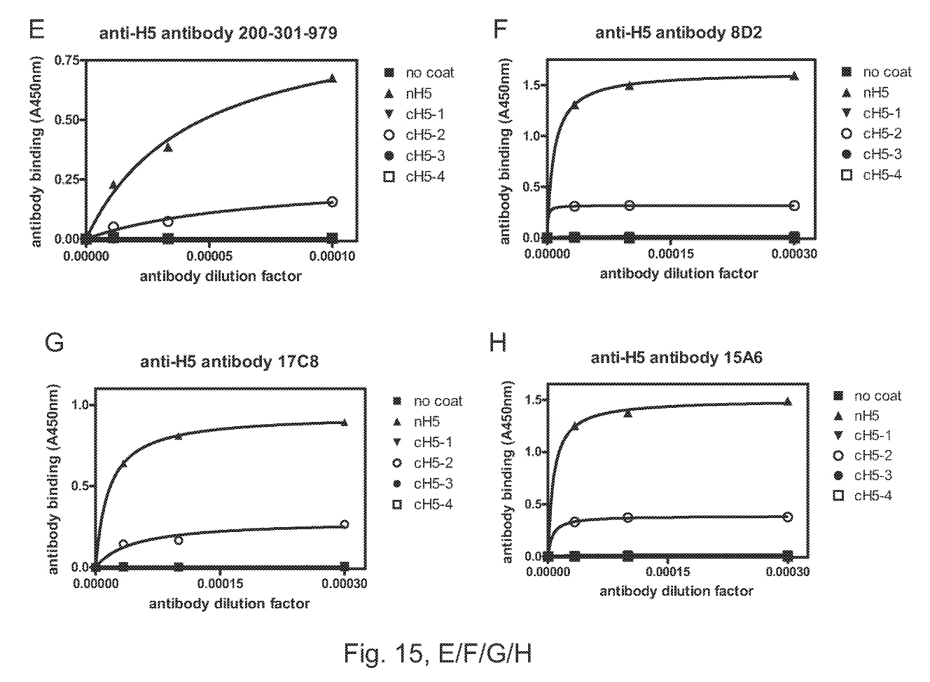

As described above, non-treated H5 of H5N1 strain A/VN/1203/04 comprises various appearances upon subjecting nH5 to four different misfolding procedures. Cross-beta parameters differ amongst CH5-1 to 4, as well as the size and shape of multimers, as seen on TEM images (FIG. 9). Now, the binding of eight mouse monoclonal anti-H5 antibodies is assessed in an ELISA. Antibodies used for this analyses are depicted above and include 200-301-975 to -979 (Rockland) and 8D2, 17C8 and 15A6 (HyTest). These first five antibodies neutralize H5N1 A/VN/1203/04, and all eight antibodies inhibit H5N1 induced hemagglutination, according to the supplied datasheets. In FIG. 15 binding of dilution series of the eight antibodies is shown for the five H5 variants. It is clearly seen that nH5 exposed most epitopes for all eight antibodies. Furthermore, CH5-1, -3 and -4 do not show any antibody binding with all eight antibodies, with the applied parameter settings. In contrast, seven out of the eight tested antibodies bind to CH5-2. The number of binding sites is reduced when compared to nH5.

Non-treated H5 of H5N1 A/VN/1203/04 and misfolded variants that comprise cross-beta structure and exposed epitopes for functional antibodies, in the context of a multimer size suitable for immunizations, are for example implicated in vaccination trials followed by viral challenge in, for example, ferrets and/or mice. Such a vaccination trial is for example performed similarly to the protocol described for H5 of H5N1 A/HK/156/97, above. Similar parameters are analyzed.

Factor VIII

FVIII ELISA with Haemophilia Patient Plasma

Certain Haemophilia patients suffer from a qualitative shortens and/or a quantitative shortens of functional FVIII, resulting in a mild to severe bleeding tendency. As a therapeutic approach, patients receive intravenous injections with recombinant and/or plasma-derived human FVIII and/or FVIII derivatives, like for example FVIII lacking the B-domain. A drawback of this treatment approach is the induction of anti-FVIII (auto-)antibodies, also referred to as inhibitor formation, which occurs in approximately 5-30% of the patients, and which hampers effective further treatment of the underlying disease. In patent applications US2007015206, the contents of which are incorporated herein by this reference, and WO2007008070 we disclosed that proteins comprising cross-beta structure elicit an immune response due to the adjuvating properties of cross-beta conformation. Furthermore, in patent application US2007015206 we disclosed that a series of biopharmaceuticals, including FVIII, comprise protein with cross-beta conformation to various extents. In patent applications US2007015206 and WO2007008070 we demonstrated that proteins comprising cross-beta structure, being it either biopharmaceuticals, or viral proteins used as vaccine candidates, in fact induce an immune response directed to natively folded counterparts of the proteins comprising cross-beta. This demonstrates that protein formulations that comprise cross-beta harbor the risk for eliciting antibody titers directed against the native, functional protein molecules. That is to say, misfolded FVIII protein molecules in FVIII formulation meant for therapeutic use are contributing to the observed built up of an immune response against FVIII in haemophilia patients.

In the current example we demonstrate that a series of misfolded forms of human FVIII comprise molecules with cross-beta conformation and in addition harbor epitopes for anti-FVIII antibodies present in plasma from Haemophilia patients suffering from FVIII inhibiting anti-FVIII antibodies.

Protocol for Anti-FVIII Titer Determination in Haemophilia Patient Plasma, Using ELISA

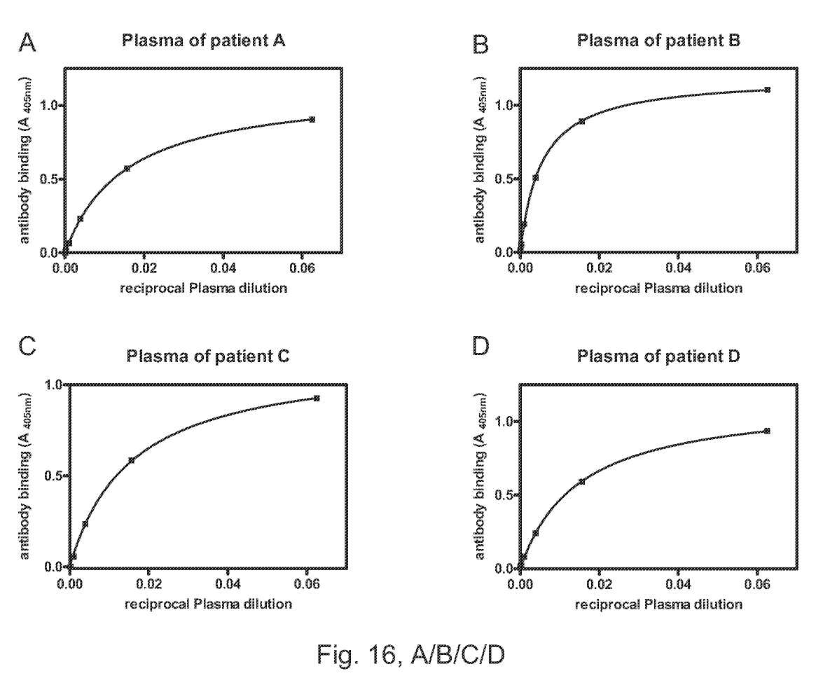

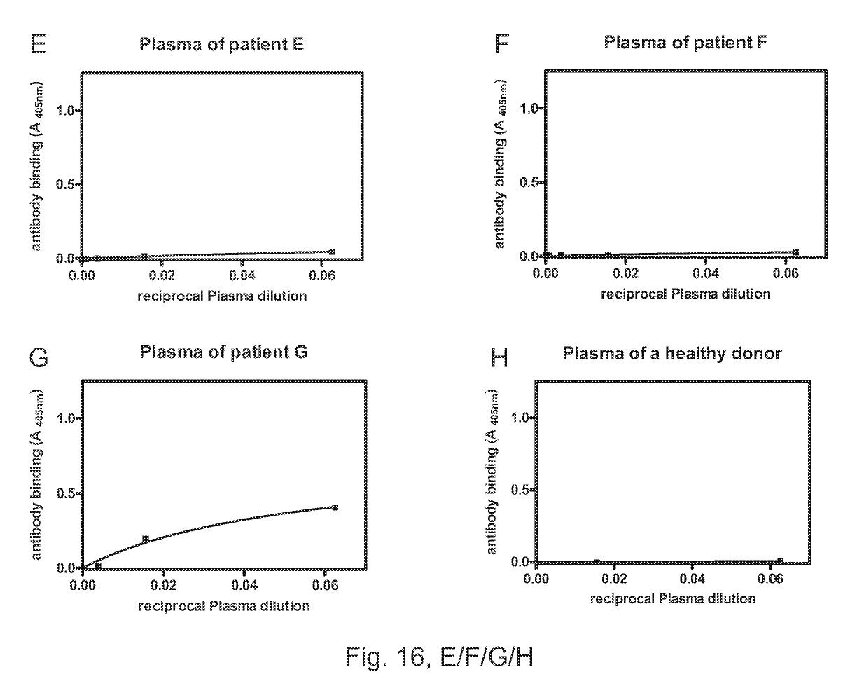

Anti-FVIII titers were determined in a fourfold dilution series starting from 1:16, to 1:65536, of plasma from seven haemophilia patients (kind gift of the University Medical Center Utrecht, Utrecht, The Netherlands). Patients A-D had tested positive in a Bethesda type of assay for anti-FVIII antibodies that inhibit FVIII, whereas patients E-G had tested negative. Plasma of one healthy donor was incorporated in the ELISAs as an additional negative control. Helixate FVIII was used as the coated antigen in the ELISAs, and was coated at 10 IE/ml, in 100 mM NaHCO₃, pH 9.6, on Microlon high-binding 96-wells plates (Greiner). Wash buffer was 50 mM Tris, 150 mM NaCl, 0.1% v/v Tween20, pH 7.0-7.4. Binding buffer for the plasma dilutions and secondary antibody was PBS with 0.1% v/v Tween20. FVIII was coated at room temperature, for 1 h, with agitation (50 μl/well). After washing, 200 μl/well Blocking Reagent (Roche) was incubated for 1 h at 4° C., with agitation. After 3 washes, the fourfold plasma dilutions series of the eight indicated plasma's (patients A-G, control donor) was incubated for 1 h at 4° C., with agitation, with 50 μl/well. After 3 washes, 50 μl/well of 1:3000 diluted Goat-anti human IgG (GAHAP-IgG; Biosource Int., catalogue number AHI0305) was incubated for 30 minutes at 4° C., with agitation. After 5 washes and subsequently two more washes with PBS only, bound GAHAP-IgG was visualized using 100 μl/well DEA-NPP-substrate (p-nitrophenyl phosphate (600 μg/ml) in DEA buffer pH 9.8 (10% v/v diethanolamine in H₂O, with 240 μM MgCl₂.6H₂O, pH adjusted with HCl)) for 1.0 minutes at room temperature, before adding 50 μl/well 2.4 M NaOH to stop the reaction. Absorbance was read at 405 nm on a Spectramax Plus384 Microplate Reader (Molecular Devices). See FIG. 16. From the titration curves it was derived that plasma diluted 1:100 or 1:200 results in signals suitable for the next series of analyses; comparison of anti-FVIII antibody binding from haemophilia patient plasmas that tested positive for the presence of FVIII inhibiting antibodies, to non-treated FVIII and various forms of FVIII subjected to misfolding conditions. Apparently, patient G has elicited antibodies against FVIII, but these antibodies are not inhibiting FVIII.

Comparison of Anti-FVIII Antibody Binding from Hemophilia Patient Plasmas that Tested Positive for the Presence of FVIII Inhibiting Antibodies, to Non-Treated FVIII and Various Forms of FVIII Subjected to Misfolding Conditions

See for the nine different forms of FVIII that were included in these examples the section above: Misfolding of FVIII, and FIG. 11 for an overview of the relative amounts of cross-beta amongst the FVIII variants. From the cross-beta analyses it is concluded that FVIII subjected to misfolding procedures 4-6 comprises an increased content of cross-beta conformation (cFVIII-4 to 6), when compared to FVIII. Further, detailed structural analyses are performed by measuring for example ThS fluorescence, ANS fluorescence, circular dichroism, binding of tPA, factor XII, BiP, IgIV and finger domains of fibronectin in ELISAs. Furthermore, FVIII variants are for example subjected to SEC analyses and particle size analyses using for example TEM imaging and ultracentrifugation.

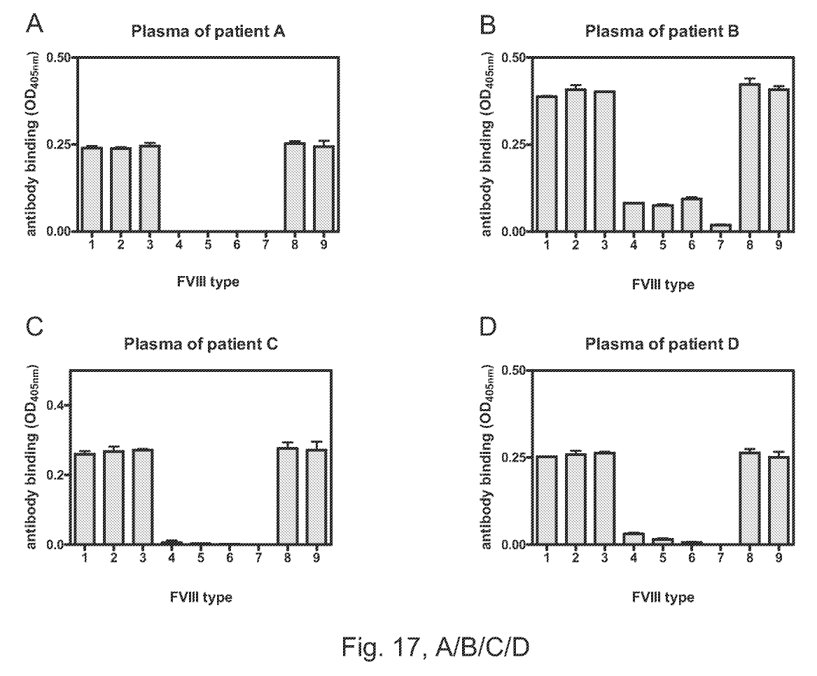

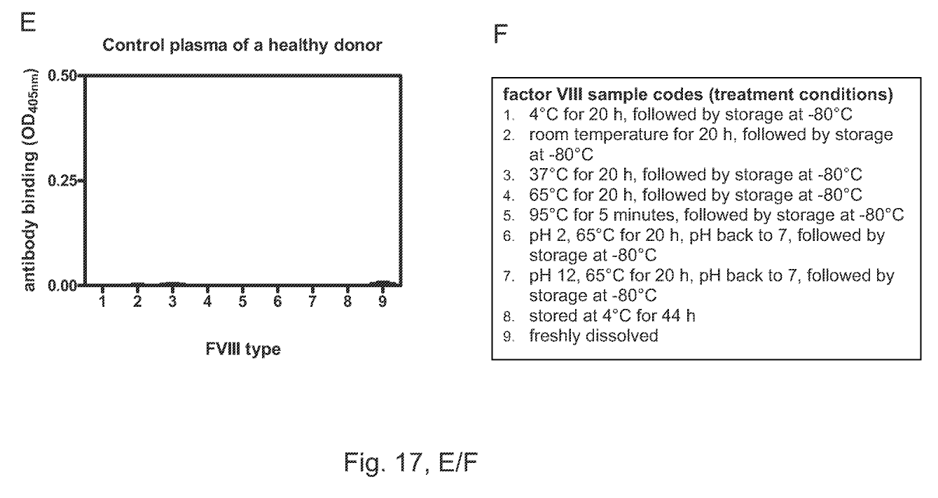

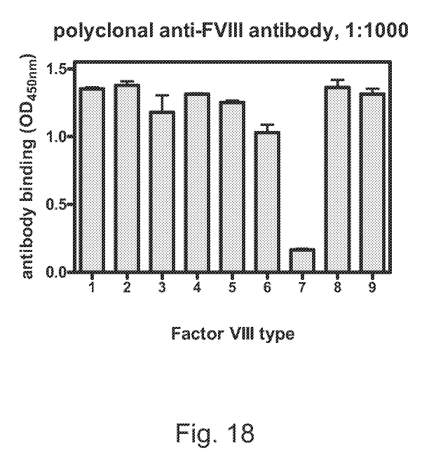

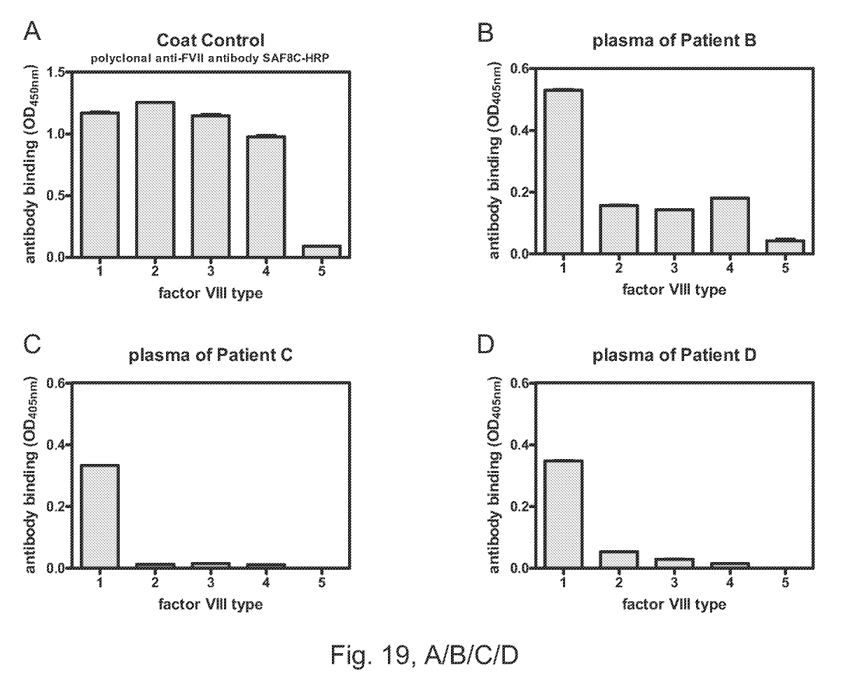

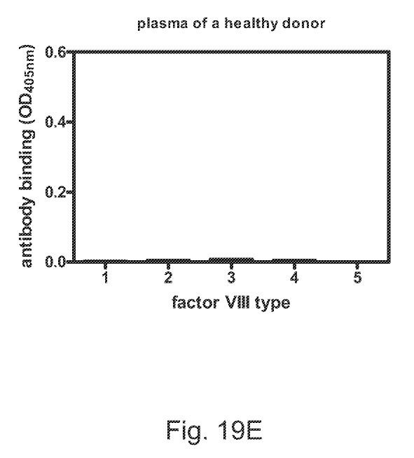

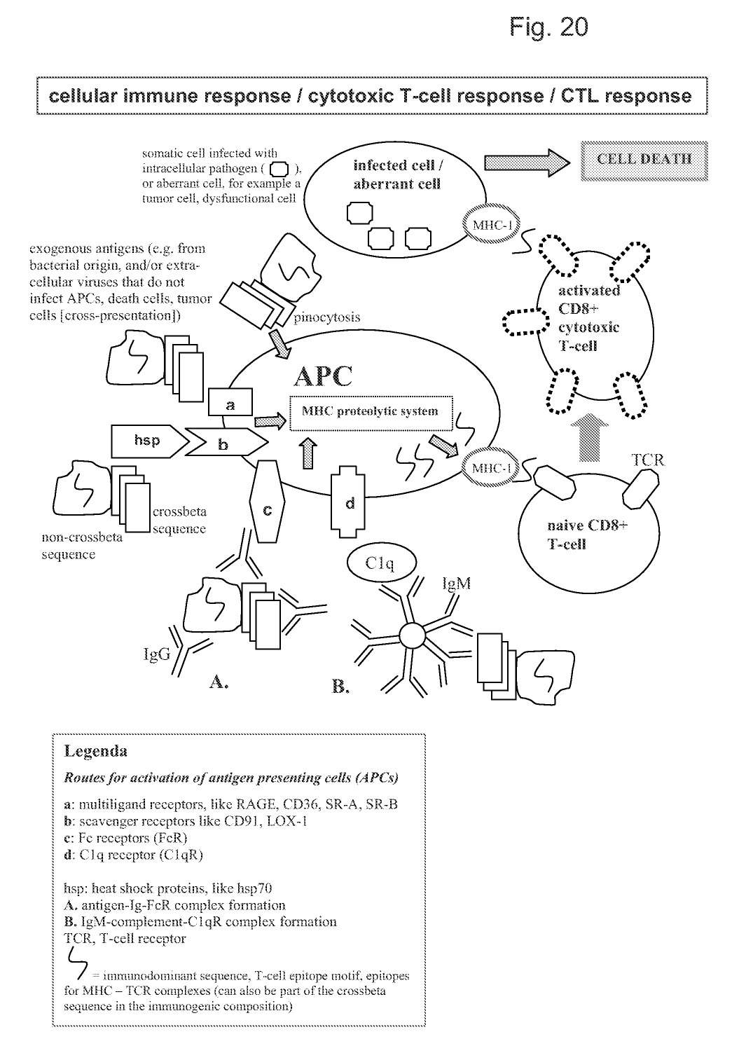

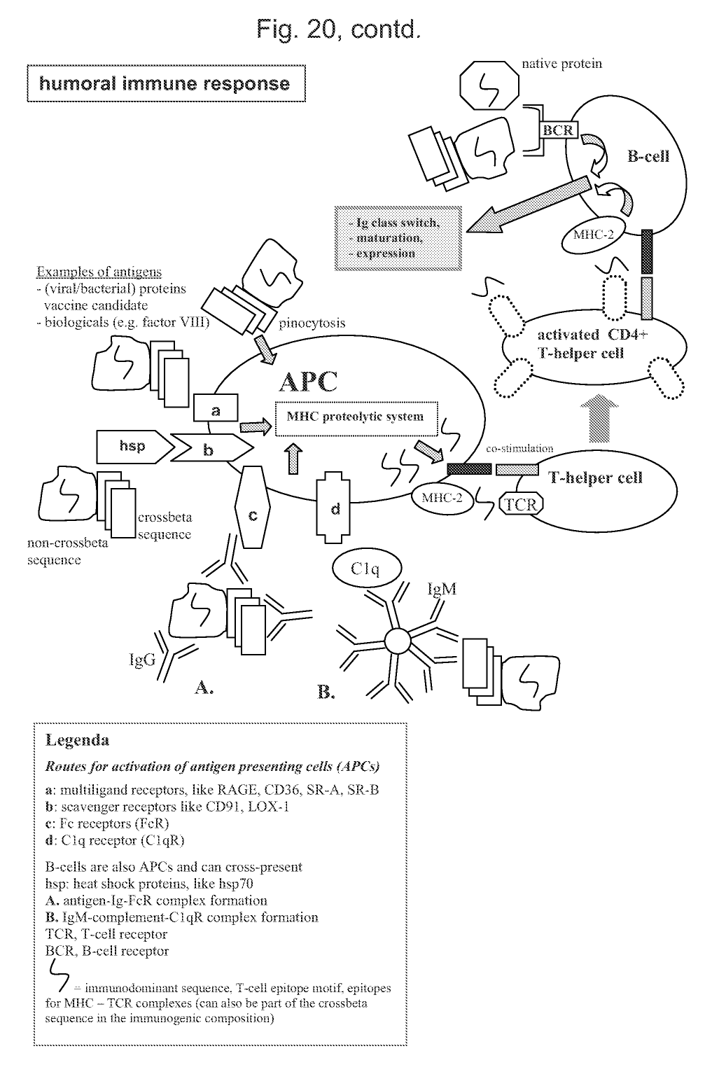

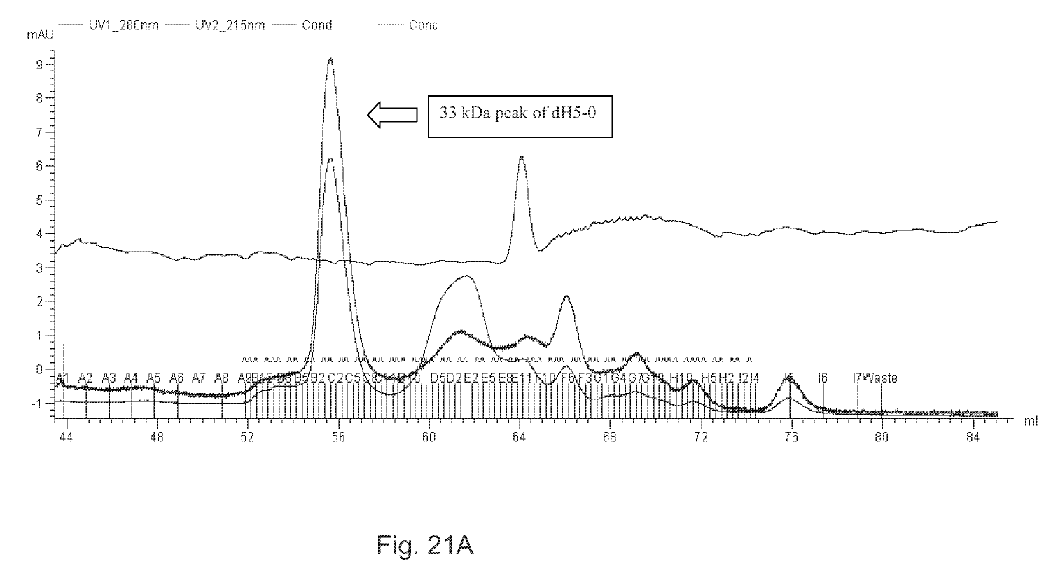

Binding of anti-FVIII antibodies from Hemophilia patient plasma with FVIII inhibiting antibodies, to the nine FVIII variants was assessed using ELISA. The ELISA was essentially performed as described above. Now, all nine variants were immobilized on ELISA plates, and overlayed with 1:100 or 1:200 diluted plasma. See FIGS. 17-19. From the experiment depicted in FIGS. 18 and 19 it is concluded that most likely cFVIII-7 does not coat efficiently to the ELISA plate and/or the FVIII conformation is changed in a way that antibodies bind with reduced efficacy. It is observed that the increase in cross-beta content, seen for cFVIII-4 to 6 is accompanied by reduced anti-FVIII antibody binding from patient plasma, though significant binding is still observed for patient B and cFVIII4 and 5, and patient D and cFVIII-4. These observations are confirmed in a second experiment using 1:100 diluted plasma and a selection of FVIII variants (cFVIII-1, 4-7) and a selection of plasma's (patient B, C, D, healthy donor). See FIG. 19. These data show that at least for patients B and D FVIII preparations with exposed epitopes to which FVIII inhibitory antibodies can be elicited, combined with immunogenic cross-beta conformation, may have contributed to induction of the inhibitory anti-FVIII antibodies that are determined in plasma of these patients.

In subsequent studies, for example FVIII preparations are produced with alternative appearances of cross-beta conformation combined with exposed epitopes for FVIII inhibiting antibodies, upon subjecting FVIII to various additional misfolding procedures, like for example prolonged incubation of FVIII at 4° C., at room temperature and at 37° C. In time, samples of these FVIII incubations are subjected to various cross-beta assays and structure determinations aiming at providing insight in multimer size and distribution. In addition, binding of patient antibodies is monitored in time. Based on these analyses, it is depicted which molecules with varying combinations of cross-beta, multimer size and antibody binding capacity are selected for immunization trials. Preferably, FVIII variants are included in the immunization trials that comprise combinations of cross-beta conformation or not, that is incorporated in monomers up to for example 1000-mers, and that expose or do not expose epitopes for FVIII inhibiting antibodies. For example, mice are immunized, for example transgenic mice with human FVIII, preferably mice deficient for murine FVIII. A typical example of an immunization experiment is depicted below:

Ten mice/group. Pre-immune serum collection at day −2. Intravenous injections of 200 μl doses. Dose of 1 IE/mouse. Injections at day 0/14/28/42. Additional blood draws at day 14/28/42/49 for collection of serum. Groups: 1, placebo; 2, FVIII; 3, cross-beta FVIII variant A; 4, cross-beta FVIII variant B; 5, 50% FVIII+50% cross-beta FVIII variant A; 6, 50% FVIII+50% cross-beta FVIII variant B, with cross-beta FVIII variant A comprising exposed epitopes for inhibiting anti-FVIII antibodies, and comprising soluble oligomers, and with cross-beta FVIII variant B lacking epitopes for FVIII inhibiting antibodies to a relatively large extent, and comprising insoluble oligomers to a large extent. For example, cross-beta FVIII variant A is cFVIII-4 or 5, and cross-beta FVIII variant B is cFVIII-6 or 7. The sera or plasma are analyzed for the presence of FVIII inhibiting antibodies, for example in a Bethesda assay. Furthermore, the sera or plasma are analyzed for their capacity to compete for binding to FVIII with the patient sera or plasma A-D, which comprise FVIII inhibiting antibodies. In this way, information is obtained about the contribution of various parameter ratios with respect to exposure of epitopes for FVIII neutralizing antibodies, cross-beta content and appearance, and multimer size and multimer size distribution, to the ability to induce anti-FVIII antibodies that inhibit FVIII.

### Example H5

With this example it is demonstrated that the combination of certain cross-beta structures in H5 protein and a certain amount of exposed epitopes for functional antibodies is required for inducing a protecting immune response in mice.

### Theoretical Considerations: Estimated Size and Surface of H5 Multimers

The average van der Waals radius of the 20 amino acids is approximately 0.3 nm, or 3 Å. The approximate average volume of an amino acid is 110 Å³. The approximate average surface of an amino acid residue is 28 Å², or 0.28 nm². The approximate average mass of an amino acid residue is 120 Da. From these numbers it is estimated that using the 1.000 kDa MW cut-off filter, at maximum protein assemblies comprising approximately 8500 amino acid residues flow through the filter. This maximum size corresponds to a maximum protein surface on for example a TEM image, of 2400 nm². Assuming a spherical or squaric arrangement of the protein multimer, this corresponds to protein structures with a radius of approximately 27 nm, or 50×50 nm squares, respectively, on TEM images. With H5 appearing on the SEC column and on SDS-PA gel as amongst others, 33 kDa and 75 kDa molecules, multimers of up to 30 or 13 H5 monomers will flow through the 1.000 kDa filter, at maximum. By approximation, on average, 1 nm² corresponds to 3.6 amino acid residues or 430 Da, and 1 kDa corresponds to 2.3 nm².

With this approximate numbers it is possible to calculate the number of H5 monomers that appear in multimers, as seen for example under the direct light microscope, in SEC fractions, on TEM images and on SDS-PA gels. These considerations also apply for any other molecular assembly of one or more protein molecules, like for example ovalbumin, E2 and factor VIII.

Endotoxin measurement. The endotoxin content of H5 as supplied by Protein Sciences was measured at 25 μg/ml (diluted in sterile PBS), the concentration of H5 at which vaccination will occur. The Endosafe cartridge had a sensitivity of 5-0.05 EU/ml (Sanbio, The Netherlands).

The endotoxin level is 0.152 EU/ml. The endotoxin level of the dilution buffer PBS is <0.050 EU/ml.

Methods for Preparing Structural Variants of H5 which Comprise Cross-Beta

Recombinantly produced heamagglutinin 5 (H5) protein of H5N1 strain A/Vietnam/1203/04 (A/VN/1203/04) was purchased from Protein Sciences. The stock concentration was 1 mg/ml (determined with the BCA method (Pierce)) in 10 mM sodium phosphate, pH 7.1, 171 mM NaCl, 0.005% Tween20. H5 is stored at 4° C. The H5 stock as supplied is referred to as cross-beta H5-0, or dH5-0, i.e., H5 that comprises cross-beta structure of arbitrarily chosen type 0. Handlings with H5 solutions are performed under sterile conditions in a flow cabinet. When dH5-0 is ultracentrifuged for 1 h at 100,000*g (4° C.), 62% of the H5 remains in the supernatant; 38% is pelleted. Therefore, 62% of the dH5-0 is designated as soluble H5, 38% as insoluble protein.

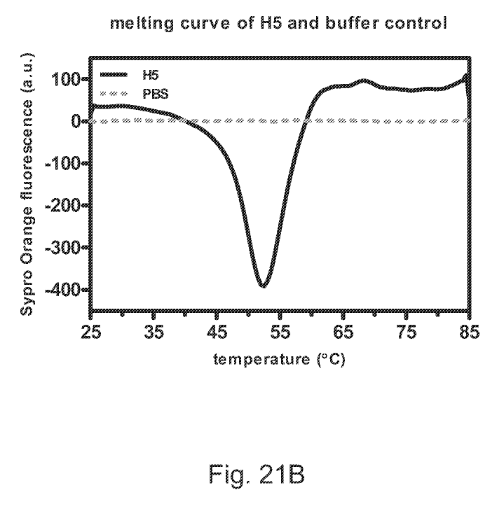

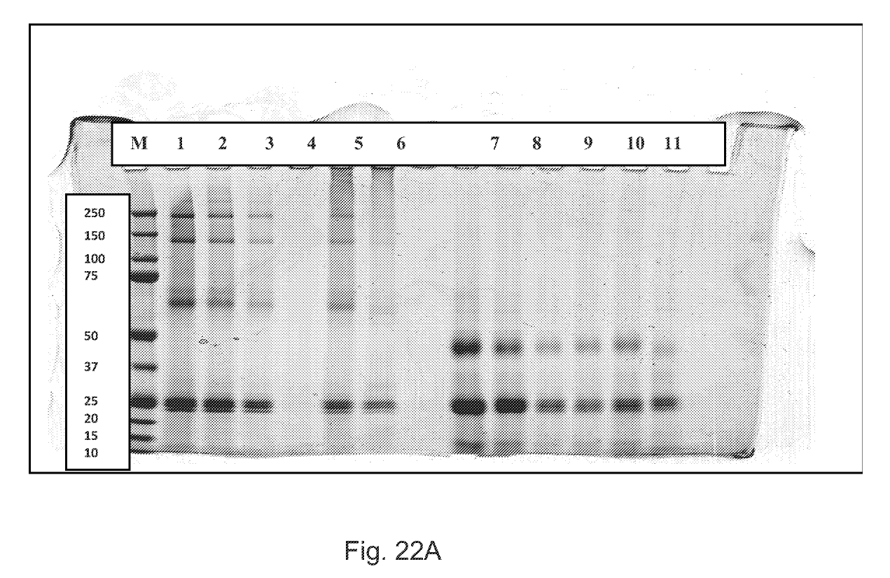

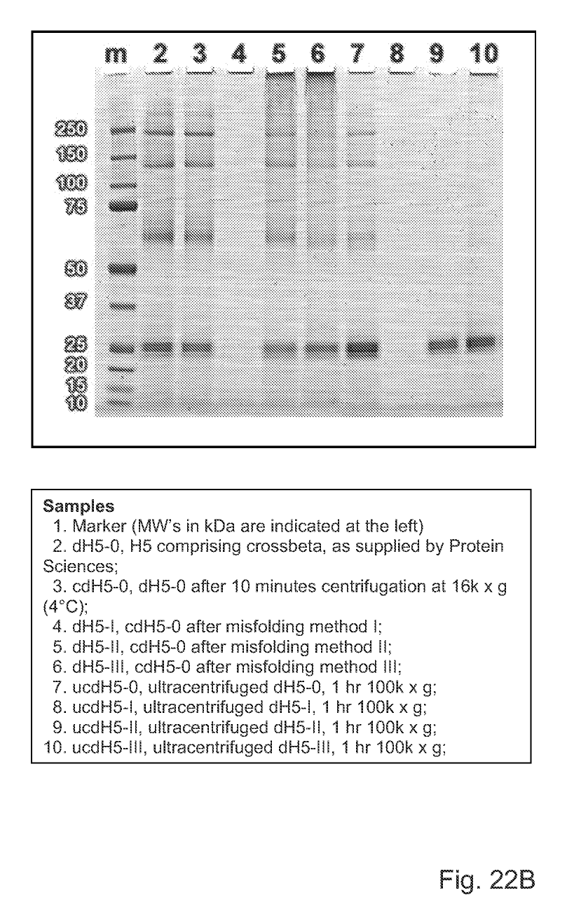

The dH5-0 protein solution is analyzed as supplied and in addition after applying a routine centrifugation step, i.e., 10 minutes centrifugation at 16,000-18,000*g, at 4° C., in a rotor with fixed angle. The dH5-0 after this standard centrifugation step is referred to as cdH5-0, cross-beta H5 after centrifugation. For analysis and vaccination trials, the supernatant of cdH5-0 is used. After the centrifugation run a white pellet becomes visible, indicative for the present of insoluble H5 aggregates. An aliquot of 175 μl of the dH5-0 is subjected to size exclusion chromatography on an analytical superdex75 10/30 column (GE Healthcare) by Roland Romijn (U-ProteinExpress, Utrecht, The Netherlands), using an Äkta explorer (GE Healthcare). In FIG. 21A it is seen that one main peak is retained by the SEC column. Calculation of the molecular weight, based on a known calibration curve of the column, revealed that 65% of the loaded dH5-0 eluted as a 33 kDa protein. The remaining protein fraction eluted as proteins with molecular masses of 4 kDa or smaller. Noteworthy, on SDS-PA gel with non-reducing conditions, the eluted 33 kDa dH5-0 fraction appeared with the same protein band pattern as the dH5-0 starting material (See FIG. 22A for dH5-0). Under reducing conditions, both dH5-0 starting material and the 33 kDa dH5-0 fraction appear as two bands of approximately 24 and 48 kDa. Either the four bands with MWs>50 kDa are co-eluted with the main 33 kDa dH5-0 band and are visualized on gel, or dH5-0 stably aggregates after the SEC run into multimers that do not dissociate upon heating in sample buffer with SDS.

Additionally, for several analyses dH5-0 and other misfolded H5 samples comprising cross-beta structure are ultracentrifuged for 1 h at 100,000*g, at 4° C., using a rotor with swing-out buckets. The supernatants of these ultracentrifuged H5 samples are used for analyses and are referred to as ucdH5-0 or udH5-0, and ucdH5-I/II/III or udH5-I/II/III.

Ultrafiltrated dH5-0, referred to as fdH5-0, is obtained by filtering cdH5-0 for 10 minutes at 16,000*g through a Vivaspin 500 PrNo VS0161, 1×10⁶ Da MW cut-off filter, at 4° C. The flow-through of the filter is used for subsequent analyses and immunizations, and comprises H5 monomers/oligomers with a molecular weight of approximately ≦1.000 kDa. The fraction of dH5-0 that is poured through the filter, i.e., fdH5-0, is 80% of the starting material, as determined with the BCA method after three consecutive filtrations. Therefore, the dH5-0 comprises approximately 20% protein multimers with a molecular mass of >1.000 kDa.

Preparation of Misfolded dH5-I Comprising Cross-Beta Structure

dH5-I (heat cycling at pH 7) is produced from dH5-0 supernatant after centrifugation for 10 minutes at 16,000*g (4° C.), i.e., cdH5-0. The H5 concentration is 1 mg/ml. From a 5 M NaCl stock an amount is added to cdH5-0 in order to adjust the NaCl concentration to that of dH5-II (see below). The cdH5-0 is divided in 100 μL aliquots in a 200-μl PCR plate (BioRad, 96 well, cat nr 2239441) and placed in a thermal cycler (Biorad, MyIQ). The cdH5-0 is incubated at 25° C. for 20 seconds and subsequently heated from 25° C. to 85° C., ramp 0.1° C./s, followed by a 20 s incubation at 85° C. This cycle is repeated twice (total cycles is three). The program finishes with cooling at 4° C. for 2 minutes. The dH5-I aliquots are combined and again divided into aliquots in Eppendorf 500 μL cups. Aliquots of 50 μg dH5-I/vial are stored at −20° C.

Before misfolding the protein solution looks clear, after heat denaturation the sample appears white turbid. After freezing-thawing and subsequent centrifugation a pellet is visible. After ultracentrifugation for 1 h at 100,000*g (4° C.), 37% of the H5 remains in the supernatant.

Preparation of Misfolded dH5-II Comprising Cross-Beta Structure

dH5-II (heat cycling at pH 2) is produced from dH5-0 supernatant after centrifugation for 10 minutes at 16,000*g (4° C.), i.e., cdH5-0. The H5 concentration is 1 mg/ml. The pH of cdH5-0 is lowered to pH 2 by addition of HCl from a 15% (v/v) stock in H₂O. Then it is divided into 100 μL per cup in PCR strips (BioRad, 96 well, cat nr 2239441) and placed in a MyIQ RT-PCR cycler (Biorad). The misfolding program is the same as used for preparing dH5-I (see above). Subsequently, dH5-II aliquots are combined and the pH is adjusted back to pH 7 by addition of NaOH solution from a 5 M stock. Then, dH5-II is aliquoted again and stored at −20° C.

Before misfolding the cdH5-0 solution at pH 2 appears clear, after heat denaturation and adjusting the pH back to 7, the dH5-II sample appears slightly turbid. After freezing-thawing and subsequent centrifugation a pellet is visible. After ultracentrifugation for 1 h at 100,000*g (4° C.), 41% of the H5 remains in the supernatant.

Preparation of Misfolded dH5-III Comprising Cross-Beta Structure

dH5-III (prolonged incubation at 5° C. below the melting temperature of dH5-0) is produced from cdH5-0. The H5 concentration is 1 mg/ml. For this, the melting temperature of cdH5-0 at 1 mg/ml was determined using the MyiQ cycler. 0.7 μl Sypro Orange 5000× stock (Sigma) is added to 70 μl cdH5-0 and the sample is heated from 25° C. to 85° C. The ramp rate is set to 0.1° C./min. At each temperature increment of 0.5° C. the Sypro Orange fluorescence is measured at 490 nm (excitation) and 575 nm (emission). The melting temperature was 52.5° C. (See FIG. 21B). Subsequently, cdH5-0 is incubated for approximately 16 h at 47.5° C., i.e., 5° C. below the cdH5-0 melting temperature. Aliquots of dH5-III are then stored at −20° C. Before misfolding the cdH5-0 solution was clear, after prolonged incubation at a temperature of 5° C. below the cdH5-0 melting temperature, the sample is still clear. After freezing-thawing and subsequent centrifugation no pellet is visible. After ultracentrifugation for 1 h at 100,000*g (4° C.), 45% of the H5 remains in the supernatant and is the soluble dH5-III fraction.

### Visual Inspection of H5 Samples Before/after Various Treatments

In Table 1 the results of the visual inspection of the six H5 forms is summarized.

Transmission electron microscopy imaging with H5 forms with/without ultracentrifugation. The various H5 forms are subjected to TEM analysis. The dH5-0, dH5-I, dH5-II and dH5-III forms are analyzed directly, and their supernatants after ultracentrifugation for 1 h at 100,000*g (4° C.) are imaged. PBS served as a negative control and gave an empty image, as expected. The dH5-0 appeared with a background of many non-uniformly shaped protein assemblies of approximately 25×25 nm to 100×100 nm, corresponding to molecular H5 assemblies of approximately 270-4300 kDa (approximately 4-57H5 monomers of 75 kDa). Also large, branched aggregates with strings of protein assemblies are seen. The branches are approximately 100 to 400 nm thick and approximately 2 to 5 μm in length. Upon ultracentrifugation of dH5-0, many string-like protein assemblies are seen, with bead-like subunits. Many have dimensions of approximately 25×50 nm, a few are approximately 100×100 nm up to 400×800 nm. The cdH5-0 appears very similar to udH5-0, with the exception that also larger protein assemblies are seen with dimensions of approximately 1500×1500 nm. The fdH5-0 appears with a background of uniformly shaped relatively tiny protein structures with undefined, though relatively small size and shape. A few relatively large protein structures are seen, which are composed of strings of protein assemblies. These structures have tree-like appearances with branches, and are approximately 400×4000 nm in size. The dH5-I comprises relatively a few but large and dense protein assemblies composed of spherical protein building blocks. The building blocks are connected in branched strings with approximate dimensions of 500×5000 nm. Hardly any H5 is seen in structures apart from the large branched strings. Upon ultracentrifugation, an empty image is obtained, indicated that all dH5-I structures seen before ultracentrifugation are insoluble and pelleted. The dH5-II is seen as amorphous and large protein assemblies with approximate sizes of 3×3 μm. The protein assemblies appear as loosely connected structures. The structures are composed of smaller non-uniformly shaped low-density protein assemblies, which are also seen freely. These building blocks are approximately 50×50 to 100×100 rum in size. Upon ultracentrifugation, the supernatant is fully clear on the TEM image. This shows that H5 multimers are insoluble and pelleted upon ultracentrifugation. The dH5-III is presented on the TEM image as a relatively high number of two types of protein assemblies with a relatively small size of approximately 25×25 nm and approximately 50×50 nm. Upon ultracentrifugation, again many small protein assemblies are seen in the supernatant, on the TEM image. The approximate sizes of the multimers are mostly 20×20 nm with a few protein assemblies of approximately 100×100 nm in size. Apparently, the protein assemblies are soluble and are not pelleted upon ultracentrifugation.

### Analysis of H5 Forms on SDS-PA Gel Under Reducing and Non-Reducing Conditions

The six H5 structural variants were analyzed on an SDS-PA gel, both with and without a pretreatment in the presence of reducing agent DTT. See FIG. 22A. When comparing the three H5 forms dH5-0, cdH5-0 and fdH5-0 it appears that the number of molecules with a molecular weight of >50 kDa decreases in the order dH5-0>cdH5-0>fdH5-0. It is of note that the protein assemblies that are visible stayed intact after heating for 10 minutes at 100° C. Upon adding DTT during heating, the three H5 forms appear similarly on gel. The dH5-I variant does not enter the gel when non-reducing conditions are applied, indicative for the presence of relatively large multimers that resist heating at 100° C. in the presence of SDS. Upon adding DTT during heating, these multimers dissociate and appear on the gel similarly to the other H5 forms. The dH5-II and dH5-III comprise a relatively high content of multimers with a molecular mass >250 kDa, with large multimers that do not enter the gel, when non-reducing conditions are applied. Under reducing conditions, the H5 forms appear similarly as the other structural variants. These data show that dH5-I comprises relatively the largest multimers, with dH5-II and dH5-III comprising more and higher order multimers than dH5-0 and cdH5-0, and with fdH5-0 comprising least multimers.

SDS-PAGE with H5 Samples Before/after Ultracentrifugation

The dH5-0, dH5-I, dH5-II and dH5-III are subjected to ultracentrifugation for 1 h at 100,000*g (4° C.). This ultracentrifugation is accepted as a procedure for separation of insoluble protein molecules from the soluble fraction that will remain in the supernatant. Together with starting material and cdH5-0, these ultracentrifuged samples are analyzed on an SDS-PA gel. See FIG. 22B. The dH5-0 starting material and cdH5-0 appear in a similar fashion; five protein bands with molecular weights of approximately 25, 60, 140, 240 and 350 kDa. Upon ultracentrifugation of dH5-0, the 25 kDa band becomes more dominant, when the same total amount of H5 is loaded onto the gel (correction factor determined based on BCA protein concentration determination), and when compared to dH5-0 and cdH5-0. The dH5-I sample is not visible on gel at all. Apparently, dH5-I comprises molecular assemblies or multimers that are too large to enter the gel, and that are tightly kept together by relatively strong forces. Interestingly, approximately 37% of the dH5-I stayed in solution upon ultracentrifugation. Apparently, this 37% of the dH5-I molecules is composed of multimers that can not be visualized on the SDS-PA gel. Both dH5-II and dH5-III comprise the same H5 bands as dH5-0 and cdH5-0, when analyzed before ultracentrifugation. In addition, high molecular weight bands are seen in both H5 forms, indicative for the presence of multimers that are tightly kept together. After ultracentrifugation, for both dH5-II and dH5-III all multimer bands and H5 bands with MWs>50 kDa are not seen anymore, indicating that those H5 molecules are pelleted upon ultracentrifugation.

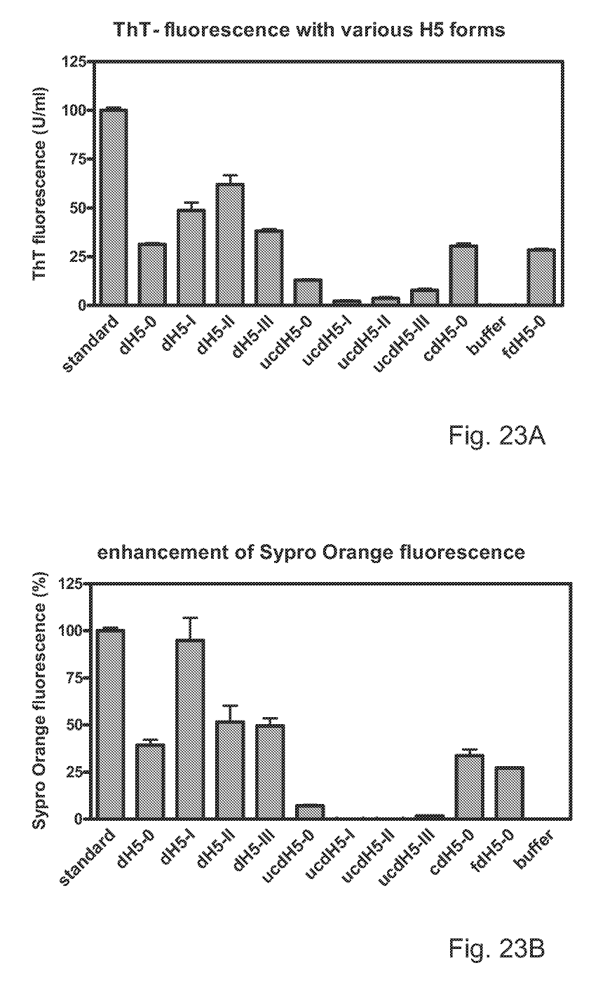

Thioflavin T fluorescence. Binding of Thioflavin T and subsequent enhancement of its fluorescence intensity upon binding to a protein is a measure for the presence of cross-beta structure which comprises stacked beta sheets. For measuring the enhancement of Thioflavin T fluorescence, H5 samples were tested at 100 μg/ml final dilution. Dilution buffer was PBS. Negative control was PBS, positive control was 100 μg/ml standard misfolded protein solution, i.e., dOVA standard. dOVA standard is obtained by cyclic heating from 25 to 85° C. (6° C./minute) of a 1 mg/ml ovalbumin (Albumin from chicken egg white Grade VII, A7641-1 G, Lot 066K7020, Sigma) solution in PBS. The H5 samples cdH5-0, dH5-I, dH5-II and dH5-III are also tested after 1 h centrifugation at 100,000*g, at 4° C. Supernatant is analyzed for its protein concentration using the BCA method. Subsequently, adjusted volumes in order to test identical protein concentrations, are used in the Thioflavin T fluorescence enhancement assay. Ultracentrifuged samples are indicated with a “u.” See FIG. 23A for the data. The dH5-0, cdH5-0 and fdH5-0 display very similar fluorescence enhancement, indicative for the presence of cross-beta structure to a similar extent. Applying misfolding protocols I-III results in an increase in Thioflavin T fluorescence, and therefore an increase in cross-beta content. The highest increase is seen with dH5-II; approximately a twofold increase when compared to dH5-0. For cdH5-0 approximately 50% of the fluorescence signal remains in the supernatant after ultracentrifugation. For ucdH5-I, II, III, most of the Thioflavin T fluorescence enhancing capacity is pelleted upon ultracentrifugation, showing that most H5 molecules with cross-beta structure are assembled in insoluble multimers.

Enhancement of Sypro Orange fluorescence. Sypro Orange is a probe that fluoresces upon binding to misfolded proteins. As a measure for the relative content of misfolded proteins, enhancement of Sypro Orange fluorescence is tested with H5 samples at 25 μg/ml final dilution. Dilution buffer was PBS. Negative control was PBS, positive control was 100 μg/ml dOVA standard. The H5 samples cdH5-0, dH5-I, dH5-II and dH5-III are also tested after 1 h centrifugation at 100,000*g, at 4° C. Supernatant is analyzed for its protein concentration using the BCA method. Subsequently, adjusted volumes in order to test identical protein concentrations, are used in the Sypro Orange fluorescence enhancement assay. Ultracentrifuged samples are indicated with a “u.” See FIG. 23B for the data. The cdH5-0 and fdH5-0 samples display a somewhat lower fluorescence enhancement than their starting material dH5-0. This indicates that after centrifugation for 10 minutes at 16,000*g a fraction of misfolded dH5-0 is pelleted, and that after filtration a fraction of dH5-0 with a molecular weight of >1.000 kDa is retained by the filter and has misfolded protein characteristics. Applying misfolding protocols I-III results in an increase in Sypro Orange fluorescence, that is most pronounced for dH5-I. Compared to the starting material, the Sypro Orange fluorescence is about doubled. For cdH5-0 approximately 25% of the fluorescence signal remains in the supernatant after ultracentrifugation. For ucdH5-I, II, III, most if not all of the Sypro Orange fluorescence enhancing capacity is pelleted upon ultracentrifugation. As seen in the Thioflavin T fluorescence measurement (See FIG. 23A), the supernatant of dH5-III comprises relatively the most misfolded protein, compared to dH5-I and dH5-II.

### Binding of Fibronectin Finger 4-5 to H5 Forms Comprising Cross-Beta Structure

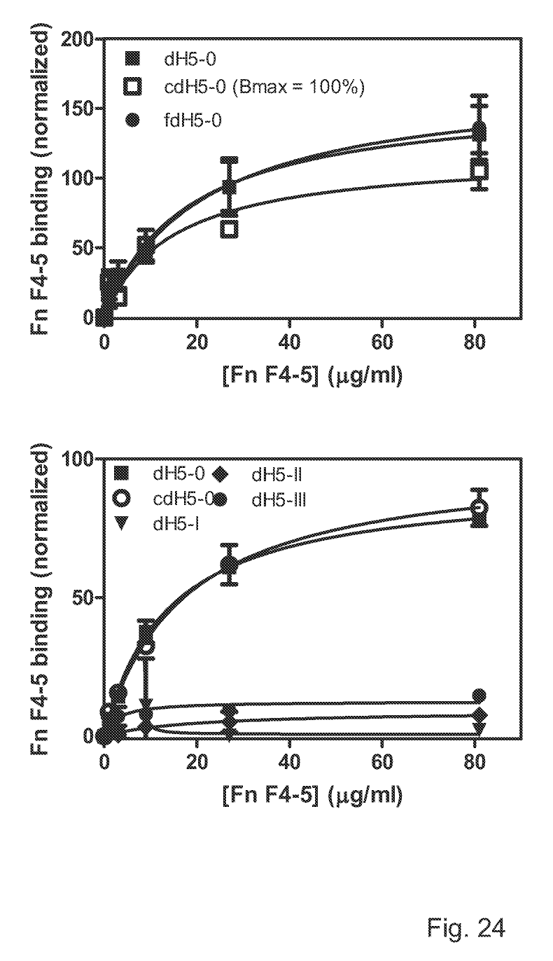

Finger domains of tPA, factor XII, hepatocyte growth factor activator and fibronectin bind to cross-beta structure in protein, when the free finger domains are contacted with proteins comprising cross-beta structure, as well as when the finger domains are part of the full-length or truncated proteins. We now assessed the binding of the fourth and fifth finger domain of fibronectin (Fn F4-5) to the various H5 forms, as depicted in FIG. 24 and Table 2. It is clear that the cross-beta H5 forms dH5-0, cdH5-0 and fdH5-0 bind Fn F4-5 to a far more extent than the dH5-I, dH5-II and dH5-III. Apparently, the increase in ThT fluorescence and Sypro orange fluorescence with these latter three forms, indicative for increased misfolding of the H5 upon the artificial exposure to denaturing conditions as described, is accompanied by a loss in the exposure of binding sites for the natural sensors of cross-beta structure, i.e., the finger domains. This shows that the nature of the cross-beta structure in terms of the molecular assembly, differs between dH5-0, cdH5-0 and fdH5-0 when compared to dH5-I, dH5-II and dH5-III.

### Binding of tPA Via its Finger Domain to Various Cross-Beta Comprising H5 Forms

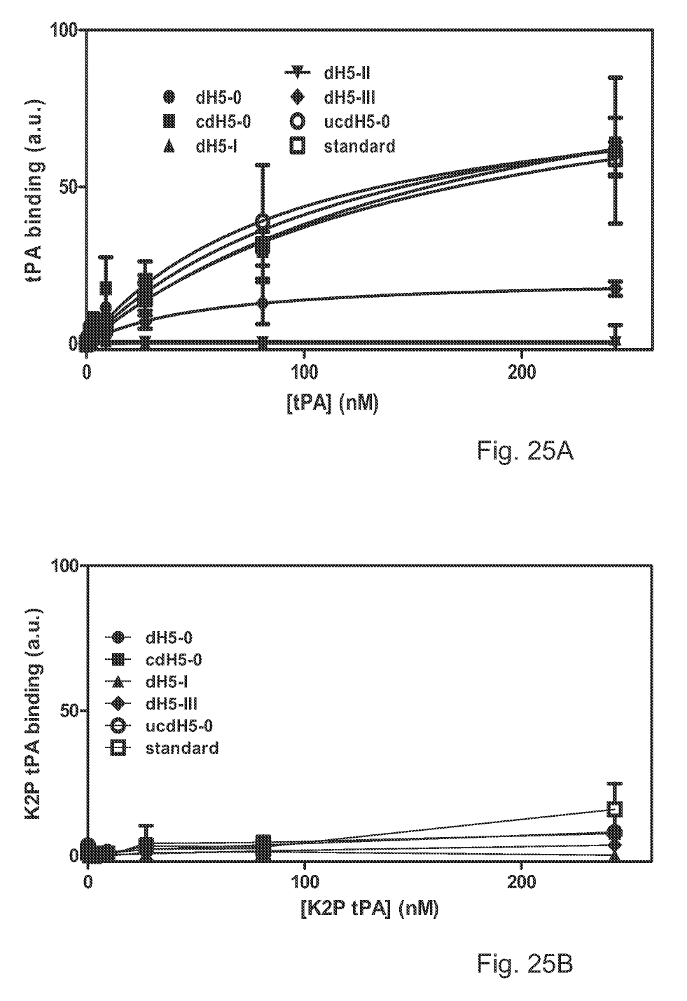

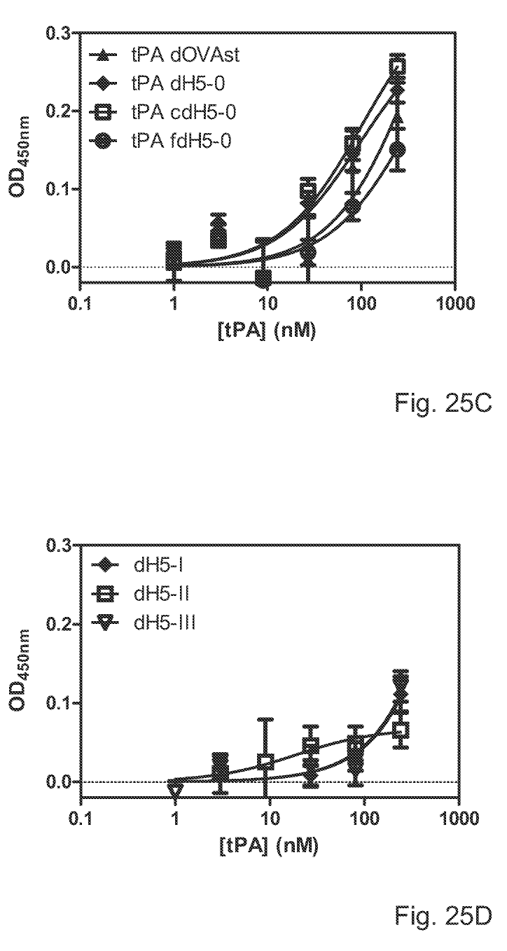

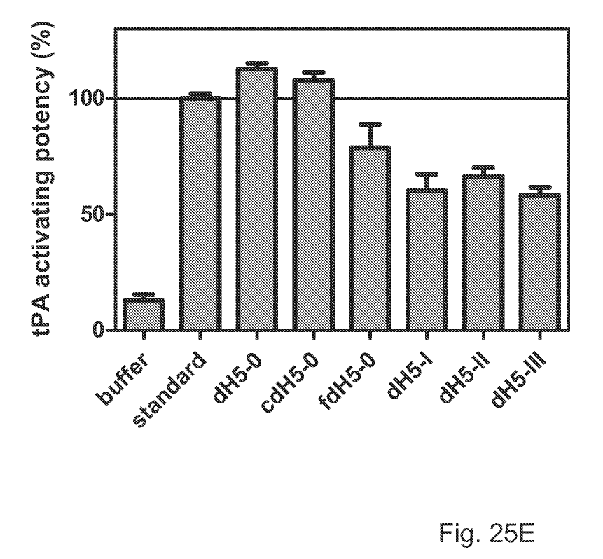

In FIGS. 25A, C and D it is seen that tPA binds to a higher order to dH5-0, cdH5-0 and fdH5-0, when compared to dH5-I, dH5-II and dH5-III, indicating that the first three forms expose more tPA binding sites than the latter three forms. Indeed, this is expressed in Bmax values, which is a relative measure for the number of binding sites: Bmax values are 0.32, 0.36 and 0.37 for dH5-0, cdH5-0 and fdH5-0, respectively, whereas the Bmax value could not be determined for dH5-I and dH5-III (too less binding sites), and Bmax is relatively low for dH5-II, i.e., 0.07. The kD values representing the affinity of tPA for the H5 forms, are 96, 102 and 342 mM for dH5-0, cdH5-0 and fdH5-0, respectively. Again, for dH5-I and dH5-III this kD value could not be determined, whereas the relatively few tPA binding sites on dH5-II bind tPA with an affinity of 19 nM. In FIG. 25A it is shown that after ultracentrifugation for 1 hour at 100,000*g of dH5-0 (depicted as “ucdH5-0”) tPA binds with similar affinity and to a similar number of binding sites, showing that the tPA binding fraction in dH5-0 is soluble. With Fn F4-5 a similar tendency with respect to the relative amount of binding sites for finger domains was seen when dH5-0, cdH5-0 and fdH5-0 are compared to dH5-I, dH5-II and dH5-III (see FIG. 24 and Table 2).

tPA/Plg Activation by H5 Samples Comprising Cross-Beta Structure.

The six H5 samples were tested for their tPA mediated plasminogen activation potency at a concentration of 50 μg/ml. The results are shown in FIG. 25E. Notably, the activation potency expressed as conversion of plasmin chromogenic substrate, of dH5-0, cdH5-0 and fdH5-0 is similar, and for all three forms higher than the plasmin activity seen with dH5-I, dH5-II and dH5-III. These potencies to activate tPA/plasminogen are in line with the tPA binding data as discussed above and depicted in FIG. 25. It is concluded that the cross-beta structures that are induced in H5 forms dH5-I, dH5-II and dH5-III have less potency to interact with tPA than the cross-beta structures present in dH5-0, cdH5-0 and fdH5-0.

Epitope Scanning with Nine Functional Monoclonal Anti-H5 Antibodies

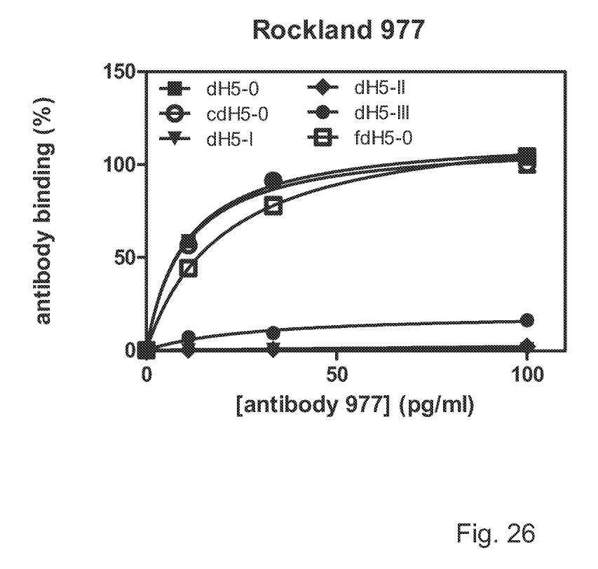

As outlined above previously, nine monoclonal mouse anti-H5 antibodies that neutralize H5N1 virus of strain A/VN/1203/04 and that inhibit hemagglutination by the virus, are used to determine whether the epitopes for these functional antibodies are exposed on the various structural H5 variants. In FIG. 26 an example is depicted of such a scanning experiment with anti-H5 antibody 977 (Rockland) and the six H5 forms. For all nine antibodies, the epitope scanning data is summarized in Table 3 and Table 4. In Table 3 the relative number of binding sites are shown for each antibody and each form of H5. In Table 4, the relative affinity of the exposed epitopes are given. It is concluded that by estimation H5 forms dH5-0, cdH5-0 and fdH5-0 expose relatively most epitopes for the functional antibodies, with on average the highest affinity binding sites, whereas on average H5 forms dH5-I, dH5-II and dH5-III expose less binding sites, and the exposed binding sites tend to be lower affinity binding sites, although not for every antibody. In conclusion, it is shown that in H5 forms dH5-0, cdH5-0 and fdH5-0, cross-beta structures are present in combination with a relatively high number of binding sites for functional antibodies, which binding sites are relatively high affinity binding sites. In contrast, in H5 forms dH5-I, dH5-II and dH5-III, the cross-beta structures are combined with less binding sites for functional antibodies, with on average lower affinity.

### Summary of Structural Data and of the Presence and Nature of Binding Sites for Functional Antibodies, for the Six H5 Structural Variants

In Table 5, the structural data as described above, and the epitope scanning data regarding the presence and nature of binding sites for functional antibodies, is summarized. Based on the analyses, by approximation the six H5 structural variants can be divided in two structural/functional groups. Based on, by estimation, similar parameters, Group I comprises dH5-0, cdH5-0 and fdH5-0. Based on, by estimation, similar appearances and parameters, Group II comprises dH5-I, dH5-II and dH5-III. These H5 forms in group I comprise cross-beta structures that at least in part appear as relatively smaller multimers, and that expose a relatively high number of tPA finger and Fn finger binding sites, with relatively high affinity. In addition, the cross-beta structures of group I H5 variants enhance ThT and Sypro orange fluorescence, although to a lesser extent than the H5 forms in group II. In group II, far less Fn F4-5 and tPA binding sites are present. Multimers appear to be larger, accompanied by increased ThT fluorescence and Sypro orange fluorescence. On average, by approximation the relative number of binding sites for functional antibodies and the relative affinity of functional antibodies for H5 variants in group I is higher than for H5 variants in group II.

Immunization of Mice with Six H5 Variants, Followed by a Challenge with H5N1 Virus

As outlined above, Balb/c mice are immunized twice, at day 0 and day 21, with a dose of 5 μg of the six H5 forms. Group 2, dH5-0; group 3, cdH5-0; group 4, fdH5-0; group 5, dH5-I; group 6, dH5-II; group 7, dH5-III. Controls are group 1, placebo (PBS), group 8, 5 μg cdH5-0 mixed with 40 times diluted alum (Adjuphos, Brenntag), and group 9, commercially available H5N₂ killed virus vaccine adjuvated with oil in water emulsion (Nobilis flu, Intervet). None of the vaccine formulations induced a visible reaction in the mice, except for the Nobilis flu vaccine, which induced palpable reactions on the flanks of the mice. At day 33 blood is drawn for titer determination (See Table 6). The total anti-H5 antibody titer of IgG and IgM isotypes is determined, in an ELISA using immobilized cdH5-0 and dilution series of the individual mouse sera. At day 42 blood is drawn for serum collection, and mice are challenged with a lethal dose of H5N1 virus of strain A/VN/1194/04. The virus dose per mouse was approximately 50 μl with a titre 9.7 log TCID₅₀/ml. During 14 days the weight of the mice was measured and the mice were clinically examined, daily. At day 56, blood is drawn from mice that survived the viral challenge, for serum collection. Presence of total anti-H5 antibodies and presence of functional anti-H5 antibodies is assessed.

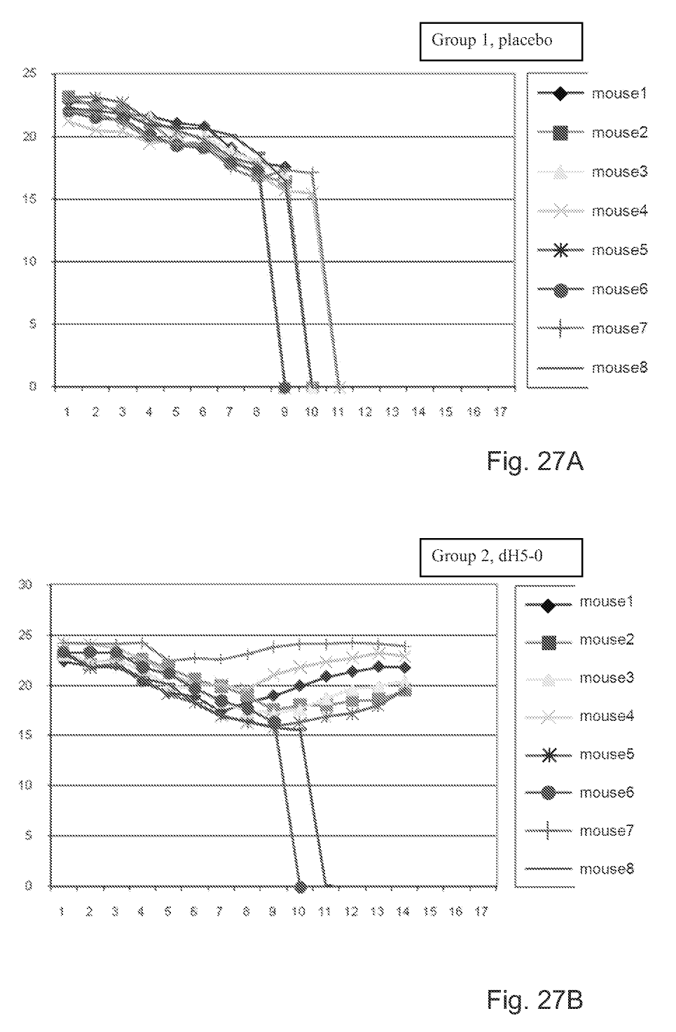

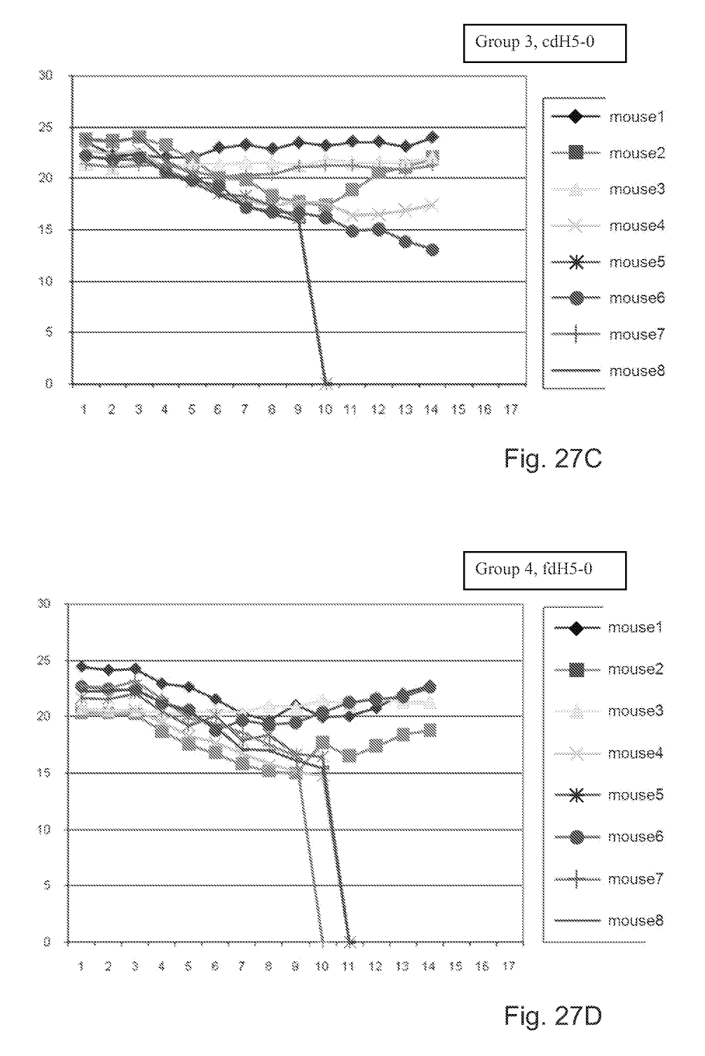

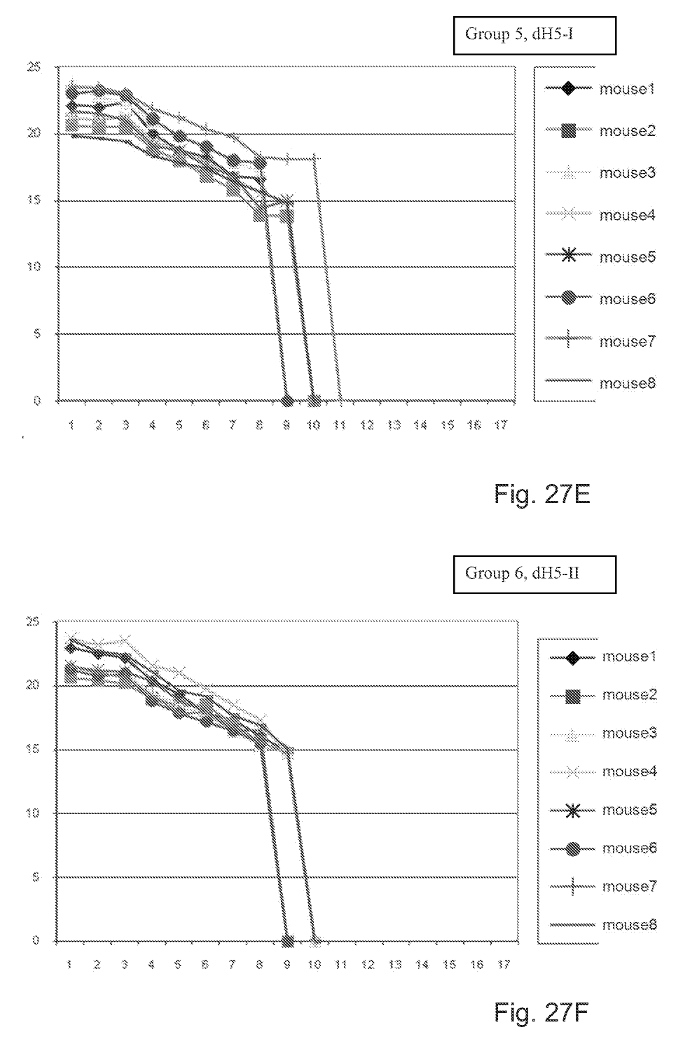

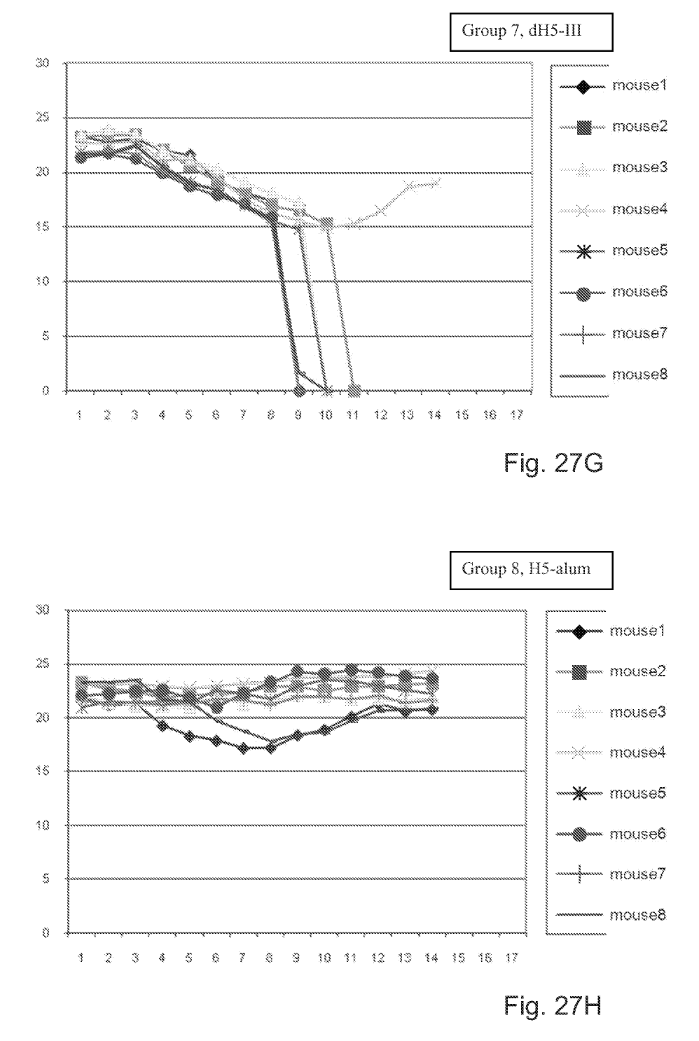

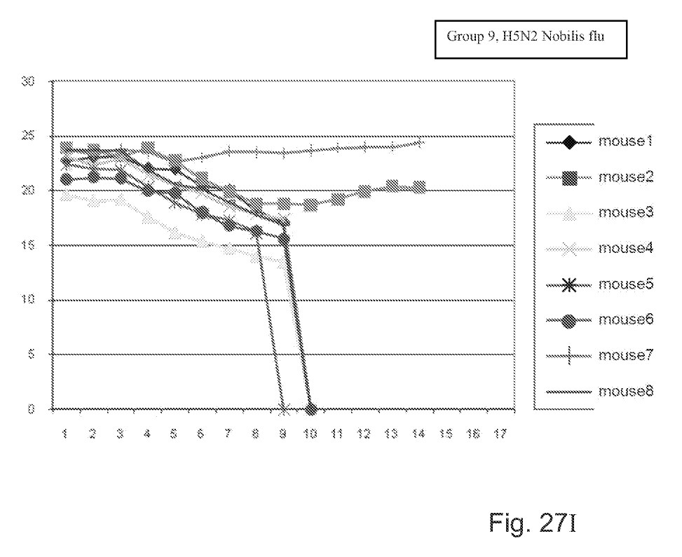

In Table 5, Table 6 and FIG. 27 the results and observations of the H5 immunizations and challenge with H5N1 virus are depicted. In Table 6, for each individual mouse its anti-H5 antibody titer in sera collected at day 12 after the second immunization and survival data are given. In FIG. 27, for each individual mouse its weight during the fourteen days post challenge infection are given, as well as the survival data. The combined data demonstrate that the various structural forms of H5 provide varying protection against viral challenge, and induce antibody titers to a varying extent. The level of protection provided upon vaccination with Nobilis flu H5N₂ as a reference, was low; survival of 2 out of 8 mice. It is probably due to the fact that the challenge virus was not homologous to the antigen in the vaccine. In contrast, the H5 antigen used in groups 2-8 is homologous to the H5 in the virus used for the challenge infection. Eight mice died in the placebo group 1, with the last mice dying at day 11 post challenge, and eight mice survived in the positive control group 8. In group 1, all mice suffered from a gradual weight loss; in group 8, two mice suffered from weight loss, but gained weight again. In group 8, all eight mice developed an anti-H5 antibody titer. The immunogenic compositions comprising dH5-0, cdH5-0 and fdH5-0 (H5 forms group I) provided better protection than dH5-I, dH5-II and dH5-III (H5 forms group II), with 6, 6 and 4 surviving mice, compared to 0, 0 and 1 surviving mice, respectively. When titers are considered, the H5 forms in group I induced titers in 8, 8 and 7 mice, compared to 1, 0 and 4 mice, when mice immunized with dH5-0, cdH5-0 and fdH5-0 are again compared to dH5-I, dH5-II and dH5-III, respectively. All mice that survived the challenge had developed an anti-H5 titer. Of the mice immunized with cross-beta H5 forms dH5-0, cdH5-0 or fdH5-0, one, three and one mice did not suffer from weight loss, respectively, whereas all mice immunized with dH5-I, dH5-II or dH5-III suffered from weight loss. Mice immunized with dH5-0, cdH5-0 or fdH5-0, that did not survive the challenge, died at day 10 (4 mice) or day 11 (4 mice), whereas mice immunized with dH5-I, dH5-II or dH5-III, that did not survive the challenge, on average died earlier, i.e., at day 9 (11 mice), day 10 (10 mice) or day 11 (2 mice).

In Table 5, the immunization and challenge data are summarized and compared for the H5 forms in group I and the H5 forms in group II. When the titer data, weight loss data and survival data are considered with respect to the cross-beta structure data and the exposed functional epitopes data, it is clear that the dH5-0, cdH5-0 and fdH5-0 are provided with a combination of i) type of cross-beta structure, ii) relative amount of cross-beta structure, iii) relative multimeric molecular distribution, iv) relative fraction of soluble molecules, and v) relative number of exposed epitopes for functional antibodies, with relative high affinity binding sites, that are beneficial for inducing protection against H5N1 infection, when compared to the combined data obtained with H5 forms dH5-I, dH5-II and dH5-III. These latter three forms induced less protection against H5N1 infection, and structural and functional parameters differed from those seen with dH5-0, cdH5-0 and fdH5-0.

### Example

### E2

Immunization of Pigs with Various E2 Cross-Beta Structural Variants, Followed by a Challenge with Classical Swine Fever Virus (CSFV).

See the example text above for a general outline of the experimental approach.

E2 purification. E2 in cell culture supernatant was obtained frozen at −20° C. from Central Veterinary Institute (CVI, Lelystad, the Netherlands), and labeled by CVI as follows: CGF E2 marker vaccine, Batch: E20-98-A001, Datum 23-2-98. The volume is ˜300 ml. Purification has been performed by R. Romijn (U-ProteinExpress, Utrecht, NL). Thawed supernatant was centrifuged for 10 minutes at 5500*g, at 4° C., and subsequently dialyzed against PBS (Gibco, 20012; 1.54 mM KH₂PO₄, 155.2 mM NaCl, 2.7 mM Na₂HPO₄-7H₂O, pH 7.2). The endotoxin level of undialyzed supernatant was assessed using an Endosafe PTS apparatus, and was 0.296 EU/ml. Two 149.5 ml aliquots were dialyzed against 800 ml PBS at 4° C. After 5 hours the PBS was replaced by fresh PBS and dialysis was continued overnight at 4° C.

First, an affinity purification has been performed using an anti-E2 antibody column. For this purpose, monoclonal anti-E2 antibody V3 (Prionics, The Netherlands) was coupled to CNBr-activated Sepharose 4 Fast Flow (GE Healthcare), according to the manufacturers protocol. Approximately 20 mg V3 was coupled to 13.5 ml Sepharose. Antibody 39.5 is V3 labeled with horse raddish peroxidase (Prionics, The Netherlands), and is used as outlined below. The running buffer was PBS and after loading the dialyzed supernatant, bound E2 was eluted with 0.1 M glycine pH 2.5. Fractions of 2 ml were collected in 2 ml Eppendorf cups containing 100 μl 1 M Tris (pH not adjusted).

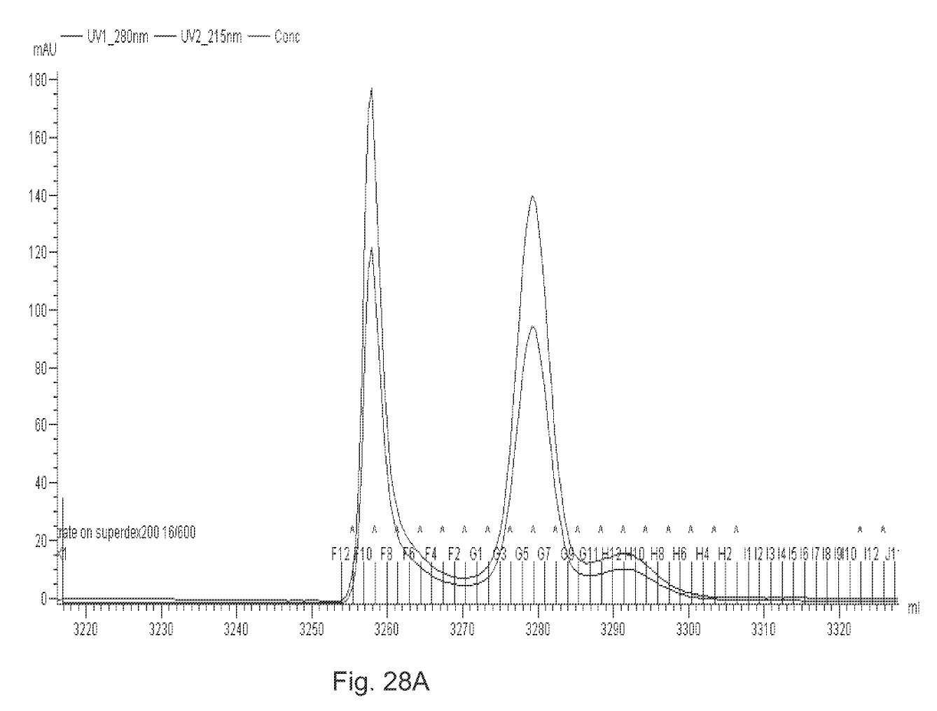

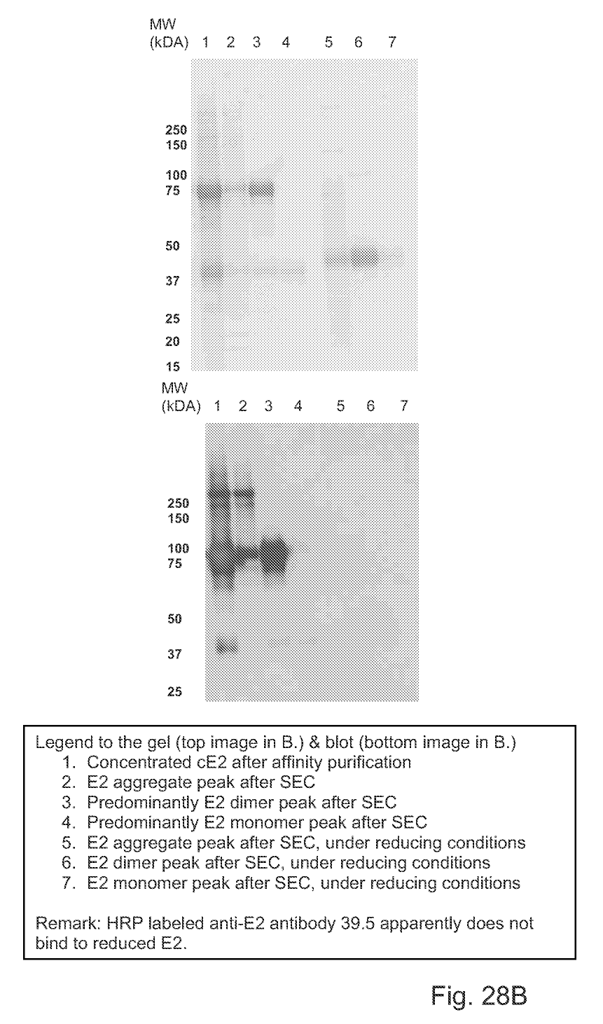

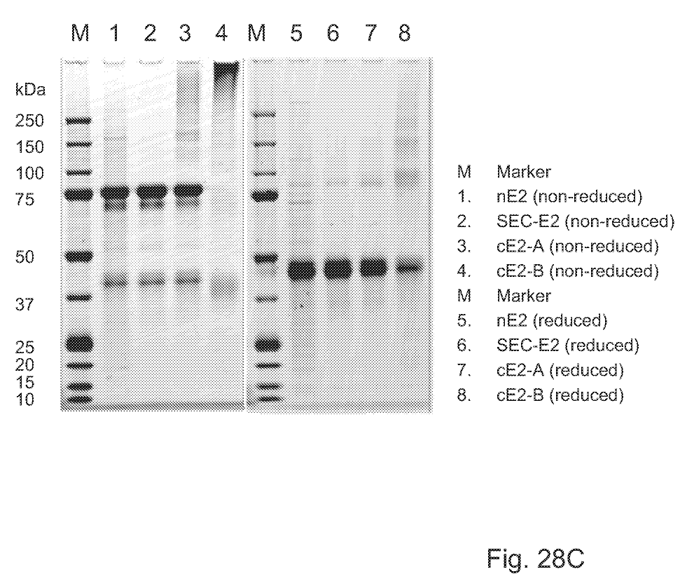

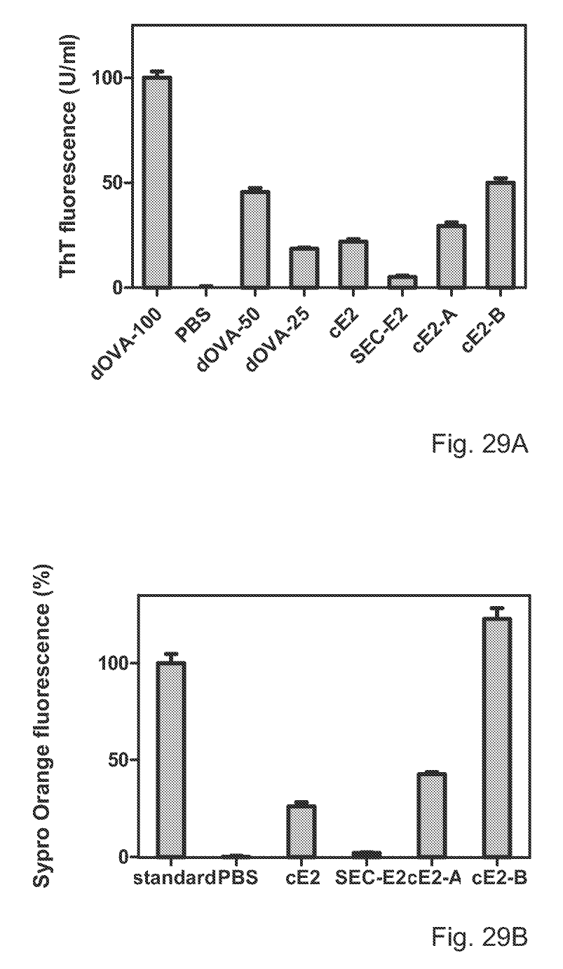

After affinity purification, cross-beta E2, referred to as cE2, is obtained. The cE2 is dialyzed against PBS and appeared as an approximately 100% pure protein on a Coomassie stained polyacryl-amide gel. The 8.3 mg cE2 was subsequently concentrated to 7.9 mg/ml using a Vivaspin20 10 kDa filter (4° C., 4800*g; Sartorius). A fraction of the cE2 was aliquoted and stored at −20° C. Another fraction of the cE2 was applied to a preparative size exclusion chromatography (SEC) column (Superdex200 16/600; GE Healthcare) and fractionated using an Äkta purifier (GE Healthcare). The running buffer was PBS. See FIG. 28A. Approximately 41% of the E2 eluted as aggregates that were not retained by the SEC column. On non-reducing SDS-PA gel E2 monomers and dimers are seen, as well as multimers with higher molecular weight. On a Western blot with anti-E2 antibody 39.5, these monomers, dimers and higher order multimers are detected and proven to be E2. Approximately 52% of the protein eluted predominantly as disulphide-bonded dimers with a molecular weight of approximately 86 kDa, with a fraction as monomers, with a molecular weight of approximately 43 kDa. Approximately 7% of the cE2 eluted as monomers (See FIG. 28B.). The 59% of cE2 that eluted as E2 monomers and dimers, according to the SDS-PAGE and Western blot analysis, was pooled and is from now on referred to as cross-beta E2 form SEC-E2. The concentration is 131 μg/ml 100% pure SEC-E2, as determined with the BCA method. On the Coomassie stained gel and the Western blot it is seen that after 10 minutes heating at 100° C. in sample buffer with sodium dodecyl sulphate, the cE2 still comprises oligomers with molecular seizes of >150 kDa, which indicates that tetramers and higher order multimers are present.

Misfolding procedures applied to cross-beta E2 form SEC-E2. After affinity purification of E2 using the anti-E2 antibody V3 column and subsequently the SEC column, SEC-E2 is used in two misfolding procedures to prepare alternative misfolded forms of E2 comprising cross-beta structure: cE2-A and cE2-B.

cE2-A preparation. For preparation of cE2-A, SEC-E2 was divided in 100 μL aliquots in PCR cups and placed in a thermal cycler (Biorad, MyIQ). The SEC-E2 was incubated at 25° C. for 20 seconds and subsequently heated from 25° C. to 85° C., ramp 0.1° C./s, followed by a 20 s incubation at 85° C. This cycle is repeated twice (total cycles is three). The program finishes with cooling at 4° C. for 2 minutes. The cE2-A aliquots are combined and again divided into aliquots in Eppendorf cups. Aliquots are stored at −20° C.

cE2-B preparation. For preparation of cE2-B, SEC-E2 was divided over five 1.5 ml Eppendorf cups; 1.3 ml/cup. The SEC-E2 was heated for 1 h at 95° C. in a thermo block. After heating, aliquots were recombined and mixed. Then, the cE2-B was again aliquoted in Eppendorf cups and stored at −20° C.

PTS LAL Assay. The endotoxin levels of the four E2 samples were determined with the PTS Endosafe (Sanbio, The Netherlands). The E2 samples were diluted to indicated concentrations and the endotoxin level was calculated for the final formulation at 16 μg/ml E2, which is used during the immunizations of pigs that are enrolled in the CSFV challenge experiment. The results are shown in Table 7.

Analysis of various structural forms of E2 comprising cross-beta structure. The various structural forms of E2 were analyzed in
An ELISA with three virus neutralizing mouse monoclonal anti-E2 antibodies,
An ELISA with pig immune sera obtained after immunization with placebo/cE2/cross-beta E2-OVA/E2 in double-oil-in-water adjuvant (E2-DOE),
A ThT fluorescence enhancement assay,
A Sypro Orange fluorescence enhancement assay,
The tPA/plasminogen activation assay,
A TEM imaging experiment,
Direct light microscopy analysis,
A Fn F4-5 ELISA, and
A tPA/K2P-tPA ELISA in the presence of εACA.

TEM imaging. TEM images were taken with the four E2 samples cE2, SEC-E2, cE2-A, cE2-B and PBS negative control. No protein structural features were seen on the negative control image. The cE2 appeared as large amorphous aggregates with dimensions of approximately 50×50 nm up to approximately 500×500 nm. No smaller protein structures are observed. In cross-beta E2 form SEC-E2, relatively a few particulate like aggregates are seen, that seem dense in nature and have dimensions of approximately 25×25 nm. In addition, it appears that numerous smaller protein structures are present in SEC-E2, that cover the full image. Dimensions are approximately 20×20 nm or 20×100 nm. Apparently resulted the formulation and storage procedure in the reappearance of E2 aggregates, because initially SEC-E2 comprised E2 monomers and dimers which eluted from the SEC column (See FIG. 28A). Now, on the TEM image aggregate structures are observed with molecular dimensions that exceed the size of monomers/dimers. In cE2-A, several relatively dense aggregates are seen with dimensions of approximately 200×200 nm and 100×100 nm. In addition, these larger aggregates are embedded in a background of numerous relatively smaller aggregates with dimensions of about 30×30 nm. In cE2-B hardly any aggregates are seen, except for relatively few aggregates with dimensions of approximately 30×30 nm.

Direct light microscopy. No aggregates were visible under the direct light microscope for any of the samples.

SDS-PAGE analysis under reducing and non-reducing conditions. An SDS-PAGE analysis was performed with the four cross-beta comprising E2 samples cE2, SEC-E2, cE2-A and cE2-B, and samples were analyzed after heating in reducing and non-reducing sample buffer. The results after Coomassie stain are shown in FIG. 28C. The non-reducing gel shows that after SEC purification high molecular aggregates are absent in SEC-E2, when compared to the cE2 starting material. Aggregates re-appear upon cyclic heat induced misfolding (sample cE2-A). cE2-B only appears with relatively large aggregates that hardly enter the gel with non-reducing conditions. The gel with samples after heating in the presence of DTT shows that almost all aggregates can be reduced into one band with the molecular mass of an E2 monomer. The E2 molecules in aggregates of cE2-B are relatively tightly bound as upon heating under reducing conditions, still some high molecular bands remain visible. Likely, on TEM images no cE2-B was visualized due to a too large multimeric arrangement that did not immobilized onto the TEM grids.

ThT fluorescence. ThT fluorescence enhancement was determined with the various cross-beta comprising E2 forms at 50 μg/ml. The results are shown in FIG. 29A.

The results show that upon SEC purification (SEC-E2), the ThT fluorescence enhancement is lowered compared to cE2, from which SEC-E2 was obtained after SEC. Furthermore, the ThT fluorescence enhancement is increased upon applying heat induced misfolding procedures to SEC-E2. Heat induced misfolding for 1 h at 95° C. (cE2-B) results in higher ThT signals than cyclic heating from 25° C. to 85° C. (cE2-A).

Sypro Orange fluorescence enhancement with E2 forms. The fluorescence enhancement of Sypro Orange was determined with the four cross-beta comprising E2 forms at 25 μg/ml E2, in PBS. The results in FIG. 29B. show a similar trend as is seen with the ThT fluorescence measurement described above. Upon SEC the fluorescence signal is drastically lowered. Upon heat induced misfolding the Sypro Orange signal is increased. Again cE2-B has a higher signal than cE2-A.

tPA/plasminogen activation assay. tPA mediated plasminogen activation was determined with the tPA/plasminogen assay using a chromogenic substrate for plasmin. The four E2 samples are tested for their potency to activate tPA/plasminogen with their cross-beta structure present in the molecules. E2 is tested at 50 μg/ml final concentration.

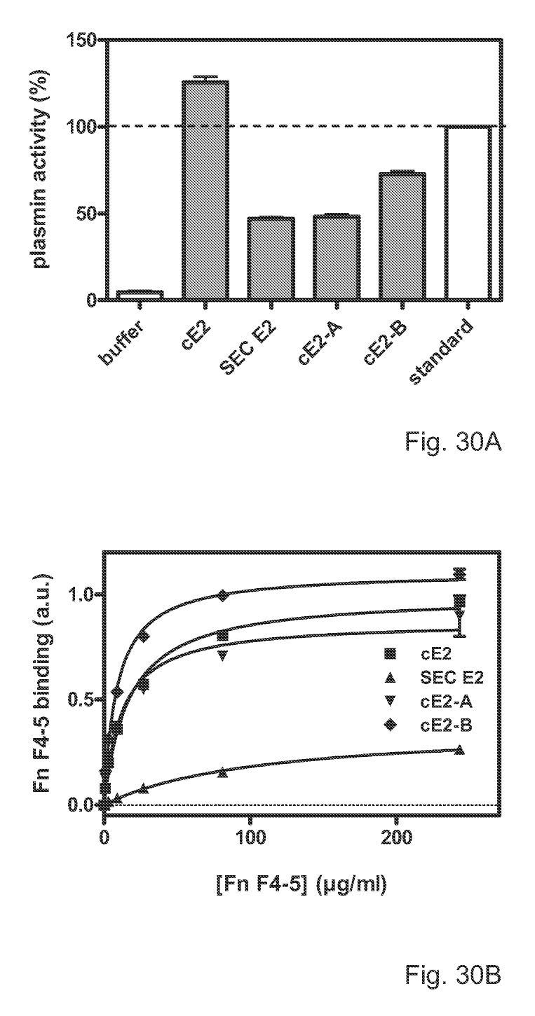

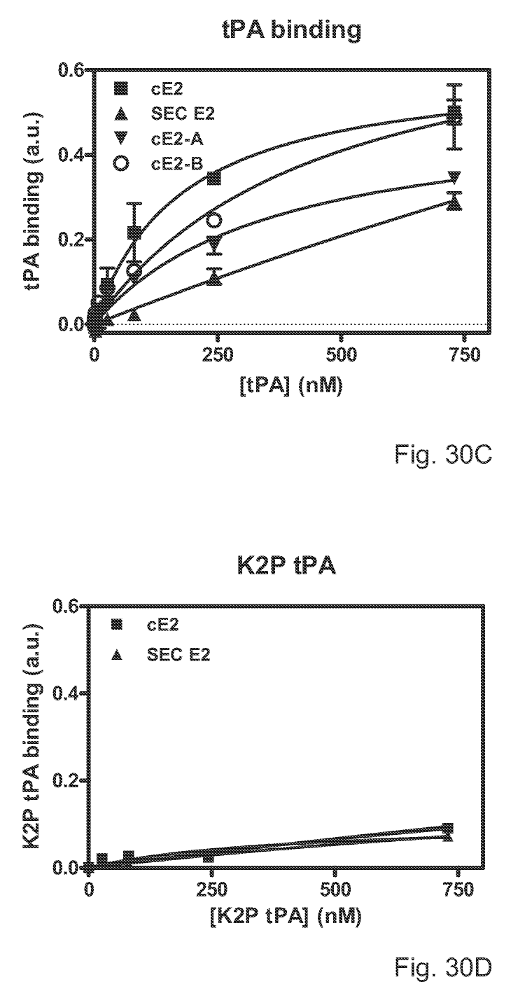

The results in FIG. 30A show that the cross-beta comprising affinity purified E2 (cE2) has the most tPA/plasminogen activating potency. After SEC purification, with SEC-E2 only one third of this activity remains. When SEC-E2 is applied to misfolding procedures, an increase in tPA/plasminogen activating potency is observed for cE2-B, but not for cE2-A.

Fn F4-5 ELISA. Binding of Fn F4-5 to the four forms of E2 was assessed in an ELISA experiment. The E2 samples were coated onto ELISA plates and overlayed with a concentration series of Fn F4-5, which comprises a C-terminal FLAG-tag. Binding of Fn F4-5 is monitored upon binding of HRP-tagged anti-FLAG antibody, followed by TMB stain. The results of one out of two experiments are shown in FIG. 30B. This figure shows that cE2 and cE2-A bind with similar characteristics to Fn F4-5, i.e., the number of binding sites for Fn F4-5 and the affinity of Fn F4-5 for cE2 and cE2-A are similar. Binding of Fn F4-5 to cE2-B resembles the binding of Fn F4-5 to cE2 and cE2-A, although the number of Fn F4-5 binding sites on cE2-B is slightly higher. Binding of Fn F4-5 to SEC-E2 however differs significantly from the binding to the other three E2 forms. The number of Fn F4-5 binding sites is much lower, i.e., about one third, and the affinity of Fn F4-5 for binding sites on SEC-E2 is much lower, i.e., approximately 6-10× lower.

tPA and K2P-tPA ELISA. Binding of tPA (Actilyse, Boehringer-Ingelheim) and K2P tPA (Reteplase, Boehringer-Ingelheim) to the four E2 forms was determined in an ELISA set-up. The E2 forms were immobilized on an ELISA plate and overlayed with a concentration series of tPA or K2P tPA in PBS with 0.1% Tween20 and the lysine/arginine analogue 10 μM i-amino caproic acid (εACA). The εACA is added to direct binding of tPA to cross-beta and to avoid additional binding of tPA or K2P tPA to lysine/arginine residues via the Kringle2 domain. The results of one out of two experiments are shown in FIG. 30C, D. Of the E2 forms tested cE2 binds with the highest affinity to tPA, whereas tPA binds with the lowest affinity to SEC-E2 (FIG. 30C). The affinity of tPA for cE2-A and cE2-B has a value between that for cE2 and SEC-E2. The affinity of tPA for cE2-B is higher than the affinity for cE2-A. Also the number of tPA binding sites on immobilized cE2-B is higher than that for cE2-A. These data, together with the data obtained with ThT fluorescence, Sypro Orange fluorescence and tPA activation experiments, indicate that cE2-B comprises more or different cross-beta structures than cE2-A. For all four samples it is observed that K2P tPA, that lacks the cross-beta binding finger domain, did virtually not bind to E2 (See, FIG. 30D).

Epitope Scanning ELISA with Three HRP Labeled CSFV Neutralizing Monoclonal Anti-E2 Antibodies.

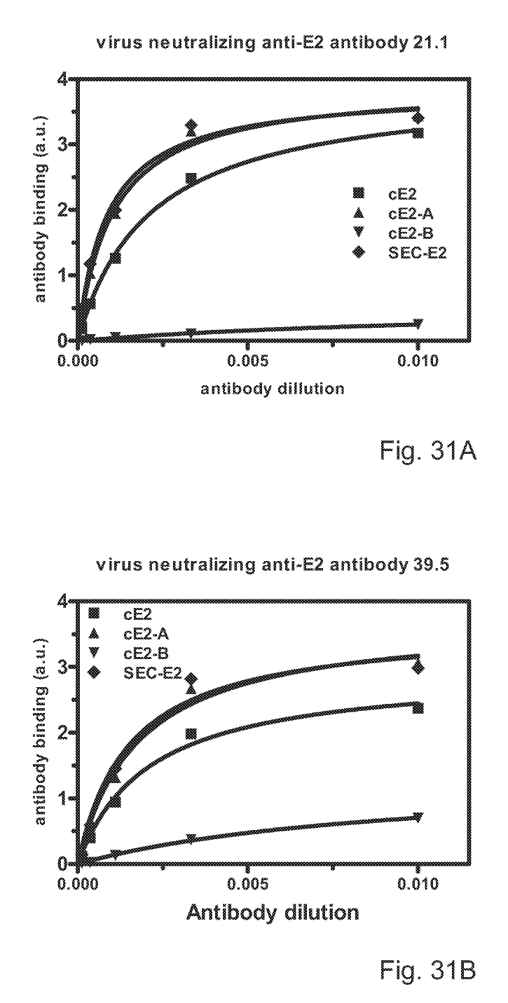

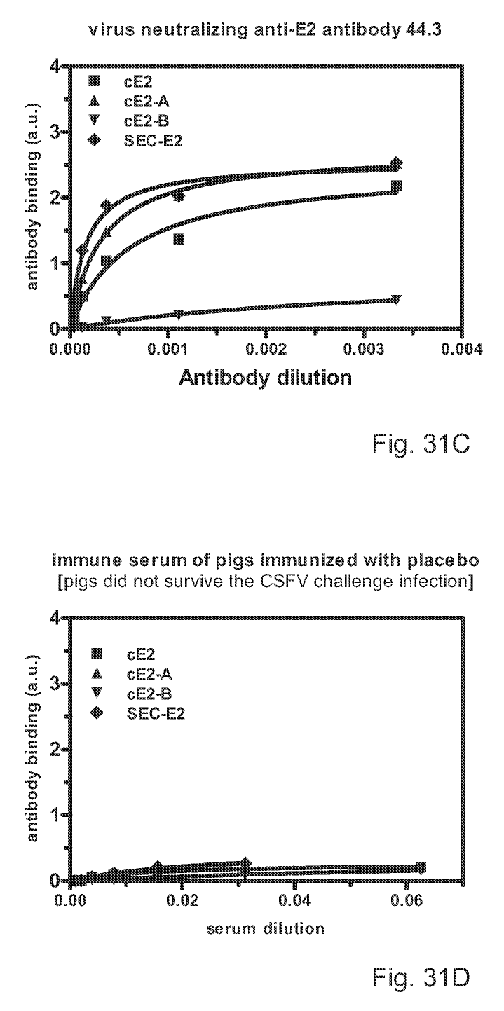

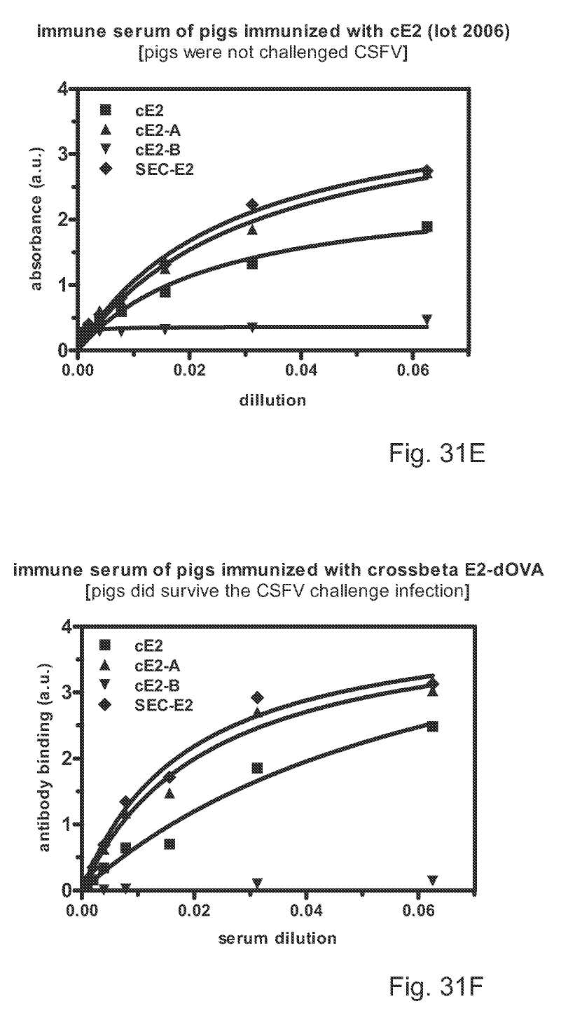

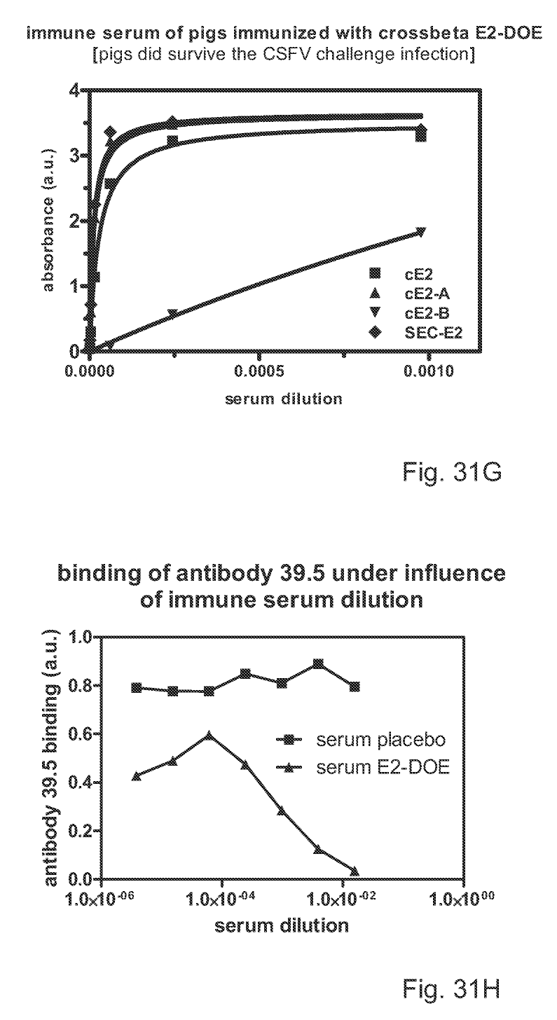

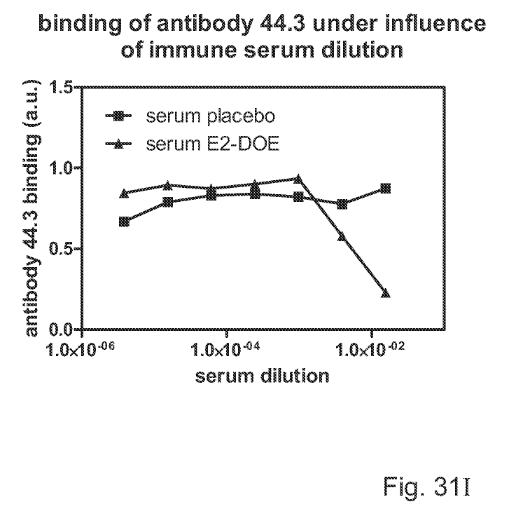

Analysis of exposure of functional epitopes on E2 forms. In an ELISA lay-out, it is assessed whether the various cross-beta comprising forms of E2 expose binding sites for CSFV neutralizing antibodies 21.1, 39.5 and 44.4, and for immune serum obtained from pigs that were immunized with various forms of E2 (See FIG. 31A.-G.). The immune sera were obtained during an immunization/CSFV challenge trial as outlined in patent application WO2007008070. Pigs immunized with placebo did not survive a challenge infection with CSFV; pigs immunized with E2-DOE all six survived the challenge infection. Pigs immunized with a different batch of cE2 which was covalently coupled to ovalbumin and subsequently applied to cross-beta inducing procedures, survived the CSFV challenge, as did the pigs that were immunized with cE2 adjuvated with double oil in water emulsion according to a commercialized protocol (CVI). The cE2 used for this previously disclosed immunization/challenge trial was from a different lot than the cE2 used for the currently disclosed experiments. It is seen that cE2-B hardly exposes epitopes for the functional monoclonals, neither for the anti-E2 antibodies in immune serum. Epitopes are similarly in number exposed in SEC-E2 and cE2-A, whereas somewhat less epitopes are accessible for the functional antibodies in cE2.

Subsequently, binding of virus neutralizing mouse monoclonal antibodies 39.5 and 44.3 to nE2 under influence of a dilution series of pooled pig serum obtained after immunization with placebo/PBS or with cE2 adjuvated with double oil in water emulsion according to a commercialized protocol, was assessed in an ELISA lay-out. The cE2 was coated and the two monoclonal antibodies 39.5 and 44.3 were contacted with the cE2 at a concentration that gave approximately half-maximum binding, as determined in the antibody binding experiment outlined above. A dilution series of immune serum obtained from pigs that were immunized with either placebo (buffer, PBS) or E2 in double oil in water adjuvant, was added to the half-maximum binding concentration of the functional monoclonals. The immune sera were again obtained from a previous immunization/challenge trial as outlined in patent application WO2007008070. Binding of the monoclonals 39.5 and 44.3 was assessed. See FIGS. 31H and I. Anti-E2 antibody titers in the immune sera are depicted in FIGS. 31D and G. With the data it is shown that pigs which survived a challenge with CSFV had an anti-E2 antibody titer, and the obtained pig immune serum competes for binding sites on cE2 with virus neutralizing antibodies 39.5 and 44.3. Serum from pigs of the placebo group, which did not survive the challenge, does not inhibit the binding of the functional monoclonal antibodies.

With this information we now know that pigs that survive a challenge with CSFV have antibodies that compete for binding sites on cE2 with virus neutralizing monoclonal antibodies. Therefore, the monoclonal functional antibodies are used for selection of E2 forms that expose the epitopes for the functional antibodies, and thus the epitopes that are bound by antibodies in immune serum of pigs that survive a CSFV challenge infection.

Immunization of Pigs with Various Cross-Beta Comprising Structural Forms of E2, and Subsequent Challenge with Classical Swine Fever Virus

Five groups of six pigs and one group of five pigs (group 2) were immunized with 32 μg recombinant E2/animal or with placebo (PBS, Test group T01). For the vaccination, antigens were applied as depicted in Table 8. Thirty-six male pigs were used, at first, but the sixth animal in group 2 died before the start of the study, at day −2, and could not be replaced anymore. Pigs were housed at the facilities of CVI. The pigs were approximately 6 weeks old at vaccination, and were free of antibodies against CSFV. Pigs were randomly allotted to a vaccine group or control group. The animals were fed, and could drink water ad libitum. At day 0 and 21 the pigs were immunized intramuscular with 2.0 ml test sample, once on the left and once on the right, approximately 2-5 cm behind the ear. Antigens were prepared and formulated by Cross-beta Biosciences, except for test item 3, used for group 3, i.e., E2 adjuvated with DOE. This test item was formulated freshly at the day of the vaccinations, by personel of CVI, according to an internal SOP.

Challenge with CSFV strain Brescia 456610. On day 42 the 35 pigs were inoculated intranasally with a dose of 200 LD₅₀ of the highly virulent CSFV strain Brescia 456610.

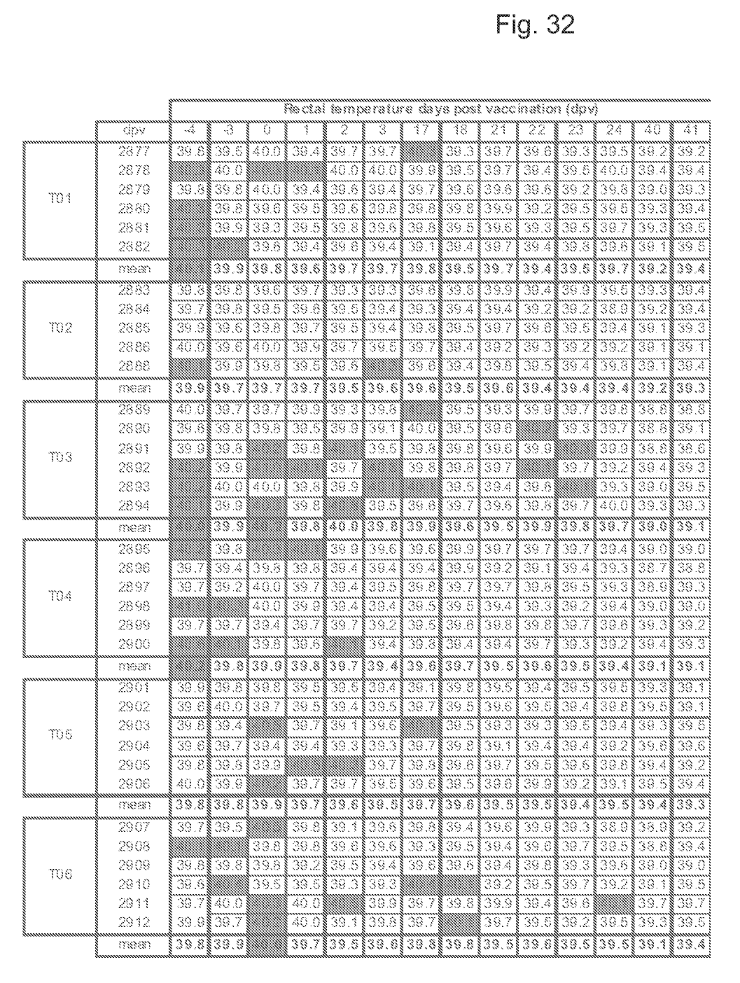

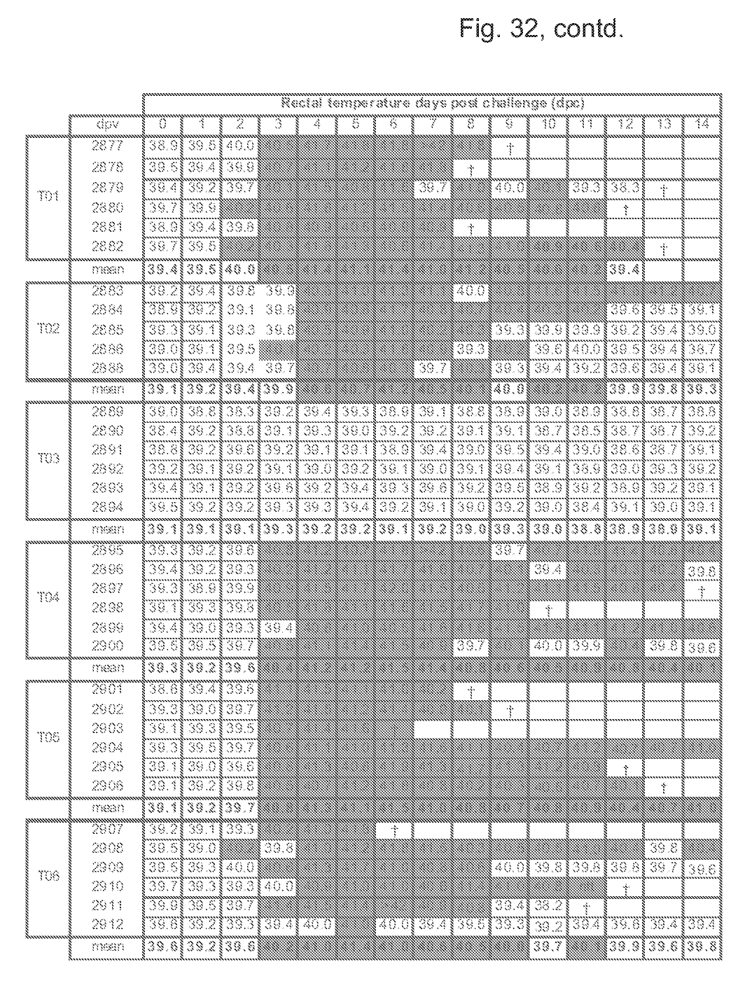

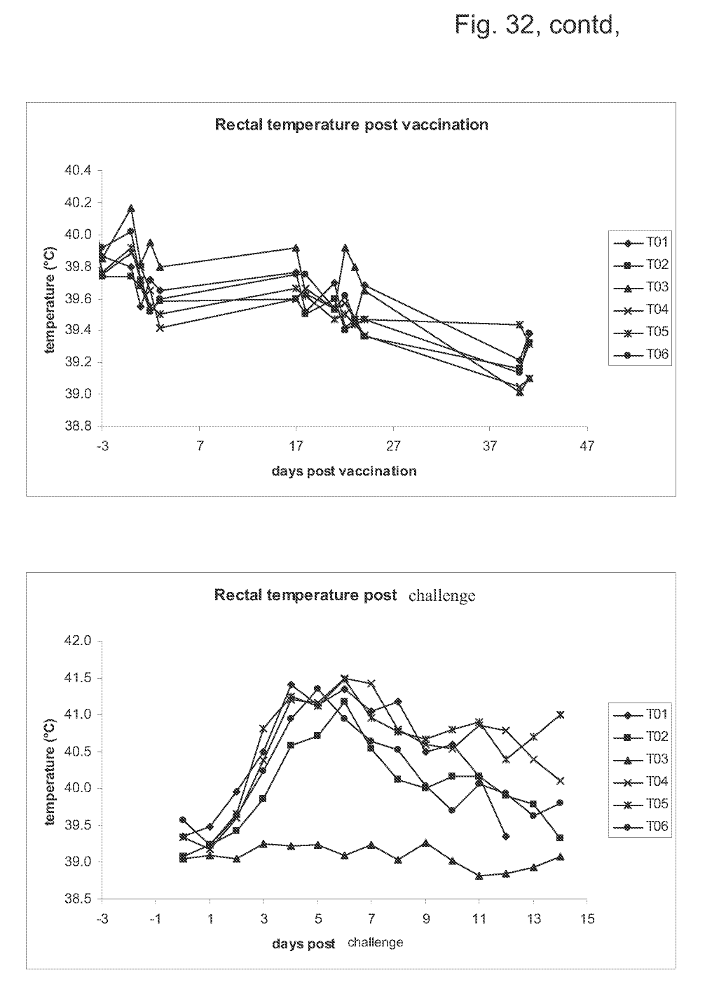

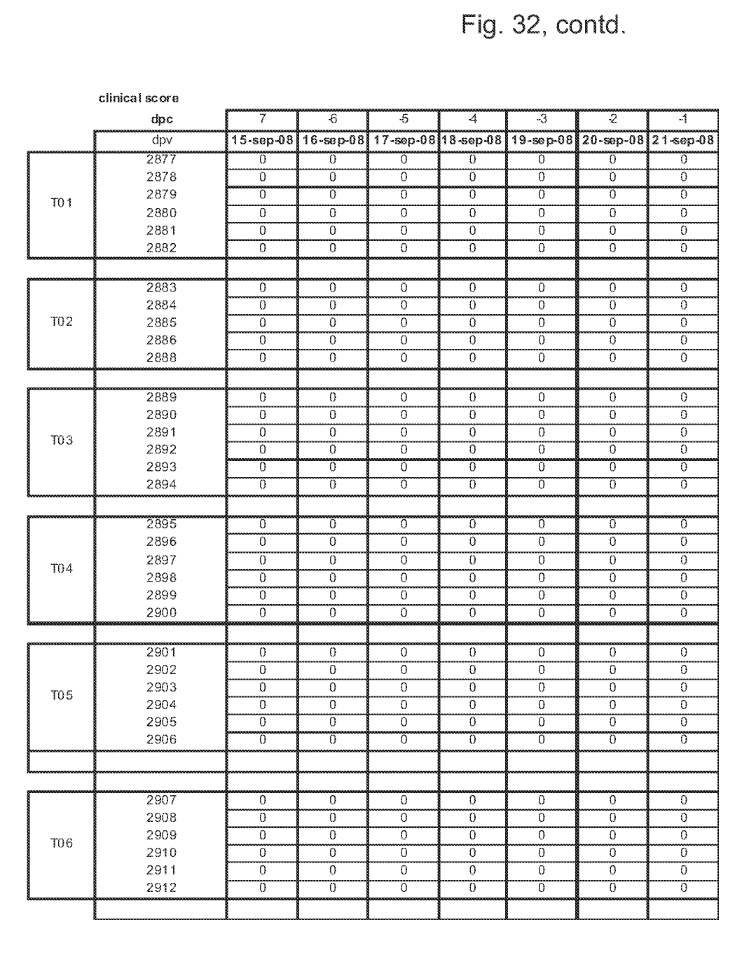

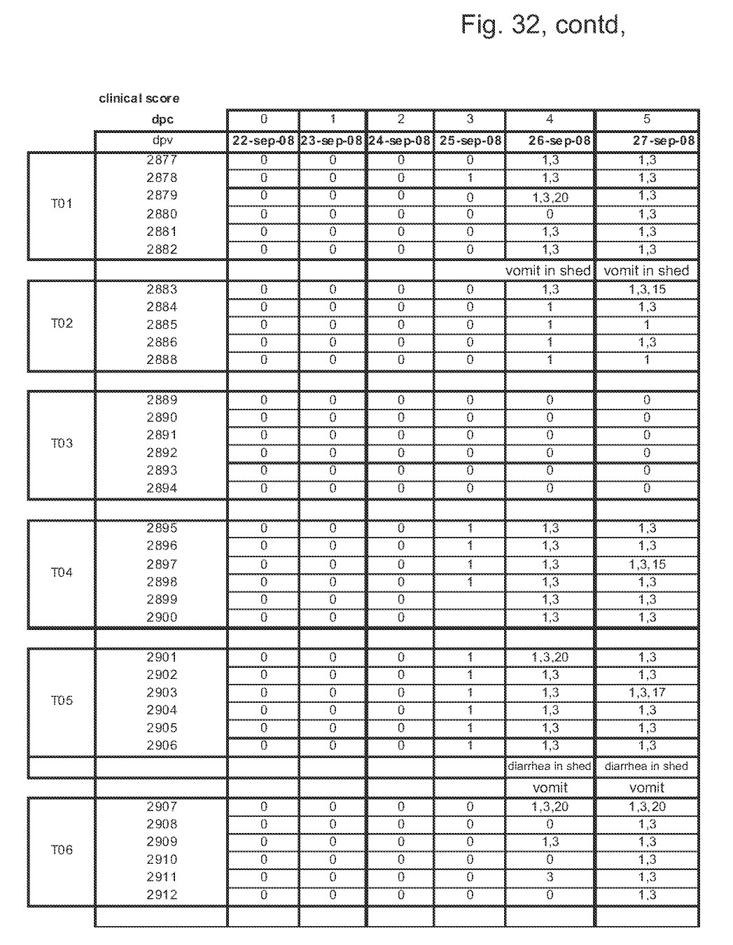

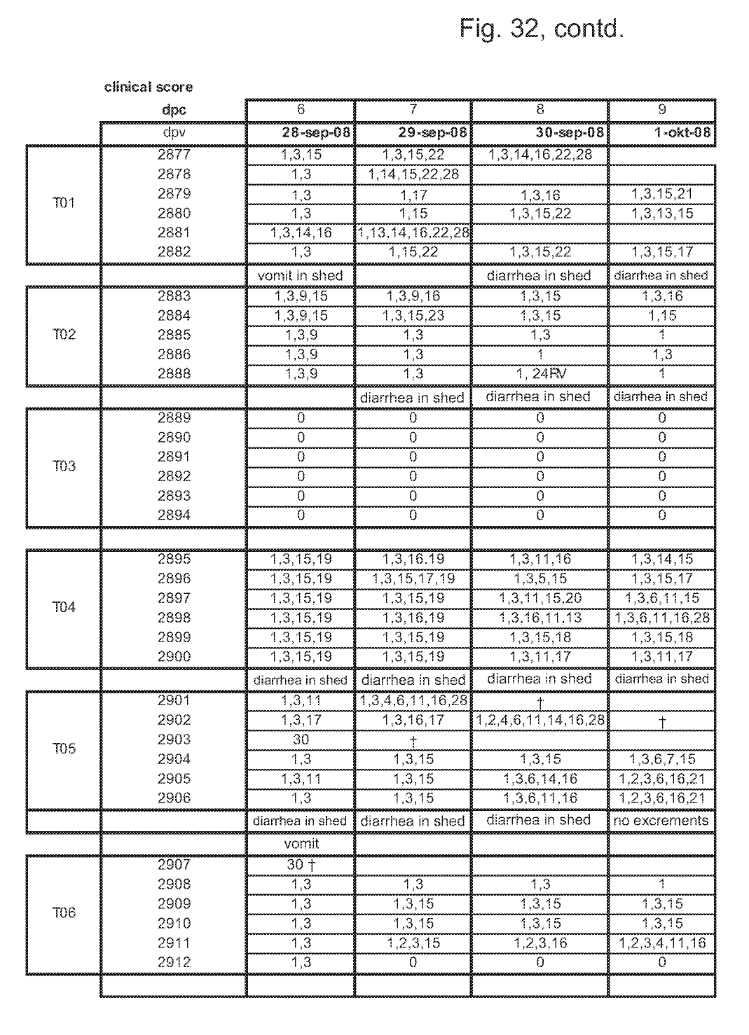

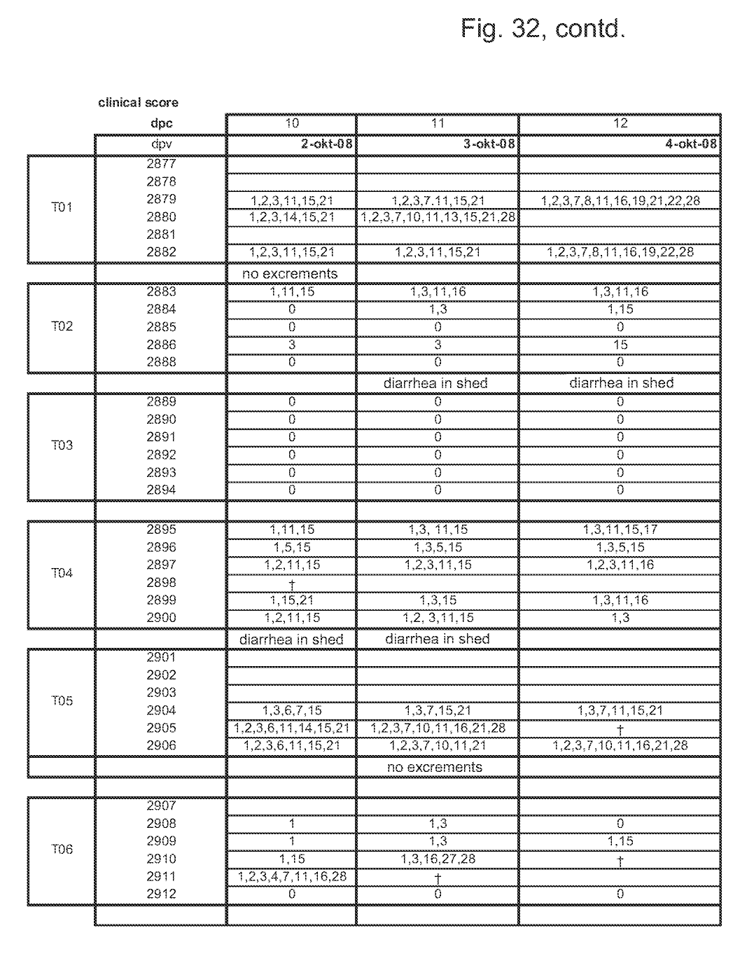

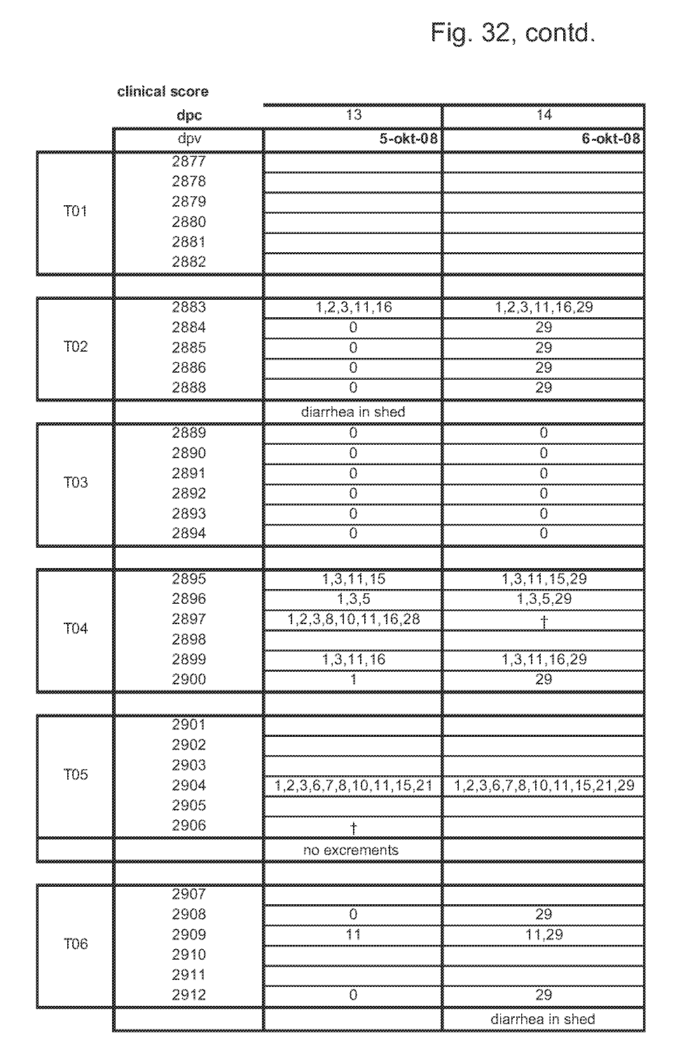

Evaluation and examination. Anal temperature was measured starting 4 days before the first immunization and during the challenge until the end of the experiment (day 56) (see FIG. 32). Fever was defined as a temperature above 40° C. In the days pre-challenge, in group 3 on average more pigs suffered from fever during more days, compared to the other groups. No fever was measured during the challenge period in group 3 (cE2-DOE). In group 2, upon viral challenge, the five pigs suffered from fever, which ended at day 9 post challenge for 2 pigs, at day 10 for a subsequent pig and at day 12 for a fourth pig. For groups 1, 4 and 5 fever remained, whereas for group 6 also at day 9 the temperature declined.

During the course of the whole study the animals were monitored once each day, which is outlined in FIG. 32. Survival is also indicated in the clinical scoring tables. Clinical signs are defined as:
0. No clinical signs
1. slow/tired/reduced responsiveness,
2. retarded growth, thin (waste),
3. decreased appetite, no appetite,
4. punctual bleedings in the skin
5. pale
6. red skin
7. red spots on the ears,
8. blue coloring of legs
9. blue coloring of nose
10. blue coloring of waste/tail
11. skin necrosis
12. conjunctivitis
13. nasal discharge (runny nose),
14. shivering,
15. unstable walking, hind legs
16. pig is unable to stand without assistance,
17. diarrhea
18. dry excrement
19. impairment of the respiratory system,
20. vomiting
21. snoring or sniffing breathing,
22. red eyes
23. kind of epileptic attack, falling, not reacting, shivering
24. lame
28. euthanasia

Pigs in positive group 3 did not suffer from clinical symptoms and all six survived the CSFV challenge. The pigs in placebo group 1 suffered on average from 6 clinical symptoms, when still surviving. Pigs died at day 8 (2), 9 (1), 12 (1) and 13 (2). Comparing groups 2, 4-6, immunized with various forms of cross-beta E2, reveals that on average pigs in groups 2 and 6 suffered from less clinical symptoms than pigs in groups 4 and 5. Analyzing survival reveals a somewhat different picture. Pigs did not die in group 2, pigs did die at day 10 (1), day 14 (1) in group 4, with four survivors, at day 7 (1), 8 (1), 9 (1), 12 (1), 13 (1) in group 5, with one survivor, at day 6 (1), day 11 (1) and day 12 (1) in group 6, with three survivors. In terms of survival upon challenge protection was provided according to cE2 (5/5)>cE2-A (4/6)>SEC-E2 (3/6)>cE2-B (1/6).

Blood samples for serum collection were taken at regular intervals including day 0, 7, 14, 21, 28, 35, 42 (challenge), 49 and 56 (end of challenge period). Sera was subsequently obtained after centrifugation and stored frozen.

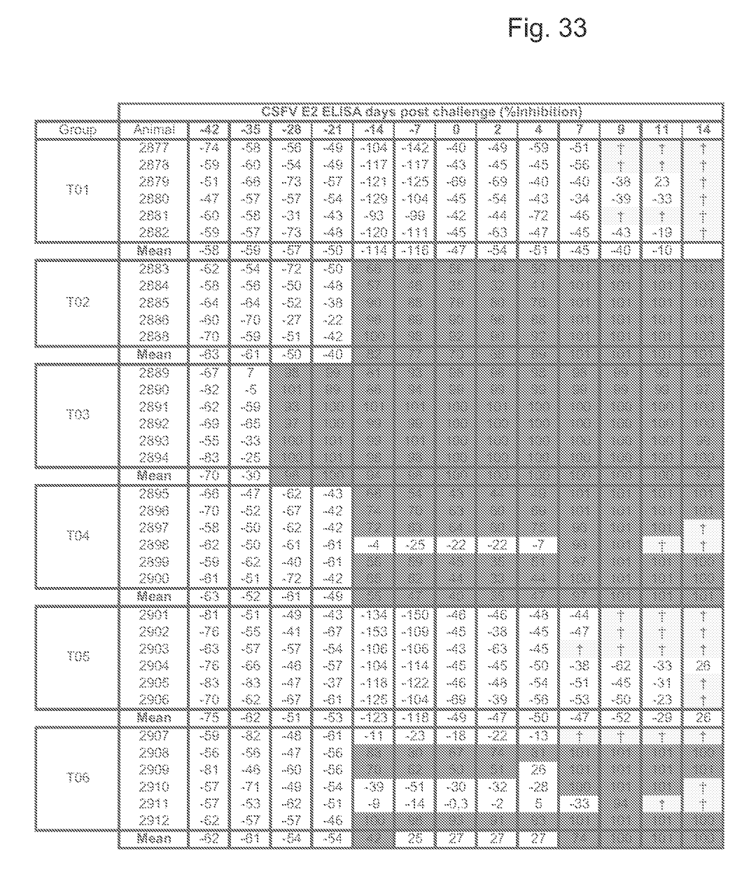

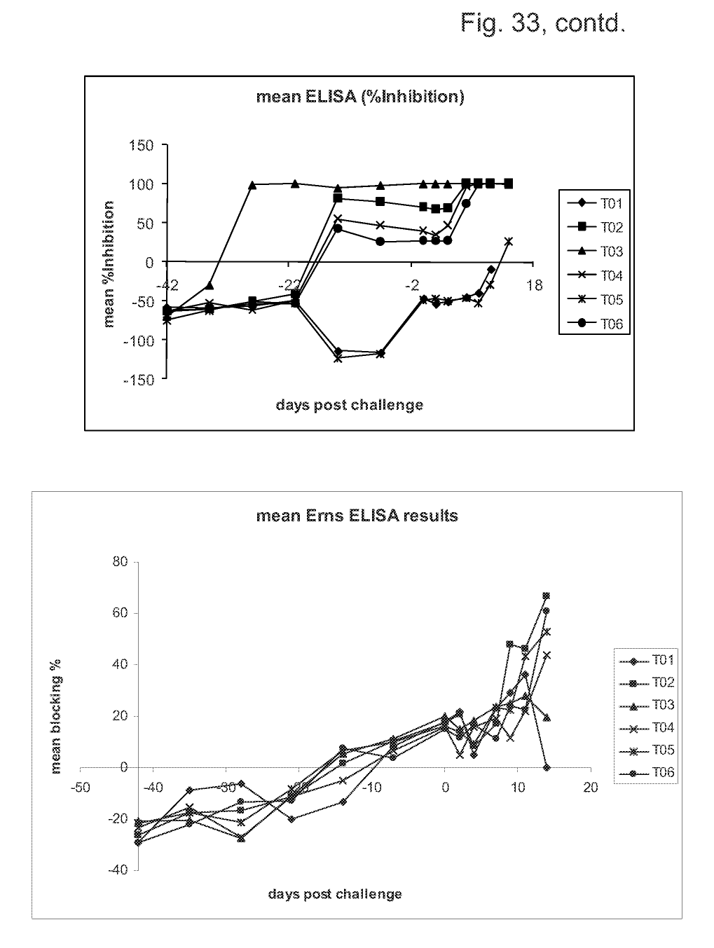

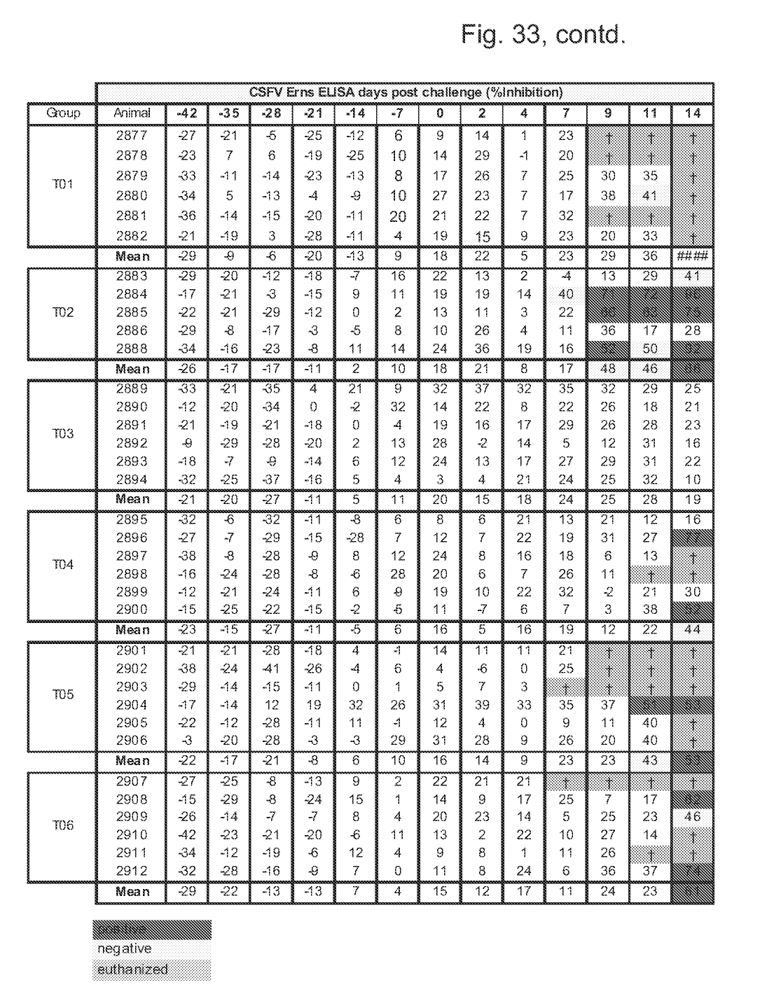

Anti E2 antibody titers were assessed by CV1, using the Ceditest CSFV kit (Prionics, the Netherlands). Results in FIG. 33 depict that two immunizations induced anti-E2 titers in 5/5, 6/6, 5/6, 0/6 and 3/6 pigs in groups 2, 3, 4, 5, 6, respectively. In general, pigs that developed a titer survived the subsequent challenge with CSFV, with pig 2897 in group 4 being the exception; a titer is determined, but still the pig did not survive, although it survived up to the final day of the challenge period. In FIG. 33, also anti-Ems titers are displayed. Titers against this CSFV glycoprotein are a measure for titers against the virus particle, and are assessed by CV1 using the Bommeli CHECKIT-CSF-MARKER Test Kit. At day 9, three out of five pigs in group 2 (cE2 antigen) developed a titer, whereas in other groups titers developed two to five days later, if at all in this period. No titers developed in pigs in group 3 (cE2-DOE).

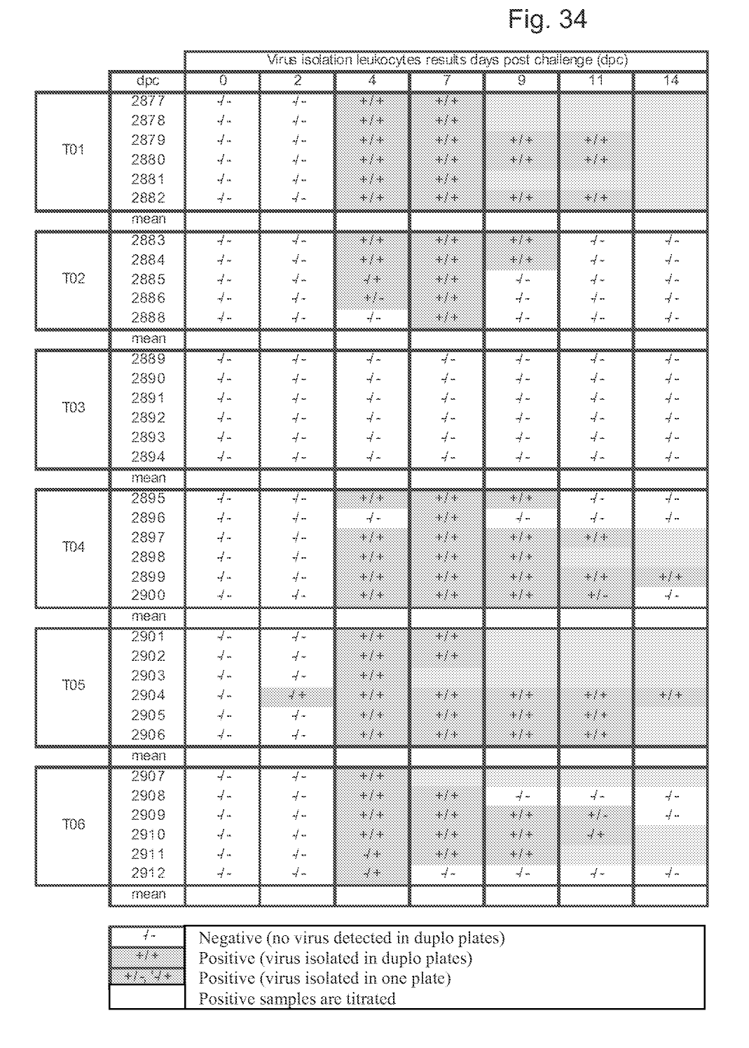

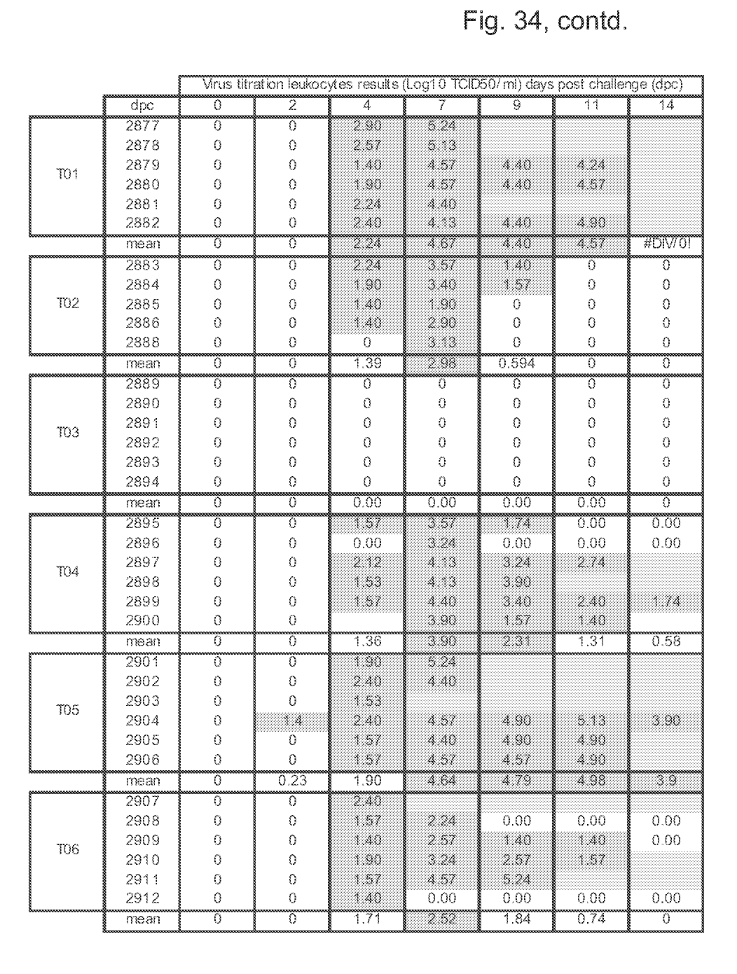

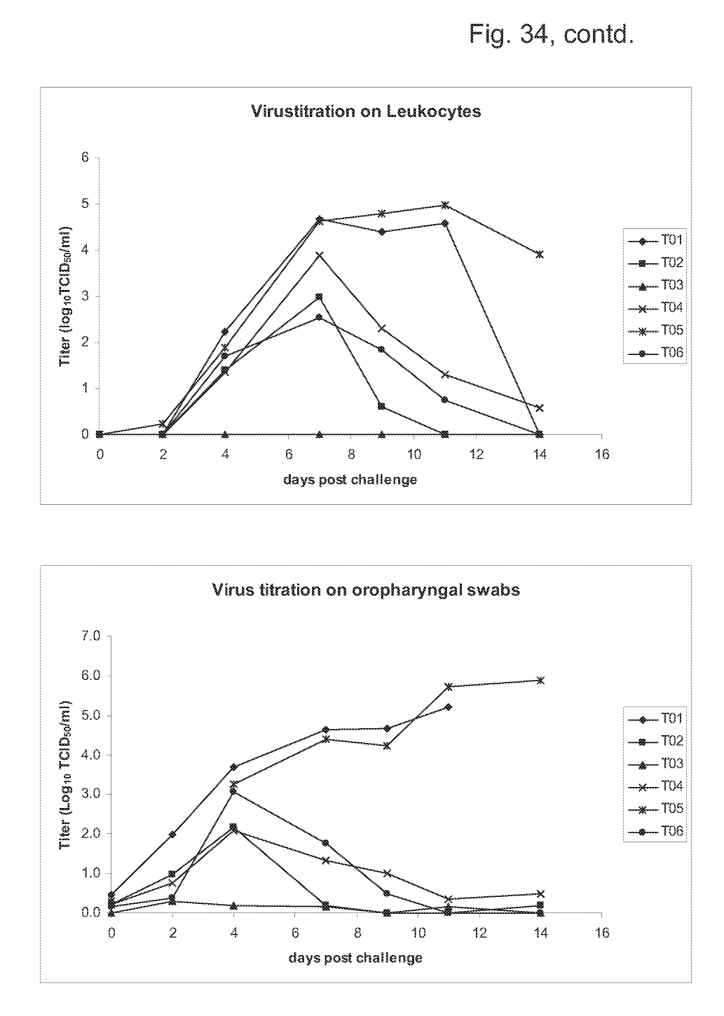

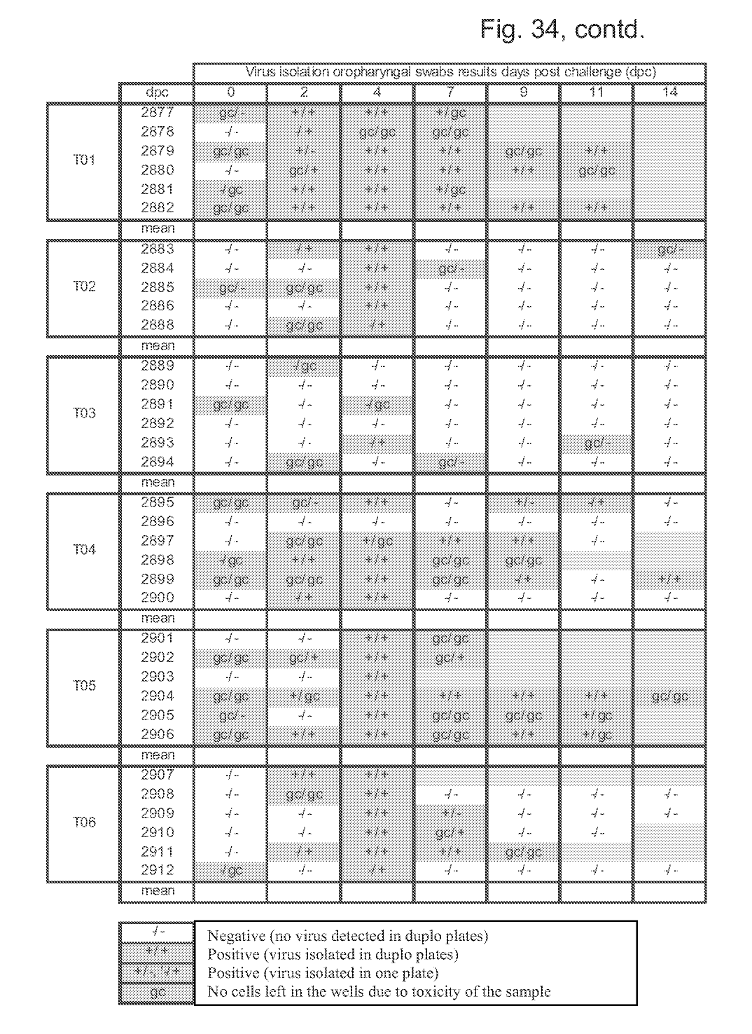

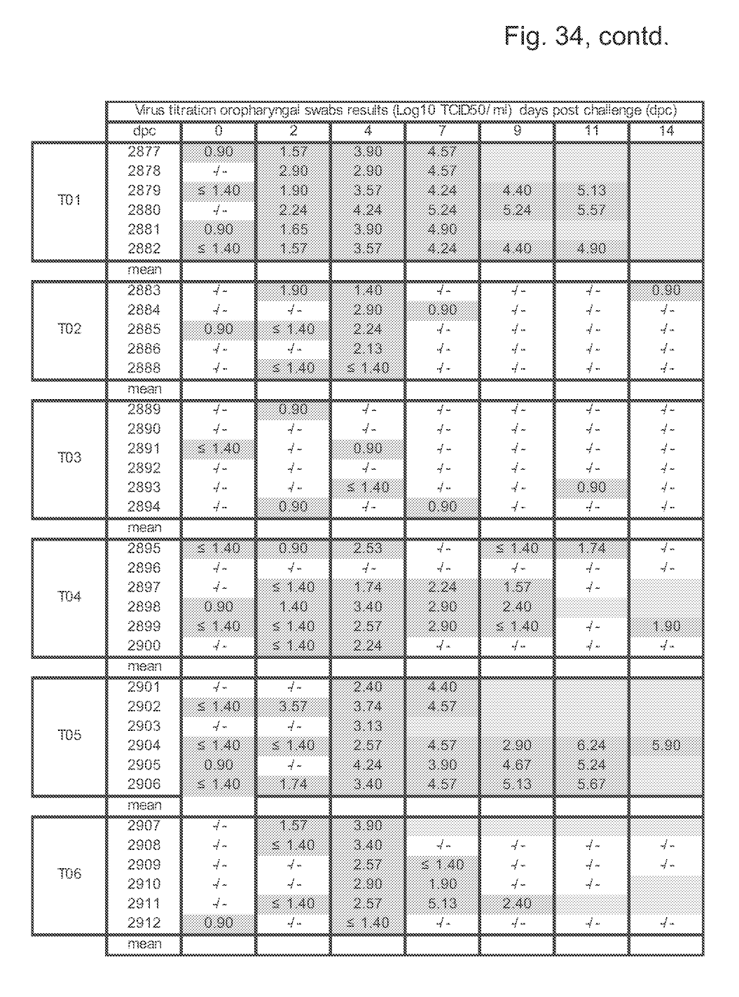

Virus isolation from leucocytes and from oropharyngal swabs was performed by CV1, according to standard procedures at CV1. For the virus isolation from leucocytes, first the presence of virus was assessed, followed by a titration experiment with positive samples. In FIG. 34 it seen that virus is present in leucocytes from day 4 post-challenge on, in pigs in groups 1, 2, 4-6. In group 2, all pigs are free of leucocytes at day 11 post challenge. In groups 4-6 pigs have on average leucocytes free of virus at a later stage, or still virus is detected in leucocytes at day 14 (final day of the challenge period). Similar results are seen with the virus isolation data obtained with oropharyngal swabs. In conclusion, based on the virus load in leucocytes and in oropharyngal swabs, and the survival data, the cE2 antigen provided the best protection when compared with the other three cross-beta comprising E2 variants cE2-A, cE2-B and SEC-E2.

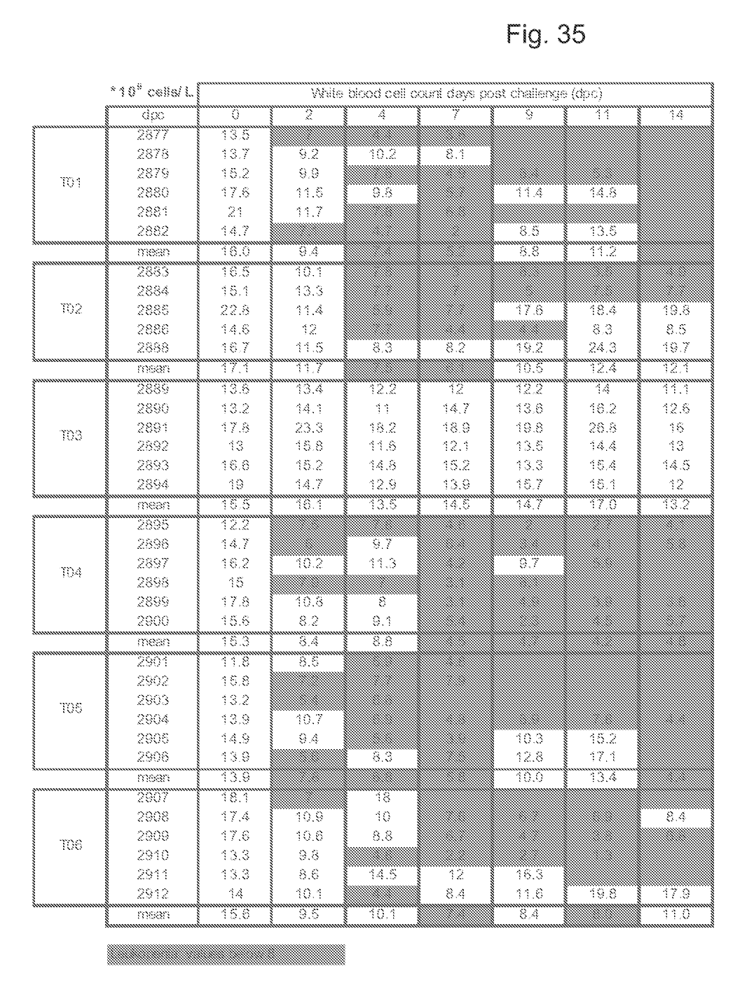

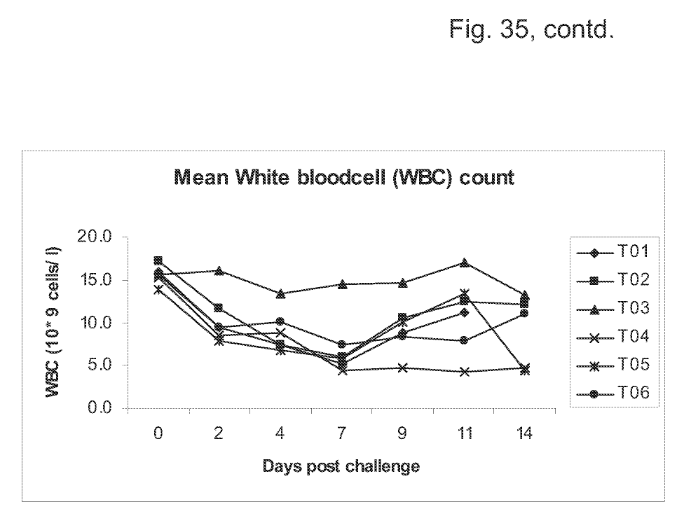

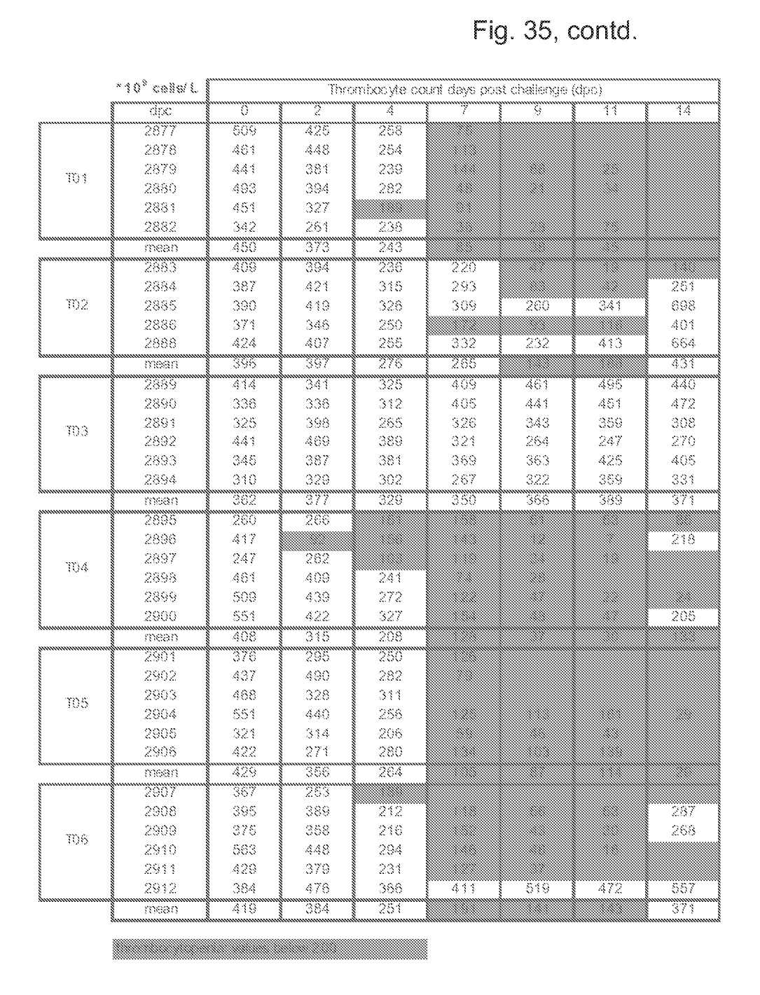

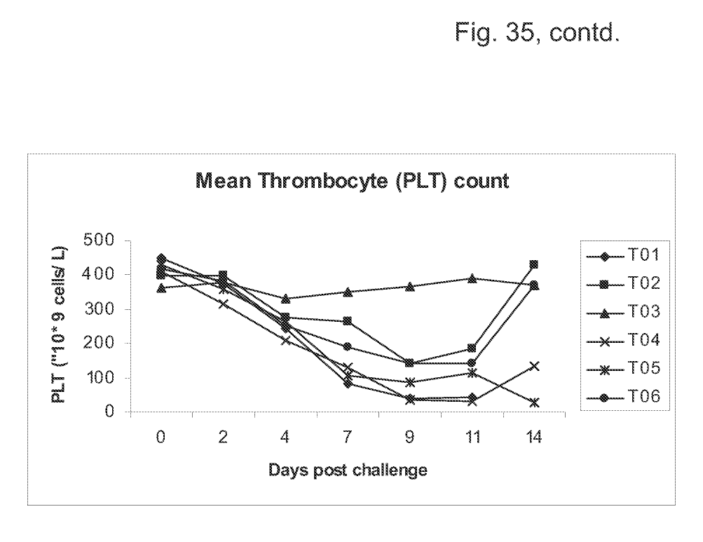

During the post-challenge period, white blood cells and thrombocytes in blood are counted for all surviving pigs at day 0, 2, 4, 7, 9, 11 and 14 (end of challenge period). See FIG. 35 for the data, which are collected and processed by CV1 (Lelystad, the Netherlands). On average it is seen that when comparing the pigs immunized with any of the four cross-beta E2 variants in group 2, 4-6, less pigs in group 2 and 6 suffer shorter and less severely from a drop in white blood cell count and thrombocyte count.

### Concluding Remarks

Based on the cross-beta structural data and on the exposure of epitopes for virus neutralizing antibodies, it was expected that cE2-B would provide pigs with relatively less protection against CSFV challenge, compared to other cross-beta comprising E2 forms (See FIG. 31A-C). The combination of cross-beta structure with a decreased number of exposed epitopes for functional antibodies in cE2-B, when compared to other E2 forms, is at the basis of this assumption. Indeed, when now comparing the clinical data and the titer data, cross-beta E2 form cE2-B proved to poorly induce protection, with one surviving pig that was still critically ill at day 14 post-challenge. The cE2 form proved to provide relatively the best protection; 5/5 pigs survived the challenge and on average clinical data showed a somewhat less severe disease process, when compared to the other three cross-beta E2 forms. The SEC-E2 form induced an immune response that resulted in comparable clinical parameters, although three out of six pigs did not survive the challenge. The cE2-A protected 4/6 pigs from lethality, and clinical parameters indicate that pigs were relatively more ill than pigs immunized with cE2. In conclusion, comparing the cE2 with the cE2-A and SEC-E2, it is evident that cE2 is provided with a better combination of type and appearance of cross-beta structure in cross-beta structure comprising E2 molecules, in combination with exposed epitopes for functional antibodies.

### Example

### Factor VIII

Factor VIII structural variants with varying cross-beta content and varying number of exposed epitopes for factor VIII inhibiting antibodies induce factor VIII inhibiting antibodies in mice to various extent.

As described above, a series of factor VIII structural variants comprising cross-beta structure, referred to as cross-beta factor VIII forms, are prepared from Helixate recombinant human factor VIII. Factor VIII monomer has a molecular mass of approximately 280 kDa, comprising 2332 amino acid residues, with eight disulfide bonds and 22 (potential) N-linked carbohydrates.

### Factor VIII Structures Comprising Cross-Beta Structure and Exposing Epitopes for Factor VIII Neutralizing Antibodies Induce Neutralizing Antibodies in Mice

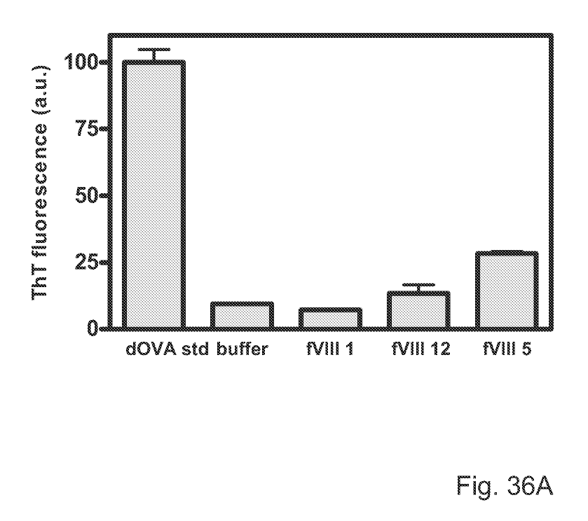

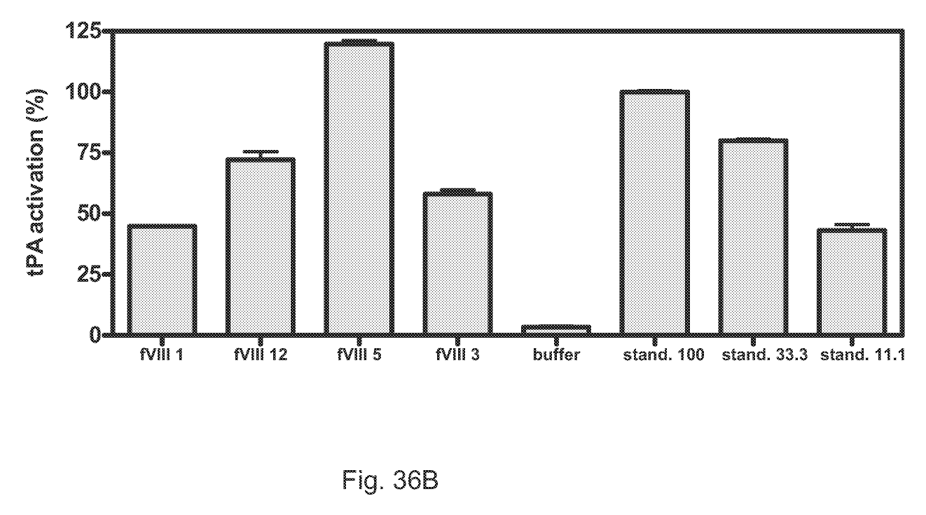

For immunizations, a modified version of cross-beta factor VIII form 3 is prepared; cross-beta factor VIII form 12, incubated prolonged for 1 week, instead of for 20 h, at 37° C. after dissolving, followed by storage at 4° C. This cross-beta form 12 is compared with cross-beta forms 1 and 5 in the ThT fluorescence enhancement assay (FIG. 36A) and with cross-beta factor VIII forms 1, 3 and 5 in the tPA/plasminogen activation assay (FIG. 36B). It appears that the relative cross-beta content in cross-beta fVIII forms 1, 12 and 5 is approximately 50, 75 and 125%, based on the tPA/Plg activation assay and compared to a misfolded ovalbumin standard, with a similar relative cross-beta content amongst the three cross-beta factor VIII forms 1, 12 and 5 deduced from the ThT fluorescence enhancement assay; relative cross-beta content 7, 13 and 28 compared to the misfolded ovalbumin standard.

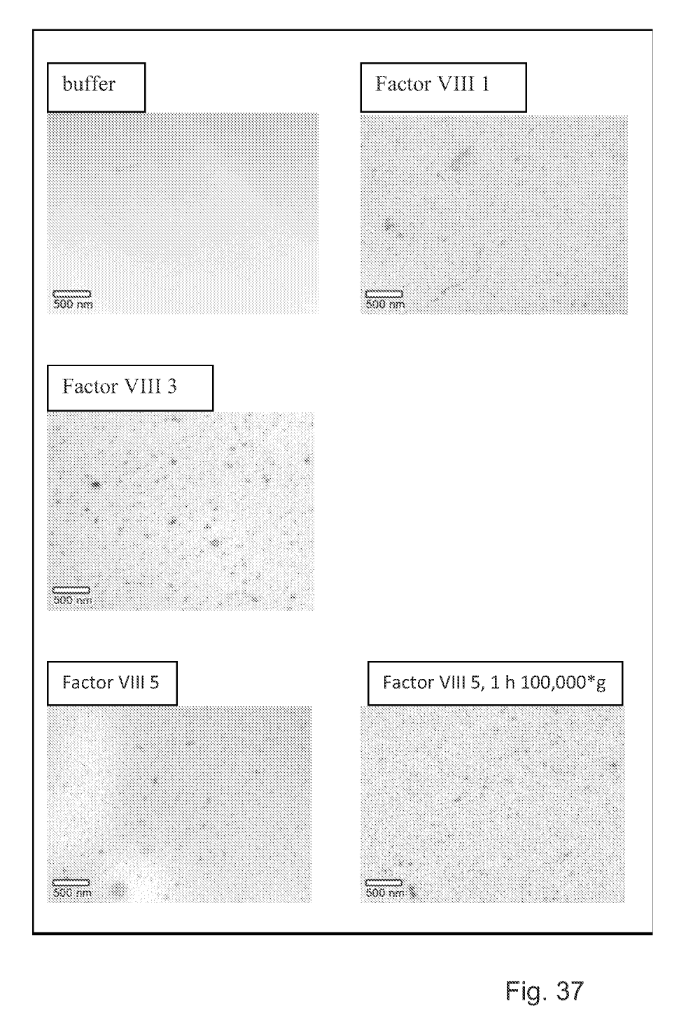

In addition to the structural data for cross-beta fVIII forms as outlined above, TEM images are taken for cross-beta forms 1, 3 (preparation comparable to cross-beta form 12) and 5, as well as negative control PBS (See FIG. 37). From the images it is seen that forms 1 and 3 comprise a background of relatively small protein assemblies, with an approximate size of 5-10 nm, which would fit a factor VIII monomer. For cross-beta form 5 these abundant assemblies have larger dimensions of approximately 10-20 nm, corresponding to factor VIII dimers of 4664 amino acid residues. Form 1 comprises a number of factor VIII structures with dimensions of approximately 10-20 nm, which appear as relatively loosely assembled molecules. For cross-beta form 3, a higher number of assemblies with this approximate size is seen, together with a few somewhat larger structures, now with a relatively more dense appearance. Form 5 also appears as a few structures with an approximate size of 25-50 nm, corresponding to factor VIII trimers up to 12-mers. Upon ultracentrifugation for 1 hour at 100,000*g, the appearance of cross-beta factor VIII form 5 does not change optically. In summary, the relative size of factor VIII assemblies is in the order: cross-beta factor VIII form 5>cross-beta form 3 (relatively dense structures)=cross-beta form 1.

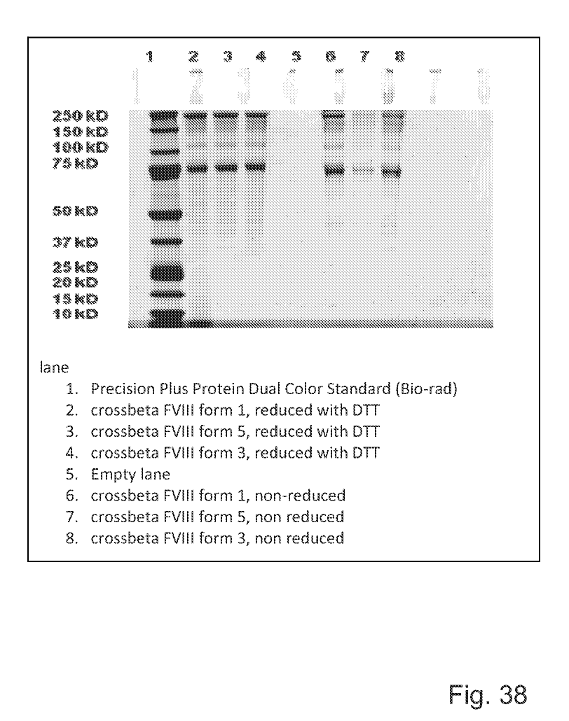

In FIG. 38 an analysis using SDS-PA gel electrophoresis with the cross-beta factor VIII forms 1, 3 and 5 is given. It appears that under non-reducing conditions, cross-beta form 5 comprises a relatively high amount of multimers that do not enter the gel. Form 3 also comprises a fraction of multimers that do not enter the gel, although to a lesser extent than seen with form 5. Compared to form 1, in form 3 and 5 a smear of factor VIII multimers with a molecular size of larger than 250 kDa is seen. Under reducing conditions, all three cross-beta factor VIII forms appear similarly on gel with main protein bands at approximately 75 kDa and 250 kDa. The 250 kDa band corresponds with the factor VIII monomer.

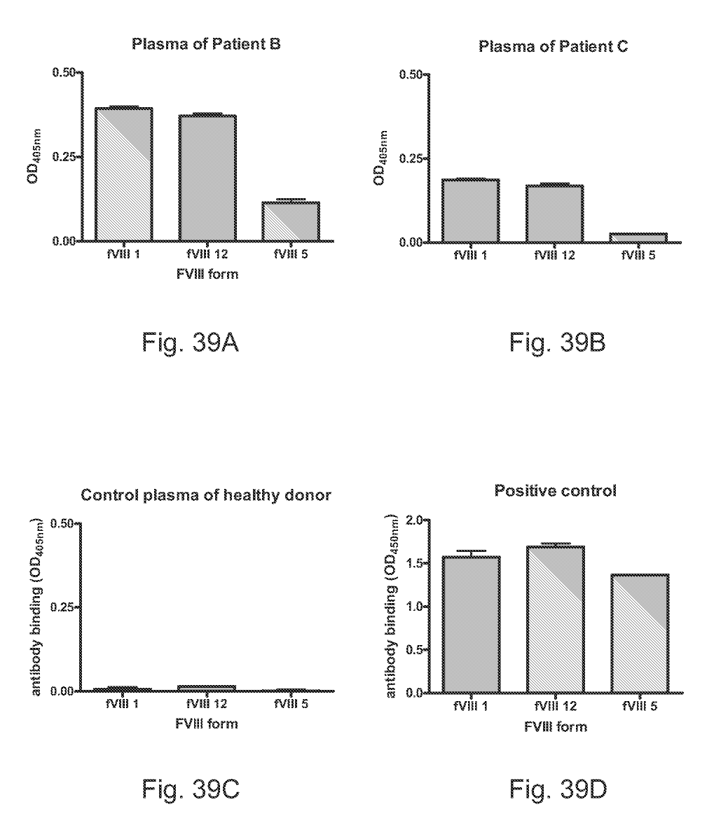

Furthermore, the cross-beta factor VIII forms 1, 5 and 12 are compared for their relative exposure of epitopes for factor VIII inhibiting antibodies in human haemophilia patient plasma (FIG. 39).

See the general outline of an immunization trial with mice and various cross-beta forms of factor VIII. According to the general experimental outline, four groups of five mice were immunized intravenously for four times. Antigens used were as depicted in the legend to Table 10. The selection of these three cross-beta forms of factor VIII is based on the following criteria. In FIG. 36 it is seen that factor VIII forms 1, 12 and 5 all three comprise cross-beta structure, though to a varying extent in the order 5>12>1. In FIG. 12 it is depicted that the cross-beta structure in factor VIII form 5 is present as approximately 45% insoluble molecules. It is generally accepted in the field of protein misfolding research that the soluble protein fraction is obtained upon an equivalent of centrifugation for 1 hour at 100,000*g, for example 30 minutes at 200,000*g. From FIGS. 17A-D, 19A-D and FIG. 39, it is depicted that cross-beta factor VIII form 1 exposes relatively the largest number of epitopes for factor VIII inhibiting antibodies present in human haemophelia patient plasma, whereas the number of epitopes is to some extent decreased in cross-beta factor VIII form 3 and 12, and exposure of epitopes is strongly decreased in cross-beta factor VIII form 5. In summary, the relative number of exposed epitopes for factor VIII inhibiting antibodies is in the order cross-beta factor VIII form 1≈form 3, form 12>form 5. See also FIG. 39.

Plasma of the mice was collected at day 56 after the first immunization (immunizations at day 0, 14, 26 and 42). Titers against freshly dissolved factor VIII are determined and given in Table 10. At day 97, 55 days after the final immunization, plasma was again collected for analysis of the presence of factor VIII inhibiting antibodies. In a Bethesda assay that is applicable for the use with mouse plasma (developed at Good Biomarker Sciences, Leiden, the Netherlands), the presence and relative amount of antibodies in the mouse immune plasmas that inhibit factor VIII in human plasma, was assessed and given as Bethesda units per ml plasma (BU/ml). Values are given in Table 10. From Table 10 it is clearly seen that cross-beta factor VIII form 1, which comprises cross-beta structure and relatively the most epitopes for factor VIII inhibiting antibodies present in human haemophilia patient plasmas, induces antibody titers in five out of five mice, that inhibit human factor VIII. Cross-beta Factor VIII form 12, comprising relatively more cross-beta structure and a comparable number of epitopes for factor VIII inhibiting antibodies, induces anti-fVIII titers in two out of five mice (a titer of 16 is considered as negative, because one mouse in the placebo PBS group is presented with a titer of 16), which titers are comprising factor VIII inhibiting antibodies. Cross-beta Factor VIII form 5, comprising relatively the most cross-beta structures in on average the largest molecular assemblies which in part are insoluble, and comprising far less epitopes at the molecular surface, if any, for factor VIII inhibiting antibodies, induces titers in four out of five mice, but which titers are not comprising human factor VIII inhibiting antibodies.

From these data it is concluded that the combination of cross-beta structure in factor VIII and exposed epitopes for factor VIII inhibiting antibodies, as in cross-beta factor VIII form 1 and in form 12, is required for eliciting factor VIII inhibiting antibodies in an animal. Cross-beta Factor VIII form 5 comprises immunogenic cross-beta structures, as expressed by the anti-fVIII titers, but comprises hardly any exposed epitopes for factor VIII inhibiting antibodies. Indeed, cross-beta factor VIII form 5 induces an antibody response but these antibodies turn out not to be functional antibodies, i.e., factor VIII inhibiting antibodies, in accordance with the strongly reduced exposure of epitopes in the factor VIII antigen used for immunizations. This demonstrated the necessity of a combination of immunogenic cross-beta structure and exposed and available epitopes for functional antibodies, in an immunogenic composition for induction of functional antibody titers. Based on the molecular size distribution, multimers of up to factor VIII 12-mers are capable of eliciting an immune response.

### Example

### Ovalbumin

This example illustrates the ability to generate and select immunogenic compounds comprising a cross-beta structure and epitopes for antibodies capable of inducing an humoral response.

Study design. Ovalbumin was used as test protein and antigen. Cross-beta structure was induced in OVA in three different ways. Exposure of epitopes for a series of anti-OVA antibodies was scanned and compared. Mice were immunized with OVA, comprising relatively low cross-beta structure content (nOVA) or with three cross-beta OVA forms comprising increased numbers of cross-beta structure. In sera the antibody titer against nOVA was determined.

Preparation of Cross-Beta Variants of Ova. Four Different Forms of Ova comprising cross-beta structure, termed nOVA, dOVA-1, dOVA-2 and dOVA-3, were prepared according to examples of procedures to induce cross-beta structure described in this application and described below, and were compared in this example.

Cross-beta nOVA. OVA was dissolved in PBS to a concentration of 1.0 mg/mL. The solution was kept for 20 min at 37° C. in a water bath and subsequently for 10 min on the roller device (at room temperature). Aliquots were stored at −80° C. This cross-beta OVA form is referred to as nOVA, cross-beta nOVA or nOVA standard.

Method for inducing cross-beta structure: dOVA-1. OVA was dissolved at 5.2 mg/ml in HBS buffer (20 mM Hepes, 137 mM NaCl, 4 mM KCl). To dissolve OVA the solution was incubated for 20 min in a water bath at 37° C. and 10 min on a roller device at RT. The solution appeared clear. 5 M HCl is added to 2% of the total volume. The solution was mixed by swirling. The solution was incubated for 40 minutes at 37° C. (water bath). The solution appeared white/turbid. 5 M NaOH stock (2% of the volume) was added to neutralize the solution. The solution was mixed by swirling. The visual appearance of the solution remained turbid. Samples were aliquoted and stored at −80° C.

Method for inducing cross-beta structure: dOVA-2. OVA was dissolved in PBS to a concentration of 1.0 mg/mL. The solution was kept for 20 min at 37° C. in a water bath and subsequently for 10 min on the roller device (at room temperature). 200 μl aliquots in PCR cups were heat-treated in a PCR machine (MJ Research, PTC-200) (from 30° C. to 85° C. in steps of 5° C. per min). This cycle was repeated 4 times (in total 5 cycles). The samples were subsequently cooled to 4° C. The solutions were pooled, divided in 100 μL aliquots and stored at −80° C.

Method for inducing cross-beta structure: dOVA-3. OVA was dissolved in PBS to a concentration of 1 mg/ml and subsequently incubated for ten minutes at 37° C. followed by ten minutes RT incubation on a roller device. 200 μL aliquots were incubated in PCR strips (total 5.5 mL) at 75° C. in MyiQ real time PCR, BIORAD ΔT=one minute at 25° C., 25° C. to 75° C., ramp rate 0.1° C./second, incubation time approximately 16 h at 75° C., without cooling.

Endotoxin measurement. The endotoxin content of OVA was measured at 20 μg/mL (diluted in sterile PBS). The Endosafe cartridge had a sensitivity of 5-0.05 EU/mL (Sanbio, The Netherlands). The endotoxin levels are shown in table 11. The endotoxin level of the dilution buffer PBS is checked regularly and is below 0.050 EU/mL. Mice were immunized with 5 μg of cross-beta OVAs per mouse. The amount of endotoxins in 5 μg is calculated from the endotoxin level determined at 20 μg/mL.

### Structural Analysis of Cross-Beta OVA Variants

Visual inspection by eye and under a microscope, of various OVA forms. Table 12 describes the appearance of nOVA and the three dOVAs by eye. It is observed that dOVA-1 and dOVA-3 comprise insoluble OVA multimers as the solution is no longer clear upon treatment.

Transmission electron microscopy imaging (TEM) with OVA forms. The various OVA forms are subjected to TEM analysis. Table 13 summarizes the analysis. It is seen that multimeric OVA structures are induced by all three treatments. Aggregates are observed that vary in size in all dOVA variants, indicating the presence of cross-beta structure. In nOVA no aggregates are visible on the TEM image.

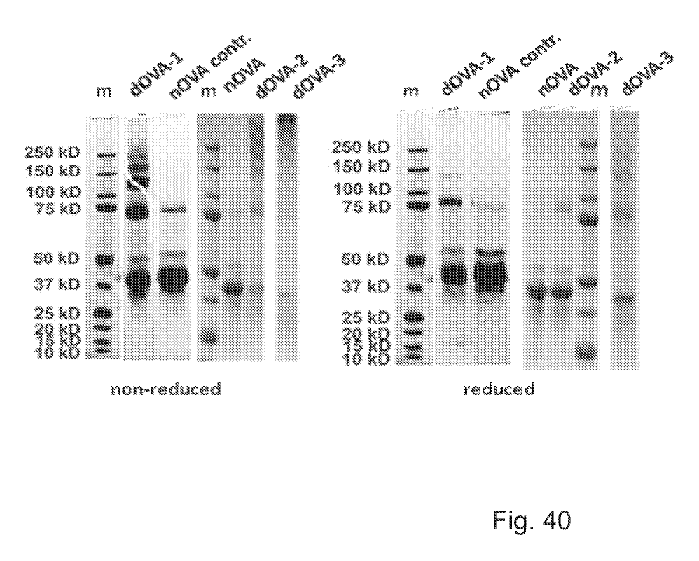

SDS-PAGE analysis of the OVA samples. FIG. 40 shows the analysis of the four OVA samples by SDS-PAGE gel electrophoresis under non-reducing and reducing conditions. The nOVA sample appears as a prominent band at around 40 kDa. A less prominent band is observed at 75 kDa, this band disappears upon reduction. All dOVA forms comprise the same OVA bands as nOVA, albeit in lower or much lower amount depending on the treatment condition used to induce cross-beta structure. In addition, high molecular weight bands are seen in all three cross-beta dOVA forms, indicative for the presence of multimers that do not separate under the conditions of SDS-PAGE analysis. dOVA-2 and dOVA-3 display as a smear of higher molecular weight bands, these bands run higher in the gel than the high molecular weight bands of dOVA-1. Upon reduction part of the high molecular bands disappear to the 40 kDa band. In conclusion, the various dOVA samples comprise different multimeric properties and more multimers compared to nOVA.

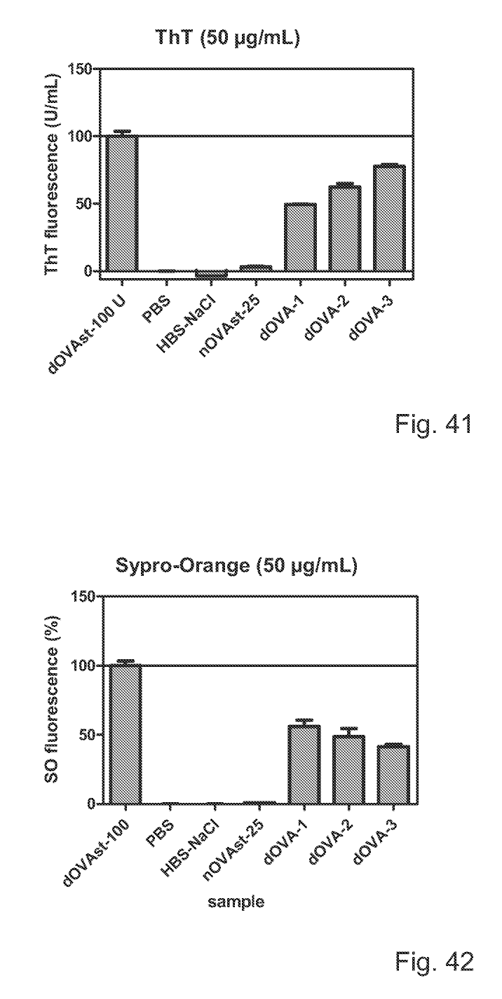

Enhancement of Thioflavin T fluorescence under influence of various OVA forms. Binding of Thioflavin T and subsequent enhancement of its fluorescence intensity upon binding to a protein is a measure for the presence of cross-beta structure which comprises stacked beta sheets. For measuring the enhancement of Thioflavin T fluorescence, OVA samples were tested at 50 μg/ml final dilution. Dilution buffer was PBS. Negative control was PBS, positive control was 100 U/ml standard (reference) misfolded protein solution, i.e., dOVA standard. dOVA standard is obtained by cyclic heating from 30 to 85° C. in increments of 5° C./minute a 1 mg/ml OVA (ovalbumin from chicken egg white Grade VII, A7641-1G, Lot 066K7020, Sigma) solution in PBS. FIG. 41 shows the analysis of OVA samples with ThT. Applying the three outlined cross-beta inducing procedures results in an increase in Thioflavin T fluorescence, compared to nOVA. The highest increase is seen with dOVA-3; approximately a 25-fold increase when compared to nOVA. dOVA-1 and dOVA-2 are increased 15 and 19 times respectively compared to nOVA (Table 14).

Enhancement of Sypro Orange fluorescence. Sypro Orange is a probe that fluoresces upon binding to misfolded proteins. As a measure for the relative content of proteins comprising cross-beta structure, enhancement of Sypro Orange fluorescence is tested with OVA samples at 50 μg/ml final dilution. Dilution buffer was PBS. Negative control was PBS, positive control was 100 μg/ml dOVA standard. The results are shown in FIG. 42 and Table 15. Applying misfolding results in an increase in Sypro Orange fluorescence. The highest increase is seen with dOVA-1; approximately a 60-fold increase when compared to nOVA. dOVA-2 and dOVA-3 are increased 55 and 45 times respectively. The trend is now opposite from the ThT data.

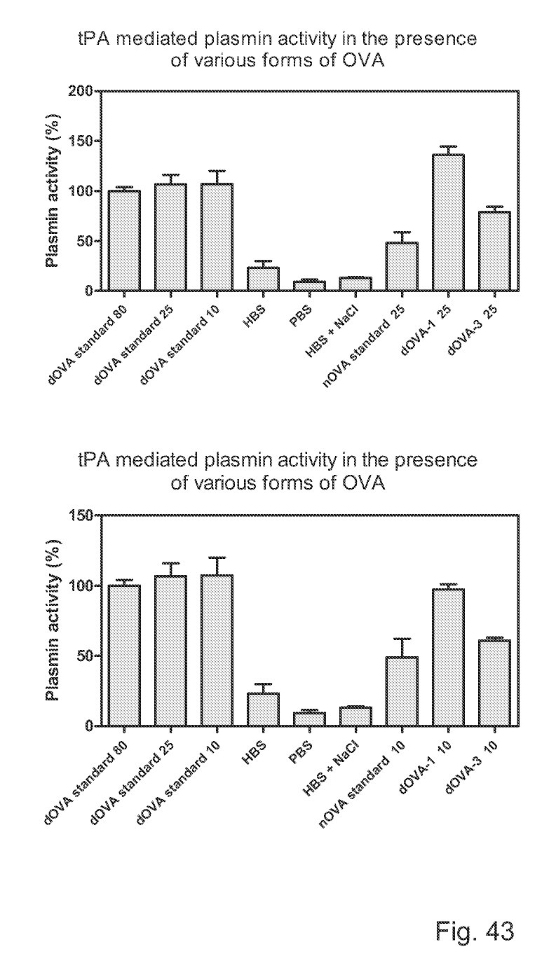

Stimulation of tPA-mediated plasminogen activation by OVA samples. The OVA samples were tested for their tPA mediated plasminogen activation potency at a concentration of 25 and 10 μg/ml. The results are shown in FIG. 43 and Table 16. The activation potency expressed as conversion of plasmin chromogenic substrate is higher for all dOVA forms compared to nOVA upon applying cross-beta inducing methods, and is highest for dOVA-1 and dOVA-2 (identical to dOVA standard used as reference in these and other studies).

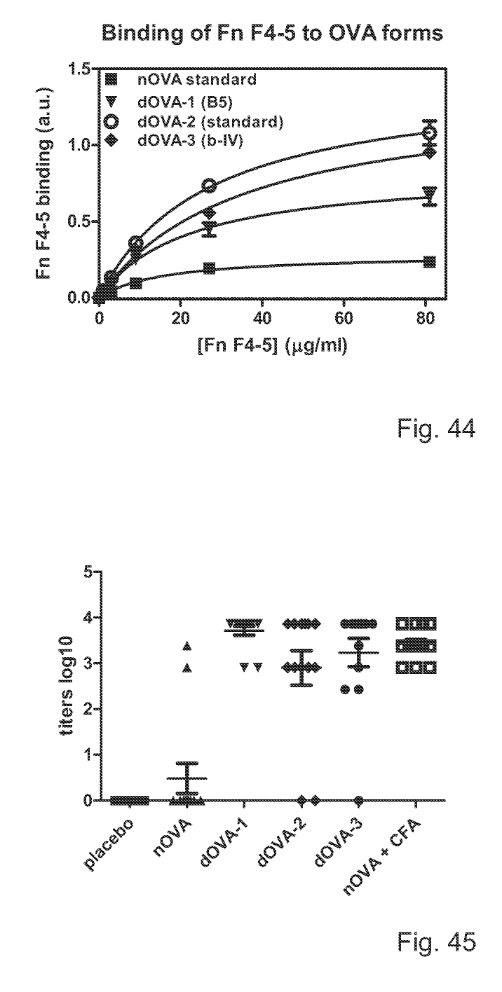

Binding of Fn F4-5 to various forms of OVA, as determined in an ELISA with immobilized forms of OVA. FIG. 44 shows the results of an ELISA to determine the binding of FN4-5 to OVA samples. Table 17 shows the Bmax and kD. Upon misfolding for all samples Bmax is increased up to 5 times (for dOVA-2 and dOVA-3). For dOVA-1 Bmax is increased by a factor of 2. kD does not change much upon misfolding, for samples dOVA-2 and dOVA-3 the kD is increased by a factor of 2. For dOVA-1 the kD value stays the same or is increased by a factor of 1.4. In general one can state that upon increased formation of cross-beta structure in OVA more binding sites for FnF4-5 are created, but the affinity is not changed.

Binding of monoclonal antibodies to various forms of OVA, as determined in an ELISA with immobilized forms of OVA. Tables 18 and 19 show the results (Bmax and kD) of binding analysis by ELISA of several antibodies to nOVA and the dOVA samples.

Immune activating potential of various forms of cross-beta OVA in vivo. The immune-activating potential of cross-beta structural variants of OVA were determined in vivo. Therefore, groups of 13 mice were immunized subcutaneously 4 times with 5 μg OVA/100 μl at weekly intervals. Four days after the last immunization anti-OVA antibody titers were determined. The secondary antibody used binds to both IgG and IgM. Table 20 shows the OVA samples that were used to immunize each different group. Group 1 did not receive an OVA sample, but only buffer (placebo group).

Humoral response. Total anti-OVA IgG and IgM titers present in the serum on day 25 was highest in the groups immunized with dOVA forms and comparable to the levels observed after immunization in the presence of complete Freund's adjuvant (CFA, FIG. 45). The highest titers were observed in mice immunized with dOVA-1, even titer higher than 7290 (see Table 21; titers in 13/13 mice). The Cross-beta nOVA form induces titers in 2/13 mice. Taken together, dOVAs with varying cross-beta structures and varying amounts of cross-beta structures, induce IgG/IgM response comparable to those induced by OVA+CFA, and are much more efficient in inducing an IgG/IgM response in vivo compared to nOVA which comprises a relatively low cross-beta structure content.

IgG/IgM ELISA. Antibody titers were determined for each individual serum against OVA using enzyme-linked immunosorbent assay (ELISA). Briefly, OVA was coated on 96-well plates (655092, Greiner Microlon) at a concentration of 1 μg/ml in 0.1 M Sodium Carbonate, pH 9.5. All incubations were performed for one hour at room temperature (RT) intermitted with five repeated washes with PBS/0.1% Tween20. The wells were blocked with 200 μl of blocking buffer (Roche Block) washed and subsequently incubated with dilutions of the sera. As positive controls, monoclonal anti-OVA IgG (A6075, Sigma) was included in each plate. Total IgG was determined using rabbit-anti-mouse peroxidase labeled-conjugate (PO₂₆₀, DakoCytomation) followed by incubation with TMB substrate (tebu Bio laboratories). Reaction was stopped using 2 M H₂SO₄. Final titers were determined after subtraction of the no-coat controls. The titer was determined as the reciprocal of the dilution factor that resulted in a signal above the mean signal plus 2 times the standard deviation of the placebo group.

### Concluding Remarks

Based on the varying cross-beta structural data, the relative exposure of epitopes for anti-OVA antibodies and the measured anti-OVA antibody titers in mouse sera upon immunization with cross-beta nOVA, cross-beta dOVA-1, cross-beta dOVA-2 and cross-beta dOVA-3, the following conclusions are drawn. Cross-beta nOVA comprises relatively less cross-beta structures which appear as invisible OVA molecular assemblies, compared to the three other cross-beta OVA variants. There is no data showing that nOVA comprises multimers, except for dimers seen on SDS-PA gel. The other three cross-beta dOVA variants 1-3 comprise various amounts of cross-beta structure and comprise multimers as seen on SDS-PA gel and TEM images. All four cross-beta forms of OVA comprise exposed epitopes for a series of anti-OVA antibodies. Upon immunization of mice, the three dOVA forms 1-3 are far more potent in inducing an humoral response than the cross-beta form nOVA, which comprises a relatively low content of cross-beta structure, which appears in relatively low molecular weight OVA assemblies.

TABLE 1
Visual inspection by eye and under a microscope, of various H5 forms

| | | Appearance of H5 solution under a |
|---|---|---|
| crossbeta H5 | Visual appearance | direct light microscope |
| sample | of H5 solution | (supernatant after centrifugation) |
| dH5-0 | Clear | Many bubble/crystal-like |
| | | appearances; colorless |
| cdH5-0 | Clear | relatively small aggregates |
| dH5-I | Turbid | Uniformly distributed amorphous |
| | | shaped aggregates, relatively large |
| dH5-II | Slightly turbid | Uniformly distributed amorphous |
| | | shaped aggregates, smaller than for |
| | | dH5-I |
| dH5-III | Clear | Uniformly distributed amorphous |
| | | shaped aggregates, relatively small |
| ucdH5-0 | Clear, no pellet | Uniformly distributed amorphous |
| | observed | aggregates, relatively small |
| ucdH5-I | Supernatant is | amorphous aggregates |
| | clear, big pellet | |
| ucdH5-II | Supernatant is | Small (tiny) aggregates |
| | clear, small pellet | |
| ucdH5-III | Supernatant is | Clear |
| | clear, small pellet | |

TABLE 2
Binding of Fn F4-5 to various forms of H5: binding sites and
affinities

| | Normalized number of | Normalized affinity, |
|---|---|---|
| H5 form | binding sites, Bmax (%) | kD (%) |
| dH5-0^{†} | 114 | 103 |
| cdH5-0 | 100 | 100 |
| fdH5-0 | 146 | 69 |
| dH5-I | 1 | 0 |
| dH5-II | 9 | 88 |
| dH5-III | 13 | 6 |

†The values for dH5-0 are average values of two measurements
Remark:
a Bmax > 100% indicates that the H5 form exposes more binding sites for Fn F4-5 than cdH5-0. A kD < 100% indicates that the H5 form exposes binding sites for Fn F4-5 for which Fn F4-5 has lower affinity.

TABLE 3
Binding of functional monoclonals to H5 structural variants
Scaled antibody binding (relative number of binding sites Bmax, a.u.)

| cross-beta | Rockland | Rockland | Rockland | Rockland | Rockland | Rockland | HyTest | HyTest | HyTEst |
|---|---|---|---|---|---|---|---|---|---|
| H5 variant | 975 | 976 | 977 | 978 | 979 | 980 | 8D2 | 17C6 | 15A6 |
| dH5-0 | 98 | 106 | 103 | 106 | 100 | 98 | 100 | 94 | 102 |
| cdH5-0 | 100 | 100 | 100 | 100 | 100 | 100 | 100 | 100 | 100 |
| fdH5-0 | 102 | 105 | 103 | 108 | 104 | 103 | 99 | 119 | 116 |
| dH5-I | 28 | 21 | 0 | 3 | 3 | 29 | 7 | 5 | 7 |
| dH5-II | 98 | 31 | 0 | 64 | 26 | 95 | 85 | 40 | 71 |
| dH5-III | 104 | 70 | 18 | 77 | 64 | 77 | 99 | 79 | 88 |

TABLE 4
Binding of functional monoclonals to H5 structural variants
Scaled antibody binding (relative affinity kD, a.u.)

| crossbeta H5 | Rockland | Rockland | Rockland | Rockland | Rockland | Rockland | HyTest | HyTest | HyTEst |
|---|---|---|---|---|---|---|---|---|---|
| variant | 975 | 976 | 977 | 978 | 979 | 980 | 8D2 | 17C6 | 15A6 |
| dH5-0 | 102 | 88 | 98 | 85 | 103 | 117 | 84 | 148 | 97 |
| cdH5-0 | 100 | 100 | 100 | 100 | 100 | 100 | 100 | 100 | 100 |
| fdH5-0 | 103 | 105 | 93 | 107 | 97 | 114 | 125 | 128 | 204 |
| dH5-I | 47 | 0 | 0 | 115 | 125 | 4 | 34 | 51 | 105 |
| dH5-II | 79 | 132 | 0 | 31 | 83 | 19 | 36 | 34 | 31 |
| dH5-III | 78 | 127 | 35 | 112 | 126 | 90 | 72 | 68 | 76 |

TABLE 5
Summary of structural data and of the presence and nature of
binding sites for functional antibodies, and summary of the
anti-H5 titer data and survival data upon H5N1 challenge,
for the six H5 structural variants

| | H5 forms group I | H5 forms group II |
|---|---|---|
| | (dH5-0, cdH5-0, | (dH5-I, dH5-II, |
| | fdH5-0) | dH5-III) |
| Visual inspection/TEM | Relatively less and | More and larger |
| imaging/SDS-PAGE/solubility | smaller aggregates, | aggregates, |
| of multimers | >50% soluble | <50% soluble |
| ThT fluorescence | +/− | Increased |
| Sypro orange fluorescence | +/− | increased |
| tPA and Fn F4-5 binding, | Relatively high | decreased |
| tPA/PIg activation | | |
| Functional antibody binding | Relatively high | Relatively low |
| (number of binding sites and | | |
| affinity) | | |
| Anti-H5 antibody titers | 23/24 mice | 5/24 mice |
| Survival upon H5N1 challenge | 16/24 mice | 1/24 mice |
| No weight loss upon challenge | 5/24 mice | None/24 mice |
| Day of dying upon challenge (# | Day 10 (4), | Day 9 (11), |
| of mice) | day 11 (4) | day 10 (10), |
| | | day 11 (2) |

TABLE 6
Total anti-H5 IgG/IgM titer and survival data of mice at the final day
of the H5N1 challenge

| placebo | | | dH5-0 | | | cdH5-0 | | |
|---|---|---|---|---|---|---|---|---|
| Group- | | | Group- | | | Group- | | |
| mouse # | Liter | survival | mouse # | Titer | Survival | mouse # | Titer | Survival |
| 1-1 | 0 | | 2-1 | 8100 | + | 3-1 | 8100 | + |
| 1-2 | 0 | | 2-2 | 8100 | + | 3-2 | 8100 | + |
| 1-3 | 0 | | 2-3 | 900 | + | 3-3 | 72900 | + |
| 1-4 | 0 | | 2-4 | 8100 | + | 3-4 | 300 | + |
| 1-5 | 0 | | 2-5 | 8100 | + | 3-5 | 300 | |
| 1-6 | 0 | | 2-6 | 100 | | 3-6 | 900 | + |
| 1-7 | 0 | | 2-7 | 8100 | + | 3-7 | 8100 | + |
| 1-8 | 0 | | 2-8 | 900 | | 3-8 | 2700 | |
| fdH5-0 | | | dH5-I | | | dH5-II | | |
| Group- | | | Group- | | | Group- | | |
| mouse # | Titer | survival | mouse # | Titer | Survival | mouse # | Titer | Survival |
| 4-1 | 300 | + | 5-1 | 0 | | 6-1 | 0 | |
| 4-2 | 8100 | + | 5-2 | 0 | | 6-2 | 0 | |
| 4-3 | 8100 | + | 5-3 | 0 | | 6-3 | 0 | |
| 4-4 | 300 | | 5-4 | 900 | | 6-4 | 0 | |
| 4-5 | 300 | | 5-5 | 0 | | 6-5 | 0 | |
| 4-6 | 300 | + | 5-6 | 0 | | 6-6 | 0 | |
| 4-7 | 0 | | 5-7 | 0 | | 6-7 | 0 | |
| 4-8 | 900 | | 5-8 | 0 | | 6-8 | 0 | |
| | | | cdH5-0 + | | | Nobilis | | |
| dH5-III | | | alum | | | flu H5N2 | | |
| Group- | | | Group- | | | Group- | | |
| mouse # | Titer | survival | mouse # | Titer | Survival | mouse # | Titer | Survival |
| 7-1 | 300 | | 8-1 | 8100 | + | 9-1 | 0 | |
| 7-2 | 8100 | | 8-2 | 72900 | + | 9-2 | 0 | + |
| 7-3 | 0 | | 8-3 | ≧218700 | + | 9-3 | 0 | |
| 7-4 | 8100 | + | 8-4 | 72900 | + | 9-4 | 0 | |
| 7-5 | 0 | | 8-5 | 8100 | + | 9-5 | 0 | |
| 7-6 | 0 | | 8-6 | ≧218700 | + | 9-6 | 0 | |
| 7-7 | 0 | | 8-7 | ≧218700 | + | 9-7 | 900 | + |
| 7-8 | 8100 | | 8-8 | 8100 | + | 9-8 | 0 | |

Antigens:
1. Placebo;
2. non-treated H5 (dH5-0);
3. centrifuged H5 (cdH5-0);
4. ultrafiltrated dH5-0 (fdH5-0);
5. dH5-I;
6. dH5-II;
7. dH5-III;
8. cdH5-0 + alum;
9. H5N2 Nobilis flu
A ‘+’ sign indicates that the mouse survived the challenge with H5N1 virus; no sign indicates that the mouse did not survive the challenge. In FIG. 27 it is shown at which stage of the challenge period the mice died.
A total anti-H5 antibody titer of antibodies of the IgG and IgM type is given as the highest serum dilution that still gave an optical density signal higher than the averaged background signal + 2x the standard deviation of the eight sera of the placebo group 1, at that same dilution.

TABLE 7
Endotoxin levels of affinity purified crossbeta E2 (cE2), SEC-E2 and
crossbeta comprising E2 samples (cE2-A and cE2-B)

| | | Endotoxin level |
|---|---|---|
| | Endotoxin level | calculated |
| Sample | Measured (EU/ml) | for 16 μg/ml (EU/ml) |
| cE2 | 2.79 (at 16 μg/ml) | 2.79 |
| SEC-E2 | 3.78 (at 8 μg/ml) | 7.56 |
| cE2-A | 3.95 (at 4 μg/ml) | 15.8 |
| cE2-B | 2.27 (at 4 μg/ml) | 9.08 |

TABLE 8
In vivo medical examination data of pigs challenged with CSFV after
two immunizations

| Treatment | | Test article | Status pigs day 6 | Status pigs day 9 | Status pigs day 14 |
|---|---|---|---|---|---|
| (Test Article) | | information | post challenge | post challenge | post challenge |
| TO1 | Sham (PBS) | buffer | 2†, leukopenia | 3†, ill, walking, 40.0, | 4† at day 11, 6† at |
| | | | | 40.5, 41.0° C. | day 12 |
| TO2^{†} | cE2 | V3 | 5x very ill, | All 5 recovering, | 4 almost completely |
| | | monoclonal | leukopenia | eating, increased | healthy, the 5^{th} pig |
| | | anti-E2 | | mobility | with a paralyzed hind |
| | | purified | | | foot |
| TO3 | cE2 in DOE | V3 | 6x healthy, no | 6x healthy, no | 6x healthy, no |
| | | monoclonal | leukopenia | remarks | remarks |
| | | anti-E2 | | | |
| | | purified, | | | |
| | | adjuvated | | | |
| | | with DOE | | | |
| TO4 | cE2-A | Epitopes | 6x very ill, | 1†, fairly ill, diarrhea, | 2† at day 13, |
| | | present, | leukopenia | high T, low food | remaining 4 pigs |
| | | prepared | | intake, paralyzed | recovering with still |
| | | from SEC- | | hind legs | some remaining |
| | | E2, 3x heat- | | | health problems |
| | | cycled 25-85° C. | | | |
| TO5 | cE2-B | Few | 2† 4x very ill, | 3†, fairly ill, 2 pigs | 5† at day 12, |
| | | epitopes | leukopenia | can hardly stand | remaining pig is very |
| | | present, | | upright, no food | ill |
| | | prepared | | intake, no manure | |
| | | from SEC- | | visible | |
| | | E2, heated | | | |
| | | for 1 h at | | | |
| | | 95° C. | | | |
| TO6 | SEC-E2 | Epitopes | 1†, relatively less | 1†, 1 pig critically ill | 3†, 3 recovered, |
| | | present, E2 | leucopenia, pigs | (neurological | healthy pigs |
| | | monomer/ | less ill than those | problems, thin, | |
| | | dimer peak | in 2, 4, 5 | coloring, paralyzing), | |
| | | after affinity | | pig 2912 = | |
| | | purification | | recovered, other 3 | |
| | | and SEC | | pigs: renewed | |
| | | (retained | | interest in food, are | |
| | | fraction), 10 min. | | moving again | |
| | | 16,000* g | | | |
| | | before use | | | |

†group TO2, or group 2 started with 5 pigs
Remark:
the challenge phase was terminated at day 14, 06Oct08, by euthanizing all animals that survived the CSFV challenge.

TABLE 9
Total anti-E2 IgG titers & survival at day 14 post challenge

| | | | Total anti-E2 IgG | |
|---|---|---|---|---|
| | | | titer (duplicate | Survival at |
| # in titer | | Group at | determination | day 14 |
| ELISA | Pig number | CVI | when indicated) | post challenge |
| 1-1 | 2877 | 1 | —/— | − |
| 1-2 | 2878 | 1 | —/— | − |
| 1-3 | 2879 | 1 | —/— | − |
| 1-4 | 2880 | 1 | —/— | − |
| 1-5 | 2881 | 1 | — | − |
| 1-6 | 2882 | 1 | — | − |
| 2-1 | 2883 | 2 | 512/512 | + |
| 2-2 | 2884 | 2 | 1024/512 | + |
| 2-3 | 2885 | 2 | 2048/1024 | + |
| 2-4 | 2886 | 2 | 1024/512 | + |
| [symbol] | Died before | 2 | Not applicable | Not applicable |
| | exp. | | | |
| 2-6 | 2888 | 2 | 2048/1024 | + |
| 3-1 | 2889 | 3 | 262144 | + |
| 3-2 | 2890 | 3 | 1048576 | + |
| 3-3 | 2891 | 3 | 262144 | + |
| 3-4 | 2892 | 3 | 1048576 | + |
| 3-5 | 2893 | 3 | 262144 | + |
| 3-6 | 2894 | 3 | 262144 | + |
| 4-1 | 2895 | 4 | 1024/512 | + |
| 4-2 | 2896 | 4 | 1024/512 | + |
| 4-3 | 2897 | 4 | 2048/512 | − |
| 4-4 | 2898 | 4 | 512/64 | − |
| 4-5 | 2899 | 4 | 1024/>2048 | + |
| 4-6 | 2900 | 4 | 1024/512 | + |
| 5-1 | 2901 | 5 | — | − |
| 5-2 | 2902 | 5 | — | − |
| 5-3 | 2903 | 5 | — | − |
| 5-4 | 2904 | 5 | — | + |
| 5-5 | 2905 | 5 | — | − |
| 5-6 | 2906 | 5 | — | − |
| 6-1 | 2907 | 6 | — | − |
| 6-2 | 2908 | 6 | 512 | + |
| 6-3 | 2909 | 6 | 512 | + |
| 6-4 | 2910 | 6 | — | − |
| 6-5 | 2911 | 6 | — | − |
| 6-6 | 2912 | 6 | 1024 | + |

^{†}group 2 started with 5 pigs at day 0 (one pig died at day −1)
Remark:
the challenge phase was terminated at day 14, 06Oct08, by euthanizing all animals that survived the CSFV challenge.

TABLE 10
Factor VIII inhibition by antibodies and total anti-factor VIII
antibody titers induced by crossbeta factor VIII forms

| Mouse | Anti-fVIII titer | fVIII inhibition |
|---|---|---|
| (Group, #) | (IgG and IgM) | (Bethesda units/ml) |
| 1-1 | 4096 | 1.9 |
| 1-2 | 64 | 1.1 |
| 1-3 | 16384 | 5.6 |
| 1-4 | 16384 | >10 |
| 1-5 | 4096 | 1.3 |
| 12-1 | 0 | <0.1 |
| 12-2 | 4096 | 4.3 |
| 12-3 | 16384 | 2.7 |
| 12-4 | 0 | 0.3 |
| 12-5 | 16 | 0.3 |
| 5-1 | 1024 | <0.1 |
| 5-2 | 4096 | 0.3 |
| 5-3 | 16 | <0.1 |
| 5-4 | 1024 | <0.1 |
| 5-5 | 256 | <0.1 |
| buffer-1 | 0 | 0.3 |
| buffer-2 | 16 | 0.3 |
| buffer-3 | 0 | 0.2 |
| buffer-4 | 0 | 0.2 |
| buffer-5 | 0 | <0.1 |

Legend to the samples:
1. Crossbeta Factor VIII kept at 4° C. for 20 hours, after dissolving; stored at −80° C.
12. Crossbeta Factor VIII kept at 37° C. for 1 week, after dissolving; stored at 4° C.
5. Crossbeta factor VIII heated at 95° C. for 5 minutes, after dissolving; stored at −80° C.
Buffer is PBS, and used as placebo antigen. [fVIII] = 40 μg/ml or 200 ie/ml
REMARK
Generally, a value for factor VIII inhibition of <0.5 Bethesda units/ml, or BU/ml, is assigned as ‘negative' → no factor VIII inhibiting antibodies detected. Scale: 0 BU refers to 0% factor VIII inhibition; 1 BU = 50% inhibition; 2 BU = 75% inhibition; 3 BU = 87.5% inhibition; and so on.
REMARK
An anti-factor VIII antibody titer is given as the highest plasma dilution that gave an optical density signal higher than the background signal + 2x the standard deviation of the ELISA plate blank. Dilution series started with 1:16 plasma dilution, and a titer of >16 is considered positive.

TABLE 11
Endotoxin level of various OVA forms

| | Endotoxin Level | Endotoxin level of 5 μg |
|---|---|---|
| Sample | (EU/ml) | OVA |
| nOVA | 2.19 | 0.55 |
| dOVA-1 | 5.08 | 1.27 |
| dOVA-2 | 3.03 | 0.758 |
| dOVA-3 | 1.26 | 0.315 |

TABLE 12
Visual inspection of various OVA forms

| | Appearance of OVA | Appearance of OVA solution |
|---|---|---|
| Sample | solution | after one freeze/thaw cycle |
| nOVA | Clear | Clear |
| dOVA-1 | Turbid, and big pellet after | A bit turbid, |
| | 16.000 g | big flakes visible |
| dOVA-2 | Clear | Clear |
| dOVA-3 | Clear | A bit turbid |

TABLE 13
Analysis of OVA multimerization by Transmission Electron
Micrsocopy

| Sample | Appearance of OVA solution |
|---|---|
| Buffer | Empty view |
| nOVA | Empty view |
| dOVA-1 | heterogenous picture, size variation: from small to medium |
| | size aggregates, cloudy appearance, also elongated structures |
| | (fibre like) spotted but not in every |
| dOVA-2 | heterogenous picture, size variation: from small to medium |
| | size aggregates, cloudy appearance |
| dOVA-3 | reasonable uniform picture, size variation: from small to |
| | medium size aggregates, cloudy appearance, very open |
| | structure |

TABLE 14
Enhancement of Thioflavin T fluorescence under influence of
various crossbeta OVA forms

| Sample | ThT fluorescence (U/mL) |
|---|---|
| dOVA st-100 | 100.00 |
| PBS | 0.00 |
| HBS-NaCl | −3.47 |
| nOVA | 3.31 |
| dOVA-1 | 49.39 |
| dOVA-2 | 62.47 |
| dOVA-3 | 77.74 |

TABLE 15
Enhancement of Sypro Orange fluorescence under influence of
various OVA forms

| Sample | SO fluorescence (U/mL) |
|---|---|
| dOVA reference | 100.00 |
| standard | |
| PBS | 0.00 |
| HBS-NaCl | 0.04 |
| nOVA | 0.90 |
| dOVA-1 | 56.06 |
| dOVA-2 | 48.58 |
| dOVA-3 | 41.44 |

TABLE 16
tPA activation potency of crossbeta OVA samples

| | Activation at | |
|---|---|---|
| OVA form | 25 μg/mL | Activation at 10 μg/mL |
| dOVA-2 80* | 100.00 | 100.00 |
| HBS | 23.35 | |
| PBS | 9.25 | |
| HBS + NaCl | 13.21 | |
| nOVA | 48.14 | 48.78 |
| dOVA-1 | 136.13 | 97.40 |
| dOVA-2 | 106.69 | 107.22 |
| dOVA-3 | 79.04 | 60.91 |

*Reference: Fluorescent signal set at 100%. Other samples are compared with this reference sample.

TABLE 17
Binding of Fn F4-5 to various crossbeta forms of OVA: binding sites
and affinities

| | Normalized number | |
|---|---|---|
| | of binding sites, | Normalized |
| H5 form | Bmax (%) | affinity, kD (%) |
| nOVA | 100.00 | 100.00 |
| dOVA-1 | 291.23 | 136.70 |
| dOVA-2 | 471.10 | 217.06 |

Remark:
a Bmax > 100% indicates that the OVA form exposes more binding sites for Fn F4-5 than nOVA. A kD > 100% indicates that the OVA form exposes binding sites for Fn F4-5 for which Fn F4-5 has lower affinity.

TABLE 18
Binding of functional monoclonals to OVA structural variants

| | Scaled antibody binding (relative number of binding sites Bmax, a.u.) | | | | | | |
|---|---|---|---|---|---|---|---|
| | Antibody | | | | | | |
| | HYB | HYB | HYB | Sigma | MP | MP | |
| OVA variant | 099-01 | 099-02 | 099-09 | A6075 | 55303 | 55304 | Sigma C6534 |
| nOVA | 1.461 | 2.072 | 2.024 | | 0.9760 | 1.494 | 0.9423 |
| dOVA-1 | 1.5 | 2.629 | 1.937 | 1.637 | 1.600 | 0.9278 | 0.9367 |
| dOVA-2 | 0.6330 | 1.844 | 1.780 | | 1.075 | 1.689 | 0.9891 |
| dOVA-3 | 0.3565 | 0.1731 | 0.05829 | | 1.025 | 1.753 | 0.9779 |

TABLE 19
Binding of functional monoclonals to OVA structural variants

| | Scaled antibody binding (relative affinity kD, a.u.) | | | | | | |
|---|---|---|---|---|---|---|---|
| | Antibody | | | | | | |
| | HYB | HYB | | | | | |
| OVA | 099- | 099- | HYB | Sigma | MP | MP | Sigma |
| variant | 01 | 02 | 099-09 | A6075 | 55303 | 55304 | C6534 |
| nOVA | 98.49 | 103.0 | 71.15 | | 184.1 | 107.1 | 84.94 |
| dOVA- | 115.3 | 153.9 | 127.4 | 20.83 | 90.18 | 103 | 44.05 |
| 1 | | | | | | | |
| dOVA- | 129.4 | 117.9 | 85.12 | | 364.1 | 481.6 | 221.1 |
| 2 | | | | | | | |
| dOVA- | 80.87 | 154.4 | 164.4 | | 332.4 | 524.0 | 286.7 |
| 3 | | | | | | | |

TABLE 20
Antigen and immunization scheme

| Group | ovalbumin - | |
|---|---|---|
| (n = 10 + 3 | 4 weekly doses | |
| mice) | 5 μg | Description |
| 1 | Placebo | PBS |
| 2 | nOVA | OVA standard 1 mg/ml in PBS |
| 3 | dOVA-1 | High pH, 37° C., 40 min (dOVA-B5) |
| 4 | dOVA-2 | dOVA standard 1 mg/ml |
| 5 | dOVA-3 | 75° C., o/n (dOVA-b-IV) |
| 6 | nOVA + | OVA standard 1 mg/ml in PBS |
| | Freund's Adjuvant | |

TABLE 21
Antibody titers of individual mice

| antigen | mouse # | titer | antigen | Mouse # | titer | antigen | Mouse # | titer |
|---|---|---|---|---|---|---|---|---|
| Placebo | 386131 | <30 | nOVA | 386128 | <30 | dOVA-1 | 386117 | >7290 |
| | 386132 | <30 | | 386129 | 810 | | 386118 | >7290 |
| | 386133 | <30 | | 386130 | <30 | | 386119 | >7290 |
| | 386144 | <30 | | 386154 | <30 | | 386141 | 7290 |
| | 386145 | <30 | | 386155 | <30 | | 386142 | 810 |
| | 386146 | <30 | | 386156 | <30 | | 386143 | 810 |
| | 386147 | <30 | | 386160 | <30 | | 386157 | >7290 |
| | 386148 | <30 | | 386161 | <30 | | 386158 | 7290 |
| | 386149 | <30 | | 386162 | <30 | | 386159 | >7290 |
| | 386150 | <30 | | 386163 | 2430 | | 386173 | >7290 |
| | 386151 | <30 | | 386164 | <30 | | 386174 | >7290 |
| | 386152 | <30 | | 386165 | <30 | | 386175 | 7290 |
| | 386153 | <30 | | 386166 | <30 | | 386189 | 7290 |
| dOVA-2 | 386124 | 810 | dOVA-3 | 386115 | >7290 | nOVA + Freunds | 386116 | 2430 |
| | 386125 | 810 | | 386121 | >7290 | | 386120 | 2430 |
| | 386126 | <30 | | 386123 | >7290 | | 386122 | 7290 |
| | 386180 | >7290 | | 386193 | >7290 | | 386136 | 2430 |
| | 386181 | <30 | | 386194 | >7290 | | 386137 | >7290 |
| | 386182 | >7290 | | 386195 | 2430 | | 386138 | 2430 |
| | 386183 | 810 | | 386196 | 270 | | 386139 | 810 |
| | 386184 | 810 | | 386197 | >7290 | | 386140 | 810 |
| | 386187 | >7290 | | 386198 | >7290 | | 386185 | 2430 |
| | 386188 | 810 | | 386199 | <30 | | 386186 | 2430 |
| | 386190 | >7290 | | 386203 | 810 | | 386200 | >7290 |
| | 386191 | >7290 | | 386204 | 270 | | 386201 | >7290 |
| | 386192 | >7290 | | 386205 | >7290 | | 386202 | 810 |

1. A method for producing an immunogenic composition comprising at least one peptide, polypeptide, protein, glycoprotein, protein-DNA complex, protein-membrane complex and/or lipoprotein, the method comprising:
providing a composition with at least one crossbeta structure and determining:
whether a binding compound capable of specifically binding an epitope of the peptide, polypeptide, protein, glycoprotein, protein-DNA complex, protein-membrane complex and/or lipoprotein is capable of specifically binding the immunogenic composition;
whether the degree of multimerization of the peptide, polypeptide, protein, glycoprotein, protein-DNA complex, protein-membrane complex and/or lipoprotein in the composition allows recognition of an epitope of the peptide, polypeptide, protein, glycoprotein, protein-DNA complex, protein-membrane complex and/or lipoprotein by an animal's immune system;
whether between 4-75% of the peptide, polypeptide, protein, glycoprotein, protein-DNA complex, protein-membrane complex and/or lipoprotein content of the composition is in a conformation comprising crossbeta structures; and/or
whether the at least one crossbeta structure comprises a property allowing recognition of an epitope of the peptide, polypeptide, protein, glycoprotein, protein-DNA complex, protein-membrane complex and/or lipoprotein by an animal's immune system.

2. The method according to claim 1, comprising determining whether a binding molecule comprising an antibody or antibody fragment, capable of specifically binding an epitope of the peptide, polypeptide, protein, glycoprotein, protein-DNA complex, protein-membrane complex and/or lipoprotein, is capable of specifically binding the immunogenic composition or a component of the immunogenic composition.

3. The method according to claim 1, comprising determining whether the immunogenic composition and/or crossbeta structure is capable of specifically binding a crossbeta structure binding compound, tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor, RAGE, CD36, CD40, LOX-1, TLR2, TLR4, a crossbeta-specific antibody, a crossbeta-specific IgG, a crossbeta-specific IgM, IgIV, an enriched fraction of IgIV capable of specifically binding a crossbeta structure, Low density lipoprotein Related Protein (LRP), LRP Cluster II, LRP Cluster IV, Scavenger Receptor B-I (SR BI), SR A, chrysamine G, a chaperone, a heat shock protein, HSP70, HSP60, HSP90, gp95, calreticulin, a chaperonin, a chaperokine and/or a stress protein.

4. The method according to claim 1, further comprising selecting an immunogenic composition capable of specifically binding a binding molecule comprising an antibody or antibody fragment, which binding molecule is capable of specifically binding an epitope of the peptide, polypeptide, protein, glycoprotein, protein-DNA complex, protein-membrane complex, and/or lipoprotein.

5. The method according to claim 1, further comprising selecting an immunogenic composition wherein the degree of multimerization of the peptide, polypeptide, protein, glycoprotein, protein-DNA complex, protein-membrane complex and/or lipoprotein in the composition allows recognition of an epitope of the peptide, polypeptide, protein, glycoprotein, protein-DNA complex, protein-membrane complex and/or lipoprotein by an animal's immune system.

6. The method according to claim 1, further comprising:
selecting an immunogenic composition wherein between 4 and 75% of the peptide, polypeptide, protein, glycoprotein, protein-DNA complex, protein-membrane complex and/or lipoprotein content thereof is in a conformation comprising crossbeta structures.

7. The method according to claim 1, further comprising selecting an immunogenic composition which comprises a crossbeta structure capable of specifically binding a crossbeta structure binding compound, tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor, RAGE, CD36, CD40, LOX-1, TLR2, TLR4, a crossbeta-specific antibody, crossbeta-specific IgG, crossbeta-specific IgM, IgIV, an enriched fraction of IgIV capable of specifically binding a crossbeta structure, Low density lipoprotein Related Protein (LRP), LRP Cluster II, LRP Cluster IV, Scavenger Receptor B-I (SR BI), SR A, chrysamine G, a chaperone, a heat shock protein, HSP70, HSP60, HSP90, gp95, calreticulin, a chaperonin, a chaperokine, and/or a stress protein.

8. An in vitro method for selecting, from a plurality of immunogenic compositions comprising at least one peptide and/or polypeptide and/or protein and/or glycoprotein and/or lipoprotein and/or protein-DNA complex and/or protein-membrane complex with a crossbeta structure, one or more immunogenic compositions having a greater chance of being capable of eliciting a protective prophylactic immune response and/or a therapeutic immune response in vivo, as compared to the other immunogenic compositions of the plurality of immunogenic compositions, the method comprising:
selecting, from the plurality of immunogenic compositions, an immunogenic composition:
capable of specifically binding an antibody or antibody fragment, which is capable of specifically binding an epitope of the peptide, polypeptide, protein, glycoprotein, protein-DNA complex, protein-membrane complex and/or lipoprotein;
wherein the degree of multimerization of the peptide, polypeptide, protein, glycoprotein, protein-DNA complex, protein-membrane complex and/or lipoprotein in the composition allows recognition of an epitope of the peptide, polypeptide, protein, glycoprotein, protein-DNA complex, protein-membrane complex and/or lipoprotein by an animal's immune system;
wherein between 4-75% of the peptide, polypeptide, protein, glycoprotein, protein-DNA complex, protein-membrane complex and/or lipoprotein content of the composition is in a conformation comprising crossbeta structures; and/or
which comprises a crossbeta structure capable of specifically binding a crossbeta structure binding compound, tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor, RAGE, CD36, CD40, LOX-1, TLR2, TLR4, a crossbeta-specific antibody, crossbeta-specific IgG, crossbeta-specific IgM, IgIV, an enriched fraction of IgIV capable of specifically binding a crossbeta structure, Low density lipoprotein Related Protein (LRP), LRP Cluster II, LRP Cluster IV, Scavenger Receptor B-I (SR BI), SR A, chrysamine G, a chaperone, a heat shock protein, HSP70, HSP60, HSP90, gp95, calreticulin, a chaperonin, a chaperokine and/or a stress protein.

9. The method according to claim 1, further comprising:
selecting an immunogenic composition capable of specifically binding at least two antibodies or antibody fragments, which themselves are capable of specifically binding at least two different epitopes of the peptide, polypeptide, protein, glycoprotein, protein-DNA complex, protein-membrane complex and/or lipoprotein.

10. The method according to claim 1, further comprising:
selecting an immunogenic composition capable of specifically binding at least one antibody or antibody fragment, which is capable of providing a protective prophylactic and/or a therapeutic immune response in a subject in vivo.

11. The method according to claim 1, wherein the crossbeta structure is induced in at least part of the peptide, polypeptide, protein, glycoprotein, protein-DNA complex, protein-membrane complex, and/or lipoprotein.

12. The method according to claim 1, wherein the at least one peptide, polypeptide, protein, glycoprotein, protein-DNA complex, protein-membrane complex and/or lipoprotein is subjected to a crossbeta inducing procedure, a change of pH, salt concentration, reducing agent concentration, temperature, buffer, and/or chaotropic agent concentration.

13. The method according to claim 1, wherein the at least one peptide, polypeptide, protein, glycoprotein, protein-DNA complex, protein-membrane complex and/or lipoprotein is coupled to a crossbeta comprising compound.

14. The method according to claim 1, wherein the epitope of the peptide, polypeptide, protein, glycoprotein, protein-DNA complex, protein-membrane complex and/or lipoprotein is surface-exposed when the peptide, polypeptide, protein, glycoprotein, protein-DNA complex, protein-membrane complex and/or lipoprotein is in its native conformation.

15. The method according to claim 4, further comprising:
producing a vaccine comprising the selected immunogenic composition.

16. (canceled)

17. An immunogenic composition selected and/or produced with the method according to claim 1.

18. (canceled)

19. A vaccine for the prophylaxis and/or treatment of a disorder caused by a pathogen, tumor, cardiovascular disease, atherosclerosis, amyloidosis, autoimmune disease, graft-versus-host rejection and/or transplant rejection, said vaccine comprising:
the immunogenic composition of claim 17.

20. A method for at least in part preventing and/or counteracting a disorder caused by a pathogen, tumor, cardiovascular disease, atherosclerosis, amyloidosis, autoimmune disease, graft-versus-host rejection and/or transplant rejection in a subject, the method comprising:
administering to a subject diagnosed to be in need thereof a therapeutically effective amount of the immunogenic composition of claim 17.

21. The method according claim 20, wherein the subject is a human individual.

22. A method for improving an immunogenic composition, the composition comprising at least one peptide, polypeptide, protein, glycoprotein, protein-DNA complex, protein-membrane complex and/or lipoprotein, the method comprising:
providing the composition with at least one crossbeta structure, and
selecting an immunogenic composition:
capable of specifically binding an antibody or antibody fragment, which antibody or antibody fragment is capable of specifically binding an epitope of the peptide, polypeptide, protein, glycoprotein, protein-DNA complex, protein-membrane complex and/or lipoprotein;
wherein the degree of multimerization of the peptide, polypeptide, protein, glycoprotein, protein-DNA complex, protein-membrane complex and/or lipoprotein in the composition allows recognition of an epitope of the peptide, polypeptide, protein, glycoprotein, protein-DNA complex, protein-membrane complex and/or lipoprotein by an animal's immune system;
wherein between 4-75% of the peptide, polypeptide, protein, glycoprotein, protein-DNA complex, protein-membrane complex and/or lipoprotein content of the composition is in a conformation comprising crossbeta structures; and/or
capable of specifically binding a crossbeta structure binding compound, tPA, BiP, factor XII, fibronectin, hepatocyte growth factor activator, at least one finger domain of tPA, at least one finger domain of factor XII, at least one finger domain of fibronectin, at least one finger domain of hepatocyte growth factor activator, Thioflavin T, Thioflavin S, Congo Red, CD14, a multiligand receptor, RAGE, CD36, CD40, LOX-1, TLR2, TLR4, a crossbeta-specific antibody, crossbeta-specific IgG, crossbeta-specific IgM, IgIV, an enriched fraction of IgIV capable of specifically binding a crossbeta structure, Low density lipoprotein Related Protein (LRP), LRP Cluster II, LRP Cluster IV, Scavenger Receptor B-I (SR BI), SR A, chrysamine G, a chaperone, a heat shock protein, HSP70, HSP60, HSP90, gp95, calreticulin, a chaperonin, a chaperokine, and/or a stress protein.

23. (canceled)

24. A reconvalescent serum and/or antibody capable of at least in part preventing and/or counteracting a pathology and/or a disorder, the reconvalescent serum, and/or antibody obtainable by:
immunizing an animal with the immunogenic composition of claim 17, and, subsequently,
harvesting the reconvalescent serum and/or antibody from the animal.

25. The reconvalescent serum and/or antibody of claim 24 wherein said reconvalescent serum and/or antibody is a vaccine.

26. A method for obtaining a reconvalescent serum and/or an antibody capable of at least in part preventing and/or counteracting a pathology and/or a disorder, the method comprising:
producing an immunogenic composition with the method according to claim 1, using a binding molecule that comprises an antibody or an antibody fragment capable of specifically binding an epitope of interest of the peptide, polypeptide, protein, glycoprotein, protein-DNA complex, protein-membrane complex, and/or lipoprotein present in the immunogenic composition;
immunizing an animal with the immunogenic composition; and
harvesting reconvalescent serum and/or an antibody from the animal.

27. The method according to claim 26, further comprising preparing a composition comprising an antibody or antibody fragment, capable of at least in part preventing and/or counteracting the pathology and/or disorder.

28. The method according to claim 27, wherein the antibody or antibody fragment is coupled to an antigen for immune complex vaccination.

29. A FAPI vaccine with improved capability of at least in part preventing and/or counteracting a pathology and/or a disorder, obtainable by the method according to claim 27.

30. An immune complex vaccine product obtainable by the method according to claim 28.

31. (canceled)

32. (canceled)

33. The immunogenic composition of claim 17, further comprising a suitable carrier.

34. The method according to claim 1, comprising determining whether monomers and/or multimers of the peptide, polypeptide, protein, glycoprotein, protein-DNA complex, protein-membrane complex and/or lipoprotein in the immunogenic composition have dimensions in the range of 0.5 nm to 1000 μm range, 0.5 nm to 100 μm range, 1 nm to 5 μm range, or 3-2000 m range.

35. A process for producing an immunogenic composition comprising a peptide, the process comprising:
providing a composition comprising a peptide with a crossbeta structure; and then determining whether:
a binding compound able to specifically bind an epitope of the peptide is also able to specifically bind the composition;
the peptide's degree of multimerization in the composition allows for recognition of an epitope of the peptide by an animal's immune system;
between 4 and 75% of the peptide content of the composition is in a conformation comprising crossbeta structures; or
the crossbeta structure comprises a property allowing for recognition of an epitope by an animal's immune system,
wherein one or more of such determinations is indicative of the composition being an immunogenic composition.
